(19) United States
(12) Patent Application Publication
(10) Pub. No.: US 2024/0301379 A1
(43) Pub. Date: Sep. 12, 2024

(54) EFFECTOR PROTEINS AND USES THEREOF

(71) Applicant: Mammoth Biosciences, Inc., Brisbane, CA (US)

(72) Inventors: Lucas Benjamin HARRINGTON, San Francisco, CA (US); David PAEZ-ESPINO, Concord, CA (US); Benjamin Julius RAUCH, San Francisco, CA (US); Stepan TYMOSHENKO, Sacramento, CA (US)

(73) Assignee: Mammoth Biosciences, Inc., Brisbane, CA (US)

(21) Appl. No.: 18/676,562

(22) Filed: May 29, 2024

Related U.S. Application Data
(63) Continuation of application No. PCT/US2022/080258, filed on Nov. 21, 2022.
(60) Provisional application No. 63/371,023, filed on Aug. 10, 2022.
(60) Provisional application No. 63/284,339, filed on Nov. 30, 2021.

(51) Int. Cl. C12N 9/22; C12N 15/11; C12N 15/90; C12Q 1/6844

(52) U.S. Cl. CPC C12N 9/22; C12N 15/11; C12N 15/907; C12Q 1/6844; C12N 2310/20

(57) ABSTRACT

Provided herein are compositions, systems, and methods comprising effector proteins and uses thereof. These effector proteins may be characterized as CRISPR-associated (Cas) proteins. Various compositions, systems, and methods of the present disclosure may leverage the activities of these effector proteins for the modification, detection, and engineering of nucleic acids.

### CROSS-REFERENCE TO RELATED APPLICATIONS

This application is a continuation of International Patent Application No. PCT/US2022/080258, filed Nov. 21, 2022, which claims priority to U.S. Provisional Application No. 63/284,339, filed Nov. 30, 2021, and U.S. Provisional Application No. 63/371,023, filed Aug. 10, 2022, each of which are incorporated herein by reference in their entireties.

### DESCRIPTION OF THE TEXT FILE SUBMITTED ELECTRONICALLY

The contents of the electronic sequence listing (MABI_020_02 US_SeqList_ST26.xml; Size: 63,840,257 bytes; and Date of Creation: May 28, 2024) are herein incorporated by reference in its entirety.

### BACKGROUND

Programmable nucleases are proteins that bind and cleave nucleic acids in a sequence-specific manner. A programmable nuclease may bind a target region of a nucleic acid and cleave the nucleic acid within the target region or at a position adjacent to the target region. In some embodiments, a programmable nuclease is activated when it binds a target region of a nucleic acid to cleave regions of the nucleic acid that are near, but not adjacent to the target region. A programmable nuclease, such as a CRISPR-associated (Cas) protein, may be coupled to a guide nucleic acid that imparts activity or sequence selectivity to the programmable nuclease. In general, guide nucleic acids comprise a CRISPR RNA (crRNA) that is at least partially complementary to a target nucleic acid. In some cases, guide nucleic acids comprise a trans-activating crRNA (tracrRNA), at least a portion of which interacts with the programmable nuclease. In some cases, a tracrRNA is provided separately from the crRNA and hybridizes to a portion of the crRNA that does not hybridize to the target nucleic acid. In other cases, the tracrRNA and crRNA are linked as a single guide RNA.

Programmable nucleases may cleave nucleic acids, including single stranded RNA (ssRNA), double stranded DNA (dsDNA), and single-stranded DNA (ssDNA). Programmable nucleases may provide cis cleavage activity, nickase activity, or a combination thereof. Cis cleavage activity is cleavage of a target nucleic acid that is hybridized to a guide nucleic acid, wherein cleavage occurs within or directly adjacent to the region of the target nucleic acid that is hybridized to guideRNA.

Programmable nucleases may be modified to have reduced nuclease or nickase activity relative to its unmodified version, but retain their sequence selectivity. For instance, amino acid residues of the programmable nuclease that impart catalytic activity to the programmable nuclease may be substituted with an alternative amino acid that does not impart catalytic activity to the programmable nuclease. The term, “effector protein,” is used herein and throughout to encompass both programmable nucleases and modified versions thereof that may not necessarily have nuclease activity.

While certain programmable nucleases may be used to edit and detect nucleic acid molecules in a sequence specific manner, challenging biological and sample conditions (e.g., high viscosity, metal chelating) may limit their accuracy and effectiveness. There is thus a need for systems and methods that employ programmable nucleases having specificity and efficiency across a wide range of biological and sample conditions.

### SUMMARY

The present disclosure provides polypeptides, such as effector proteins, compositions, systems, and methods comprising effector proteins and uses thereof. In some instances, compositions, systems, and methods comprise guide nucleic acids or uses thereof. Compositions, systems and methods disclosed herein may leverage nucleic acid modifying activities such as nucleic acid editing (e.g., cis cleavage activity) of these effector proteins for the modification, detection and engineering of target nucleic acids. Editing may comprise: insertion, deletion, substitution, or a combination thereof of one or more nucleotides or amino acids. Modification activities also includes cleavage activity, such as cis cleavage activity, nicking activity, and/or nuclease activity. In some instances, compositions, systems and methods are useful for the editing the sequence of target nucleic acids. In some instances, compositions, systems and methods are useful for the detection of target nucleic acids. In some instances, compositions, systems and methods are useful for the treatment of a disease or disorder. The disease or disorder may be associated with one or more mutations in the target nucleic acid.

### I. Certain Embodiments

In some embodiments, the disclosure provides a composition comprising an effector protein and a guide nucleic acid, wherein the effector protein comprises an amino acid sequence that is at least 75%, at least 80%, at least 85%, at least 90%, at least 95%, at least 98%, at least 99% or 100% identical to any one of SEQ ID NOS: 1-10,484 or 15,022-24,165. In some embodiments, the disclosure provides a composition comprising an effector protein and a guide nucleic acid, wherein the amino acid sequence of the effector protein is at least 75%, at least 80%, at least 85%, at least 90%, at least 95%, at least 98%, at least 99% or 100% identical to any one of SEQ ID NOS: 1-10,484 or 15,022-24,165.

In some embodiments, the disclosure provides a composition comprising an effector protein and a guide nucleic acid, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, about 1400, about 1420, about 1440, about 1460, about 1480, about 1490, about 1500, about 1520, about 1540, about 1560, about 1580, about 1600, about 1620, about 1640, about 1660, about 1680, about 1700, about 1720, about 1740, about 1760, about 1780, about 1800, about 1820, about 1840, about 1860, about 1880, about 1900, or about 1920 contiguous amino acids of a sequence selected from SEQ ID NOS: 1-10,484 or 15,022-24,165.

In some embodiments, the disclosure provides a composition comprising an effector protein and a guide nucleic acid, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, 1250-1350, 1301-1400, 1350-1450, 1401-1500, 1450-1550, 1501-1600, 1550-1650, 1601-1700, 1650-1750, 1701-1800, 1850-1950, 1801-1900, or 1850-1950 of a sequence selected from SEQ ID NOS: 1-10,484 or 15,022-24,165.

In some embodiments, the disclosure provides a composition comprising an effector protein and a guide nucleic acid, wherein the effector protein comprises an amino acid sequence that is at least 90%, at least 95%, or 100% identical to a portion of a sequence selected from SEQ ID NOS: 1-10,484 or 15,022-24,165, and wherein the length of the portion is at least about 100, at least about 200, at least about 300, at least about 400, at least about 500, or at least about 600 linked amino acids in length. In some embodiments, the portion of the sequence is about 30%, about 40%, about 50%, about 60%, about 70%, about 80%, or about 90% of a sequence selected from SEQ ID NOS: 1-10,484 or 15,022-24,165.

In some embodiments, the disclosure provides a composition comprising an effector protein, and a guide nucleic acid, wherein a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1 of TABLE 1; and b) at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B1 of TABLE 1, wherein the sequence from Column A1 and the sequence from Column B1 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B1 of TABLE 1, wherein the sequence from Column A1 and the sequence from Column B1 are in the same row of TABLE 1.

In some embodiments, the disclosure provides a composition comprising an effector protein, and an engineered guide nucleic acid, wherein a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A2 of TABLE 1; and b) at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B2 of TABLE 1, wherein the sequence from Column A2 and the sequence from Column B2 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B2 of TABLE 1, wherein the sequence from Column A2 and the sequence from Column B2 are in the same row of TABLE 1.

In some embodiments, the disclosure provides a composition comprising an effector protein, and an engineered guide nucleic acid, wherein a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A3 of TABLE 1; and b) at least a portion of the engineered guide nucleic acid comprises a nucleobase sequence that is: i) at least 50%, at least 55%, at least 60%, at least 65%, at least 70%, at least 75%, at least 80%, at least 85%, at least 90%, at least 95%, or 100% identical to a sequence selected from Column B3 of TABLE 1, wherein the sequence from Column A3 and the sequence from Column B3 are in the same row of TABLE 1, or ii) at least 50%, at least 55%, at least 60%, at least 65%, at least 70%, at least 75%, at least 80%, at least 85%, at least 90%, at least 95%, or 100% reverse complementary to a sequence selected from Column B3 of TABLE 1, wherein the sequence from Column A3 and the sequence from Column B3 are in the same row of TABLE 1.

In some embodiments, the disclosure provides a composition comprising an effector protein, and an engineered guide nucleic acid, wherein the amino acid sequence of the effector protein is at least 50% identical to a sequence selected from Column A1 of TABLE 1, and wherein at least a portion of the engineered guide nucleic acid comprises a nucleobase sequence that is at least 50% identical or at least 50% reverse complementary to a sequence selected from Column B1 of TABLE 1, wherein the sequence from Column A1 and the sequence from Column B1 are in the same row of TABLE 1.

In some embodiments, the disclosure provides a composition comprising an effector protein, and an engineered guide nucleic acid, wherein the amino acid sequence of the effector protein is at least 60% identical to a sequence selected from Column A1 of TABLE 1, and wherein at least a portion of the engineered guide nucleic acid comprises a nucleobase sequence that is at least 60% identical or at least 60% reverse complementary to a sequence selected from Column B1 of TABLE 1, wherein the sequence from Column A1 and the sequence from Column B1 are in the same row of TABLE 1. In some embodiments, the disclosure provides a composition comprising an effector protein, and an engineered guide nucleic acid, wherein the amino acid sequence of the effector protein is at least 70% identical to a sequence selected from Column A1 of TABLE 1, and wherein at least a portion of the engineered guide nucleic acid comprises a nucleobase sequence that is at least 70% identical or at least 70% reverse complementary to a sequence selected from Column B1 of TABLE 1, wherein the sequence from Column A1 and the sequence from Column B1 are in the same row of TABLE 1. In some embodiments, the disclosure provides a composition comprising an effector protein, and an engineered guide nucleic acid, wherein the amino acid sequence of the effector protein is at least 80% identical to a sequence selected from Column A1 of TABLE 1, and wherein at least a portion of the engineered guide nucleic acid comprises a nucleobase sequence that is at least 80% identical or at least 80% reverse complementary to a sequence selected from Column B1 of TABLE 1, wherein the sequence from Column A1 and the sequence from Column B1 are in the same row of TABLE 1.

In some embodiments, the disclosure provides a composition comprising an effector protein, and an engineered guide nucleic acid, wherein the amino acid sequence of the effector protein is at least 90% identical to a sequence selected from Column A1 of TABLE 1, and wherein at least a portion of the engineered guide nucleic acid comprises a nucleobase sequence that is at least 90% identical or at least 90% reverse complementary to a sequence selected from Column B1 of TABLE 1, wherein the sequence from Column A1 and the sequence from Column B1 are in the same row of TABLE 1.

In some embodiments, the disclosure provides a composition comprising an effector protein, and an engineered guide nucleic acid, wherein the amino acid sequence of the effector protein is at least 95% identical to a sequence selected from Column A1 of TABLE 1, and wherein at least a portion of the engineered guide nucleic acid comprises a nucleobase sequence that is at least 95% identical or at least 95% reverse complementary to a sequence selected from Column B1 of TABLE 1, wherein the sequence from Column A1 and the sequence from Column B1 are in the same row of TABLE 1.

In some embodiments, the disclosure provides a composition comprising an effector protein, and an engineered guide nucleic acid, wherein the amino acid sequence of the effector protein is 100% identical to a sequence selected from Column A1 of TABLE 1, and wherein at least a portion of the engineered guide nucleic acid comprises a nucleobase sequence that is 100% identical or 100% reverse complementary to a sequence selected from Column B1 of TABLE 1, wherein the sequence from Column A1 and the sequence from Column B1 are in the same row of TABLE 1. In some embodiments, the disclosure provides a composition comprising an effector protein, and an engineered guide nucleic acid, wherein the amino acid sequence of the effector protein is at least 50% identical to a sequence selected from Column A2 of TABLE 1, and wherein at least a portion of the engineered guide nucleic acid comprises a nucleobase sequence that is at least 50% identical or at least 50% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is at least 60% identical to a sequence selected from Column A2 of TABLE 1, and wherein at least a portion of the engineered guide nucleic acid comprises a nucleobase sequence that is at least 60% identical or at least 60% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is at least 70% identical to a sequence selected from Column A2 of TABLE 1, and wherein at least a portion of the engineered guide nucleic acid comprises a nucleobase sequence that is at least 70% identical or at least 70% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is at least 80% identical to a sequence selected from Column A2 of TABLE 1, and wherein at least a portion of the engineered guide nucleic acid comprises a nucleobase sequence that is at least 80% identical or at least 80% reverse complementary to a sequence selected from Column B2 of TABLE 1, wherein the sequence from Column A2 and the sequence from Column B2 are in the same row of TABLE

In some embodiments, the disclosure provides a composition comprising an effector protein, and an engineered guide nucleic acid, wherein the amino acid sequence of the effector protein is at least 90% identical to a sequence selected from Column A2 of TABLE 1, and wherein at least a portion of the engineered guide nucleic acid comprises a nucleobase sequence that is at least 90% identical or at least 90% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is at least 95% identical to a sequence selected from Column A2 of TABLE 1, and wherein at least a portion of the engineered guide nucleic acid comprises a nucleobase sequence that is at least 95% identical or at least 95% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is 100% identical to a sequence selected from Column A2 of TABLE 1, and wherein at least a portion of the engineered guide nucleic acid comprises a nucleobase sequence that is 100% identical or 100% reverse complementary to a sequence selected from Column B2 of TABLE 1, wherein the sequence from Column A2 and the sequence from Column B2 are in the same row of TABLE 1. In some embodiments, the disclosure provides a composition comprising an effector protein, and an engineered guide nucleic acid, wherein the amino acid sequence of the effector protein is at least 50% identical to a sequence selected from Column A3 of TABLE 1, and wherein at least a portion of the engineered guide nucleic acid comprises a nucleobase sequence that is at least 50% identical or at least 50% reverse complementary to a sequence selected from Column B3 of TABLE 1, wherein the sequence from Column A3 and the sequence from Column B3 are in the same row of TABLE 1. In some embodiments, the disclosure provides a composition comprising an effector protein, and an engineered guide nucleic acid, wherein the amino acid sequence of the effector protein is at least 60% identical to a sequence selected from Column A3 of TABLE 1, and wherein at least a portion of the engineered guide nucleic acid comprises a nucleobase sequence that is at least 60% identical or at least 60% reverse complementary to a sequence selected from Column B3 of TABLE 1, wherein the sequence from Column A3 and the sequence from Column B3 are in the same row of TABLE 1. In some embodiments, the disclosure provides a composition comprising an effector protein, and an engineered guide nucleic acid, wherein the amino acid sequence of the effector protein is at least 70% identical to a sequence selected from Column A3 of TABLE 1, and wherein at least a portion of the engineered guide nucleic acid comprises a nucleobase sequence that is at least 70% identical or at least 70% reverse complementary to a sequence selected from Column B3 of TABLE 1, wherein the sequence from Column A3 and the sequence from Column B3 are in the same row of TABLE 1.

In some embodiments, the disclosure provides a composition comprising an effector protein, and an engineered guide nucleic acid, wherein the amino acid sequence of the effector protein is at least 80% identical to a sequence selected from Column A3 of TABLE 1, and wherein at least a portion of the engineered guide nucleic acid comprises a nucleobase sequence that is at least 80% identical or at least 80% reverse complementary to a sequence selected from Column B3 of TABLE 1, wherein the sequence from Column A3 and the sequence from Column B3 are in the same row of TABLE 1. In some embodiments, the disclosure provides a composition comprising an effector protein, and an engineered guide nucleic acid, wherein the amino acid sequence of the effector protein is at least 90% identical to a sequence selected from Column A3 of TABLE 1, and wherein at least a portion of the engineered guide nucleic acid comprises a nucleobase sequence that is at least 90% identical or at least 90% reverse complementary to a sequence selected from Column B3 of TABLE 1, wherein the sequence from Column A3 and the sequence from Column B3 are in the same row of TABLE 1. In some embodiments, the disclosure provides a composition comprising an effector protein, and an engineered guide nucleic acid, wherein the amino acid sequence of the effector protein is at least 95% identical to a sequence selected from Column A3 of TABLE 1, and wherein at least a portion of the engineered guide nucleic acid comprises a nucleobase sequence that is at least 95% identical or at least 95% reverse complementary to a sequence selected from Column B3 of TABLE 1, wherein the sequence from Column A3 and the sequence from Column B3 are in the same row of TABLE 1. In some embodiments, the disclosure provides a composition comprising an effector protein, and an engineered guide nucleic acid, wherein the amino acid sequence of the effector protein is 100% identical to a sequence selected from Column A3 of TABLE 1, and wherein at least a portion of the engineered guide nucleic acid comprises a nucleobase sequence that is 100% identical or 100% reverse complementary to a sequence selected from Column B3 of TABLE 1, wherein the sequence from Column A3 and the sequence from Column B3 are in the same row of TABLE 1.

In some embodiments, at least a portion of the guide nucleic acid binds the effector protein. In some embodiments, the guide nucleic acid comprises a crRNA. In some embodiments, the guide nucleic acid comprises a tracrRNA. In some embodiments, the composition does not comprise a tracrRNA. In some embodiments, the guide nucleic acid comprises a crRNA covalently linked to a tracrRNA. In some embodiments, the guide nucleic acid comprises a first sequence and a second sequence, wherein the first sequence is heterologous with the second sequence. In some embodiments, the first sequence comprises at least five amino acids and the second sequence comprises at least five amino acids. In some embodiments of the compositions provided herein, at least one of the effector protein, the guide nucleic acid, and the combination thereof, are not naturally occurring.

In some embodiments of the compositions provided herein, at least one of the effector protein and the guide nucleic acid is recombinant or engineered. In some embodiments of the compositions provided herein, the guide nucleic acid comprises a nucleotide sequence that is at least 80%, at least 85%, at least 90%, at least 95%, at least 98% or 100% identical to a nucleotide sequence selected from SEQ ID NOS: 10,485-15,015 or 24,166-31,319. In some embodiments of the compositions provided herein, the guide nucleic acid comprises at least 8, at least 9, at least 10, at least 11, at least 12, at least 13, at least 14, at least 15, at least 16, at least 17, at least 18, at least 19, or at least 20 contiguous nucleotides of a nucleotide sequence selected from SEQ ID NOS: 10,485-15,015 or 24,166-31,319. In some embodiments of the compositions provided herein, the guide nucleic acid comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence selected from SEQ ID NOS: 10,485-15,015 or 24,166-31,319. In some embodiments of the compositions provided herein, the guide nucleic acid comprises a sequence that hybridizes to a target sequence of a target nucleic acid, and wherein the target nucleic acid comprises a protospacer adjacent motif (PAM). In some embodiments of the compositions provided herein, the PAM is located within 1, 5, 10, 15, 20, 40, 60, 80 or 100 nucleotides of the 5′ end of the target sequence. In some embodiments of the compositions provided herein, the effector protein comprises a nuclear localization signal. In some embodiments of the compositions provided herein, the composition further comprises a donor nucleic acid.

In some embodiments of the compositions provided herein, the composition further comprises a fusion partner protein linked to the effector protein. In some embodiments of the compositions provided herein, the fusion partner protein is directly fused to the N terminus or C terminus of the effector protein via an amide bond. In some embodiments of the compositions provided herein, the fusion partner protein is directly fused to the N terminus or C terminus of the effector protein via a peptide linker. In some embodiments of the compositions provided herein, the fusion partner protein comprises a polypeptide selected from a deaminase, a transcriptional activator, a transcriptional repressor, or a functional domain thereof. In some embodiments of the compositions provided herein, the effector protein comprises at least one mutation that reduces its nuclease activity relative to the effector protein without the mutation as measured in a cleavage assay, optionally wherein the effector protein is a catalytically inactive nuclease. In some embodiments, any one of the compositions provided herein comprise a nucleic acid expression vector, wherein the nucleic acid vector encodes at least one of the effector protein and the guide nucleic acid of the compositions described herein. In some embodiments, any one of the compositions provided herein comprise a donor nucleic acid, optionally wherein the donor nucleic acid is encoded by the nucleic acid expression vector or an additional nucleic acid expression vector. In some embodiments, the nucleic acid expression vector is a viral vector. In some embodiments, the viral vector is an adeno associated viral (AAV) vector. In some embodiments, the virus comprises any one of the compositions herein.

In some embodiments, provided herein is a pharmaceutical composition, comprising any one of the compositions herein, and a pharmaceutically acceptable excipient. In some embodiments, provided herein is a system comprising any of the compositions described herein, and at least one detection reagent for detecting a target nucleic acid. In some embodiments, the at least one detection reagent is selected from a reporter nucleic acid, a detection moiety, an additional effector protein, or a combination thereof, optionally wherein the reporter nucleic acid comprises a fluorophore, a quencher, or a combination thereof. In some embodiments, the system further comprises at least one amplification reagent for amplifying a target nucleic acid. In some embodiments, the at least one amplification reagent is selected from the group consisting of a primer, a polymerase, a deoxynucleoside triphosphate (dNTP), a ribonucleoside triphosphate (rNTP), and combinations thereof. In some embodiments, the system further comprises a device with a chamber or solid support for containing the composition, target nucleic acid, detection reagent or combination thereof. In some embodiments, provided herein is a method of detecting a target nucleic acid in a sample, comprising the steps of: a) contacting the sample with: i) any one of the compositions described herein or any one of the systems described herein; and ii) a reporter nucleic acid comprising a detectable moiety that produces a detectable signal in the presence of the target nucleic acid and the composition or system, and b) detecting the detectable signal.

In some embodiments of the method, the reporter nucleic acid comprises a fluorophore, a quencher, or a combination thereof, and wherein the detecting comprises detecting a fluorescent signal. In some embodiments of the method, the method further comprises reverse transcribing the target nucleic acid, amplifying the target nucleic acid, in vitro transcribing the target nucleic acid, or any combination thereof. In some embodiments of the method, the method further comprises reverse transcribing the target nucleic acid and/or amplifying the target nucleic acid before contacting the sample with the composition. In some embodiments of the method, the method further comprises reverse transcribing the target nucleic acid and/or amplifying the target nucleic acid after contacting the sample with the composition. In some embodiments of the method, amplifying comprises isothermal amplification. In some embodiments of the method, the target nucleic acid is from a pathogen. In some embodiments of the method, the pathogen is a virus. In some embodiments of the method, the target nucleic acid comprises RNA. In some embodiments of the method, the target nucleic acid comprises DNA. In some embodiments, provided herein is a method of modifying a target nucleic acid, the method comprising contacting the target nucleic acid with any one of the compositions herein, or any one of the systems described herein, thereby modifying the target nucleic acid. In some embodiments of the method modifying the target nucleic acid comprises cleaving the target nucleic acid, deleting a nucleotide of the target nucleic acid, inserting a nucleotide into the target nucleic acid, substituting a nucleotide of the target nucleic acid with an alternative nucleotide or an additional nucleotide, or any combination thereof. In some embodiments, the method comprises contacting the target nucleic acid with a donor nucleic acid.

In some embodiments of the method, the target nucleic acid comprises a mutation associated with a disease. In some embodiments of the method, the disease is selected from an autoimmune disease, a cancer, an inherited disorder, an ophthalmological disorder, a metabolic disorder, or a combination thereof. In some embodiments of the method, the disease is cystic fibrosis, thalassemia, Duchenne muscular dystrophy, myotonic dystrophy Type 1, or sickle cell anemia. In some embodiments of the method, contacting the target nucleic acid comprises contacting a cell, wherein the target nucleic acid is located in the cell. In some embodiments of the method, the contacting occurs in vitro. In some embodiments of the method, the contacting occurs in vivo. In some embodiments of the method, the contacting occurs ex vivo.

In some embodiments, provided herein is a cell comprising any one of the compositions described herein. In some embodiments, provided herein is a cell modified by any one of the compositions described herein. In some embodiments, provided herein is a cell modified by any one of the embodiments of the systems described herein. In some embodiments, provided herein is a cell comprising a modified target nucleic acid, wherein the modified target nucleic acid is a target nucleic acid modified according to any one of the embodiments of the methods herein. In some embodiments, the cell is a eukaryotic cell. In some embodiments, the cell is a mammalian cell. In some embodiments, the cell is a prokaryotic cell. In some embodiments, the cell is a plant cell. In some embodiments, the cell is an animal cell. In some embodiments, the cell is a T cell, optionally wherein the T cell is a natural killer T cell (NKT). In some embodiments, the cell is a chimeric antigen receptor T cell (CAR T-cell). In some embodiments, the cell is an induced pluripotent stem cell (iPSC). In some embodiments, provided herein is a population of cells comprising any one of the compositions herein or generated using any of the methods described herein.

In some embodiments, provided herein is a method of producing a protein, the method comprising i) contacting a cell comprising a target nucleic acid with the any one of the compositions herein, thereby editing the target nucleic acid to produce a modified cell comprising a modified target nucleic acid; and ii) producing a protein from the cell that is encoded, transcriptionally affected, or translationally affected by the modified nucleic acid. In some embodiments, the method comprises administering to a subject in need thereof a composition described herein, or a cell according to any one of the compositions herein or produced using any of the methods herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### II. Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the,” as used herein, include plural references unless the context clearly dictates otherwise.

Any reference to “or” herein is intended to encompass “and/or” unless otherwise stated. As used herein, the term “and/or” includes any and all combinations of one or more of the associated listed items.

Use of the term “including” as well as other forms, such as “includes” and “included,” is not limiting.

As used herein, the term “comprise” and its grammatical equivalents specifies the presence of stated features, integers, steps, operations, elements, and/or components, but does not preclude the presence or addition of one or more other features, integers, steps, operations, elements, components, and/or groups thereof.

As used herein, the term “about” in reference to a number or range of numbers, is understood to mean the stated number and numbers +/−10% thereof, or 10% below the lower listed limit and 10% above the higher listed limit for the values listed for a range.

The terms, “percent identity,” “% identity,” and % “identical,” grammatical equivalents thereof, as used herein refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The term, “amplification” and “amplifying,” as used herein refers to a process by which a nucleic acid molecule is enzymatically copied to generate a plurality of nucleic acid molecules containing the same sequence as the original nucleic acid molecule or a distinguishable portion thereof.

The term, “base editing enzyme,” as used herein refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term, “base editor,” as used herein, refers to a fusion protein comprising a base editing enzyme fused to or linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein, e.g., a Cas effector protein.

The term, “cis cleavage,” as used herein, refers to cleavage (hydrolysis of a phosphodiester bond) of a target nucleic acid by a complex of an effector protein and a guide nucleic acid (e.g., an RNP complex), wherein at least a portion of the guide nucleic acid is hybridized to at least a portion of the target nucleic acid. Cleavage may occur within or directly adjacent to the portion of the target nucleic acid that is hybridized to the portion of the guide nucleic acid.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the “reverse complement” sequence or the “reverse complementary” sequence is understood as the sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “cleavage assay,” as used herein, refers to an assay designed to visualize, quantitate or identify cleavage of a nucleic acid. In some instances, the cleavage activity may be cis-cleavage activity. In some instances, the cleavage activity may be trans-cleavage activity.

The term, “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms, “CRISPR RNA” and “crRNA,” as used herein, refer to a type of guide nucleic acid that is RNA comprising a first sequence, often referred to as a “spacer sequence,” that is capable of hybridizing to a target sequence of a target nucleic acid and a second sequence that is capable of interacting with an effector protein either directly (by being bound by an effector protein) or indirectly crRNA (e.g., by hybridization with a second nucleic acid molecule that can be bound by an effector).

The first sequence and the second sequence are directly connected to each other or by a linker.

The term, “detectable signal,” as used herein, refers to a signal that can be detected using optical, fluorescent, chemiluminescent, electrochemical or other detection methods known in the art.

The term, “donor nucleic acid,” as used herein, refers to a nucleic acid that is (designed or intended to be) incorporated into a target nucleic acid or target sequence.

The term, “effector protein,” as used herein, refers to a protein, polypeptide, or peptide that is capable of interacting with a nucleic acid, such as a guide nucleic acid, to form a complex that contacts a target nucleic acid, wherein at least a portion of the guide nucleic acid hybridizes to a target sequence of the target nucleic acid. In some embodiments, the complex comprises multiple effector proteins. In some embodiments, the effector protein modifies the target nucleic acid when the (e.g., a RNP complex contacts the target nucleic acid. In some embodiments, the effector protein does not modify the target nucleic acid, but it is fused to a fusion partner protein that modifies the target nucleic acid. A non-limiting example of modifying a target nucleic acid is cleaving (hydrolysis) of a phosphodiester bond. Additional examples of modifying target nucleic acids are described herein.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid editing, nucleic acid modifying, nucleic acid cleaving, protein binding. The absence of the functional domain, including mutations of the functional domain, would abolish or reduce activity.

The term “functional fragment,” as used herein, refers to a fragment of a protein that retains some function relative to the entire protein. Non-limiting examples of functions are nucleic acid binding, nucleic acid editing, protein binding, nuclease activity, nickase activity, deaminase activity, demethylase activity, or acetylation activity. A functional fragment may be a recognized functional domain, e.g., a catalytic domain such as, but not limited to, a RuvC domain.

The term, “fusion effector”, “fusion protein,” and “fusion polypeptide,” as used herein, refer to a protein comprising at least two heterologous polypeptides. The fusion protein may comprise one or more effector protein and fusion partner. In some instances, an effector protein and fusion partner are not found connected to one another as a native protein or complex that occurs together in nature.

As used herein, the terms “fusion partner protein” or “fusion partner,” refer to a protein, polypeptide or peptide that is fused, or linked by a linker, to one or more effector protein. The fusion partner can impart some function to the fusion protein that is not provided by the effector protein. The fusion partner may provide a detectable signal. The fusion partner may modify a target nucleic acid, including changing a nucleobase of the target nucleic acid and making a chemical modification to one or more nucleotides of the target nucleic acid. The fusion partner may be capable of modulating the expression of a target nucleic acid. The fusion partner may inhibit, reduce, activate or increase expression of a target nucleic acid via additional proteins or nucleic acid modifications to the target sequence.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “heterologous,” as used herein, refers to at least two different polypeptide or nucleic acid sequences that are not found similarly connected to one another in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked by an amide bond to the effector protein in nature. In some instances, a heterologous protein is not encoded by a species that encodes the effector protein. In some embodiments, the heterologous protein exhibits an activity (e.g., enzymatic activity) when it is fused to the effector protein. In some embodiments, the heterologous protein exhibits increased or reduced activity (e.g., enzymatic activity) when it is fused to the effector protein, relative to when it is not fused to the effector protein. In some embodiments, the heterologous protein exhibits an activity (e.g., enzymatic activity) that it does not exhibit when it is fused to the effector protein. A guide nucleic acid may comprise “heterologous” sequences, e.g., a guide nucleic acid may comprise a first sequence and a second sequence, wherein the first sequence and the second sequence are not found covalently linked by a phosphodiester bond in nature. Thus, the first sequence is considered to be heterologous with the second sequence, and the guide nucleic acid may be referred to as a heterologous guide nucleic acid.

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The term, “linked amino acids” as used herein refers to at least two amino acids linked by an amide bond.

The term, “linker,” as used herein, refers to a covalent bond or molecule that links a first polypeptide to a second polypeptide (e.g., by an amide bond) or a first nucleic acid to a second nucleic acid (e.g., by a phosphodiester bond).

The term, “edited target nucleic acid,” as used herein, refers to a target nucleic acid, wherein the target nucleic acid has undergone an editing, for example, after contact with an effector protein. In some instances, the editing is an alteration in the sequence of the target nucleic acid. In some instances, the edited target nucleic acid comprises an insertion, deletion, or substitution of one or more nucleotides compared to the unedited target nucleic acid.

The terms, “mutation associated with a disease” and “mutation associated with a genetic disorder,” as used herein, refer to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid that is at least substantially free from at least one other feature with which it is naturally associated in nature and as found in nature, and/or contains or to a modification of that molecule (e.g., chemical modification, nucleotide sequence, or amino acid sequence) that is not present in the naturally occurring molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” as used herein, refer to an enzyme which possesses catalytic activity for nucleic acid cleavage.

The term, “nuclease activity,” as used herein, refers to catalytic activity that results in nucleic acid cleavage (e.g., ribonuclease activity (ribonucleic acid cleavage), or deoxyribonuclease activity (deoxyribonucleic acid cleavage), etc.).

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The term, “prime editing enzyme”, as used herein, refers to a protein, polypeptide, or fragment thereof that is capable of catalyzing the editing (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

The terms “protospacer adjacent motif” and “PAM,” as used herein, refer to a nucleotide sequence found in a target nucleic acid that directs an effector protein to edit the target nucleic acid at a specific location. In some instances, a PAM is required for a complex of an effector protein and a guide nucleic acid (e.g., an RNP complex) to hybridize to and edit the target nucleic acid. In some instances, the complex does not require a PAM to edit the target nucleic acid.

The term “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms (see “DNA regulatory sequences”, below).

The term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. The term, “recombinant polypeptide,” refers to a polypeptide which is not naturally occurring, e.g., is made by the artificial combination of two otherwise separated segments of amino sequence through human intervention. Thus, e.g., a polypeptide that comprises a heterologous amino acid sequence is recombinant.

The terms, “reporter” and “reporter nucleic acid,” as used herein, refer to a non-target nucleic acid molecule that can provide a detectable signal upon cleavage by an effector protein. Examples of detectable signals and detectable moieties that generate detectable signals are provided herein.

The term, “sample,” as used herein, refers to something comprising a target nucleic acid. In some instances, the sample is a biological sample, such as a biological fluid or tissue sample. In some instances, the sample is an environmental sample. The sample may be a biological sample or environmental sample that is modified or manipulated. By way of non-limiting example, samples may be modified or manipulated with purification techniques, heat, nucleic acid amplification, salts and buffers.

The term, “subject,” as used herein can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some embodiments, the subject is not necessarily diagnosed or suspected of being at high risk for the disease.

The term, “target nucleic acid,” as used herein, refers to a nucleic acid that is selected as the nucleic acid for editing, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid.

The term, “trans-activating RNA (tracrRNA)”, “transactivating RNA”, and “tracrRNA,” as used herein refers to a nucleic acid that comprises a first sequence that is capable of being non-covalently bound by an effector protein. TracrRNAs may comprise a second sequence that hybridizes to a portion of a crRNA, which may be referred to as a repeat hybridization sequence. In some embodiments, tracrRNAs are covalently linked to a crRNA.

The term, “transcriptional activator,” as used herein, refers to a polypeptide or a fragment thereof that can activate or increase transcription of a target nucleic acid molecule.

The term, “transcriptional repressor,” as used herein, refers to a polypeptide or a fragment thereof that is capable of arresting, preventing, or reducing transcription of a target nucleic acid.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “viral vector,” as used herein, refers to a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof.

### III. Introduction

Disclosed herein are compositions, systems and methods comprising at least one of:
(a) a polypeptide or a nucleic acid encoding the polypeptide; and
(b) a guide nucleic acid or a nucleic acid encoding the guide nucleic acid.

Polypeptides described herein may bind and, optionally, cleave nucleic acids in a sequence-specific manner. The term “nucleic acid” refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both. The terms “nucleotide(s)” and “nucleoside(s)” as used herein, in the context of a nucleic acid molecule having multiple residues, refer to describing the sugar and base of the residue contained in the nucleic acid molecule. Similarly, a skilled artisan could understand that linked nucleotides and/or linked nucleosides, as used in the context of a nucleic acid having multiple linked residues, are interchangeable and describe linked sugars and bases of residues contained in a nucleic acid molecule. When referring to a “nucleobase(s)”, or linked nucleobase, as used in the context of a nucleic acid molecule, it can be understood as describing the base of the residue contained in the nucleic acid molecule, for example, the base of a nucleotide, nucleosides, or linked nucleotides or linked nucleosides. A person of ordinary skill in the art when referring to nucleotides, nucleosides, and/or nucleobases would also understand the differences between RNA and DNA (generally the exchange of uridine for thymidine or vice versa) and the presence of nucleoside analogs, such as modified uridines, do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, NI-methyl pseudouridine, or 5-methoxyuridine, is considered 100% identical to AUG in that both are perfectly complementary to the same sequence (5′-CAU).

The terms “polypeptide” and “protein” refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some embodiments, when a heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

Polypeptides described herein may also cleave the target nucleic acid within a target sequence or at a position adjacent to the target sequence. In some embodiments, a polypeptide is activated when it binds a certain sequence of a nucleic acid described herein, allowing the polypeptide to cleave a region of a target nucleic acid that is near, but not adjacent to the target sequence. A polypeptide may be an effector protein, such as a CRISPR-associated (Cas) protein, which may bind a guide nucleic acid that imparts activity or sequence selectivity to the polypeptide.

The terms “cleave,” “cleaving,” and “cleavage” in the context of a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein . . .

In some embodiments, compositions, systems, and methods comprising effector proteins and guide nucleic acids comprise a first sequence, at least a portion of which interacts with a polypeptide. In some embodiments, the first sequence comprises a sequence that is similar or identical to a repeat sequence. The term “repeat sequence” refers to a sequence of nucleotides in a guide nucleic acid that is capable of, at least partially, interacting with an effector protein. In some embodiments, compositions, systems, and methods comprising effector proteins and guide nucleic acids comprise a second sequence that is at least partially complementary to a target nucleic acid, and which may be referred to as a spacer sequence. “Spacer sequence,” as used herein, refers to a nucleotide sequence in a guide nucleic acid that is capable of, at least partially, hybridizing to an equal length portion of a sequence (e.g., a target sequence) of a target nucleic acid.

Effector proteins disclosed herein may cleave nucleic acids, including single stranded RNA (ssRNA), double stranded DNA (dsDNA), and single-stranded DNA (ssDNA). Polypeptides disclosed herein may provide cis cleavage activity, nickase activity, nuclease activity, or a combination thereof. In some embodiments, the present disclosure provides a viral vector comprising a nucleic acid encoding an effector protein. Non-limiting examples of viral vectors include retroviral vectors (e.g., lentiviruses and γ-retroviruses), adenoviruses, arenaviruses, alphaviruses, adeno-associated viruses (AAVs), baculoviruses, vaccinia viruses, herpes simplex viruses and poxviruses. A viral vector may be replication competent, replication deficient or replication defective.

The compositions, systems and methods described herein are non-naturally occurring. In some embodiments, compositions, systems and methods comprise an engineered guide nucleic acid (also referred to herein as a guide nucleic acid) or a use thereof. In some embodiments, compositions, systems and methods comprise an engineered protein or a use thereof. In some embodiments, compositions, systems and methods comprise an isolated polypeptide or a use thereof. In general, compositions, methods and systems described herein are not found in nature. In some embodiments, compositions, methods and systems described herein comprise at least one non-naturally occurring component. For example, disclosed compositions, methods and systems may comprise a guide nucleic acid, wherein the sequence of the guide nucleic acid is different or modified from that of a naturally-occurring guide nucleic acid.

In some embodiments, compositions, systems, and methods comprise at least two components that do not naturally occur together. For example, disclosed compositions, systems and methods may comprise a guide nucleic acid comprising a first region, at least a portion of which, interacts with a polypeptide (e.g., a repeat sequence), and a second region that is at least partially complementary to a target nucleic acid (e.g., a spacer sequence), wherein the first region and second region do not naturally occur together. Also, by way of non-limiting example, disclosed compositions, systems, and methods may comprise a guide nucleic acid and an effector protein that do not naturally occur together. Likewise, by way of non-limiting example, disclosed compositions, systems, and methods may comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “% complementary”, “% complementarity”, “percent complementary”, “percent complementarity” and grammatical equivalents thereof in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Guide nucleic acids may comprise a first sequence and a second sequence that do not occur naturally together. For example, a guide nucleic acid may comprise a naturally-occurring repeat sequence and a spacer sequence that is complementary to a naturally-occurring eukaryotic sequence. The guide nucleic acid may comprise a repeat sequence that occurs naturally in an organism and a spacer sequence that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

In some embodiments, compositions, systems, and methods described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The term “mutation” refers to an alteration that changes an amino acid residue or a nucleotide as described herein. Such an alteration can include, for example, deletions, insertions, and/or substitutions. The mutation can refer to a change in structure of an amino acid residue or nucleotide relative to the starting or reference residue or nucleotide. A mutation of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. A mutation of a nucleotide includes, for example, changing one naturally occurring base for a different naturally occurring base, such as changing an adenine to a thymine or a guanine to a cytosine or an adenine to a cytosine or a guanine to a thymine. A mutation of a nucleotide base may result in a structural and/or functional alteration of the encoding peptide, polypeptide or protein by changing the encoded amino acid residue of the peptide, polypeptide or protein. A mutation of a nucleotide base may not result in an alteration of the amino acid sequence or function of encoded peptide, polypeptide or protein, also known as a silent mutation. Methods of mutating an amino acid residue or a nucleotide are well known.

The effector protein may also comprise at least one additional amino acid relative to the naturally-occurring effector protein. In some embodiments, the effector protein may comprise a heterologous polypeptide. For example, the effector protein may comprise an addition of a nuclear localization signal relative to the natural occurring effector protein. In some embodiments, a nucleotide sequence encoding the effector protein is codon optimized (e.g., for expression in a eukaryotic cell) relative to the naturally occurring sequence.

The term “codon optimized” refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.jp/codon.

### IV. Polypeptide Systems

### Effector Proteins

Provided herein are compositions, systems, and methods comprising one or more effector proteins or a use thereof. In some embodiments, provided herein are compositions that comprise a nucleic acid, wherein the nucleic acid encodes any of one the effector proteins described herein. The nucleic acid may be a nucleic acid expression vector. By way of non-limiting example, the nucleic acid expression vector may be a viral vector, such as an AAV vector. In general, effector proteins disclosed herein are CRISPR-associated (“Cas”) proteins.

An effector protein provided herein interacts with a guide nucleic acid to form a complex. In some embodiments, the complex interacts with a target nucleic acid. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. In some embodiments, recognition of a PAM sequence within a target nucleic acid may direct the modification activity of an effector protein.

The terms, “hybridize,” “hybridizable” and grammatical equivalents thereof, refer to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001).

Modification activity of an effector protein or an engineered protein described herein may be cleavage activity, binding activity, insertion activity, substitution activity, and the like. Modification activity of an effector protein may result in: cleavage of at least one strand of a target nucleic acid, deletion of one or more nucleotides of a target nucleic acid, insertion of one or more nucleotides into a target nucleic acid, substitution of one or more nucleotides of a target nucleic acid with an alternative nucleotide, more than one of the foregoing, or any combination thereof. In some embodiments, an ability of an effector protein to edit a target nucleic acid may depend upon the effector protein being complexed with a guide nucleic acid, the guide nucleic acid being hybridized to a target sequence of the target nucleic acid, the distance between the target sequence and a PAM sequence, or combinations thereof. A target nucleic acid comprises a target strand and a non-target strand. Accordingly, in some embodiments, the effector protein may edit a target strand and/or a non-target strand of a target nucleic acid.

The terms, “bind,” “binding,” “interact” and “interacting,” refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The modification of the target nucleic acid generated by an effector protein may, as a non-limiting example, result in modulation of the expression of the target nucleic acid (e.g., increasing or decreasing expression of the nucleic acid) or modulation of the activity of a translation product of the target nucleic acid (e.g., inactivation of a protein binding to an RNA molecule or hybridization). Accordingly, in some embodiments, provided herein are methods of editing a target nucleic acid using an effector protein of the present disclosure, or compositions or systems thereof. Also provided herein are methods of modulating expression of a target nucleic acid using an effector protein of the present disclosure, or compositions or systems thereof. Further provided herein are methods of modulating the activity of a translation product of a target nucleic acid using an effector protein of the present disclosure, or compositions or systems thereof.

In some embodiments, effector proteins disclosed herein may provide cleavage activity, such as cis cleavage activity, nickase activity, nuclease activity, or a combination thereof. In general, effector proteins described herein edit a target nucleic acid by cis cleavage activity on the target nucleic acid. Effector proteins disclosed herein may cleave nucleic acids, including single stranded RNA (ssRNA), double stranded DNA (dsDNA), and single-stranded DNA (ssDNA). An effector protein may be a modified effector protein having increased modification activity and/or increased substrate binding activity (e.g., substrate selectivity, specificity, and/or affinity). Alternatively, or in addition, an effector protein may be a catalytically inactive effector protein having reduced modification activity or no modification activity. An effector protein may recognize a protospacer adjacent motif (PAM) sequence present in the target nucleic acid, which may direct the modification activity of the effector protein. The term “nickase” refers to an enzyme that possess catalytic activity for single stranded nucleic acid cleavage of a double stranded nucleic acid. The term, “nickase activity” refers to catalytic activity that results in single stranded nucleic acid cleavage of a double stranded nucleic acid.

An effector protein may be a CRISPR-associated (“Cas”) protein. An effector protein may function as a single protein, including a single protein that is capable of binding to a guide nucleic acid and editing a target nucleic acid. Alternatively, an effector protein may function as part of a multiprotein complex, including, for example, a complex having two or more effector proteins, including two or more of the same effector proteins (e.g., dimer or multimer). An effector protein, when functioning in a multiprotein complex, may have only one functional activity (e.g., binding to a guide nucleic acid), while other effector proteins present in the multiprotein complex are capable of the other functional activity (e.g., modifying a target nucleic acid). The first and second effector proteins may be the same. The first and second effector proteins may be different. The sequences of the first and second effector proteins may be 15% to 20% identical, 20% to 25% identical, 25% to 30% identical, 30% to 35% identical, 35% to 40% identical, 40% to 45% identical, 45% to 50% identical, 50% to 55% identical, 55% to 60% identical, 60% to 65% identical, 65% to 70% identical, 70% to 75% identical, 75% to 80% identical, 80% to 85% identical, 85% to 90% identical, 90% to 95% identical, 95% to 99.9% identical, or 100% identical. An effector protein, when functioning in a multiprotein complex, may have differing and/or complementary functional activity to other effector proteins in the multiprotein complex. Multimeric complexes, and functions thereof, are described in further detail below.

Effector proteins may be a modified effector protein having reduced modification activity (e.g., a catalytically defective effector protein). Effector proteins may be a modified effector protein having no modification activity (e.g., a catalytically inactive effector protein). In some embodiments, the effector protein may have a mutation in a nuclease domain. In some embodiments, the nuclease domain is a RuvC domain. In some embodiments, the nuclease domain is an HNH domain. An HNH domain may be characterized as comprising two antiparallel β-strands connected with a loop of varying length, and flanked by an α-helix, with a metal (divalent cation) binding site between the two β-strands. A RuvC domain may be characterized by a six-stranded beta sheet surrounded by four alpha helices, with three conserved subdomains contributing catalytic to the activity of the RuvC domain.

The terms, “RuvC” and “RuvC domain,” as used herein, refer to a region of an effector protein that is capable of cleaving a target nucleic acid, and in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons.

An effector protein may be brought into proximity of a target nucleic acid in the presence of a guide nucleic acid when the guide nucleic acid includes a nucleotide sequence that is complementary with a target sequence in the target nucleic acid. The ability of an effector protein to modify a target nucleic acid may be dependent upon the effector protein being bound to a guide nucleic acid and the guide nucleic acid being hybridized to a target nucleic acid. An effector protein may recognize a protospacer adjacent motif (PAM) sequence present in the target nucleic acid, which may direct the modification activity of the effector protein.

In some instances, effector proteins comprise an amino acid sequence selected from any one of SEQ ID NOS: 1-10,484 or 15,022-24,165. In some instances, effector proteins consist essentially of an amino acid sequence selected from any one of SEQ ID NOS: 1-10,484 or 15,022-24,165. In some instances, effector proteins consist of an amino acid sequence selected from any one of SEQ ID NOS: 1-10,484 or 15,022-24,165. In some instances, effector proteins comprise an amino acid sequence that is at least 65%, identical to a sequence selected from any one of SEQ ID NOS: 1-10,484 or 15,022-24,165. In some instances, effector proteins consist of an amino acid sequence that is at least 65% identical to a sequence selected from any one of SEQ ID NOS: 1-10,484 or 15,022-24,165. In some instances, effector proteins comprise an amino acid sequence that is at least 70%, identical to a sequence selected from any one of SEQ ID NOS: 1-10,484 or 15,022-24,165. In some instances, effector proteins consist of an amino acid sequence that is at least 70% identical to a sequence selected from any one of SEQ ID NOS: 1-10,484 or 15,022-24,165. In some instances, effector proteins comprise an amino acid sequence that is at least 75%, identical to a sequence selected from any one of SEQ ID NOS: 1-10,484 or 15,022-24,165. In some instances, effector proteins consist of an amino acid sequence that is at least 75% identical to a sequence selected from any one of SEQ ID NOS: 1-10,484 or 15,022-24,165. In some instances, effector proteins comprise an amino acid sequence that is at least 80%, identical to a sequence selected from any one of SEQ ID NOS: 1-10,484 or 15,022-24,165. In some instances, effector proteins consist of an amino acid sequence that is at least 80% identical to a sequence selected from any one of SEQ ID NOS: 1-10,484 or 15,022-24,165. In some instances, effector proteins comprise an amino acid sequence that is at least 85%, identical to a sequence selected from any one of SEQ ID NOS: 1-10,484 or 15,022-24,165. In some instances, effector proteins consist of an amino acid sequence that is at least 85% identical to a sequence selected from any one of SEQ ID NOS: 1-10,484 or 15,022-24,165. In some instances, effector proteins comprise an amino acid sequence that is at least 90%, identical to a sequence selected from any one of SEQ ID NOS: 1-10,484 or 15,022-24,165. In some instances, effector proteins consist of an amino acid sequence that is at least 90% identical to a sequence selected from any one of SEQ ID NOS: 1-10,484 or 15,022-24,165. In some instances, effector proteins comprise an amino acid sequence that is at least 95%, identical to a sequence selected from any one of SEQ ID NOS: 1-10,484 or 15,022-24,165. In some instances, effector proteins consist of an amino acid sequence that is at least 95% identical to a sequence selected from any one of SEQ ID NOS: 1-10,484 or 15,022-24,165. In some instances, effector proteins comprise an amino acid sequence that is at least 97%, identical to a sequence selected from any one of SEQ ID NOS: 1-10,484 or 15,022-24,165. In some instances, effector proteins consist of an amino acid sequence that is at least 97% identical to a sequence selected from any one of SEQ ID NOS: 1-10,484 or 15,022-24,165. In some instances, effector proteins comprise an amino acid sequence that is at least 98%, identical to a sequence selected from any one of SEQ ID NOS: 1-10,484 or 15,022-24,165. In some instances, effector proteins consist of an amino acid sequence that is at least 98% identical to a sequence selected from any one of SEQ ID NOS: 1-10,484 or 15,022-24,165. In some instances, effector proteins comprise an amino acid sequence that is at least 99%, identical to a sequence selected from any one of SEQ ID NOS: 1-10,484 or 15,022-24,165. In some instances, effector proteins consist of an amino acid sequence that is at least 99% identical to a sequence selected from any one of SEQ ID NOS: 1-10,484 or 15,022-24,165. In some instances, effector proteins comprise an amino acid sequence that is 100%, identical to a sequence selected from any one of SEQ ID NOS: 1-10,484 or 15,022-24,165. In some instances, effector proteins consist of an amino acid sequence that is 100% identical to a sequence selected from any one of SEQ ID NOS: 1-10,484 or 15,022-24,165.

TABLE 1 provides illustrative amino acid sequences of effector proteins that are useful in the compositions, systems and methods described herein.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 100, at least about 120, at least about 140, at least about 160, at least about 180, at least about 200, at least about 220, at least about 240, at least about 260, at least about 280, at least about 300, at least about 320, at least about 340, at least about 360, at least about 380, at least about 400 contiguous amino acids, at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, at least about 720, at least about 740, at least about 760, at least about 780, at least about 800, at least about 820, at least about 840, at least about 860, at least about 880, at least about 900, at least about 920, at least about 940, at least about 960, at least about 980, at least about 1000, at least about 1020, at least about 1040, at least about 1060, at least about 1080, at least about 1100, at least about 1120, at least about 1140, at least about 1160, at least about 1180, or at least about 1200 contiguous amino acids of a sequence selected from any one of SEQ ID NOS: 1-10,484 or 15,022-24,165. In some instances, effector proteins comprise less than about 1900, less than about 1850, less than about 1800, less than about 1750, less than about 1700, or less than about 1650 contiguous amino acids of a sequence selected from any one of SEQ ID NOS: 1-10,484 or 15,022-24,165.

In some instances, effector proteins comprise about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, or about 1400 contiguous amino acids of a sequence selected from any one of SEQ ID NOS: 1-10,484 or 15,022-24,165.

In some instances, compositions comprise an engineered guide nucleic acid (also referred to simply as a guide nucleic acid), wherein the guide nucleic acid comprises a nucleobase sequence selected from any one of SEQ ID NOS: 10,485-15,015 or 24,166-31,319. In some instances, guide nucleic acids comprise a sequence that is complementary to a nucleobase sequence selected from any one of SEQ ID NOS: 10,485-15,015 or 24,166-31,319. In some instances, guide nucleic acids comprise a sequence that is reverse complementary to a nucleobase sequence selected from any one of SEQ ID NOS: 10,485-15,015 or 24,166-31,319. In some instances, guide nucleic acids comprise a sequence that is at least 65% identical to a nucleobase sequence selected from any one of SEQ ID NOS: 10,485-15,015 or 24,166-31,319, the complement thereof, or the reverse complement thereof. In some instances, guide nucleic acids comprise a sequence that is at least 70% identical to a nucleobase sequence selected from any one of SEQ ID NOS: 10,485-15,015 or 24,166-31,319, the complement thereof, or the reverse complement thereof. In some instances, guide nucleic acids comprise a sequence that is at least 75% identical to a nucleobase sequence selected from any one of SEQ ID NOS: 10,485-15,015 or 24,166-31,319, the complement thereof, or the reverse complement thereof. In some instances, guide nucleic acids comprise a sequence that is at least 80% identical to a nucleobase sequence selected from any one of SEQ ID NOS: 10,485-15,015 or 24,166-31,319, the complement thereof, or the reverse complement thereof. In some instances, guide nucleic acids comprise a sequence that is at least 85% identical to a nucleobase sequence selected from any one of SEQ ID NOS: 10,485-15,015 or 24,166-31,319, the complement thereof, or the reverse complement thereof. In some instances, guide nucleic acids comprise a sequence that is at least 90% identical to a nucleobase sequence selected from any one of SEQ ID NOS: 10,485-15,015 or 24,166-31,319, the complement thereof, or the reverse complement thereof. In some instances, guide nucleic acids comprise a sequence that is 100% identical to a nucleobase sequence selected from any one of SEQ ID NOS: 10,485-15,015 or 24,166-31,319, the complement thereof, or the reverse complement thereof.

In some instances, guide nucleic acids comprise at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39 or at least 40 contiguous nucleotides of a nucleobase sequence selected from any one of SEQ ID NOS: 10,485-15,015 or 24,166-31,319, the complement thereof, or the reverse complement thereof. In some instances, guide nucleic acids contain less than 32, less than 34, less than 36, less than 37, less than 38, less than 39, less than 40, less than 41, less than 42, less than 43, less than 44, or less than 45 contiguous nucleotides of any one of the nucleobase sequences selected from any one of SEQ ID NOS: 10,485-15,015 or 24,166-31,319, the complement thereof, or the reverse complement thereof. In some instances, guide nucleic acids comprise 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides of any one of the nucleobase sequences selected from any one of SEQ ID NOS: 10,485-15,015 or 24, 166-31,319, the complement thereof, or the reverse complement thereof.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A2 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A3 of TABLE 1, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 50% identical to a sequence selected from Column A1 of TABLE 1, and wherein at least a portion of the guide nucleic acid comprises a nucleobase sequence that is at least about 50% identical or at least about 50% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A1 of TABLE 1, and wherein at least a portion of the guide nucleic acid comprises a nucleobase sequence that is at least about 60% identical or at least about 60% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A1 of TABLE 1, and wherein at least a portion of the guide nucleic acid comprises a nucleobase sequence that is at least about 70% identical or at least about 70% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A1 of TABLE 1, and wherein at least a portion of the guide nucleic acid comprises a nucleobase sequence that is at least about or 80% identical or at least about 80% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A1 of TABLE 1, and wherein at least a portion of the guide nucleic acid comprises a nucleobase sequence that is at least about 90% identical or at least about 90% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A1 of TABLE 1, and wherein at least a portion of the guide nucleic acid comprises a nucleobase sequence that is at least about 95% identical or at least 95% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is 100% identical to a sequence selected from Column A1 of TABLE 1, and wherein at least a portion of the guide nucleic acid comprises a nucleobase sequence that is 100% identical or 100% reverse complementary to a sequence selected from Column B1 of TABLE 1, wherein the sequence from Column A1 and the sequence from Column B1 are in the same row of TABLE 1.

In some instances, compositions comprise an effector protein or a fusion protein thereof, and a guide nucleic acid, wherein the amino acid sequence of the effector protein is at least about 50% identical to a sequence selected from Column A2 of TABLE 1, and wherein at least a portion of the guide nucleic acid comprises a nucleobase sequence that is at least about 50% identical or at least about 50% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A2 of TABLE 1, and wherein at least a portion of the guide nucleic acid comprises a nucleobase sequence that is at least about 60% identical or at least about 60% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A2 of TABLE 1, and wherein at least a portion of the guide nucleic acid comprises a nucleobase sequence that is at least about 70% identical or at least about 70% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A2 of TABLE 1, and wherein at least a portion of the guide nucleic acid comprises a nucleobase sequence that is at least about 80% identical or at least about 80% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A2 of TABLE 1, and wherein at least a portion of the guide nucleic acid comprises a nucleobase sequence that is at least about 90% identical or at least about 90% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A2 of TABLE 1, and wherein at least a portion of the guide nucleic acid comprises a nucleobase sequence that is at least about 95% identical or at least about 95% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is 100% identical to a sequence selected from Column A2 of TABLE 1, and wherein at least a portion of the guide nucleic acid comprises a nucleobase sequence that is 100% identical or 100% reverse complementary to a sequence selected from Column B2 of TABLE 1, wherein the sequence from Column A2 and the sequence from Column B2 are in the same row of TABLE 1.

In some instances, compositions comprise an effector protein or a fusion protein thereof, and a guide nucleic acid, wherein the amino acid sequence of the effector protein is at least about 50% identical to a sequence selected from Column A3 of TABLE 1, and wherein at least a portion of the guide nucleic acid comprises a nucleobase sequence that is at least about 50% identical or at least about 50% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 60% identical to a sequence selected from Column A3 of TABLE 1, and wherein at least a portion of the guide nucleic acid comprises a nucleobase sequence that is at least about 60% identical or at least about 60% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 70% identical to a sequence selected from Column A3 of TABLE 1, and wherein at least a portion of the guide nucleic acid comprises a nucleobase sequence that is at least about 70% identical or at least about 70% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 80% identical to a sequence selected from Column A3 of TABLE 1, and wherein at least a portion of the guide nucleic acid comprises a nucleobase sequence that is at least about 80% identical or at least about 80% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 90% identical to a sequence selected from Column A3 of TABLE 1, and wherein at least a portion of the guide nucleic acid comprises a nucleobase sequence that is at least about 90% identical or at least about 90% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least about 95% identical to a sequence selected from Column A3 of TABLE 1, and wherein at least a portion of the guide nucleic acid comprises a nucleobase sequence that is at least about 95% identical or at least about 95% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is 100% identical to a sequence selected from Column A3 of TABLE 1, and wherein at least a portion of the guide nucleic acid comprises a nucleobase sequence that is 100% identical or 100% reverse complementary to a sequence selected from Column B3 of TABLE 1, wherein the sequence from Column A3 and the sequence from Column B3 are in the same row of TABLE 1.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column D1 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 50% identical to a sequence selected from Column D1 of TABLE 2, and wherein at least a portion of the guide nucleic acid comprises a nucleobase sequence that is at least about 50% identical or at least about 50% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 60% identical to a sequence selected from Column D1 of TABLE 2, and wherein at least a portion of the guide nucleic acid comprises a nucleobase sequence that is at least about 60% identical or at least about 60% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 70% identical to a sequence selected from Column D1 of TABLE 2, and wherein at least a portion of the guide nucleic acid comprises a nucleobase sequence that is at least about 70% identical or at least about 70% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 80% identical to a sequence selected from Column D1 of TABLE 2, and wherein at least a portion of the guide nucleic acid comprises a nucleobase sequence that is at least about or 80% identical or at least about 80% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 90% identical to a sequence selected from Column D1 of TABLE 2, and wherein at least a portion of the guide nucleic acid comprises a nucleobase sequence that is at least about 90% identical or at least about 90% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least about 95% identical to a sequence selected from Column D1 of TABLE 2, and wherein at least a portion of the guide nucleic acid comprises a nucleobase sequence that is at least about 95% identical or at least 95% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is 100% identical to a sequence selected from Column D1 of TABLE 2, and wherein at least a portion of the guide nucleic acid comprises a nucleobase sequence that is 100% identical or 100% reverse complementary to a sequence selected from Column C1 of TABLE 2, wherein the sequence from Column D1 and the sequence from Column C1 are in the same row of TABLE 2.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column D2 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 50% identical to a sequence selected from Column D2 of TABLE 2, and wherein at least a portion of the guide nucleic acid comprises a nucleobase sequence that is at least about 50% identical or at least about 50% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 60% identical to a sequence selected from Column D2 of TABLE 2, and wherein at least a portion of the guide nucleic acid comprises a nucleobase sequence that is at least about 60% identical or at least about 60% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 70% identical to a sequence selected from Column D2 of TABLE 2, and wherein at least a portion of the guide nucleic acid comprises a nucleobase sequence that is at least about 70% identical or at least about 70% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 80% identical to a sequence selected from Column D2 of TABLE 2, and wherein at least a portion of the guide nucleic acid comprises a nucleobase sequence that is at least about or 80% identical or at least about 80% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 90% identical to a sequence selected from Column D2 of TABLE 2, and wherein at least a portion of the guide nucleic acid comprises a nucleobase sequence that is at least about 90% identical or at least about 90% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least about 95% identical to a sequence selected from Column D2 of TABLE 2, and wherein at least a portion of the guide nucleic acid comprises a nucleobase sequence that is at least about 95% identical or at least 95% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is 100% identical to a sequence selected from Column D2 of TABLE 2, and wherein at least a portion of the guide nucleic acid comprises a nucleobase sequence that is 100% identical or 100% reverse complementary to a sequence selected from Column C2 of TABLE 2, wherein the sequence from Column D2 and the sequence from Column C2 are in the same row of TABLE 2.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column D3 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 50% identical to a sequence selected from Column D3 of TABLE 2, and wherein at least a portion of the guide nucleic acid comprises a nucleobase sequence that is at least about 50% identical or at least about 50% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 60% identical to a sequence selected from Column D3 of TABLE 2, and wherein at least a portion of the guide nucleic acid comprises a nucleobase sequence that is at least about 60% identical or at least about 60% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 70% identical to a sequence selected from Column D3 of TABLE 2, and wherein at least a portion of the guide nucleic acid comprises a nucleobase sequence that is at least about 70% identical or at least about 70% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 80% identical to a sequence selected from Column D3 of TABLE 2, and wherein at least a portion of the guide nucleic acid comprises a nucleobase sequence that is at least about or 80% identical or at least about 80% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 90% identical to a sequence selected from Column D3 of TABLE 2, and wherein at least a portion of the guide nucleic acid comprises a nucleobase sequence that is at least about 90% identical or at least about 90% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least about 95% identical to a sequence selected from Column D3 of TABLE 2, and wherein at least a portion of the guide nucleic acid comprises a nucleobase sequence that is at least about 95% identical or at least 95% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is 100% identical to a sequence selected from Column D3 of TABLE 2, and wherein at least a portion of the guide nucleic acid comprises a nucleobase sequence that is 100% identical or 100% reverse complementary to a sequence selected from Column C3 of TABLE 2, wherein the sequence from Column D3 and the sequence from Column C3 are in the same row of TABLE 2.

In some instances, compositions comprise an effector protein or a fusion protein thereof, and a guide nucleic acid, wherein the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column D4 of TABLE 2, and wherein at least a portion of the guide nucleic acid comprises a nucleobase sequence that is at least 50%, at least 55%, at least 60%, at least 65%, at least 70%, at least 75%, at least 80%, at least 85%, at least 90%, at least 95%, or 100% identical or at least 50%, at least 55%, at least 60%, at least 65%, at least 70%, at least 75%, at least 80%, at least 85%, at least 90%, at least 95%, or 100% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 50% identical to a sequence selected from Column D4 of TABLE 2, and wherein at least a portion of the guide nucleic acid comprises a nucleobase sequence that is at least about 50% identical or at least about 50% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 60% identical to a sequence selected from Column D4 of TABLE 2, and wherein at least a portion of the guide nucleic acid comprises a nucleobase sequence that is at least about 60% identical or at least about 60% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 70% identical to a sequence selected from Column D4 of TABLE 2, and wherein at least a portion of the guide nucleic acid comprises a nucleobase sequence that is at least about 70% identical or at least about 70% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 80% identical to a sequence selected from Column D4 of TABLE 2, and wherein at least a portion of the guide nucleic acid comprises a nucleobase sequence that is at least about or 80% identical or at least about 80% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 90% identical to a sequence selected from Column D4 of TABLE 2, and wherein at least a portion of the guide nucleic acid comprises a nucleobase sequence that is at least about 90% identical or at least about 90% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is at least about 95% identical to a sequence selected from Column D4 of TABLE 2, and wherein at least a portion of the guide nucleic acid comprises a nucleobase sequence that is at least about 95% identical or at least 95% reverse complementary to a sequence selected from Column C4 of TABLE 2, wherein the sequence from Column D4 and the sequence from Column C4 are in the same row of TABLE 2.

In some instances, compositions comprise an effector protein or a fusion protein thereof, and a guide nucleic acid, wherein the amino acid sequence of the effector protein is 100% identical to a sequence selected from Column D4 of TABLE 2, and wherein at least a portion of the guide nucleic acid comprises a nucleobase sequence that is 100% identical or 100% reverse complementary to a sequence selected from Column C4 of TABLE 2, wherein the sequence from Column D4 and the sequence from Column C4 are in the same row of TABLE 2.

In some instances, the portion of the guide nucleic acid is the repeat region of the guide nucleic acid. In some instances, the portion of the guide nucleic acid binds the effector protein.

TABLE 1
Effector Proteins

| A1 | B1 | A2 | B2 | A3 | B3 |
|---|---|---|---|---|---|
| SEQ ID NO: | SEQ ID NO: | SEQ ID NO: | SEQ ID NO: | SEQ ID NO: | SEQ ID NO: |
| 1 | 10485 | 1747 | 11996 | 3493 | 13520 |
| 1 | 11112 | 1748 | 11997 | 3494 | 13521 |
| 2 | 10485 | 1749 | 11998 | 3495 | 13522 |
| 2 | 11112 | 1750 | 11999 | 3496 | 13523 |
| 3 | 10486 | 1751 | 12000 | 3497 | 13524 |
| 4 | 10487 | 1752 | 12001 | 3498 | 13525 |
| 5 | 10488 | 1753 | 12001 | 3499 | 13526 |
| 6 | 10489 | 1754 | 12001 | 3500 | 13527 |
| 7 | 10489 | 1755 | 12002 | 3501 | 13528 |
| 8 | 10489 | 1756 | 12003 | 3502 | 13528 |
| 9 | 10489 | 1757 | 12004 | 3503 | 13528 |
| 10 | 10490 | 1758 | 12005 | 3504 | 13528 |
| 11 | 10491 | 1759 | 12006 | 3505 | 13529 |
| 11 | 14123 | 1760 | 12007 | 3506 | 13529 |
| 12 | 10492 | 1761 | 12008 | 3507 | 13530 |
| 12 | 12592 | 1762 | 12009 | 3508 | 13531 |
| 13 | 10493 | 1763 | 12010 | 3509 | 13532 |
| 14 | 10494 | 1764 | 12011 | 3510 | 13533 |
| 15 | 10495 | 1765 | 12012 | 3511 | 13533 |
| 16 | 10496 | 1766 | 12013 | 3512 | 13534 |
| 17 | 10497 | 1767 | 12014 | 3513 | 13535 |
| 18 | 10497 | 1768 | 12015 | 3514 | 13536 |
| 19 | 10498 | 1769 | 12016 | 3515 | 13537 |
| 20 | 10499 | 1770 | 12017 | 3515 | 13556 |
| 21 | 10500 | 1771 | 12018 | 3516 | 13538 |
| 22 | 10501 | 1772 | 12019 | 3517 | 13539 |
| 23 | 10502 | 1773 | 12020 | 3518 | 13540 |
| 24 | 10503 | 1774 | 12021 | 3519 | 13541 |
| 25 | 10504 | 1775 | 12022 | 3520 | 13541 |
| 26 | 10505 | 1776 | 12023 | 3521 | 13541 |
| 27 | 10506 | 1777 | 12024 | 3522 | 13542 |
| 28 | 10507 | 1778 | 12025 | 3523 | 13543 |
| 29 | 10508 | 1779 | 12026 | 3524 | 13544 |
| 30 | 10509 | 1780 | 12027 | 3525 | 13545 |
| 31 | 10510 | 1781 | 12028 | 3526 | 13546 |
| 32 | 10511 | 1782 | 12029 | 3527 | 13547 |
| 33 | 10511 | 1783 | 12030 | 3528 | 13548 |
| 34 | 10512 | 1784 | 12030 | 3529 | 13549 |
| 35 | 10513 | 1785 | 12031 | 3530 | 13550 |
| 36 | 10514 | 1786 | 12032 | 3531 | 13551 |
| 37 | 10515 | 1787 | 12033 | 3532 | 13552 |
| 38 | 10516 | 1788 | 12034 | 3533 | 13553 |
| 39 | 10517 | 1789 | 12035 | 3534 | 13554 |
| 40 | 10518 | 1790 | 12036 | 3535 | 13555 |
| 41 | 10519 | 1791 | 12037 | 3536 | 13557 |
| 42 | 10520 | 1792 | 12038 | 3537 | 13558 |
| 43 | 10521 | 1793 | 12039 | 3538 | 13559 |
| 44 | 10522 | 1794 | 12040 | 3539 | 13560 |
| 45 | 10523 | 1795 | 12041 | 3540 | 13561 |
| 46 | 10524 | 1796 | 12042 | 3541 | 13562 |
| 47 | 10524 | 1797 | 12043 | 3542 | 13563 |
| 48 | 10525 | 1798 | 12044 | 3543 | 13564 |
| 49 | 10526 | 1799 | 12045 | 3544 | 13565 |
| 50 | 10527 | 1800 | 12046 | 3545 | 13566 |
| 51 | 10528 | 1801 | 12046 | 3546 | 13567 |
| 52 | 10528 | 1802 | 12047 | 3547 | 13568 |
| 53 | 10529 | 1803 | 12048 | 3548 | 13569 |
| 54 | 10529 | 1804 | 12049 | 3549 | 13570 |
| 55 | 10530 | 1805 | 12050 | 3550 | 13571 |
| 56 | 10531 | 1806 | 12051 | 3551 | 13572 |
| 57 | 10532 | 1807 | 12052 | 3552 | 13572 |
| 58 | 10533 | 1808 | 12053 | 3553 | 13572 |
| 59 | 10533 | 1809 | 12054 | 3554 | 13572 |
| 60 | 10533 | 1810 | 12055 | 3555 | 13573 |
| 61 | 10534 | 1811 | 12056 | 3556 | 13574 |
| 62 | 10534 | 1812 | 12057 | 3557 | 13575 |
| 63 | 10535 | 1813 | 12058 | 3558 | 13576 |
| 64 | 10536 | 1814 | 12059 | 3559 | 13577 |
| 65 | 10537 | 1815 | 12060 | 3560 | 13578 |
| 66 | 10537 | 1816 | 12061 | 3561 | 13579 |
| 67 | 10538 | 1817 | 12062 | 3562 | 13579 |
| 68 | 10539 | 1818 | 12063 | 3563 | 13580 |
| 69 | 10539 | 1819 | 12064 | 3564 | 13581 |
| 70 | 10539 | 1820 | 12065 | 3565 | 13582 |
| 71 | 10539 | 1821 | 12066 | 3566 | 13583 |
| 72 | 10539 | 1822 | 12067 | 3567 | 13584 |
| 73 | 10539 | 1823 | 12068 | 3568 | 13585 |
| 74 | 10539 | 1824 | 12069 | 3569 | 13586 |
| 75 | 10539 | 1825 | 12070 | 3570 | 13587 |
| 76 | 10539 | 1826 | 12071 | 3571 | 13588 |
| 77 | 10539 | 1827 | 12072 | 3572 | 13589 |
| 78 | 10539 | 1828 | 12072 | 3573 | 13590 |
| 79 | 10539 | 1829 | 12073 | 3574 | 13591 |
| 80 | 10539 | 1830 | 12074 | 3575 | 13592 |
| 81 | 10539 | 1831 | 12075 | 3576 | 13594 |
| 82 | 10539 | 1832 | 12076 | 3577 | 13595 |
| 83 | 10539 | 1833 | 12077 | 3578 | 13595 |
| 84 | 10539 | 1834 | 12078 | 3579 | 13595 |
| 85 | 10539 | 1835 | 12079 | 3580 | 13596 |
| 86 | 10539 | 1836 | 12079 | 3581 | 13597 |
| 87 | 10539 | 1837 | 12080 | 3582 | 13598 |
| 88 | 10539 | 1838 | 12081 | 3583 | 13599 |
| 89 | 10539 | 1838 | 12083 | 3584 | 13600 |
| 90 | 10540 | 1839 | 12082 | 3585 | 13601 |
| 91 | 10541 | 1840 | 12083 | 3586 | 13602 |
| 91 | 10542 | 1841 | 12084 | 3587 | 13603 |
| 91 | 14888 | 1842 | 12085 | 3588 | 13604 |
| 92 | 10543 | 1843 | 12086 | 3589 | 13605 |
| 93 | 10544 | 1844 | 12087 | 3590 | 13606 |
| 94 | 10545 | 1845 | 12088 | 3591 | 13607 |
| 95 | 10545 | 1846 | 12089 | 3592 | 13607 |
| 96 | 10545 | 1847 | 12089 | 3593 | 13608 |
| 97 | 10545 | 1848 | 12089 | 3594 | 13608 |
| 98 | 10546 | 1849 | 12090 | 3595 | 13608 |
| 99 | 10547 | 1850 | 12091 | 3596 | 13609 |
| 100 | 10548 | 1851 | 12092 | 3597 | 13610 |
| 101 | 10549 | 1852 | 12093 | 3598 | 13611 |
| 102 | 10550 | 1853 | 12094 | 3599 | 13612 |
| 103 | 10551 | 1854 | 12095 | 3600 | 13613 |
| 104 | 10552 | 1854 | 13505 | 3601 | 13614 |
| 105 | 10553 | 1855 | 12096 | 3602 | 13615 |
| 106 | 10554 | 1856 | 12097 | 3603 | 13616 |
| 107 | 10555 | 1857 | 12098 | 3604 | 13617 |
| 108 | 10556 | 1858 | 12099 | 3605 | 13618 |
| 109 | 10557 | 1859 | 12100 | 3606 | 13619 |
| 110 | 10558 | 1860 | 12101 | 3607 | 13620 |
| 111 | 10559 | 1861 | 12102 | 3608 | 13621 |
| 112 | 10560 | 1862 | 12103 | 3609 | 13622 |
| 113 | 10561 | 1863 | 12104 | 3610 | 13622 |
| 114 | 10562 | 1864 | 12105 | 3611 | 13623 |
| 115 | 10563 | 1865 | 12106 | 3612 | 13624 |
| 116 | 10564 | 1866 | 12107 | 3613 | 13625 |
| 117 | 10565 | 1867 | 12108 | 3614 | 13626 |
| 118 | 10566 | 1868 | 12109 | 3615 | 13627 |
| 119 | 10567 | 1869 | 12110 | 3616 | 13628 |
| 120 | 10568 | 1870 | 12111 | 3617 | 13629 |
| 121 | 10569 | 1871 | 12112 | 3618 | 13630 |
| 122 | 10570 | 1872 | 12113 | 3619 | 13631 |
| 123 | 10571 | 1873 | 12114 | 3620 | 13632 |
| 124 | 10572 | 1874 | 12115 | 3621 | 13633 |
| 125 | 10573 | 1875 | 12116 | 3622 | 13634 |
| 126 | 10573 | 1876 | 12117 | 3623 | 13635 |
| 127 | 10573 | 1877 | 12118 | 3624 | 13635 |
| 128 | 10574 | 1878 | 12119 | 3625 | 13636 |
| 129 | 10575 | 1879 | 12120 | 3626 | 13637 |
| 130 | 10576 | 1880 | 12121 | 3627 | 13638 |
| 131 | 10576 | 1881 | 12122 | 3628 | 13638 |
| 132 | 10577 | 1882 | 12123 | 3629 | 13639 |
| 133 | 10578 | 1883 | 12124 | 3630 | 13640 |
| 134 | 10579 | 1884 | 12125 | 3631 | 13641 |
| 135 | 10580 | 1885 | 12126 | 3632 | 13642 |
| 136 | 10581 | 1886 | 12126 | 3633 | 13643 |
| 137 | 10582 | 1887 | 12127 | 3634 | 13644 |
| 138 | 10583 | 1888 | 12128 | 3635 | 13645 |
| 139 | 10584 | 1889 | 12129 | 3636 | 13646 |
| 140 | 10585 | 1890 | 12130 | 3637 | 13647 |
| 141 | 10586 | 1891 | 12131 | 3638 | 13648 |
| 142 | 10587 | 1892 | 12132 | 3639 | 13649 |
| 143 | 10587 | 1893 | 12133 | 3640 | 13650 |
| 144 | 10588 | 1894 | 12134 | 3641 | 13651 |
| 145 | 10589 | 1895 | 12135 | 3642 | 13652 |
| 146 | 10590 | 1896 | 12136 | 3643 | 13653 |
| 147 | 10591 | 1897 | 12137 | 3644 | 13654 |
| 148 | 10592 | 1898 | 12138 | 3645 | 13655 |
| 149 | 10593 | 1899 | 12139 | 3646 | 13656 |
| 150 | 10594 | 1900 | 12140 | 3647 | 13657 |
| 151 | 10595 | 1901 | 12141 | 3648 | 13658 |
| 152 | 10596 | 1902 | 12142 | 3649 | 13659 |
| 153 | 10597 | 1903 | 12143 | 3650 | 13660 |
| 154 | 10598 | 1904 | 12144 | 3651 | 13661 |
| 155 | 10599 | 1905 | 12145 | 3652 | 13662 |
| 156 | 10600 | 1906 | 12146 | 3653 | 13663 |
| 157 | 10601 | 1907 | 12147 | 3654 | 13664 |
| 158 | 10602 | 1908 | 12148 | 3655 | 13665 |
| 159 | 10603 | 1909 | 12149 | 3656 | 13666 |
| 160 | 10604 | 1910 | 12150 | 3657 | 13667 |
| 161 | 10605 | 1911 | 12151 | 3658 | 13668 |
| 162 | 10606 | 1912 | 12152 | 3659 | 13669 |
| 163 | 10607 | 1913 | 12153 | 3660 | 13670 |
| 164 | 10608 | 1913 | 12284 | 3661 | 13671 |
| 165 | 10609 | 1914 | 12153 | 3662 | 13672 |
| 166 | 10610 | 1914 | 12284 | 3663 | 13673 |
| 167 | 10611 | 1915 | 12154 | 3664 | 13674 |
| 168 | 10612 | 1916 | 12155 | 3665 | 13675 |
| 169 | 10613 | 1917 | 12156 | 3666 | 13676 |
| 170 | 10614 | 1918 | 12157 | 3667 | 13677 |
| 171 | 10615 | 1919 | 12158 | 3668 | 13678 |
| 172 | 10616 | 1920 | 12159 | 3669 | 13679 |
| 173 | 10617 | 1921 | 12160 | 3670 | 13680 |
| 174 | 10618 | 1922 | 12161 | 3671 | 13681 |
| 175 | 10619 | 1923 | 12162 | 3672 | 13682 |
| 176 | 10620 | 1924 | 12163 | 3673 | 13683 |
| 177 | 10621 | 1925 | 12164 | 3674 | 13684 |
| 178 | 10622 | 1926 | 12165 | 3675 | 13685 |
| 179 | 10623 | 1927 | 12166 | 3676 | 13686 |
| 180 | 10624 | 1928 | 12166 | 3677 | 13687 |
| 181 | 10625 | 1929 | 12167 | 3678 | 13688 |
| 182 | 10626 | 1930 | 12168 | 3679 | 13689 |
| 183 | 10627 | 1931 | 12169 | 3680 | 13690 |
| 184 | 10628 | 1932 | 12170 | 3681 | 13691 |
| 185 | 10629 | 1933 | 12171 | 3682 | 13692 |
| 186 | 10630 | 1934 | 12172 | 3683 | 13693 |
| 187 | 10631 | 1935 | 12173 | 3684 | 13694 |
| 188 | 10632 | 1936 | 12174 | 3685 | 13695 |
| 189 | 10633 | 1937 | 12175 | 3686 | 13696 |
| 190 | 10634 | 1938 | 12176 | 3687 | 13697 |
| 191 | 10635 | 1939 | 12177 | 3688 | 13698 |
| 192 | 10636 | 1940 | 12178 | 3689 | 13699 |
| 193 | 10636 | 1941 | 12179 | 3690 | 13700 |
| 194 | 10637 | 1942 | 12180 | 3691 | 13701 |
| 195 | 10637 | 1943 | 12181 | 3692 | 13702 |
| 196 | 10638 | 1944 | 12182 | 3693 | 13703 |
| 197 | 10639 | 1945 | 12183 | 3694 | 13704 |
| 198 | 10639 | 1946 | 12184 | 3695 | 13705 |
| 199 | 10640 | 1947 | 12185 | 3696 | 13706 |
| 200 | 10641 | 1948 | 12186 | 3697 | 13707 |
| 201 | 10641 | 1949 | 12187 | 3698 | 13708 |
| 202 | 10642 | 1950 | 12188 | 3699 | 13709 |
| 203 | 10643 | 1951 | 12189 | 3700 | 13710 |
| 204 | 10644 | 1952 | 12190 | 3701 | 13711 |
| 205 | 10645 | 1953 | 12191 | 3702 | 13712 |
| 206 | 10646 | 1954 | 12192 | 3703 | 13713 |
| 207 | 10647 | 1954 | 12193 | 3704 | 13714 |
| 208 | 10648 | 1954 | 14998 | 3705 | 13715 |
| 209 | 10649 | 1955 | 12192 | 3706 | 13716 |
| 210 | 10650 | 1955 | 12193 | 3707 | 13717 |
| 210 | 14873 | 1955 | 14998 | 3708 | 13718 |
| 211 | 10651 | 1956 | 12194 | 3709 | 13719 |
| 212 | 10652 | 1957 | 12194 | 3710 | 13720 |
| 213 | 10653 | 1958 | 12195 | 3711 | 13721 |
| 214 | 10654 | 1959 | 12195 | 3712 | 13722 |
| 215 | 10655 | 1959 | 12196 | 3713 | 13723 |
| 216 | 10655 | 1960 | 12197 | 3714 | 13724 |
| 217 | 10656 | 1961 | 12198 | 3715 | 13725 |
| 218 | 10657 | 1962 | 12199 | 3716 | 13726 |
| 219 | 10657 | 1963 | 12199 | 3717 | 13727 |
| 220 | 10658 | 1964 | 12200 | 3718 | 13728 |
| 221 | 10659 | 1965 | 12201 | 3719 | 13729 |
| 222 | 10660 | 1966 | 12202 | 3720 | 13730 |
| 223 | 10661 | 1967 | 12203 | 3721 | 13731 |
| 224 | 10662 | 1968 | 12204 | 3722 | 13732 |
| 225 | 10663 | 1969 | 12204 | 3723 | 13733 |
| 226 | 10664 | 1970 | 12205 | 3724 | 13734 |
| 227 | 10665 | 1971 | 12206 | 3725 | 13735 |
| 228 | 10666 | 1972 | 12207 | 3726 | 13736 |
| 229 | 10667 | 1973 | 12208 | 3727 | 13737 |
| 230 | 10668 | 1974 | 12209 | 3728 | 13738 |
| 231 | 10669 | 1975 | 12210 | 3729 | 13739 |
| 232 | 10670 | 1976 | 12211 | 3730 | 13740 |
| 233 | 10671 | 1976 | 14225 | 3731 | 13741 |
| 234 | 10672 | 1977 | 12212 | 3732 | 13742 |
| 235 | 10673 | 1978 | 12213 | 3733 | 13743 |
| 236 | 10674 | 1979 | 12213 | 3734 | 13744 |
| 237 | 10675 | 1980 | 12213 | 3735 | 13745 |
| 238 | 10676 | 1981 | 12213 | 3736 | 13746 |
| 239 | 10677 | 1982 | 12214 | 3737 | 13747 |
| 240 | 10678 | 1983 | 12215 | 3738 | 13748 |
| 241 | 10679 | 1984 | 12216 | 3739 | 13748 |
| 242 | 10680 | 1985 | 12217 | 3740 | 13748 |
| 243 | 10681 | 1986 | 12218 | 3741 | 13749 |
| 244 | 10682 | 1987 | 12219 | 3742 | 13750 |
| 245 | 10683 | 1988 | 12220 | 3743 | 13751 |
| 246 | 10684 | 1989 | 12221 | 3744 | 13752 |
| 247 | 10685 | 1990 | 12222 | 3745 | 13753 |
| 248 | 10686 | 1991 | 12223 | 3746 | 13754 |
| 249 | 10687 | 1992 | 12224 | 3747 | 13755 |
| 250 | 10688 | 1993 | 12225 | 3748 | 13756 |
| 251 | 10689 | 1994 | 12226 | 3749 | 13757 |
| 252 | 10690 | 1995 | 12227 | 3750 | 13758 |
| 253 | 10691 | 1996 | 12228 | 3751 | 13759 |
| 254 | 10692 | 1997 | 12229 | 3752 | 13760 |
| 255 | 10693 | 1998 | 12230 | 3753 | 13761 |
| 256 | 10694 | 1999 | 12231 | 3754 | 13762 |
| 257 | 10695 | 2000 | 12232 | 3755 | 13763 |
| 258 | 10696 | 2001 | 12233 | 3756 | 13764 |
| 259 | 10697 | 2002 | 12234 | 3757 | 13766 |
| 260 | 10698 | 2003 | 12235 | 3758 | 13767 |
| 261 | 10698 | 2004 | 12236 | 3759 | 13768 |
| 262 | 10699 | 2005 | 12237 | 3760 | 13769 |
| 263 | 10700 | 2006 | 12237 | 3761 | 13770 |
| 264 | 10701 | 2007 | 12238 | 3762 | 13771 |
| 265 | 10702 | 2008 | 12239 | 3763 | 13772 |
| 266 | 10703 | 2009 | 12240 | 3764 | 13773 |
| 267 | 10703 | 2010 | 12241 | 3765 | 13774 |
| 268 | 10703 | 2011 | 12242 | 3766 | 13775 |
| 269 | 10704 | 2012 | 12243 | 3767 | 13776 |
| 270 | 10705 | 2013 | 12244 | 3768 | 13777 |
| 271 | 10706 | 2014 | 12245 | 3769 | 13778 |
| 272 | 10706 | 2015 | 12246 | 3770 | 13779 |
| 273 | 10706 | 2016 | 12247 | 3771 | 13780 |
| 274 | 10707 | 2017 | 12248 | 3772 | 13781 |
| 275 | 10708 | 2018 | 12249 | 3773 | 13782 |
| 276 | 10708 | 2019 | 12250 | 3774 | 13782 |
| 277 | 10708 | 2020 | 12251 | 3775 | 13783 |
| 278 | 10709 | 2021 | 12252 | 3776 | 13784 |
| 279 | 10710 | 2022 | 12253 | 3777 | 13785 |
| 280 | 10710 | 2023 | 12254 | 3778 | 13786 |
| 281 | 10710 | 2024 | 12255 | 3779 | 13787 |
| 282 | 10710 | 2025 | 12256 | 3780 | 13788 |
| 283 | 10710 | 2026 | 12257 | 3781 | 13789 |
| 284 | 10710 | 2027 | 12258 | 3782 | 13790 |
| 285 | 10710 | 2028 | 12259 | 3783 | 13791 |
| 286 | 10710 | 2029 | 12260 | 3784 | 13792 |
| 287 | 10711 | 2030 | 12261 | 3785 | 13793 |
| 288 | 10712 | 2031 | 12262 | 3786 | 13794 |
| 289 | 10713 | 2032 | 12263 | 3787 | 13794 |
| 290 | 10714 | 2033 | 12264 | 3788 | 13795 |
| 291 | 10715 | 2034 | 12265 | 3789 | 13795 |
| 292 | 10716 | 2035 | 12266 | 3790 | 13796 |
| 293 | 10717 | 2036 | 12267 | 3791 | 13797 |
| 294 | 10717 | 2037 | 12268 | 3792 | 13798 |
| 295 | 10718 | 2038 | 12269 | 3793 | 13799 |
| 296 | 10719 | 2039 | 12270 | 3794 | 13800 |
| 297 | 10720 | 2040 | 12271 | 3795 | 13801 |
| 298 | 10721 | 2041 | 12272 | 3796 | 13802 |
| 299 | 10722 | 2042 | 12273 | 3797 | 13803 |
| 300 | 10723 | 2043 | 12274 | 3798 | 13804 |
| 301 | 10724 | 2044 | 12275 | 3799 | 13805 |
| 302 | 10725 | 2045 | 12276 | 3800 | 13806 |
| 303 | 10726 | 2046 | 12277 | 3801 | 13807 |
| 304 | 10727 | 2047 | 12278 | 3802 | 13808 |
| 305 | 10728 | 2048 | 12279 | 3803 | 13809 |
| 306 | 10729 | 2049 | 12280 | 3804 | 13810 |
| 307 | 10730 | 2050 | 12281 | 3805 | 13811 |
| 308 | 10731 | 2051 | 12281 | 3806 | 13812 |
| 309 | 10731 | 2052 | 12282 | 3807 | 13813 |
| 310 | 10732 | 2053 | 12283 | 3808 | 13814 |
| 311 | 10733 | 2054 | 12285 | 3809 | 13815 |
| 312 | 10734 | 2055 | 12286 | 3810 | 13815 |
| 313 | 10735 | 2056 | 12287 | 3811 | 13815 |
| 314 | 10736 | 2057 | 12288 | 3812 | 13816 |
| 315 | 10737 | 2058 | 12289 | 3813 | 13817 |
| 316 | 10738 | 2059 | 12290 | 3814 | 13817 |
| 317 | 10739 | 2060 | 12291 | 3815 | 13817 |
| 318 | 10740 | 2061 | 12292 | 3816 | 13817 |
| 319 | 10741 | 2062 | 12293 | 3817 | 13817 |
| 320 | 10742 | 2063 | 12294 | 3818 | 13817 |
| 321 | 10743 | 2064 | 12295 | 3819 | 13817 |
| 322 | 10744 | 2065 | 12296 | 3820 | 13818 |
| 323 | 10745 | 2066 | 12297 | 3821 | 13818 |
| 324 | 10746 | 2067 | 12298 | 3822 | 13819 |
| 325 | 10747 | 2068 | 12299 | 3823 | 13820 |
| 326 | 10748 | 2069 | 12300 | 3824 | 13821 |
| 327 | 10748 | 2070 | 12301 | 3825 | 13821 |
| 328 | 10749 | 2071 | 12302 | 3826 | 13822 |
| 329 | 10750 | 2072 | 12303 | 3827 | 13822 |
| 330 | 10751 | 2073 | 12304 | 3828 | 13822 |
| 331 | 10752 | 2074 | 12305 | 3829 | 13822 |
| 332 | 10753 | 2075 | 12306 | 3830 | 13823 |
| 333 | 10754 | 2076 | 12307 | 3831 | 13824 |
| 334 | 10755 | 2077 | 12308 | 3832 | 13825 |
| 335 | 10756 | 2078 | 12309 | 3833 | 13825 |
| 335 | 10758 | 2079 | 12310 | 3834 | 13826 |
| 336 | 10757 | 2080 | 12311 | 3835 | 13827 |
| 337 | 10757 | 2081 | 12312 | 3836 | 13828 |
| 338 | 10759 | 2082 | 12313 | 3837 | 13829 |
| 339 | 10760 | 2083 | 12314 | 3838 | 13830 |
| 340 | 10761 | 2084 | 12315 | 3839 | 13831 |
| 341 | 10762 | 2085 | 12316 | 3840 | 13832 |
| 342 | 10763 | 2086 | 12317 | 3841 | 13833 |
| 343 | 10764 | 2087 | 12318 | 3842 | 13834 |
| 344 | 10765 | 2088 | 12319 | 3843 | 13835 |
| 345 | 10766 | 2089 | 12320 | 3844 | 13836 |
| 346 | 10767 | 2090 | 12321 | 3845 | 13836 |
| 347 | 10768 | 2091 | 12321 | 3846 | 13837 |
| 348 | 10769 | 2092 | 12322 | 3847 | 13838 |
| 349 | 10770 | 2093 | 12322 | 3848 | 13839 |
| 350 | 10771 | 2094 | 12323 | 3849 | 13840 |
| 351 | 10772 | 2095 | 12324 | 3850 | 13841 |
| 352 | 10773 | 2096 | 12325 | 3851 | 13842 |
| 353 | 10774 | 2097 | 12326 | 3852 | 13843 |
| 354 | 10775 | 2098 | 12327 | 3853 | 13844 |
| 355 | 10776 | 2099 | 12328 | 3854 | 13845 |
| 356 | 10777 | 2100 | 12329 | 3855 | 13846 |
| 357 | 10777 | 2101 | 12329 | 3856 | 13847 |
| 358 | 10778 | 2102 | 12330 | 3857 | 13848 |
| 359 | 10779 | 2103 | 12331 | 3858 | 13849 |
| 360 | 10780 | 2104 | 12332 | 3859 | 13850 |
| 361 | 10780 | 2105 | 12333 | 3860 | 13851 |
| 362 | 10781 | 2106 | 12334 | 3861 | 13852 |
| 363 | 10782 | 2107 | 12335 | 3862 | 13853 |
| 364 | 10783 | 2108 | 12336 | 3863 | 13854 |
| 365 | 10784 | 2109 | 12337 | 3864 | 13855 |
| 366 | 10785 | 2110 | 12338 | 3865 | 13856 |
| 367 | 10785 | 2111 | 12339 | 3866 | 13857 |
| 368 | 10785 | 2112 | 12340 | 3867 | 13858 |
| 369 | 10786 | 2113 | 12341 | 3868 | 13859 |
| 370 | 10786 | 2114 | 12342 | 3869 | 13860 |
| 371 | 10787 | 2115 | 12343 | 3870 | 13861 |
| 372 | 10788 | 2116 | 12344 | 3871 | 13862 |
| 373 | 10789 | 2117 | 12345 | 3872 | 13863 |
| 374 | 10790 | 2118 | 12346 | 3873 | 13864 |
| 375 | 10791 | 2119 | 12347 | 3874 | 13865 |
| 376 | 10792 | 2120 | 12348 | 3875 | 13866 |
| 377 | 10793 | 2121 | 12349 | 3876 | 13867 |
| 378 | 10794 | 2122 | 12350 | 3877 | 13867 |
| 379 | 10794 | 2123 | 12351 | 3878 | 13868 |
| 380 | 10795 | 2124 | 12352 | 3879 | 13869 |
| 381 | 10796 | 2125 | 12353 | 3880 | 13870 |
| 382 | 10797 | 2126 | 12354 | 3881 | 13871 |
| 383 | 10798 | 2127 | 12355 | 3882 | 13872 |
| 384 | 10799 | 2128 | 12356 | 3883 | 13873 |
| 385 | 10800 | 2129 | 12357 | 3884 | 13874 |
| 386 | 10801 | 2130 | 12358 | 3885 | 13875 |
| 387 | 10802 | 2131 | 12359 | 3886 | 13876 |
| 388 | 10803 | 2132 | 12360 | 3887 | 13877 |
| 389 | 10804 | 2133 | 12361 | 3888 | 13878 |
| 390 | 10805 | 2134 | 12362 | 3889 | 13879 |
| 391 | 10806 | 2134 | 14621 | 3890 | 13880 |
| 392 | 10807 | 2134 | 14627 | 3891 | 13881 |
| 393 | 10808 | 2135 | 12363 | 3892 | 13881 |
| 394 | 10809 | 2136 | 12364 | 3893 | 13882 |
| 395 | 10810 | 2137 | 12365 | 3894 | 13883 |
| 396 | 10811 | 2138 | 12366 | 3895 | 13884 |
| 397 | 10812 | 2139 | 12367 | 3896 | 13885 |
| 398 | 10812 | 2140 | 12367 | 3897 | 13886 |
| 399 | 10813 | 2141 | 12368 | 3898 | 13887 |
| 400 | 10814 | 2142 | 12369 | 3899 | 13888 |
| 401 | 10814 | 2143 | 12370 | 3900 | 13889 |
| 402 | 10815 | 2144 | 12371 | 3901 | 13889 |
| 403 | 10815 | 2145 | 12372 | 3902 | 13889 |
| 404 | 10815 | 2146 | 12373 | 3903 | 13890 |
| 405 | 10815 | 2147 | 12374 | 3904 | 13891 |
| 406 | 10816 | 2148 | 12374 | 3905 | 13892 |
| 407 | 10817 | 2149 | 12375 | 3906 | 13893 |
| 408 | 10818 | 2150 | 12376 | 3907 | 13893 |
| 409 | 10819 | 2151 | 12377 | 3908 | 13893 |
| 410 | 10820 | 2152 | 12378 | 3909 | 13894 |
| 411 | 10821 | 2153 | 12379 | 3910 | 13895 |
| 412 | 10822 | 2154 | 12380 | 3911 | 13896 |
| 413 | 10823 | 2155 | 12381 | 3912 | 13897 |
| 414 | 10824 | 2156 | 12382 | 3913 | 13897 |
| 415 | 10825 | 2157 | 12383 | 3914 | 13897 |
| 416 | 10826 | 2158 | 12384 | 3915 | 13897 |
| 417 | 10827 | 2159 | 12385 | 3916 | 13898 |
| 418 | 10828 | 2160 | 12386 | 3917 | 13899 |
| 419 | 10829 | 2161 | 12387 | 3918 | 13900 |
| 420 | 10829 | 2162 | 12388 | 3919 | 13901 |
| 421 | 10830 | 2163 | 12389 | 3920 | 13902 |
| 422 | 10831 | 2164 | 12390 | 3921 | 13903 |
| 423 | 10832 | 2165 | 12391 | 3922 | 13904 |
| 424 | 10833 | 2165 | 14453 | 3923 | 13905 |
| 425 | 10834 | 2166 | 12392 | 3924 | 13906 |
| 426 | 10835 | 2166 | 14417 | 3925 | 13907 |
| 427 | 10836 | 2167 | 12393 | 3926 | 13907 |
| 428 | 10837 | 2168 | 12394 | 3927 | 13908 |
| 429 | 10838 | 2169 | 12395 | 3928 | 13908 |
| 430 | 10839 | 2170 | 12396 | 3929 | 13909 |
| 431 | 10840 | 2171 | 12397 | 3930 | 13909 |
| 432 | 10841 | 2172 | 12398 | 3931 | 13910 |
| 433 | 10842 | 2173 | 12399 | 3932 | 13911 |
| 434 | 10843 | 2174 | 12400 | 3933 | 13912 |
| 435 | 10844 | 2175 | 12401 | 3934 | 13913 |
| 436 | 10845 | 2176 | 12402 | 3935 | 13914 |
| 437 | 10846 | 2177 | 12403 | 3936 | 13915 |
| 438 | 10847 | 2178 | 12404 | 3937 | 13915 |
| 439 | 10848 | 2179 | 12405 | 3938 | 13916 |
| 440 | 10849 | 2180 | 12406 | 3939 | 13917 |
| 441 | 10850 | 2181 | 12407 | 3940 | 13918 |
| 441 | 10851 | 2182 | 12408 | 3941 | 13919 |
| 442 | 10852 | 2183 | 12409 | 3942 | 13919 |
| 443 | 10853 | 2184 | 12410 | 3943 | 13919 |
| 444 | 10854 | 2185 | 12411 | 3944 | 13920 |
| 445 | 10855 | 2186 | 12412 | 3945 | 13920 |
| 446 | 10856 | 2187 | 12413 | 3946 | 13921 |
| 447 | 10857 | 2188 | 12414 | 3947 | 13921 |
| 448 | 10858 | 2189 | 12415 | 3948 | 13921 |
| 449 | 10859 | 2190 | 12416 | 3949 | 13921 |
| 450 | 10860 | 2191 | 12417 | 3950 | 13921 |
| 451 | 10861 | 2192 | 12418 | 3951 | 13921 |
| 452 | 10862 | 2193 | 12418 | 3952 | 13921 |
| 453 | 10862 | 2194 | 12418 | 3953 | 13921 |
| 454 | 10863 | 2195 | 12418 | 3954 | 13921 |
| 455 | 10864 | 2196 | 12419 | 3955 | 13921 |
| 456 | 10865 | 2197 | 12420 | 3956 | 13921 |
| 457 | 10866 | 2197 | 13189 | 3957 | 13921 |
| 458 | 10867 | 2198 | 12421 | 3958 | 13921 |
| 459 | 10868 | 2199 | 12422 | 3959 | 13921 |
| 460 | 10869 | 2200 | 12423 | 3960 | 13921 |
| 461 | 10870 | 2201 | 12424 | 3961 | 13921 |
| 462 | 10871 | 2202 | 12425 | 3962 | 13921 |
| 463 | 10872 | 2203 | 12426 | 3963 | 13921 |
| 464 | 10873 | 2204 | 12427 | 3964 | 13922 |
| 465 | 10874 | 2205 | 12428 | 3965 | 13922 |
| 466 | 10875 | 2206 | 12429 | 3966 | 13922 |
| 467 | 10876 | 2207 | 12430 | 3967 | 13923 |
| 468 | 10877 | 2208 | 12431 | 3968 | 13924 |
| 469 | 10878 | 2209 | 12432 | 3969 | 13925 |
| 470 | 10878 | 2210 | 12433 | 3970 | 13926 |
| 471 | 10879 | 2211 | 12434 | 3971 | 13927 |
| 472 | 10880 | 2212 | 12436 | 3972 | 13927 |
| 473 | 10881 | 2213 | 12437 | 3973 | 13927 |
| 474 | 10882 | 2214 | 12438 | 3974 | 13927 |
| 475 | 10883 | 2215 | 12439 | 3975 | 13927 |
| 476 | 10884 | 2216 | 12440 | 3976 | 13928 |
| 477 | 10885 | 2217 | 12441 | 3977 | 13929 |
| 478 | 10886 | 2218 | 12442 | 3978 | 13930 |
| 479 | 10887 | 2219 | 12443 | 3979 | 13931 |
| 480 | 10887 | 2220 | 12444 | 3980 | 13932 |
| 481 | 10888 | 2221 | 12445 | 3981 | 13932 |
| 482 | 10889 | 2222 | 12446 | 3982 | 13932 |
| 483 | 10890 | 2223 | 12447 | 3983 | 13933 |
| 484 | 10891 | 2224 | 12448 | 3984 | 13934 |
| 485 | 10892 | 2225 | 12449 | 3985 | 13934 |
| 486 | 10893 | 2226 | 12450 | 3986 | 13934 |
| 487 | 10894 | 2227 | 12451 | 3987 | 13934 |
| 488 | 10895 | 2228 | 12452 | 3988 | 13935 |
| 489 | 10895 | 2229 | 12453 | 3989 | 13936 |
| 490 | 10896 | 2230 | 12453 | 3990 | 13937 |
| 49 | 10897 | 2231 | 12454 | 3991 | 13938 |
| 492 | 10898 | 2232 | 12455 | 3992 | 13939 |
| 493 | 10899 | 2233 | 12455 | 3993 | 13940 |
| 494 | 10900 | 2234 | 12456 | 3994 | 13941 |
| 495 | 10901 | 2235 | 12457 | 3995 | 13942 |
| 496 | 10902 | 2236 | 12458 | 3996 | 13943 |
| 497 | 10903 | 2237 | 12459 | 3997 | 13944 |
| 498 | 10904 | 2238 | 12460 | 3998 | 13945 |
| 499 | 10905 | 2239 | 12461 | 3999 | 13946 |
| 500 | 10906 | 2240 | 12462 | 4000 | 13947 |
| 501 | 10907 | 2241 | 12463 | 4001 | 13948 |
| 502 | 10908 | 2242 | 12464 | 4002 | 13949 |
| 503 | 10909 | 2243 | 12465 | 4003 | 13950 |
| 504 | 10910 | 2244 | 12466 | 4004 | 13950 |
| 505 | 10911 | 2245 | 12467 | 4005 | 13951 |
| 506 | 10911 | 2246 | 12468 | 4006 | 13951 |
| 507 | 10912 | 2247 | 12469 | 4007 | 13952 |
| 508 | 10913 | 2248 | 12470 | 4008 | 13953 |
| 509 | 10914 | 2249 | 12471 | 4009 | 13954 |
| 510 | 10915 | 2250 | 12472 | 4010 | 13955 |
| 511 | 10916 | 2251 | 12473 | 4011 | 13956 |
| 512 | 10917 | 2252 | 12474 | 4012 | 13957 |
| 513 | 10918 | 2253 | 12475 | 4013 | 13958 |
| 514 | 10919 | 2254 | 12477 | 4014 | 13959 |
| 515 | 10920 | 2255 | 12478 | 4015 | 13960 |
| 516 | 10921 | 2255 | 14171 | 4016 | 13961 |
| 517 | 10922 | 2256 | 12479 | 4017 | 13962 |
| 518 | 10923 | 2257 | 12480 | 4018 | 13963 |
| 519 | 10924 | 2258 | 12481 | 4019 | 13964 |
| 520 | 10925 | 2259 | 12482 | 4020 | 13964 |
| 521 | 10926 | 2260 | 12483 | 4021 | 13965 |
| 522 | 10927 | 2261 | 12484 | 4022 | 13966 |
| 523 | 10928 | 2262 | 12485 | 4023 | 13967 |
| 524 | 10929 | 2263 | 12486 | 4024 | 13968 |
| 525 | 10930 | 2264 | 12487 | 4025 | 13969 |
| 526 | 10931 | 2265 | 12488 | 4026 | 13970 |
| 527 | 10932 | 2266 | 12489 | 4027 | 13971 |
| 528 | 10933 | 2267 | 12490 | 4028 | 13972 |
| 528 | 10934 | 2268 | 12491 | 4029 | 13973 |
| 529 | 10935 | 2269 | 12491 | 4030 | 13974 |
| 530 | 10936 | 2270 | 12492 | 4031 | 13975 |
| 531 | 10937 | 2271 | 12493 | 4032 | 13976 |
| 532 | 10938 | 2272 | 12494 | 4033 | 13977 |
| 533 | 10939 | 2273 | 12495 | 4034 | 13978 |
| 534 | 10940 | 2274 | 12496 | 4035 | 13979 |
| 535 | 10941 | 2275 | 12497 | 4036 | 13980 |
| 536 | 10942 | 2276 | 12498 | 4037 | 13982 |
| 537 | 10943 | 2277 | 12499 | 4038 | 13982 |
| 538 | 10944 | 2278 | 12500 | 4039 | 13983 |
| 539 | 10945 | 2279 | 12501 | 4040 | 13984 |
| 540 | 10945 | 2280 | 12502 | 4041 | 13985 |
| 541 | 10945 | 2281 | 12503 | 4042 | 13986 |
| 542 | 10945 | 2282 | 12504 | 4043 | 13987 |
| 543 | 10945 | 2283 | 12505 | 4044 | 13988 |
| 544 | 10946 | 2284 | 12505 | 4045 | 13988 |
| 545 | 10947 | 2285 | 12506 | 4046 | 13989 |
| 546 | 10948 | 2286 | 12507 | 4047 | 13990 |
| 547 | 10949 | 2287 | 12508 | 4048 | 13991 |
| 548 | 10950 | 2288 | 12509 | 4049 | 13991 |
| 549 | 10950 | 2289 | 12510 | 4050 | 13991 |
| 550 | 10951 | 2289 | 12511 | 4051 | 13992 |
| 551 | 10952 | 2290 | 12512 | 4052 | 13993 |
| 552 | 10953 | 2291 | 12513 | 4053 | 13994 |
| 553 | 10954 | 2292 | 12514 | 4054 | 13995 |
| 554 | 10955 | 2293 | 12515 | 4055 | 13996 |
| 555 | 10956 | 2294 | 12516 | 4056 | 13997 |
| 556 | 10957 | 2295 | 12517 | 4057 | 13998 |
| 557 | 10958 | 2296 | 12518 | 4058 | 13999 |
| 558 | 10959 | 2297 | 12519 | 4059 | 14000 |
| 559 | 10960 | 2298 | 12520 | 4060 | 14001 |
| 560 | 10961 | 2299 | 12521 | 4061 | 14002 |
| 561 | 10962 | 2300 | 12522 | 4062 | 14003 |
| 562 | 10963 | 2301 | 12523 | 4063 | 14004 |
| 563 | 10964 | 2302 | 12524 | 4064 | 14005 |
| 564 | 10965 | 2303 | 12525 | 4065 | 14006 |
| 565 | 10966 | 2304 | 12526 | 4066 | 14007 |
| 566 | 10966 | 2305 | 12527 | 4067 | 14008 |
| 567 | 10967 | 2306 | 12528 | 4068 | 14009 |
| 568 | 10968 | 2307 | 12529 | 4069 | 14010 |
| 569 | 10969 | 2308 | 12530 | 4070 | 14011 |
| 570 | 10970 | 2309 | 12531 | 4071 | 14012 |
| 571 | 10970 | 2310 | 12532 | 4072 | 14013 |
| 572 | 10971 | 2311 | 12533 | 4073 | 14014 |
| 573 | 10972 | 2312 | 12534 | 4074 | 14015 |
| 574 | 10973 | 2313 | 12535 | 4075 | 14016 |
| 575 | 10974 | 2314 | 12536 | 4076 | 14017 |
| 576 | 10974 | 2315 | 12537 | 4077 | 14018 |
| 577 | 10975 | 2316 | 12538 | 4078 | 14019 |
| 578 | 10976 | 2317 | 12539 | 4079 | 14020 |
| 579 | 10977 | 2318 | 12540 | 4080 | 14021 |
| 580 | 10978 | 2319 | 12541 | 4081 | 14022 |
| 581 | 10978 | 2320 | 12542 | 4082 | 14023 |
| 582 | 10979 | 2321 | 12543 | 4083 | 14024 |
| 583 | 10980 | 2322 | 12543 | 4084 | 14025 |
| 584 | 10981 | 2323 | 12544 | 4085 | 14025 |
| 585 | 10982 | 2324 | 12545 | 4086 | 14026 |
| 586 | 10983 | 2325 | 12546 | 4087 | 14027 |
| 587 | 10984 | 2326 | 12547 | 4088 | 14027 |
| 588 | 10985 | 2327 | 12548 | 4089 | 14027 |
| 589 | 10986 | 2328 | 12549 | 4090 | 14028 |
| 590 | 10986 | 2329 | 12550 | 4091 | 14029 |
| 591 | 10987 | 2330 | 12551 | 4092 | 14030 |
| 592 | 10988 | 2331 | 12552 | 4093 | 14031 |
| 593 | 10989 | 2332 | 12553 | 4094 | 14032 |
| 594 | 10990 | 2333 | 12554 | 4095 | 14033 |
| 595 | 10991 | 2334 | 12555 | 4096 | 14034 |
| 596 | 10991 | 2335 | 12555 | 4097 | 14035 |
| 597 | 10991 | 2336 | 12556 | 4098 | 14036 |
| 598 | 10991 | 2337 | 12557 | 4099 | 14037 |
| 599 | 10991 | 2338 | 12558 | 4100 | 14038 |
| 600 | 10991 | 2339 | 12559 | 4101 | 14039 |
| 601 | 10992 | 2340 | 12560 | 4102 | 14040 |
| 602 | 10993 | 2341 | 12561 | 4103 | 14041 |
| 603 | 10994 | 2342 | 12562 | 4104 | 14042 |
| 604 | 10995 | 2343 | 12563 | 4105 | 14043 |
| 605 | 10995 | 2344 | 12563 | 4106 | 14044 |
| 606 | 10996 | 2345 | 12563 | 4107 | 14045 |
| 607 | 10997 | 2346 | 12564 | 4108 | 14046 |
| 608 | 10998 | 2347 | 12564 | 4109 | 14047 |
| 609 | 10999 | 2348 | 12564 | 4110 | 14048 |
| 610 | 11000 | 2349 | 12564 | 4111 | 14049 |
| 611 | 11001 | 2350 | 12564 | 4112 | 14050 |
| 612 | 11002 | 2351 | 12564 | 4113 | 14051 |
| 613 | 11003 | 2352 | 12564 | 4114 | 14052 |
| 614 | 11003 | 2353 | 12564 | 4115 | 14053 |
| 615 | 11004 | 2354 | 12564 | 4116 | 14054 |
| 616 | 11005 | 2355 | 12564 | 4117 | 14055 |
| 617 | 11006 | 2356 | 12565 | 4118 | 14056 |
| 618 | 11006 | 2357 | 12566 | 4119 | 14057 |
| 619 | 11006 | 2358 | 12567 | 4120 | 14058 |
| 620 | 11006 | 2359 | 12568 | 4121 | 14059 |
| 621 | 11007 | 2360 | 12569 | 4122 | 14060 |
| 621 | 11627 | 2361 | 12570 | 4123 | 14060 |
| 622 | 11008 | 2362 | 12571 | 4124 | 14060 |
| 623 | 11008 | 2363 | 12572 | 4125 | 14061 |
| 624 | 11008 | 2364 | 12573 | 4126 | 14062 |
| 625 | 11008 | 2365 | 12573 | 4127 | 14063 |
| 626 | 11009 | 2366 | 12574 | 4128 | 14064 |
| 627 | 11010 | 2367 | 12574 | 4129 | 14065 |
| 628 | 11011 | 2368 | 12574 | 4130 | 14065 |
| 629 | 11012 | 2369 | 12574 | 4131 | 14066 |
| 630 | 11013 | 2370 | 12575 | 4132 | 14067 |
| 631 | 11014 | 2371 | 12576 | 4133 | 14068 |
| 632 | 11015 | 2372 | 12577 | 4134 | 14069 |
| 633 | 11016 | 2373 | 12578 | 4135 | 14070 |
| 634 | 11017 | 2374 | 12578 | 4136 | 14071 |
| 635 | 11018 | 2375 | 12578 | 4137 | 14072 |
| 636 | 11019 | 2376 | 12579 | 4138 | 14073 |
| 637 | 11020 | 2377 | 12580 | 4139 | 14074 |
| 638 | 11021 | 2378 | 12581 | 4140 | 14075 |
| 639 | 11022 | 2379 | 12582 | 4141 | 14076 |
| 640 | 11023 | 2380 | 12582 | 4142 | 14077 |
| 641 | 11024 | 2381 | 12583 | 4143 | 14078 |
| 642 | 11025 | 2382 | 12584 | 4144 | 14079 |
| 643 | 11026 | 2383 | 12586 | 4145 | 14080 |
| 644 | 11027 | 2384 | 12587 | 4146 | 14081 |
| 645 | 11028 | 2385 | 12588 | 4147 | 14082 |
| 646 | 11029 | 2386 | 12589 | 4148 | 14083 |
| 647 | 11030 | 2387 | 12590 | 4149 | 14083 |
| 648 | 11031 | 2388 | 12591 | 4150 | 14084 |
| 649 | 11032 | 2389 | 12593 | 4151 | 14085 |
| 650 | 11033 | 2390 | 12594 | 4152 | 14086 |
| 651 | 11034 | 2391 | 12596 | 4153 | 14086 |
| 652 | 11035 | 2392 | 12597 | 4154 | 14087 |
| 653 | 11036 | 2393 | 12598 | 4155 | 14088 |
| 654 | 11037 | 2394 | 12599 | 4156 | 14089 |
| 654 | 11735 | 2395 | 12600 | 4157 | 14090 |
| 655 | 11038 | 2396 | 12601 | 4158 | 14091 |
| 656 | 11039 | 2397 | 12602 | 4159 | 14092 |
| 656 | 13162 | 2398 | 12603 | 4160 | 14093 |
| 657 | 11040 | 2399 | 12604 | 4161 | 14094 |
| 658 | 11041 | 2400 | 12605 | 4162 | 14095 |
| 659 | 11042 | 2401 | 12606 | 4163 | 14096 |
| 660 | 11043 | 2402 | 12607 | 4164 | 14097 |
| 661 | 11044 | 2403 | 12607 | 4165 | 14098 |
| 662 | 11045 | 2404 | 12608 | 4166 | 14099 |
| 663 | 11046 | 2405 | 12609 | 4167 | 14100 |
| 663 | 13409 | 2406 | 12610 | 4168 | 14100 |
| 664 | 11047 | 2407 | 12611 | 4169 | 14101 |
| 665 | 11048 | 2408 | 12612 | 4170 | 14102 |
| 665 | 13593 | 2409 | 12613 | 4171 | 14103 |
| 666 | 11049 | 2410 | 12613 | 4172 | 14104 |
| 667 | 11050 | 2411 | 12614 | 4173 | 14105 |
| 668 | 11051 | 2412 | 12615 | 4174 | 14106 |
| 669 | 11052 | 2413 | 12616 | 4175 | 14107 |
| 670 | 11053 | 2414 | 12616 | 4176 | 14107 |
| 671 | 11054 | 2415 | 12617 | 4177 | 14108 |
| 672 | 11055 | 2416 | 12618 | 4178 | 14109 |
| 673 | 11056 | 2417 | 12619 | 4179 | 14110 |
| 674 | 11057 | 2418 | 12620 | 4180 | 14111 |
| 675 | 11058 | 2419 | 12621 | 4181 | 14111 |
| 676 | 11059 | 2420 | 12621 | 4182 | 14112 |
| 677 | 11060 | 2421 | 12621 | 4183 | 14113 |
| 678 | 11061 | 2422 | 12622 | 4184 | 14114 |
| 679 | 11062 | 2423 | 12623 | 4185 | 14115 |
| 680 | 11063 | 2424 | 12623 | 4186 | 14116 |
| 681 | 11064 | 2425 | 12624 | 4187 | 14117 |
| 682 | 11064 | 2426 | 12625 | 4188 | 14118 |
| 683 | 11065 | 2427 | 12626 | 4189 | 14119 |
| 684 | 11066 | 2428 | 12626 | 4190 | 14120 |
| 685 | 11067 | 2429 | 12627 | 4191 | 14121 |
| 686 | 11068 | 2430 | 12628 | 4192 | 14122 |
| 687 | 11069 | 2431 | 12629 | 4193 | 14122 |
| 688 | 11070 | 2432 | 12630 | 4194 | 14122 |
| 689 | 11071 | 2433 | 12631 | 4195 | 14122 |
| 690 | 11072 | 2434 | 12632 | 4196 | 14122 |
| 691 | 11073 | 2435 | 12633 | 4197 | 14122 |
| 692 | 11074 | 2436 | 12634 | 4198 | 14122 |
| 693 | 11075 | 2437 | 12635 | 4199 | 14124 |
| 694 | 11076 | 2438 | 12636 | 4200 | 14125 |
| 695 | 11077 | 2439 | 12637 | 4201 | 14127 |
| 696 | 11077 | 2440 | 12638 | 4202 | 14128 |
| 697 | 11078 | 2441 | 12639 | 4203 | 14128 |
| 698 | 11079 | 2442 | 12640 | 4204 | 14129 |
| 699 | 11080 | 2443 | 12641 | 4205 | 14130 |
| 700 | 11081 | 2444 | 12641 | 4206 | 14131 |
| 701 | 11082 | 2445 | 12642 | 4207 | 14132 |
| 702 | 11083 | 2446 | 12643 | 4208 | 14133 |
| 703 | 11084 | 2447 | 12644 | 4209 | 14133 |
| 704 | 11085 | 2448 | 12645 | 4210 | 14134 |
| 705 | 11086 | 2449 | 12646 | 4211 | 14135 |
| 706 | 11087 | 2450 | 12647 | 4212 | 14136 |
| 707 | 11088 | 2451 | 12648 | 4213 | 14137 |
| 708 | 11089 | 2452 | 12649 | 4214 | 14138 |
| 709 | 11090 | 2453 | 12650 | 4215 | 14139 |
| 710 | 11091 | 2454 | 12651 | 4216 | 14140 |
| 711 | 11091 | 2455 | 12652 | 4217 | 14141 |
| 712 | 11092 | 2456 | 12653 | 4218 | 14142 |
| 713 | 11093 | 2457 | 12654 | 4219 | 14143 |
| 714 | 11094 | 2458 | 12655 | 4220 | 14144 |
| 715 | 11095 | 2459 | 12656 | 4221 | 14145 |
| 716 | 11096 | 2460 | 12657 | 4222 | 14145 |
| 717 | 11097 | 2461 | 12658 | 4223 | 14146 |
| 718 | 11098 | 2462 | 12659 | 4224 | 14147 |
| 719 | 11099 | 2463 | 12660 | 4225 | 14148 |
| 720 | 11100 | 2464 | 12661 | 4226 | 14149 |
| 721 | 11100 | 2465 | 12662 | 4227 | 14149 |
| 722 | 11101 | 2466 | 12663 | 4228 | 14150 |
| 723 | 11102 | 2467 | 12664 | 4229 | 14151 |
| 724 | 11103 | 2468 | 12665 | 4230 | 14152 |
| 725 | 11104 | 2469 | 12666 | 4231 | 14153 |
| 726 | 11105 | 2470 | 12667 | 4232 | 14154 |
| 727 | 11106 | 2471 | 12668 | 4233 | 14155 |
| 728 | 11107 | 2471 | 12677 | 4234 | 14156 |
| 729 | 11108 | 2472 | 12669 | 4235 | 14157 |
| 730 | 11109 | 2473 | 12670 | 4236 | 14158 |
| 731 | 11110 | 2474 | 12671 | 4237 | 14158 |
| 732 | 11111 | 2475 | 12672 | 4238 | 14159 |
| 732 | 14274 | 2476 | 12673 | 4239 | 14160 |
| 732 | 14361 | 2477 | 12673 | 4240 | 14161 |
| 733 | 11113 | 2478 | 12673 | 4241 | 14161 |
| 734 | 11114 | 2479 | 12674 | 4242 | 14161 |
| 735 | 11114 | 2480 | 12675 | 4243 | 14162 |
| 736 | 11115 | 2481 | 12676 | 4244 | 14163 |
| 737 | 11116 | 2482 | 12678 | 4245 | 14164 |
| 738 | 11117 | 2483 | 12679 | 4246 | 14164 |
| 739 | 11118 | 2484 | 12680 | 4247 | 14164 |
| 740 | 11119 | 2485 | 12681 | 4248 | 14165 |
| 741 | 11120 | 2486 | 12682 | 4249 | 14166 |
| 742 | 11121 | 2487 | 12683 | 4250 | 14167 |
| 743 | 11122 | 2488 | 12684 | 4251 | 14168 |
| 744 | 11123 | 2489 | 12685 | 4252 | 14169 |
| 745 | 11123 | 2490 | 12686 | 4253 | 14170 |
| 746 | 11124 | 2491 | 12687 | 4254 | 14172 |
| 747 | 11125 | 2492 | 12688 | 4255 | 14172 |
| 748 | 11126 | 2493 | 12689 | 4256 | 14173 |
| 749 | 11127 | 2494 | 12690 | 4257 | 14174 |
| 750 | 11128 | 2495 | 12691 | 4258 | 14174 |
| 751 | 11129 | 2496 | 12692 | 4259 | 14176 |
| 751 | 11741 | 2497 | 12693 | 4260 | 14177 |
| 752 | 11130 | 2498 | 12694 | 4261 | 14178 |
| 752 | 11139 | 2499 | 12695 | 4262 | 14179 |
| 753 | 11131 | 2500 | 12696 | 4263 | 14180 |
| 754 | 11132 | 2501 | 12697 | 4264 | 14180 |
| 755 | 11133 | 2502 | 12698 | 4265 | 14181 |
| 756 | 11134 | 2503 | 12699 | 4266 | 14181 |
| 757 | 11135 | 2504 | 12699 | 4267 | 14182 |
| 758 | 11136 | 2505 | 12700 | 4268 | 14183 |
| 759 | 11137 | 2506 | 12701 | 4269 | 14184 |
| 760 | 11138 | 2507 | 12702 | 4270 | 14185 |
| 761 | 11140 | 2508 | 12703 | 4271 | 14186 |
| 762 | 11141 | 2509 | 12704 | 4272 | 14187 |
| 763 | 11142 | 2510 | 12705 | 4273 | 14188 |
| 764 | 11143 | 2511 | 12706 | 4274 | 14188 |
| 765 | 11144 | 2512 | 12707 | 4275 | 14189 |
| 766 | 11145 | 2513 | 12708 | 4276 | 14190 |
| 767 | 11146 | 2514 | 12709 | 4277 | 14191 |
| 768 | 11147 | 2515 | 12710 | 4278 | 14191 |
| 769 | 11148 | 2516 | 12710 | 4279 | 14191 |
| 770 | 11149 | 2517 | 12711 | 4280 | 14191 |
| 770 | 11478 | 2518 | 12711 | 4281 | 14191 |
| 771 | 11150 | 2519 | 12712 | 4282 | 14192 |
| 772 | 11151 | 2520 | 12713 | 4283 | 14192 |
| 773 | 11152 | 2521 | 12714 | 4284 | 14193 |
| 774 | 11153 | 2522 | 12715 | 4285 | 14193 |
| 775 | 11154 | 2523 | 12716 | 4286 | 14193 |
| 776 | 11154 | 2524 | 12717 | 4287 | 14194 |
| 777 | 11155 | 2524 | 14370 | 4288 | 14195 |
| 777 | 13993 | 2524 | 14496 | 4289 | 14196 |
| 778 | 11156 | 2525 | 12718 | 4290 | 14197 |
| 779 | 11157 | 2526 | 12719 | 4291 | 14198 |
| 780 | 11158 | 2527 | 12720 | 4292 | 14199 |
| 781 | 11159 | 2528 | 12721 | 4293 | 14200 |
| 782 | 11160 | 2529 | 12722 | 4294 | 14201 |
| 782 | 12585 | 2530 | 12722 | 4295 | 14202 |
| 783 | 11161 | 2531 | 12723 | 4296 | 14203 |
| 784 | 11162 | 2532 | 12724 | 4297 | 14204 |
| 785 | 11163 | 2533 | 12724 | 4298 | 14205 |
| 786 | 11164 | 2534 | 12725 | 4299 | 14206 |
| 787 | 11165 | 2535 | 12726 | 4300 | 14206 |
| 788 | 11166 | 2536 | 12727 | 4301 | 14207 |
| 789 | 11167 | 2537 | 12728 | 4302 | 14208 |
| 790 | 11168 | 2538 | 12729 | 4303 | 14209 |
| 791 | 11169 | 2539 | 12730 | 4304 | 14210 |
| 792 | 11170 | 2540 | 12730 | 4305 | 14211 |
| 793 | 11171 | 2541 | 12730 | 4306 | 14212 |
| 794 | 11172 | 2542 | 12731 | 4307 | 14213 |
| 795 | 11173 | 2543 | 12732 | 4308 | 14214 |
| 796 | 11174 | 2544 | 12733 | 4309 | 14215 |
| 797 | 11175 | 2545 | 12733 | 4310 | 14216 |
| 798 | 11176 | 2546 | 12734 | 4311 | 14217 |
| 799 | 11177 | 2547 | 12735 | 4312 | 14218 |
| 800 | 11178 | 2548 | 12736 | 4313 | 14219 |
| 801 | 11179 | 2549 | 12737 | 4314 | 14220 |
| 802 | 11180 | 2550 | 12738 | 4315 | 14221 |
| 803 | 11181 | 2551 | 12739 | 4316 | 14221 |
| 804 | 11181 | 2552 | 12740 | 4317 | 14222 |
| 805 | 11182 | 2553 | 12741 | 4318 | 14223 |
| 806 | 11183 | 2554 | 12741 | 4319 | 14224 |
| 807 | 11184 | 2555 | 12742 | 4320 | 14226 |
| 808 | 11185 | 2556 | 12743 | 4321 | 14227 |
| 809 | 11186 | 2557 | 12744 | 4322 | 14228 |
| 810 | 11187 | 2558 | 12745 | 4323 | 14229 |
| 811 | 11188 | 2559 | 12746 | 4324 | 14230 |
| 812 | 11189 | 2560 | 12747 | 4325 | 14231 |
| 813 | 11190 | 2561 | 12747 | 4326 | 14232 |
| 814 | 11191 | 2562 | 12747 | 4327 | 14233 |
| 815 | 11192 | 2563 | 12748 | 4328 | 14234 |
| 816 | 11193 | 2564 | 12749 | 4329 | 14235 |
| 817 | 11194 | 2565 | 12750 | 4330 | 14236 |
| 818 | 11195 | 2566 | 12751 | 4331 | 14237 |
| 819 | 11196 | 2567 | 12751 | 4332 | 14238 |
| 820 | 11197 | 2568 | 12752 | 4333 | 14239 |
| 821 | 11198 | 2569 | 12753 | 4334 | 14239 |
| 822 | 11199 | 2570 | 12754 | 4335 | 14239 |
| 823 | 11199 | 2571 | 12755 | 4336 | 14240 |
| 824 | 11200 | 2572 | 12756 | 4337 | 14241 |
| 825 | 11201 | 2573 | 12757 | 4338 | 14242 |
| 826 | 11202 | 2574 | 12758 | 4339 | 14243 |
| 827 | 11203 | 2575 | 12759 | 4340 | 14244 |
| 828 | 11204 | 2576 | 12759 | 4341 | 14245 |
| 829 | 11205 | 2577 | 12759 | 4342 | 14246 |
| 830 | 11206 | 2578 | 12759 | 4343 | 14247 |
| 831 | 11207 | 2579 | 12759 | 4344 | 14248 |
| 832 | 11208 | 2580 | 12759 | 4345 | 14248 |
| 833 | 11208 | 2581 | 12760 | 4346 | 14248 |
| 834 | 11208 | 2582 | 12761 | 4347 | 14248 |
| 835 | 11209 | 2583 | 12762 | 4348 | 14248 |
| 836 | 11210 | 2584 | 12763 | 4349 | 14249 |
| 837 | 11211 | 2585 | 12764 | 4350 | 14250 |
| 838 | 11211 | 2586 | 12765 | 4351 | 14251 |
| 839 | 11211 | 2587 | 12766 | 4352 | 14252 |
| 840 | 11212 | 2588 | 12767 | 4353 | 14253 |
| 841 | 11213 | 2589 | 12768 | 4354 | 14254 |
| 842 | 11214 | 2590 | 12769 | 4355 | 14255 |
| 843 | 11215 | 2591 | 12770 | 4356 | 14256 |
| 844 | 11216 | 2592 | 12771 | 4357 | 14257 |
| 844 | 11423 | 2593 | 12772 | 4358 | 14258 |
| 845 | 11217 | 2594 | 12773 | 4359 | 14259 |
| 846 | 11218 | 2595 | 12773 | 4360 | 14260 |
| 847 | 11219 | 2596 | 12774 | 4361 | 14261 |
| 848 | 11220 | 2597 | 12775 | 4362 | 14261 |
| 849 | 11221 | 2598 | 12776 | 4363 | 14262 |
| 850 | 11222 | 2598 | 12777 | 4364 | 14263 |
| 851 | 11223 | 2599 | 12777 | 4365 | 14264 |
| 852 | 11224 | 2600 | 12777 | 4366 | 14264 |
| 853 | 11225 | 2601 | 12777 | 4367 | 14265 |
| 854 | 11226 | 2602 | 12778 | 4368 | 14265 |
| 855 | 11227 | 2603 | 12779 | 4369 | 14266 |
| 856 | 11227 | 2604 | 12780 | 4370 | 14267 |
| 857 | 11227 | 2605 | 12781 | 4371 | 14268 |
| 857 | 12435 | 2606 | 12782 | 4372 | 14269 |
| 858 | 11227 | 2607 | 12783 | 4373 | 14270 |
| 859 | 11227 | 2608 | 12784 | 4374 | 14271 |
| 860 | 11227 | 2609 | 12785 | 4375 | 14271 |
| 861 | 11227 | 2610 | 12786 | 4376 | 14272 |
| 862 | 11227 | 2611 | 12787 | 4377 | 14273 |
| 863 | 11228 | 2612 | 12788 | 4378 | 14275 |
| 864 | 11229 | 2613 | 12789 | 4379 | 14276 |
| 865 | 11230 | 2614 | 12790 | 4380 | 14277 |
| 866 | 11231 | 2615 | 12791 | 4381 | 14277 |
| 867 | 11232 | 2616 | 12792 | 4382 | 14278 |
| 868 | 11233 | 2617 | 12793 | 4383 | 14279 |
| 869 | 11234 | 2618 | 12794 | 4384 | 14280 |
| 870 | 11235 | 2619 | 12795 | 4385 | 14281 |
| 871 | 11236 | 2620 | 12796 | 4386 | 14281 |
| 872 | 11237 | 2620 | 13765 | 4387 | 14282 |
| 873 | 11238 | 2621 | 12797 | 4388 | 14283 |
| 874 | 11239 | 2622 | 12798 | 4389 | 14284 |
| 875 | 11240 | 2623 | 12799 | 4390 | 14285 |
| 876 | 11241 | 2624 | 12800 | 4391 | 14286 |
| 877 | 11242 | 2625 | 12801 | 4392 | 14287 |
| 878 | 11243 | 2626 | 12802 | 4393 | 14287 |
| 879 | 11244 | 2627 | 12803 | 4394 | 14287 |
| 880 | 11245 | 2628 | 12804 | 4395 | 14288 |
| 881 | 11246 | 2629 | 12805 | 4396 | 14288 |
| 882 | 11247 | 2630 | 12806 | 4397 | 14289 |
| 883 | 11248 | 2631 | 12807 | 4398 | 14289 |
| 884 | 11249 | 2632 | 12808 | 4399 | 14289 |
| 885 | 11250 | 2633 | 12809 | 4400 | 14290 |
| 886 | 11251 | 2634 | 12810 | 4401 | 14291 |
| 887 | 11252 | 2635 | 12811 | 4402 | 14292 |
| 888 | 11253 | 2636 | 12812 | 4403 | 14293 |
| 889 | 11254 | 2637 | 12813 | 4404 | 14294 |
| 890 | 11255 | 2638 | 12814 | 4405 | 14295 |
| 891 | 11256 | 2639 | 12814 | 4406 | 14296 |
| 892 | 11257 | 2640 | 12815 | 4407 | 14296 |
| 893 | 11257 | 2641 | 12816 | 4408 | 14297 |
| 894 | 11257 | 2642 | 12817 | 4409 | 14298 |
| 895 | 11258 | 2643 | 12818 | 4410 | 14299 |
| 896 | 11259 | 2644 | 12819 | 4411 | 14300 |
| 897 | 11260 | 2645 | 12820 | 4412 | 14301 |
| 898 | 11261 | 2646 | 12821 | 4413 | 14302 |
| 899 | 11262 | 2647 | 12822 | 4414 | 14303 |
| 900 | 11262 | 2648 | 12823 | 4415 | 14304 |
| 901 | 11263 | 2649 | 12823 | 4416 | 14305 |
| 902 | 11263 | 2650 | 12824 | 4417 | 14306 |
| 903 | 11263 | 2651 | 12825 | 4418 | 14307 |
| 904 | 11264 | 2652 | 12826 | 4419 | 14308 |
| 905 | 11265 | 2653 | 12827 | 4420 | 14309 |
| 906 | 11266 | 2654 | 12828 | 4421 | 14310 |
| 907 | 11267 | 2655 | 12829 | 4422 | 14311 |
| 908 | 11268 | 2656 | 12830 | 4423 | 14312 |
| 909 | 11269 | 2657 | 12831 | 4424 | 14313 |
| 910 | 11270 | 2658 | 12832 | 4425 | 14313 |
| 911 | 11271 | 2659 | 12833 | 4426 | 14313 |
| 912 | 11272 | 2660 | 12834 | 4427 | 14313 |
| 913 | 11273 | 2661 | 12835 | 4428 | 14314 |
| 914 | 11274 | 2662 | 12836 | 4429 | 14315 |
| 915 | 11275 | 2663 | 12836 | 4430 | 14316 |
| 916 | 11276 | 2664 | 12837 | 4431 | 14316 |
| 917 | 11277 | 2665 | 12838 | 4432 | 14317 |
| 918 | 11278 | 2666 | 12839 | 4433 | 14318 |
| 919 | 11279 | 2667 | 12840 | 4434 | 14319 |
| 920 | 11280 | 2668 | 12841 | 4435 | 14320 |
| 921 | 11281 | 2669 | 12842 | 4436 | 14321 |
| 922 | 11282 | 2670 | 12843 | 4437 | 14322 |
| 923 | 11282 | 2671 | 12844 | 4438 | 14323 |
| 924 | 11283 | 2672 | 12845 | 4439 | 14324 |
| 925 | 11284 | 2673 | 12846 | 4440 | 14325 |
| 926 | 11285 | 2674 | 12847 | 4441 | 14326 |
| 927 | 11285 | 2675 | 12848 | 4442 | 14326 |
| 928 | 11285 | 2676 | 12849 | 4443 | 14327 |
| 929 | 11285 | 2677 | 12850 | 4444 | 14328 |
| 930 | 11285 | 2678 | 12851 | 4445 | 14329 |
| 931 | 11285 | 2679 | 12852 | 4446 | 14330 |
| 932 | 11285 | 2680 | 12853 | 4447 | 14331 |
| 933 | 11286 | 2681 | 12854 | 4448 | 14332 |
| 934 | 11287 | 2682 | 12855 | 4449 | 14333 |
| 935 | 11288 | 2683 | 12856 | 4450 | 14334 |
| 936 | 11288 | 2684 | 12856 | 4451 | 14335 |
| 937 | 11288 | 2684 | 14923 | 4452 | 14336 |
| 938 | 11288 | 2685 | 12857 | 4453 | 14337 |
| 939 | 11289 | 2686 | 12858 | 4454 | 14338 |
| 940 | 11289 | 2687 | 12859 | 4455 | 14339 |
| 941 | 11290 | 2688 | 12860 | 4456 | 14340 |
| 942 | 11291 | 2689 | 12861 | 4457 | 14341 |
| 943 | 11292 | 2690 | 12862 | 4458 | 14342 |
| 944 | 11293 | 2691 | 12863 | 4459 | 14343 |
| 945 | 11294 | 2692 | 12864 | 4460 | 14344 |
| 946 | 11295 | 2693 | 12865 | 4461 | 14345 |
| 947 | 11295 | 2694 | 12865 | 4462 | 14346 |
| 948 | 11296 | 2695 | 12866 | 4463 | 14347 |
| 949 | 11297 | 2696 | 12867 | 4464 | 14348 |
| 950 | 11298 | 2697 | 12867 | 4465 | 14349 |
| 951 | 11299 | 2698 | 12868 | 4466 | 14350 |
| 952 | 11300 | 2699 | 12869 | 4467 | 14351 |
| 953 | 11301 | 2700 | 12870 | 4468 | 14352 |
| 954 | 11302 | 2701 | 12871 | 4468 | 14956 |
| 955 | 11303 | 2702 | 12872 | 4469 | 14353 |
| 956 | 11304 | 2703 | 12873 | 4470 | 14354 |
| 957 | 11305 | 2704 | 12874 | 4471 | 14355 |
| 958 | 11306 | 2705 | 12875 | 4472 | 14356 |
| 959 | 11307 | 2706 | 12876 | 4473 | 14357 |
| 960 | 11308 | 2707 | 12877 | 4474 | 14358 |
| 961 | 11309 | 2708 | 12878 | 4475 | 14359 |
| 962 | 11310 | 2709 | 12879 | 4476 | 14360 |
| 963 | 11311 | 2710 | 12880 | 4477 | 14362 |
| 964 | 11312 | 2711 | 12881 | 4478 | 14363 |
| 965 | 11313 | 2712 | 12882 | 4479 | 14363 |
| 966 | 11314 | 2713 | 12883 | 4480 | 14364 |
| 967 | 11315 | 2714 | 12884 | 4481 | 14365 |
| 968 | 11316 | 2715 | 12885 | 4482 | 14366 |
| 969 | 11317 | 2716 | 12885 | 4483 | 14367 |
| 970 | 11318 | 2717 | 12886 | 4484 | 14367 |
| 970 | 12476 | 2718 | 12887 | 4485 | 14368 |
| 971 | 11319 | 2719 | 12888 | 4486 | 14369 |
| 972 | 11320 | 2720 | 12889 | 4487 | 14371 |
| 973 | 11321 | 2721 | 12890 | 4488 | 14372 |
| 974 | 11322 | 2722 | 12891 | 4489 | 14373 |
| 975 | 11323 | 2723 | 12892 | 4490 | 14373 |
| 976 | 11324 | 2724 | 12893 | 4491 | 14374 |
| 977 | 11324 | 2725 | 12894 | 4492 | 14374 |
| 978 | 11324 | 2726 | 12895 | 4493 | 14375 |
| 979 | 11325 | 2727 | 12896 | 4494 | 14376 |
| 980 | 11325 | 2728 | 12897 | 4495 | 14377 |
| 981 | 11326 | 2729 | 12898 | 4496 | 14378 |
| 982 | 11326 | 2729 | 12899 | 4497 | 14379 |
| 983 | 11327 | 2730 | 12900 | 4498 | 14380 |
| 984 | 11327 | 2731 | 12901 | 4499 | 14381 |
| 985 | 11327 | 2732 | 12902 | 4500 | 14382 |
| 986 | 11328 | 2733 | 12903 | 4501 | 14383 |
| 987 | 11329 | 2734 | 12904 | 4502 | 14384 |
| 988 | 11330 | 2735 | 12905 | 4503 | 14385 |
| 989 | 11331 | 2736 | 12906 | 4504 | 14386 |
| 990 | 11332 | 2737 | 12906 | 4505 | 14387 |
| 991 | 11333 | 2738 | 12907 | 4506 | 14388 |
| 992 | 11334 | 2739 | 12907 | 4507 | 14389 |
| 993 | 11335 | 2740 | 12908 | 4508 | 14390 |
| 994 | 11336 | 2741 | 12909 | 4509 | 14390 |
| 995 | 11337 | 2742 | 12910 | 4510 | 14390 |
| 996 | 11338 | 2743 | 12911 | 4511 | 14390 |
| 997 | 11339 | 2744 | 12912 | 4512 | 14391 |
| 998 | 11340 | 2745 | 12913 | 4513 | 14392 |
| 999 | 11341 | 2746 | 12914 | 4514 | 14393 |
| 1000 | 11341 | 2747 | 12914 | 4515 | 14394 |
| 1001 | 11342 | 2748 | 12915 | 4516 | 14395 |
| 1002 | 11343 | 2749 | 12916 | 4517 | 14396 |
| 1003 | 11344 | 2750 | 12917 | 4518 | 14397 |
| 1004 | 11345 | 2751 | 12918 | 4519 | 14398 |
| 1005 | 11346 | 2752 | 12919 | 4520 | 14398 |
| 1006 | 11347 | 2753 | 12920 | 4521 | 14399 |
| 1007 | 11348 | 2754 | 12921 | 4522 | 14400 |
| 1008 | 11349 | 2755 | 12922 | 4523 | 14401 |
| 1009 | 11350 | 2756 | 12923 | 4524 | 14401 |
| 1010 | 11351 | 2757 | 12924 | 4525 | 14402 |
| 1011 | 11352 | 2758 | 12925 | 4526 | 14402 |
| 1012 | 11353 | 2759 | 12926 | 4527 | 14403 |
| 1013 | 11354 | 2760 | 12927 | 4528 | 14403 |
| 1014 | 11355 | 2761 | 12928 | 4529 | 14404 |
| 1015 | 11356 | 2762 | 12929 | 4530 | 14405 |
| 1016 | 11357 | 2763 | 12930 | 4531 | 14406 |
| 1017 | 11358 | 2764 | 12931 | 4532 | 14407 |
| 1018 | 11359 | 2765 | 12932 | 4533 | 14408 |
| 1019 | 11360 | 2766 | 12933 | 4534 | 14409 |
| 1020 | 11361 | 2767 | 12934 | 4535 | 14410 |
| 1021 | 11362 | 2768 | 12935 | 4536 | 14411 |
| 1022 | 11363 | 2769 | 12936 | 4537 | 14412 |
| 1023 | 11364 | 2770 | 12937 | 4538 | 14413 |
| 1024 | 11365 | 2771 | 12939 | 4539 | 14414 |
| 1025 | 11366 | 2772 | 12940 | 4540 | 14415 |
| 1026 | 11367 | 2773 | 12940 | 4541 | 14416 |
| 1027 | 11368 | 2774 | 12941 | 4542 | 14417 |
| 1028 | 11369 | 2775 | 12942 | 4543 | 14417 |
| 1029 | 11370 | 2776 | 12943 | 4544 | 14418 |
| 1030 | 11371 | 2777 | 12944 | 4545 | 14419 |
| 1031 | 11372 | 2778 | 12945 | 4546 | 14419 |
| 1032 | 11373 | 2779 | 12946 | 4547 | 14419 |
| 1033 | 11374 | 2780 | 12947 | 4548 | 14420 |
| 1034 | 11375 | 2781 | 12948 | 4549 | 14421 |
| 1035 | 11376 | 2782 | 12949 | 4550 | 14422 |
| 1036 | 11377 | 2783 | 12950 | 4551 | 14423 |
| 1037 | 11378 | 2784 | 12951 | 4552 | 14424 |
| 1038 | 11379 | 2785 | 12952 | 4553 | 14425 |
| 1039 | 11380 | 2786 | 12953 | 4554 | 14425 |
| 1040 | 11380 | 2787 | 12954 | 4555 | 14425 |
| 1041 | 11380 | 2788 | 12954 | 4556 | 14425 |
| 1042 | 11381 | 2789 | 12955 | 4557 | 14425 |
| 1043 | 11382 | 2790 | 12956 | 4558 | 14426 |
| 1044 | 11383 | 2791 | 12956 | 4559 | 14427 |
| 1045 | 11384 | 2792 | 12956 | 4560 | 14428 |
| 1046 | 11385 | 2793 | 12957 | 4561 | 14429 |
| 1047 | 11386 | 2794 | 12958 | 4562 | 14430 |
| 1048 | 11387 | 2795 | 12959 | 4563 | 14431 |
| 1049 | 11388 | 2795 | 13149 | 4564 | 14432 |
| 1050 | 11389 | 2796 | 12960 | 4565 | 14433 |
| 1051 | 11390 | 2797 | 12961 | 4566 | 14434 |
| 1052 | 11391 | 2798 | 12962 | 4567 | 14435 |
| 1053 | 11392 | 2799 | 12962 | 4567 | 14436 |
| 1054 | 11393 | 2799 | 12963 | 4568 | 14437 |
| 1055 | 11394 | 2799 | 14904 | 4569 | 14438 |
| 1056 | 11395 | 2800 | 12964 | 4570 | 14439 |
| 1057 | 11396 | 2801 | 12965 | 4571 | 14440 |
| 1058 | 11397 | 2802 | 12966 | 4572 | 14441 |
| 1059 | 11398 | 2803 | 12967 | 4573 | 14442 |
| 1060 | 11399 | 2804 | 12968 | 4574 | 14443 |
| 1061 | 11400 | 2805 | 12969 | 4575 | 14444 |
| 1062 | 11401 | 2806 | 12970 | 4576 | 14445 |
| 1063 | 11402 | 2807 | 12970 | 4577 | 14446 |
| 1064 | 11402 | 2808 | 12970 | 4578 | 14447 |
| 1064 | 11472 | 2809 | 12971 | 4579 | 14448 |
| 1065 | 11403 | 2810 | 12972 | 4580 | 14449 |
| 1066 | 11404 | 2811 | 12972 | 4581 | 14450 |
| 1067 | 11405 | 2812 | 12972 | 4582 | 14451 |
| 1068 | 11406 | 2813 | 12972 | 4583 | 14452 |
| 1069 | 11407 | 2814 | 12972 | 4584 | 14454 |
| 1070 | 11408 | 2815 | 12973 | 4585 | 14455 |
| 1071 | 11409 | 2816 | 12974 | 4586 | 14456 |
| 1072 | 11409 | 2817 | 12975 | 4587 | 14457 |
| 1073 | 11410 | 2818 | 12976 | 4588 | 14458 |
| 1074 | 11411 | 2819 | 12976 | 4589 | 14459 |
| 1075 | 11412 | 2820 | 12977 | 4590 | 14460 |
| 1076 | 11413 | 2821 | 12977 | 4591 | 14461 |
| 1077 | 11414 | 2822 | 12978 | 4592 | 14462 |
| 1078 | 11415 | 2823 | 12979 | 4593 | 14463 |
| 1079 | 11415 | 2824 | 12980 | 4594 | 14464 |
| 1080 | 11415 | 2825 | 12981 | 4595 | 14465 |
| 1081 | 11416 | 2826 | 12982 | 4596 | 14466 |
| 1082 | 11417 | 2827 | 12983 | 4597 | 14467 |
| 1083 | 11418 | 2828 | 12984 | 4597 | 14891 |
| 1084 | 11418 | 2829 | 12985 | 4598 | 14468 |
| 1085 | 11418 | 2830 | 12986 | 4599 | 14469 |
| 1086 | 11418 | 2831 | 12987 | 4600 | 14470 |
| 1087 | 11418 | 2832 | 12988 | 4601 | 14471 |
| 1088 | 11419 | 2833 | 12989 | 4602 | 14471 |
| 1089 | 11420 | 2834 | 12990 | 4603 | 14472 |
| 1090 | 11421 | 2835 | 12991 | 4604 | 14473 |
| 1091 | 11422 | 2836 | 12992 | 4605 | 14474 |
| 1092 | 11424 | 2837 | 12993 | 4606 | 14475 |
| 1093 | 11425 | 2838 | 12994 | 4607 | 14476 |
| 1094 | 11426 | 2839 | 12995 | 4608 | 14477 |
| 1095 | 11427 | 2840 | 12996 | 4609 | 14477 |
| 1096 | 11428 | 2841 | 12997 | 4610 | 14477 |
| 1097 | 11429 | 2842 | 12998 | 4611 | 14478 |
| 1098 | 11430 | 2843 | 12999 | 4612 | 14479 |
| 1099 | 11431 | 2844 | 13000 | 4613 | 14479 |
| 1100 | 11432 | 2845 | 13001 | 4614 | 14480 |
| 1101 | 11433 | 2846 | 13002 | 4615 | 14481 |
| 1102 | 11434 | 2847 | 13003 | 4616 | 14482 |
| 1103 | 11435 | 2848 | 13004 | 4617 | 14483 |
| 1104 | 11436 | 2849 | 13005 | 4618 | 14484 |
| 1105 | 11437 | 2849 | 13006 | 4619 | 14485 |
| 1106 | 11438 | 2850 | 13007 | 4620 | 14486 |
| 1107 | 11438 | 2851 | 13008 | 4621 | 14487 |
| 1108 | 11438 | 2852 | 13009 | 4622 | 14488 |
| 1109 | 11439 | 2853 | 13010 | 4623 | 14489 |
| 1110 | 11440 | 2854 | 13011 | 4624 | 14489 |
| 1111 | 11441 | 2855 | 13012 | 4625 | 14490 |
| 1112 | 11442 | 2856 | 13013 | 4626 | 14491 |
| 1113 | 11443 | 2857 | 13014 | 4627 | 14492 |
| 1114 | 11444 | 2858 | 13015 | 4628 | 14493 |
| 1115 | 11445 | 2859 | 13016 | 4629 | 14494 |
| 1116 | 11446 | 2860 | 13017 | 4630 | 14495 |
| 1117 | 11447 | 2861 | 13018 | 4631 | 14497 |
| 1118 | 11448 | 2862 | 13019 | 4632 | 14498 |
| 1119 | 11449 | 2863 | 13020 | 4633 | 14499 |
| 1120 | 11450 | 2864 | 13020 | 4634 | 14500 |
| 1121 | 11451 | 2865 | 13021 | 4635 | 14501 |
| 1122 | 11452 | 2866 | 13022 | 4636 | 14502 |
| 1123 | 11453 | 2867 | 13023 | 4637 | 14502 |
| 1124 | 11454 | 2868 | 13024 | 4638 | 14503 |
| 1125 | 11455 | 2869 | 13024 | 4639 | 14504 |
| 1126 | 11456 | 2870 | 13024 | 4639 | 14505 |
| 1127 | 11457 | 2871 | 13024 | 4640 | 14506 |
| 1128 | 11458 | 2872 | 13024 | 4641 | 14507 |
| 1129 | 11459 | 2873 | 13024 | 4642 | 14508 |
| 1130 | 11460 | 2874 | 13024 | 4643 | 14509 |
| 1131 | 11461 | 2875 | 13024 | 4643 | 14509 |
| 1132 | 11462 | 2876 | 13024 | 4643 | 14677 |
| 1133 | 11463 | 2877 | 13024 | 4644 | 14509 |
| 1134 | 11464 | 2878 | 13024 | 4644 | 14509 |
| 1135 | 11465 | 2879 | 13024 | 4644 | 14677 |
| 1136 | 11465 | 2880 | 13025 | 4645 | 14510 |
| 1137 | 11466 | 2881 | 13026 | 4646 | 14511 |
| 1138 | 11467 | 2882 | 13027 | 4647 | 14512 |
| 1139 | 11468 | 2883 | 13027 | 4648 | 14513 |
| 1140 | 11469 | 2884 | 13027 | 4649 | 14514 |
| 1141 | 11469 | 2885 | 13027 | 4650 | 14515 |
| 1142 | 11469 | 2886 | 13027 | 4651 | 14516 |
| 1143 | 11469 | 2887 | 13028 | 4652 | 14517 |
| 1144 | 11469 | 2888 | 13029 | 4653 | 14518 |
| 1145 | 11469 | 2889 | 13030 | 4654 | 14519 |
| 1146 | 11470 | 2890 | 13031 | 4655 | 14519 |
| 1147 | 11471 | 2891 | 13031 | 4656 | 14520 |
| 1148 | 11473 | 2892 | 13032 | 4657 | 14521 |
| 1149 | 11473 | 2893 | 13033 | 4658 | 14521 |
| 1150 | 11473 | 2894 | 13034 | 4659 | 14522 |
| 1151 | 11473 | 2895 | 13035 | 4660 | 14523 |
| 1152 | 11474 | 2896 | 13036 | 4661 | 14524 |
| 1153 | 11474 | 2897 | 13037 | 4662 | 14525 |
| 1154 | 11475 | 2898 | 13038 | 4663 | 14526 |
| 1155 | 11476 | 2899 | 13039 | 4664 | 14527 |
| 1156 | 11477 | 2900 | 13040 | 4665 | 14528 |
| 1157 | 11479 | 2901 | 13041 | 4666 | 14529 |
| 1158 | 11480 | 2902 | 13042 | 4667 | 14530 |
| 1159 | 11481 | 2903 | 13043 | 4668 | 14531 |
| 1160 | 11482 | 2904 | 13044 | 4669 | 14532 |
| 1161 | 11483 | 2905 | 13045 | 4670 | 14533 |
| 1162 | 11484 | 2906 | 13045 | 4671 | 14534 |
| 1163 | 11485 | 2907 | 13045 | 4672 | 14535 |
| 1164 | 11486 | 2908 | 13045 | 4673 | 14536 |
| 1165 | 11487 | 2909 | 13046 | 4674 | 14536 |
| 1166 | 11488 | 2910 | 13047 | 4675 | 14537 |
| 1167 | 11489 | 2911 | 13047 | 4676 | 14538 |
| 1168 | 11490 | 2912 | 13047 | 4677 | 14539 |
| 1169 | 11491 | 2913 | 13047 | 4678 | 14539 |
| 1170 | 11492 | 2914 | 13047 | 4679 | 14540 |
| 1171 | 11492 | 2915 | 13047 | 4680 | 14541 |
| 1172 | 11492 | 2916 | 13048 | 4681 | 14542 |
| 1173 | 11492 | 2917 | 13048 | 4682 | 14543 |
| 1174 | 11492 | 2918 | 13048 | 4683 | 14543 |
| 1175 | 11492 | 2919 | 13048 | 4684 | 14543 |
| 1176 | 11492 | 2920 | 13049 | 4685 | 14544 |
| 1177 | 11492 | 2921 | 13050 | 4686 | 14544 |
| 1178 | 11493 | 2922 | 13051 | 4687 | 14545 |
| 1179 | 11494 | 2923 | 13052 | 4688 | 14545 |
| 1180 | 11495 | 2924 | 13052 | 4689 | 14546 |
| 1181 | 11496 | 2925 | 13053 | 4690 | 14547 |
| 1182 | 11496 | 2926 | 13054 | 4691 | 14548 |
| 1183 | 11497 | 2927 | 13055 | 4692 | 14549 |
| 1184 | 11498 | 2928 | 13056 | 4693 | 14549 |
| 1185 | 11498 | 2929 | 13056 | 4694 | 14550 |
| 1186 | 11498 | 2930 | 13056 | 4695 | 14551 |
| 1187 | 11498 | 2931 | 13056 | 4696 | 14552 |
| 1188 | 11498 | 2932 | 13057 | 4697 | 14553 |
| 1189 | 11498 | 2933 | 13058 | 4698 | 14554 |
| 1190 | 11498 | 2934 | 13058 | 4699 | 14554 |
| 1191 | 11498 | 2935 | 13058 | 4700 | 14555 |
| 1192 | 11498 | 2936 | 13058 | 4701 | 14555 |
| 1193 | 11499 | 2937 | 13059 | 4702 | 14555 |
| 1194 | 11500 | 2938 | 13059 | 4703 | 14555 |
| 1195 | 11501 | 2939 | 13059 | 4704 | 14556 |
| 1196 | 11502 | 2940 | 13060 | 4705 | 14557 |
| 1197 | 11503 | 2941 | 13061 | 4706 | 14558 |
| 1198 | 11504 | 2942 | 13062 | 4707 | 14559 |
| 1199 | 11505 | 2943 | 13062 | 4708 | 14560 |
| 1200 | 11505 | 2944 | 13062 | 4709 | 14561 |
| 1201 | 11505 | 2945 | 13062 | 4710 | 14562 |
| 1202 | 11506 | 2946 | 13062 | 4711 | 14563 |
| 1203 | 11507 | 2947 | 13063 | 4712 | 14564 |
| 1204 | 11508 | 2948 | 13063 | 4713 | 14565 |
| 1205 | 11509 | 2949 | 13063 | 4714 | 14566 |
| 1206 | 11510 | 2950 | 13064 | 4715 | 14567 |
| 1207 | 11511 | 2951 | 13064 | 4716 | 14568 |
| 1208 | 11512 | 2952 | 13064 | 4717 | 14568 |
| 1209 | 11513 | 2953 | 13064 | 4718 | 14568 |
| 1210 | 11514 | 2954 | 13064 | 4719 | 14568 |
| 1211 | 11515 | 2955 | 13064 | 4720 | 14568 |
| 1212 | 11516 | 2956 | 13065 | 4721 | 14568 |
| 1213 | 11517 | 2957 | 13065 | 4722 | 14568 |
| 1214 | 11518 | 2958 | 13066 | 4723 | 14568 |
| 1215 | 11519 | 2959 | 13066 | 4724 | 14568 |
| 1216 | 11520 | 2960 | 13066 | 4725 | 14569 |
| 1217 | 11521 | 2961 | 13067 | 4726 | 14570 |
| 1218 | 11522 | 2962 | 13067 | 4727 | 14571 |
| 1219 | 11523 | 2963 | 13067 | 4728 | 14572 |
| 1220 | 11524 | 2964 | 13067 | 4729 | 14573 |
| 1221 | 11525 | 2965 | 13068 | 4730 | 14574 |
| 1222 | 11526 | 2966 | 13068 | 4731 | 14575 |
| 1223 | 11527 | 2967 | 13068 | 4732 | 14576 |
| 1224 | 11528 | 2968 | 13069 | 4733 | 14577 |
| 1225 | 11529 | 2969 | 13070 | 4734 | 14577 |
| 1226 | 11530 | 2970 | 13071 | 4735 | 14578 |
| 1227 | 11531 | 2971 | 13071 | 4736 | 14579 |
| 1228 | 11532 | 2972 | 13072 | 4737 | 14580 |
| 1229 | 11533 | 2973 | 13072 | 4738 | 14581 |
| 1230 | 11534 | 2974 | 13072 | 4739 | 14581 |
| 1231 | 11535 | 2975 | 13072 | 4740 | 14582 |
| 1232 | 11535 | 2976 | 13072 | 4741 | 14583 |
| 1233 | 11536 | 2977 | 13072 | 4742 | 14584 |
| 1234 | 11537 | 2978 | 13072 | 4743 | 14585 |
| 1235 | 11538 | 2979 | 13073 | 4744 | 14586 |
| 1236 | 11539 | 2980 | 13073 | 4745 | 14587 |
| 1237 | 11540 | 2981 | 13073 | 4746 | 14588 |
| 1238 | 11541 | 2982 | 13073 | 4747 | 14589 |
| 1239 | 11542 | 2983 | 13074 | 4748 | 14590 |
| 1240 | 11543 | 2984 | 13075 | 4749 | 14591 |
| 1241 | 11544 | 2985 | 13076 | 4750 | 14592 |
| 1241 | 14175 | 2986 | 13077 | 4751 | 14593 |
| 1242 | 11545 | 2987 | 13078 | 4752 | 14594 |
| 1243 | 11546 | 2988 | 13079 | 4753 | 14595 |
| 1244 | 11547 | 2989 | 13080 | 4754 | 14596 |
| 1245 | 11548 | 2990 | 13081 | 4755 | 14597 |
| 1246 | 11549 | 2991 | 13082 | 4756 | 14598 |
| 1247 | 11550 | 2992 | 13083 | 4757 | 14599 |
| 1248 | 11551 | 2993 | 13084 | 4758 | 14600 |
| 1249 | 11552 | 2994 | 13085 | 4759 | 14601 |
| 1250 | 11553 | 2995 | 13086 | 4760 | 14602 |
| 1251 | 11554 | 2996 | 13086 | 4761 | 14603 |
| 1252 | 11555 | 2997 | 13087 | 4762 | 14603 |
| 1253 | 11556 | 2998 | 13088 | 4762 | 14963 |
| 1254 | 11557 | 2999 | 13089 | 4763 | 14604 |
| 1255 | 11558 | 3000 | 13090 | 4764 | 14605 |
| 1256 | 11559 | 3001 | 13091 | 4765 | 14606 |
| 1257 | 11560 | 3001 | 15000 | 4766 | 14606 |
| 1257 | 11621 | 3002 | 13091 | 4767 | 14607 |
| 1258 | 11561 | 3002 | 15000 | 4768 | 14608 |
| 1259 | 11562 | 3003 | 13092 | 4769 | 14609 |
| 1260 | 11563 | 3004 | 13093 | 4770 | 14610 |
| 1261 | 11564 | 3005 | 13094 | 4771 | 14611 |
| 1262 | 11564 | 3006 | 13095 | 4772 | 14612 |
| 1263 | 11564 | 3007 | 13096 | 4773 | 14612 |
| 1264 | 11565 | 3008 | 13097 | 4774 | 14613 |
| 1265 | 11565 | 3009 | 13098 | 4775 | 14614 |
| 1266 | 11565 | 3010 | 13099 | 4776 | 14615 |
| 1267 | 11566 | 3011 | 13100 | 4777 | 14616 |
| 1268 | 11567 | 3012 | 13102 | 4778 | 14617 |
| 1269 | 11568 | 3013 | 13103 | 4779 | 14618 |
| 1270 | 11569 | 3014 | 13104 | 4780 | 14619 |
| 1271 | 11569 | 3015 | 13105 | 4781 | 14620 |
| 1272 | 11570 | 3016 | 13106 | 4782 | 14622 |
| 1273 | 11571 | 3017 | 13107 | 4783 | 14623 |
| 1274 | 11572 | 3018 | 13108 | 4784 | 14624 |
| 1275 | 11573 | 3019 | 13109 | 4785 | 14625 |
| 1276 | 11574 | 3020 | 13110 | 4786 | 14626 |
| 1277 | 11575 | 3021 | 13111 | 4787 | 14626 |
| 1278 | 11576 | 3022 | 13112 | 4788 | 14628 |
| 1279 | 11577 | 3023 | 13113 | 4789 | 14628 |
| 1280 | 11578 | 3024 | 13114 | 4790 | 14629 |
| 1281 | 11579 | 3025 | 13115 | 4791 | 14629 |
| 1282 | 11580 | 3026 | 13116 | 4792 | 14630 |
| 1283 | 11581 | 3027 | 13117 | 4793 | 14631 |
| 1284 | 11582 | 3028 | 13118 | 4794 | 14632 |
| 1284 | 12938 | 3029 | 13118 | 4795 | 14633 |
| 1285 | 11583 | 3030 | 13119 | 4796 | 14634 |
| 1286 | 11583 | 3031 | 13120 | 4797 | 14635 |
| 1287 | 11584 | 3032 | 13120 | 4798 | 14636 |
| 1288 | 11585 | 3033 | 13120 | 4799 | 14637 |
| 1289 | 11585 | 3034 | 13120 | 4800 | 14638 |
| 1290 | 11586 | 3035 | 13121 | 4801 | 14639 |
| 1291 | 11587 | 3036 | 13122 | 4802 | 14640 |
| 1292 | 11588 | 3037 | 13123 | 4803 | 14641 |
| 1293 | 11589 | 3038 | 13124 | 4804 | 14641 |
| 1294 | 11590 | 3039 | 13125 | 4805 | 14641 |
| 1295 | 11591 | 3040 | 13126 | 4806 | 14642 |
| 1296 | 11592 | 3041 | 13127 | 4807 | 14643 |
| 1297 | 11593 | 3042 | 13128 | 4808 | 14644 |
| 1298 | 11594 | 3043 | 13129 | 4809 | 14644 |
| 1299 | 11595 | 3044 | 13130 | 4810 | 14644 |
| 1300 | 11596 | 3045 | 13131 | 4811 | 14644 |
| 1301 | 11597 | 3046 | 13132 | 4812 | 14644 |
| 1302 | 11598 | 3047 | 13133 | 4813 | 14644 |
| 1303 | 11599 | 3048 | 13134 | 4814 | 14644 |
| 1304 | 11600 | 3049 | 13135 | 4815 | 14644 |
| 1305 | 11601 | 3050 | 13136 | 4816 | 14644 |
| 1306 | 11602 | 3051 | 13137 | 4817 | 14644 |
| 1307 | 11603 | 3052 | 13138 | 4818 | 14644 |
| 1308 | 11603 | 3053 | 13139 | 4819 | 14644 |
| 1309 | 11604 | 3054 | 13140 | 4820 | 14644 |
| 1310 | 11605 | 3055 | 13141 | 4821 | 14644 |
| 1311 | 11606 | 3056 | 13141 | 4822 | 14644 |
| 1312 | 11607 | 3057 | 13142 | 4823 | 14644 |
| 1313 | 11608 | 3058 | 13143 | 4824 | 14645 |
| 1314 | 11609 | 3059 | 13144 | 4825 | 14645 |
| 1315 | 11610 | 3060 | 13145 | 4826 | 14646 |
| 1316 | 11611 | 3061 | 13146 | 4827 | 14646 |
| 1317 | 11612 | 3062 | 13146 | 4828 | 14647 |
| 1318 | 11613 | 3063 | 13147 | 4829 | 14648 |
| 1319 | 11614 | 3064 | 13148 | 4830 | 14649 |
| 1320 | 11615 | 3065 | 13149 | 4831 | 14650 |
| 1321 | 11616 | 3066 | 13150 | 4832 | 14650 |
| 1322 | 11617 | 3067 | 13151 | 4833 | 14650 |
| 1323 | 11617 | 3068 | 13152 | 4834 | 14650 |
| 1323 | 14025 | 3069 | 13153 | 4835 | 14651 |
| 1324 | 11618 | 3070 | 13153 | 4836 | 14652 |
| 1325 | 11619 | 3071 | 13154 | 4837 | 14653 |
| 1326 | 11620 | 3072 | 13155 | 4838 | 14654 |
| 1327 | 11622 | 3073 | 13156 | 4839 | 14654 |
| 1328 | 11623 | 3074 | 13156 | 4840 | 14655 |
| 1329 | 11624 | 3075 | 13157 | 4841 | 14656 |
| 1330 | 11624 | 3076 | 13158 | 4842 | 14657 |
| 1331 | 11625 | 3077 | 13159 | 4843 | 14658 |
| 1332 | 11625 | 3078 | 13160 | 4844 | 14659 |
| 1333 | 11625 | 3079 | 13161 | 4845 | 14660 |
| 1334 | 11625 | 3080 | 13162 | 4846 | 14661 |
| 1335 | 11626 | 3081 | 13163 | 4847 | 14662 |
| 1336 | 11628 | 3082 | 13163 | 4848 | 14663 |
| 1337 | 11629 | 3083 | 13163 | 4849 | 14664 |
| 1338 | 11630 | 3084 | 13164 | 4850 | 14665 |
| 1339 | 11631 | 3085 | 13164 | 4851 | 14666 |
| 1340 | 11632 | 3086 | 13164 | 4852 | 14667 |
| 1341 | 11633 | 3087 | 13165 | 4853 | 14668 |
| 1342 | 11634 | 3088 | 13166 | 4854 | 14669 |
| 1343 | 11635 | 3089 | 13167 | 4855 | 14670 |
| 1344 | 11636 | 3090 | 13168 | 4856 | 14671 |
| 1345 | 11637 | 3091 | 13169 | 4857 | 14672 |
| 1346 | 11638 | 3092 | 13170 | 4858 | 14673 |
| 1347 | 11639 | 3093 | 13171 | 4859 | 14674 |
| 1348 | 11640 | 3094 | 13171 | 4860 | 14675 |
| 1349 | 11641 | 3095 | 13172 | 4861 | 14676 |
| 1350 | 11642 | 3096 | 13173 | 4862 | 14678 |
| 1351 | 11643 | 3097 | 13174 | 4863 | 14679 |
| 1352 | 11644 | 3098 | 13175 | 4864 | 14680 |
| 1353 | 11645 | 3099 | 13176 | 4865 | 14681 |
| 1354 | 11646 | 3100 | 13176 | 4866 | 14682 |
| 1355 | 11647 | 3101 | 13177 | 4867 | 14683 |
| 1356 | 11648 | 3102 | 13178 | 4868 | 14684 |
| 1357 | 11649 | 3103 | 13179 | 4869 | 14685 |
| 1358 | 11650 | 3104 | 13180 | 4870 | 14686 |
| 1359 | 11651 | 3105 | 13181 | 4871 | 14687 |
| 1360 | 11652 | 3106 | 13182 | 4872 | 14688 |
| 1361 | 11653 | 3107 | 13183 | 4873 | 14689 |
| 1362 | 11654 | 3108 | 13184 | 4873 | 14690 |
| 1363 | 11654 | 3109 | 13185 | 4874 | 14691 |
| 1364 | 11655 | 3110 | 13186 | 4875 | 14692 |
| 1365 | 11656 | 3111 | 13187 | 4876 | 14692 |
| 1366 | 11657 | 3112 | 13188 | 4877 | 14692 |
| 1367 | 11658 | 3113 | 13190 | 4878 | 14692 |
| 1368 | 11659 | 3114 | 13191 | 4879 | 14692 |
| 1369 | 11660 | 3115 | 13192 | 4880 | 14693 |
| 1370 | 11661 | 3116 | 13193 | 4881 | 14694 |
| 1371 | 11662 | 3117 | 13194 | 4882 | 14695 |
| 1372 | 11663 | 3118 | 13194 | 4883 | 14696 |
| 1373 | 11664 | 3119 | 13195 | 4884 | 14696 |
| 1374 | 11665 | 3120 | 13196 | 4885 | 14697 |
| 1375 | 11666 | 3121 | 13197 | 4886 | 14698 |
| 1376 | 11667 | 3122 | 13198 | 4887 | 14699 |
| 1377 | 11668 | 3123 | 13199 | 4888 | 14700 |
| 1378 | 11669 | 3124 | 13201 | 4889 | 14701 |
| 1379 | 11670 | 3125 | 13202 | 4890 | 14702 |
| 1380 | 11671 | 3126 | 13203 | 4891 | 14702 |
| 1381 | 11672 | 3127 | 13204 | 4892 | 14703 |
| 1382 | 11673 | 3128 | 13205 | 4893 | 14704 |
| 1383 | 11674 | 3129 | 13206 | 4894 | 14705 |
| 1384 | 11675 | 3130 | 13207 | 4895 | 14706 |
| 1385 | 11676 | 3131 | 13208 | 4896 | 14707 |
| 1386 | 11677 | 3132 | 13209 | 4897 | 14708 |
| 1387 | 11678 | 3133 | 13210 | 4898 | 14709 |
| 1388 | 11679 | 3134 | 13211 | 4899 | 14710 |
| 1389 | 11680 | 3135 | 13212 | 4900 | 14711 |
| 1390 | 11681 | 3136 | 13213 | 4901 | 14712 |
| 1391 | 11682 | 3137 | 13214 | 4902 | 14713 |
| 1392 | 11683 | 3138 | 13215 | 4903 | 14713 |
| 1393 | 11683 | 3139 | 13216 | 4904 | 14713 |
| 1394 | 11684 | 3140 | 13217 | 4905 | 14714 |
| 1394 | 13101 | 3141 | 13218 | 4906 | 14715 |
| 1395 | 11685 | 3142 | 13219 | 4907 | 14716 |
| 1396 | 11686 | 3143 | 13220 | 4908 | 14717 |
| 1397 | 11687 | 3144 | 13221 | 4909 | 14718 |
| 1398 | 11688 | 3145 | 13222 | 4910 | 14719 |
| 1399 | 11689 | 3146 | 13223 | 4911 | 14720 |
| 1400 | 11690 | 3147 | 13223 | 4912 | 14721 |
| 1400 | 14960 | 3148 | 13224 | 4913 | 14722 |
| 1401 | 11690 | 3149 | 13225 | 4914 | 14722 |
| 1401 | 14960 | 3150 | 13226 | 4915 | 14723 |
| 1402 | 11691 | 3151 | 13227 | 4916 | 14724 |
| 1403 | 11692 | 3152 | 13228 | 4917 | 14725 |
| 1404 | 11693 | 3153 | 13229 | 4918 | 14726 |
| 1405 | 11694 | 3154 | 13230 | 4919 | 14727 |
| 1406 | 11695 | 3155 | 13231 | 4920 | 14727 |
| 1407 | 11696 | 3156 | 13232 | 4921 | 14728 |
| 1408 | 11697 | 3157 | 13233 | 4922 | 14729 |
| 1409 | 11698 | 3158 | 13234 | 4923 | 14730 |
| 1410 | 11699 | 3159 | 13235 | 4924 | 14730 |
| 1411 | 11700 | 3160 | 13236 | 4925 | 14730 |
| 1412 | 11701 | 3161 | 13237 | 4926 | 14730 |
| 1413 | 11702 | 3162 | 13238 | 4927 | 14731 |
| 1414 | 11703 | 3163 | 13239 | 4928 | 14732 |
| 1415 | 11704 | 3164 | 13239 | 4929 | 14733 |
| 1416 | 11705 | 3165 | 13240 | 4930 | 14734 |
| 1417 | 11706 | 3166 | 13240 | 4931 | 14734 |
| 1418 | 11707 | 3167 | 13241 | 4932 | 14735 |
| 1419 | 11708 | 3168 | 13241 | 4933 | 14736 |
| 1420 | 11709 | 3169 | 13241 | 4934 | 14737 |
| 1421 | 11710 | 3170 | 13242 | 4935 | 14738 |
| 1422 | 11711 | 3171 | 13243 | 4936 | 14739 |
| 1423 | 11712 | 3172 | 13244 | 4937 | 14740 |
| 1424 | 11712 | 3173 | 13245 | 4938 | 14741 |
| 1425 | 11713 | 3174 | 13245 | 4939 | 14742 |
| 1426 | 11714 | 3175 | 13246 | 4940 | 14742 |
| 1427 | 11715 | 3176 | 13247 | 4941 | 14742 |
| 1428 | 11716 | 3177 | 13248 | 4942 | 14742 |
| 1429 | 11717 | 3178 | 13249 | 4943 | 14743 |
| 1429 | 12595 | 3179 | 13250 | 4944 | 14744 |
| 1430 | 11718 | 3180 | 13251 | 4945 | 14745 |
| 1431 | 11719 | 3181 | 13252 | 4946 | 14746 |
| 1432 | 11720 | 3182 | 13253 | 4947 | 14747 |
| 1433 | 11721 | 3183 | 13254 | 4948 | 14748 |
| 1434 | 11722 | 3184 | 13254 | 4949 | 14749 |
| 1435 | 11723 | 3185 | 13255 | 4950 | 14750 |
| 1436 | 11724 | 3186 | 13256 | 4951 | 14751 |
| 1437 | 11725 | 3187 | 13257 | 4952 | 14752 |
| 1438 | 11726 | 3187 | 13319 | 4953 | 14753 |
| 1439 | 11727 | 3188 | 13258 | 4954 | 14754 |
| 1440 | 11728 | 3189 | 13259 | 4955 | 14755 |
| 1441 | 11729 | 3189 | 13260 | 4956 | 14756 |
| 1442 | 11730 | 3190 | 13261 | 4957 | 14757 |
| 1443 | 11731 | 3191 | 13262 | 4958 | 14758 |
| 1444 | 11732 | 3192 | 13263 | 4959 | 14759 |
| 1445 | 11733 | 3193 | 13264 | 4960 | 14760 |
| 1446 | 11734 | 3194 | 13265 | 4961 | 14761 |
| 1447 | 11736 | 3195 | 13266 | 4962 | 14762 |
| 1448 | 11737 | 3196 | 13267 | 4963 | 14763 |
| 1449 | 11738 | 3197 | 13267 | 4964 | 14764 |
| 1450 | 11738 | 3198 | 13267 | 4965 | 14765 |
| 1451 | 11739 | 3199 | 13268 | 4966 | 14766 |
| 1452 | 11740 | 3200 | 13269 | 4967 | 14767 |
| 1453 | 11742 | 3201 | 13270 | 4968 | 14767 |
| 1454 | 11743 | 3202 | 13271 | 4969 | 14768 |
| 1455 | 11744 | 3203 | 13272 | 4970 | 14769 |
| 1455 | 13200 | 3204 | 13273 | 4971 | 14770 |
| 1456 | 11745 | 3205 | 13274 | 4972 | 14771 |
| 1457 | 11746 | 3206 | 13275 | 4973 | 14772 |
| 1458 | 11747 | 3207 | 13276 | 4974 | 14773 |
| 1459 | 11748 | 3208 | 13277 | 4975 | 14774 |
| 1460 | 11749 | 3209 | 13278 | 4976 | 14775 |
| 1461 | 11750 | 3210 | 13279 | 4977 | 14776 |
| 1462 | 11751 | 3211 | 13280 | 4978 | 14777 |
| 1463 | 11752 | 3212 | 13281 | 4979 | 14778 |
| 1464 | 11753 | 3213 | 13282 | 4980 | 14779 |
| 1465 | 11754 | 3214 | 13283 | 4981 | 14780 |
| 1466 | 11754 | 3215 | 13284 | 4982 | 14781 |
| 1467 | 11755 | 3216 | 13285 | 4983 | 14782 |
| 1468 | 11756 | 3217 | 13286 | 4984 | 14783 |
| 1469 | 11757 | 3218 | 13287 | 4985 | 14783 |
| 1470 | 11758 | 3219 | 13288 | 4986 | 14784 |
| 1471 | 11758 | 3219 | 13981 | 4987 | 14785 |
| 1472 | 11759 | 3220 | 13289 | 4988 | 14785 |
| 1473 | 11760 | 3221 | 13290 | 4989 | 14786 |
| 1474 | 11761 | 3222 | 13291 | 4990 | 14787 |
| 1475 | 11762 | 3223 | 13292 | 4991 | 14788 |
| 1476 | 11763 | 3224 | 13293 | 4992 | 14789 |
| 1477 | 11764 | 3225 | 13294 | 4993 | 14790 |
| 1478 | 11765 | 3226 | 13294 | 4994 | 14791 |
| 1479 | 11766 | 3227 | 13295 | 4995 | 14792 |
| 1480 | 11767 | 3228 | 13296 | 4996 | 14792 |
| 1481 | 11768 | 3229 | 13297 | 4996 | 14795 |
| 1482 | 11769 | 3230 | 13298 | 4997 | 14792 |
| 1483 | 11770 | 3231 | 13299 | 4997 | 14794 |
| 1484 | 11771 | 3232 | 13300 | 4998 | 14792 |
| 1485 | 11772 | 3233 | 13301 | 4999 | 14792 |
| 1486 | 11773 | 3234 | 13302 | 5000 | 14792 |
| 1487 | 11774 | 3235 | 13303 | 5001 | 14792 |
| 1488 | 11775 | 3236 | 13304 | 5002 | 14792 |
| 1489 | 11776 | 3237 | 13305 | 5003 | 14792 |
| 1490 | 11777 | 3238 | 13306 | 5004 | 14792 |
| 1491 | 11778 | 3239 | 13307 | 5005 | 14792 |
| 1492 | 11779 | 3240 | 13308 | 5006 | 14792 |
| 1493 | 11780 | 3241 | 13309 | 5007 | 14792 |
| 1494 | 11781 | 3242 | 13310 | 5008 | 14792 |
| 1495 | 11782 | 3243 | 13311 | 5009 | 14792 |
| 1496 | 11783 | 3244 | 13312 | 5010 | 14792 |
| 1497 | 11784 | 3245 | 13313 | 5011 | 14792 |
| 1498 | 11785 | 3246 | 13314 | 5012 | 14793 |
| 1499 | 11785 | 3247 | 13315 | 5013 | 14794 |
| 1500 | 11785 | 3248 | 13316 | 5014 | 14794 |
| 1501 | 11785 | 3248 | 13318 | 5015 | 14794 |
| 1502 | 11786 | 3249 | 13316 | 5016 | 14796 |
| 1503 | 11787 | 3250 | 13317 | 5017 | 14797 |
| 1504 | 11788 | 3251 | 13318 | 5018 | 14797 |
| 1505 | 11789 | 3252 | 13318 | 5019 | 14797 |
| 1506 | 11790 | 3253 | 13318 | 5020 | 14797 |
| 1507 | 11791 | 3254 | 13318 | 5021 | 14797 |
| 1508 | 11792 | 3255 | 13320 | 5022 | 14798 |
| 1509 | 11793 | 3256 | 13321 | 5023 | 14799 |
| 1510 | 11794 | 3257 | 13322 | 5024 | 14800 |
| 1511 | 11795 | 3258 | 13323 | 5025 | 14801 |
| 1512 | 11796 | 3259 | 13324 | 5026 | 14802 |
| 1513 | 11797 | 3260 | 13325 | 5027 | 14803 |
| 1514 | 11798 | 3261 | 13326 | 5028 | 14804 |
| 1515 | 11799 | 3262 | 13327 | 5029 | 14805 |
| 1516 | 11800 | 3263 | 13328 | 5030 | 14806 |
| 1517 | 11801 | 3264 | 13329 | 5031 | 14807 |
| 1518 | 11802 | 3265 | 13330 | 5032 | 14808 |
| 1519 | 11803 | 3266 | 13331 | 5033 | 14809 |
| 1520 | 11804 | 3267 | 13331 | 5034 | 14810 |
| 1521 | 11805 | 3268 | 13332 | 5035 | 14811 |
| 1522 | 11806 | 3269 | 13332 | 5036 | 14811 |
| 1523 | 11806 | 3270 | 13333 | 5037 | 14811 |
| 1524 | 11807 | 3271 | 13334 | 5038 | 14812 |
| 1525 | 11808 | 3272 | 13335 | 5039 | 14813 |
| 1526 | 11809 | 3273 | 13336 | 5040 | 14814 |
| 1527 | 11810 | 3274 | 13337 | 5041 | 14815 |
| 1528 | 11811 | 3274 | 13338 | 5042 | 14816 |
| 1529 | 11812 | 3275 | 13339 | 5043 | 14817 |
| 1530 | 11813 | 3276 | 13340 | 5044 | 14818 |
| 1531 | 11814 | 3277 | 13341 | 5045 | 14819 |
| 1532 | 11815 | 3278 | 13341 | 5046 | 14819 |
| 1533 | 11816 | 3279 | 13342 | 5047 | 14819 |
| 1534 | 11817 | 3280 | 13343 | 5048 | 14820 |
| 1535 | 11818 | 3281 | 13343 | 5049 | 14821 |
| 1536 | 11819 | 3282 | 13344 | 5050 | 14822 |
| 1537 | 11820 | 3283 | 13345 | 5051 | 14823 |
| 1538 | 11821 | 3284 | 13346 | 5052 | 14824 |
| 1539 | 11822 | 3285 | 13347 | 5053 | 14825 |
| 1540 | 11823 | 3286 | 13348 | 5054 | 14826 |
| 1541 | 11824 | 3287 | 13349 | 5055 | 14827 |
| 1542 | 11825 | 3288 | 13350 | 5056 | 14828 |
| 1543 | 11826 | 3289 | 13351 | 5057 | 14829 |
| 1544 | 11827 | 3290 | 13352 | 5058 | 14830 |
| 1545 | 11827 | 3291 | 13353 | 5059 | 14831 |
| 1546 | 11828 | 3292 | 13353 | 5060 | 14832 |
| 1547 | 11829 | 3293 | 13353 | 5061 | 14833 |
| 1548 | 11830 | 3294 | 13354 | 5062 | 14834 |
| 1549 | 11830 | 3295 | 13355 | 5063 | 14835 |
| 1550 | 11831 | 3296 | 13356 | 5064 | 14836 |
| 1551 | 11832 | 3297 | 13357 | 5065 | 14837 |
| 1552 | 11832 | 3298 | 13358 | 5066 | 14838 |
| 1553 | 11833 | 3299 | 13359 | 5067 | 14838 |
| 1554 | 11834 | 3300 | 13359 | 5068 | 14838 |
| 1555 | 11835 | 3301 | 13360 | 5069 | 14839 |
| 1556 | 11836 | 3302 | 13361 | 5070 | 14840 |
| 1557 | 11836 | 3303 | 13362 | 5071 | 14841 |
| 1558 | 11837 | 3304 | 13363 | 5072 | 14842 |
| 1559 | 11838 | 3305 | 13364 | 5073 | 14843 |
| 1560 | 11839 | 3306 | 13365 | 5074 | 14843 |
| 1561 | 11840 | 3307 | 13366 | 5075 | 14843 |
| 1562 | 11841 | 3308 | 13366 | 5076 | 14844 |
| 1563 | 11842 | 3309 | 13367 | 5077 | 14845 |
| 1564 | 11843 | 3310 | 13368 | 5078 | 14846 |
| 1565 | 11844 | 3311 | 13369 | 5079 | 14847 |
| 1566 | 11845 | 3312 | 13370 | 5080 | 14847 |
| 1567 | 11846 | 3313 | 13371 | 5081 | 14848 |
| 1568 | 11847 | 3314 | 13372 | 5082 | 14849 |
| 1569 | 11848 | 3315 | 13373 | 5083 | 14850 |
| 1570 | 11849 | 3316 | 13374 | 5084 | 14851 |
| 1571 | 11850 | 3317 | 13375 | 5085 | 14852 |
| 1572 | 11851 | 3318 | 13376 | 5086 | 14852 |
| 1573 | 11852 | 3319 | 13377 | 5087 | 14852 |
| 1574 | 11853 | 3320 | 13378 | 5088 | 14852 |
| 1575 | 11854 | 3321 | 13379 | 5089 | 14852 |
| 1576 | 11855 | 3322 | 13380 | 5090 | 14853 |
| 1577 | 11856 | 3323 | 13380 | 5091 | 14853 |
| 1578 | 11857 | 3324 | 13381 | 5092 | 14854 |
| 1579 | 11858 | 3325 | 13382 | 5093 | 14855 |
| 1580 | 11858 | 3326 | 13383 | 5094 | 14856 |
| 1581 | 11858 | 3327 | 13384 | 5095 | 14857 |
| 1582 | 11858 | 3328 | 13384 | 5096 | 14858 |
| 1583 | 11858 | 3329 | 13384 | 5097 | 14859 |
| 1584 | 11859 | 3330 | 13384 | 5098 | 14860 |
| 1585 | 11859 | 3331 | 13384 | 5099 | 14860 |
| 1586 | 11860 | 3332 | 13384 | 5100 | 14861 |
| 1587 | 11861 | 3333 | 13384 | 5101 | 14861 |
| 1588 | 11861 | 3334 | 13385 | 5102 | 14862 |
| 1589 | 11861 | 3335 | 13386 | 5103 | 14863 |
| 1590 | 11862 | 3336 | 13387 | 5104 | 14863 |
| 1591 | 11863 | 3337 | 13388 | 5105 | 14863 |
| 1592 | 11864 | 3338 | 13389 | 5106 | 14863 |
| 1593 | 11865 | 3339 | 13390 | 5107 | 14863 |
| 1594 | 11866 | 3340 | 13391 | 5108 | 14863 |
| 1595 | 11867 | 3341 | 13392 | 5109 | 14863 |
| 1596 | 11868 | 3342 | 13393 | 5110 | 14863 |
| 1597 | 11869 | 3343 | 13394 | 5111 | 14863 |
| 1598 | 11870 | 3344 | 13395 | 5112 | 14863 |
| 1598 | 14126 | 3344 | 14917 | 5113 | 14863 |
| 1599 | 11871 | 3345 | 13396 | 5114 | 14863 |
| 1600 | 11872 | 3346 | 13397 | 5115 | 14864 |
| 1601 | 11873 | 3347 | 13398 | 5116 | 14865 |
| 1602 | 11874 | 3348 | 13399 | 5117 | 14866 |
| 1603 | 11875 | 3349 | 13400 | 5118 | 14867 |
| 1604 | 11876 | 3350 | 13401 | 5119 | 14868 |
| 1605 | 11877 | 3351 | 13402 | 5120 | 14869 |
| 1606 | 11878 | 3352 | 13403 | 5121 | 14869 |
| 1607 | 11879 | 3353 | 13404 | 5122 | 14869 |
| 1608 | 11880 | 3354 | 13405 | 5123 | 14869 |
| 1609 | 11880 | 3355 | 13405 | 5124 | 14870 |
| 1610 | 11880 | 3356 | 13406 | 5125 | 14871 |
| 1611 | 11880 | 3357 | 13407 | 5126 | 14872 |
| 1612 | 11880 | 3358 | 13408 | 5127 | 14874 |
| 1613 | 11880 | 3359 | 13409 | 5128 | 14875 |
| 1614 | 11880 | 3360 | 13410 | 5129 | 14876 |
| 1615 | 11881 | 3361 | 13411 | 5130 | 14877 |
| 1616 | 11882 | 3362 | 13412 | 5131 | 14878 |
| 1617 | 11883 | 3363 | 13413 | 5132 | 14879 |
| 1618 | 11884 | 3363 | 13421 | 5133 | 14880 |
| 1619 | 11885 | 3364 | 13414 | 5134 | 14881 |
| 1620 | 11886 | 3365 | 13415 | 5135 | 14882 |
| 1621 | 11887 | 3366 | 13416 | 5136 | 14883 |
| 1622 | 11888 | 3367 | 13417 | 5137 | 14884 |
| 1623 | 11889 | 3368 | 13417 | 5138 | 14885 |
| 1624 | 11890 | 3369 | 13418 | 5139 | 14886 |
| 1625 | 11891 | 3370 | 13419 | 5140 | 14887 |
| 1626 | 11892 | 3371 | 13420 | 5141 | 14889 |
| 1627 | 11892 | 3372 | 13422 | 5142 | 14890 |
| 1628 | 11893 | 3373 | 13423 | 5143 | 14892 |
| 1629 | 11894 | 3374 | 13424 | 5144 | 14893 |
| 1630 | 11895 | 3375 | 13425 | 5145 | 14894 |
| 1631 | 11896 | 3376 | 13426 | 5146 | 14895 |
| 1632 | 11897 | 3377 | 13426 | 5147 | 14896 |
| 1633 | 11898 | 3378 | 13427 | 5148 | 14897 |
| 1634 | 11899 | 3379 | 13427 | 5149 | 14898 |
| 1635 | 11900 | 3380 | 13427 | 5150 | 14899 |
| 1636 | 11901 | 3381 | 13427 | 5151 | 14900 |
| 1637 | 11901 | 3382 | 13428 | 5152 | 14901 |
| 1638 | 11902 | 3383 | 13429 | 5153 | 14902 |
| 1639 | 11903 | 3384 | 13429 | 5154 | 14903 |
| 1640 | 11904 | 3385 | 13430 | 5155 | 14905 |
| 1641 | 11905 | 3386 | 13431 | 5156 | 14906 |
| 1642 | 11906 | 3387 | 13432 | 5157 | 14907 |
| 1643 | 11907 | 3388 | 13433 | 5158 | 14908 |
| 1644 | 11908 | 3389 | 13434 | 5159 | 14909 |
| 1645 | 11909 | 3390 | 13435 | 5160 | 14910 |
| 1646 | 11910 | 3391 | 13435 | 5161 | 14911 |
| 1647 | 11911 | 3392 | 13435 | 5162 | 14911 |
| 1648 | 11912 | 3393 | 13435 | 5163 | 14912 |
| 1649 | 11913 | 3394 | 13436 | 5164 | 14913 |
| 1650 | 11914 | 3395 | 13437 | 5165 | 14914 |
| 1651 | 11915 | 3396 | 13438 | 5166 | 14915 |
| 1652 | 11916 | 3397 | 13439 | 5167 | 14916 |
| 1653 | 11917 | 3398 | 13440 | 5168 | 14918 |
| 1654 | 11918 | 3399 | 13441 | 5169 | 14919 |
| 1655 | 11919 | 3400 | 13442 | 5170 | 14920 |
| 1656 | 11920 | 3401 | 13443 | 5171 | 14921 |
| 1657 | 11921 | 3402 | 13444 | 5172 | 14921 |
| 1658 | 11922 | 3403 | 13445 | 5173 | 14922 |
| 1659 | 11922 | 3404 | 13445 | 5174 | 14924 |
| 1660 | 11922 | 3405 | 13445 | 5175 | 14925 |
| 1661 | 11922 | 3406 | 13446 | 5176 | 14926 |
| 1662 | 11922 | 3407 | 13446 | 5177 | 14927 |
| 1663 | 11922 | 3408 | 13446 | 5178 | 14928 |
| 1664 | 11922 | 3409 | 13446 | 5179 | 14929 |
| 1665 | 11922 | 3410 | 13446 | 5180 | 14930 |
| 1666 | 11923 | 3411 | 13446 | 5181 | 14931 |
| 1667 | 11924 | 3412 | 13447 | 5182 | 14932 |
| 1668 | 11925 | 3413 | 13448 | 5183 | 14933 |
| 1669 | 11926 | 3414 | 13449 | 5184 | 14934 |
| 1670 | 11927 | 3415 | 13450 | 5185 | 14935 |
| 1671 | 11928 | 3416 | 13451 | 5186 | 14935 |
| 1672 | 11929 | 3417 | 13452 | 5187 | 14936 |
| 1673 | 11930 | 3418 | 13453 | 5188 | 14937 |
| 1674 | 11931 | 3419 | 13454 | 5189 | 14938 |
| 1675 | 11932 | 3420 | 13455 | 5190 | 14939 |
| 1676 | 11933 | 3421 | 13456 | 5191 | 14940 |
| 1677 | 11933 | 3422 | 13457 | 5192 | 14941 |
| 1678 | 11933 | 3423 | 13458 | 5193 | 14942 |
| 1679 | 11934 | 3424 | 13459 | 5194 | 14943 |
| 1680 | 11935 | 3425 | 13460 | 5195 | 14944 |
| 1681 | 11936 | 3426 | 13461 | 5196 | 14945 |
| 1682 | 11937 | 3427 | 13462 | 5197 | 14946 |
| 1683 | 11938 | 3428 | 13463 | 5198 | 14947 |
| 1684 | 11939 | 3429 | 13464 | 5199 | 14948 |
| 1685 | 11940 | 3430 | 13464 | 5200 | 14949 |
| 1686 | 11941 | 3431 | 13465 | 5201 | 14950 |
| 1687 | 11942 | 3432 | 13466 | 5202 | 14951 |
| 1688 | 11943 | 3433 | 13467 | 5203 | 14952 |
| 1689 | 11944 | 3434 | 13467 | 5204 | 14953 |
| 1690 | 11945 | 3435 | 13468 | 5205 | 14954 |
| 1691 | 11946 | 3436 | 13469 | 5206 | 14955 |
| 1692 | 11947 | 3437 | 13470 | 5207 | 14957 |
| 1693 | 11948 | 3438 | 13471 | 5208 | 14958 |
| 1694 | 11949 | 3439 | 13472 | 5209 | 14959 |
| 1695 | 11950 | 3440 | 13473 | 5210 | 14961 |
| 1696 | 11951 | 3441 | 13474 | 5211 | 14962 |
| 1697 | 11951 | 3442 | 13474 | 5212 | 14964 |
| 1698 | 11952 | 3443 | 13474 | 5213 | 14965 |
| 1699 | 11953 | 3444 | 13475 | 5214 | 14966 |
| 1700 | 11954 | 3445 | 13475 | 5215 | 14967 |
| 1701 | 11955 | 3446 | 13475 | 5216 | 14968 |
| 1702 | 11956 | 3447 | 13475 | 5217 | 14969 |
| 1702 | 12762 | 3448 | 13475 | 5218 | 14970 |
| 1703 | 11957 | 3449 | 13476 | 5219 | 14971 |
| 1704 | 11958 | 3450 | 13477 | 5220 | 14972 |
| 1705 | 11959 | 3451 | 13478 | 5221 | 14973 |
| 1706 | 11960 | 3452 | 13479 | 5222 | 14974 |
| 1707 | 11961 | 3453 | 13480 | 5223 | 14975 |
| 1708 | 11962 | 3454 | 13481 | 5224 | 14976 |
| 1709 | 11963 | 3455 | 13482 | 5225 | 14977 |
| 1710 | 11964 | 3456 | 13483 | 5226 | 14978 |
| 1711 | 11965 | 3457 | 13484 | 5227 | 14979 |
| 1712 | 11966 | 3458 | 13485 | 5228 | 14980 |
| 1713 | 11967 | 3459 | 13486 | 5229 | 14981 |
| 1714 | 11968 | 3460 | 13487 | 5230 | 14982 |
| 1715 | 11969 | 3461 | 13488 | 5231 | 14983 |
| 1716 | 11970 | 3462 | 13489 | 5232 | 14984 |
| 1717 | 11971 | 3463 | 13490 | 5233 | 14985 |
| 1718 | 11972 | 3464 | 13491 | 5234 | 14986 |
| 1719 | 11973 | 3465 | 13492 | 5235 | 14987 |
| 1720 | 11974 | 3466 | 13493 | 5236 | 14988 |
| 1721 | 11975 | 3467 | 13494 | 5237 | 14989 |
| 1722 | 11976 | 3468 | 13495 | 5238 | 14990 |
| 1723 | 11976 | 3469 | 13496 | 5239 | 14991 |
| 1724 | 11976 | 3470 | 13497 | 5240 | 14992 |
| 1725 | 11976 | 3471 | 13498 | 5241 | 14993 |
| 1726 | 11977 | 3472 | 13499 | 5242 | 14994 |
| 1727 | 11978 | 3473 | 13500 | 5243 | 14995 |
| 1728 | 11979 | 3474 | 13501 | 5244 | 14996 |
| 1729 | 11980 | 3475 | 13502 | 5245 | 14997 |
| 1730 | 11980 | 3476 | 13503 | 5246 | 14999 |
| 1731 | 11981 | 3477 | 13503 | 5247 | 15001 |
| 1732 | 11982 | 3478 | 13504 | 5248 | 15002 |
| 1733 | 11983 | 3479 | 13506 | 5249 | 15003 |
| 1734 | 11984 | 3480 | 13507 | 5250 | 15004 |
| 1735 | 11985 | 3481 | 13508 | 5251 | 15005 |
| 1736 | 11986 | 3482 | 13509 | 5252 | 15006 |
| 1737 | 11987 | 3483 | 13510 | 5253 | 15007 |
| 1738 | 11988 | 3484 | 13511 | 5254 | 15008 |
| 1739 | 11989 | 3485 | 13512 | 5255 | 15009 |
| 1740 | 11989 | 3486 | 13513 | 5256 | 15010 |
| 1741 | 11990 | 3487 | 13514 | 5257 | 15011 |
| 1742 | 11991 | 3488 | 13515 | 5258 | 15012 |
| 1743 | 11992 | 3489 | 13516 | 5259 | 15013 |
| 1744 | 11993 | 3490 | 13517 | 5260 | 15014 |
| 1745 | 11994 | 3491 | 13518 | 5261 | 15015 |
| 1746 | 11995 | 3492 | 13519 | | |

TABLE 2
Effector Protein

| C1 | D1 | C2 | D2 | C3 | D3 | C4 | D4 |
|---|---|---|---|---|---|---|---|
| SEQ ID NO: | SEQ ID NO: | SEQ ID NO: | SEQ ID NO: | SEQ ID NO: | SEQ ID NO: | SEQ ID NO: | SEQ ID NO: |
| 24166 | 15022 | 26452 | 17308 | 28738 | 19594 | 31024 | 21880 |
| 24167 | 15023 | 26453 | 17309 | 28739 | 19595 | 31025 | 21881 |
| 24168 | 15024 | 26454 | 17310 | 28740 | 19596 | 31026 | 21882 |
| 24169 | 15025 | 26455 | 17311 | 28741 | 19597 | 31027 | 21883 |
| 24170 | 15026 | 26456 | 17312 | 28742 | 19598 | 31028 | 21884 |
| 24171 | 15027 | 26457 | 17313 | 28743 | 19599 | 31029 | 21885 |
| 24172 | 15028 | 26458 | 17314 | 28744 | 19600 | 31030 | 21886 |
| 24173 | 15029 | 26459 | 17315 | 28745 | 19601 | 31031 | 21887 |
| 24174 | 15030 | 26460 | 17316 | 28746 | 19602 | 31032 | 21888 |
| 24175 | 15031 | 26461 | 17317 | 28747 | 19603 | 31033 | 21889 |
| 24176 | 15032 | 26462 | 17318 | 28748 | 19604 | 31034 | 21890 |
| 24177 | 15033 | 26463 | 17319 | 28749 | 19605 | 31035 | 21891 |
| 24178 | 15034 | 26464 | 17320 | 28750 | 19606 | 31036 | 21892 |
| 24179 | 15035 | 26465 | 17321 | 28751 | 19607 | 31037 | 21893 |
| 24180 | 15036 | 26466 | 17322 | 28752 | 19608 | 31038 | 21894 |
| 24181 | 15037 | 26467 | 17323 | 28753 | 19609 | 31039 | 21895 |
| 24182 | 15038 | 26468 | 17324 | 28754 | 19610 | 31040 | 21896 |
| 24183 | 15039 | 26469 | 17325 | 28755 | 19611 | 31041 | 21897 |
| 24184 | 15040 | 26470 | 17326 | 28756 | 19612 | 31042 | 21898 |
| 24185 | 15041 | 26471 | 17327 | 28757 | 19613 | 31043 | 21899 |
| 24186 | 15042 | 26472 | 17328 | 28758 | 19614 | 31044 | 21900 |
| 24187 | 15043 | 26473 | 17329 | 28759 | 19615 | 31045 | 21901 |
| 24188 | 15044 | 26474 | 17330 | 28760 | 19616 | 31046 | 21902 |
| 24189 | 15045 | 26475 | 17331 | 28761 | 19617 | 31047 | 21903 |
| 24190 | 15046 | 26476 | 17332 | 28762 | 19618 | 31048 | 21904 |
| 24191 | 15047 | 26477 | 17333 | 28763 | 19619 | 31049 | 21905 |
| 24192 | 15048 | 26478 | 17334 | 28764 | 19620 | 31050 | 21906 |
| 24193 | 15049 | 26479 | 17335 | 28765 | 19621 | 31051 | 21907 |
| 24194 | 15050 | 26480 | 17336 | 28766 | 19622 | 31052 | 21908 |
| 24195 | 15051 | 26481 | 17337 | 28767 | 19623 | 31053 | 21909 |
| 24196 | 15052 | 26482 | 17338 | 28768 | 19624 | 31054 | 21910 |
| 24197 | 15053 | 26483 | 17339 | 28769 | 19625 | 31055 | 21911 |
| 24198 | 15054 | 26484 | 17340 | 28770 | 19626 | 31056 | 21912 |
| 24199 | 15055 | 26485 | 17341 | 28771 | 19627 | 31057 | 21913 |
| 24200 | 15056 | 26486 | 17342 | 28772 | 19628 | 31058 | 21914 |
| 24201 | 15057 | 26487 | 17343 | 28773 | 19629 | 31059 | 21915 |
| 24202 | 15058 | 26488 | 17344 | 28774 | 19630 | 31060 | 21916 |
| 24203 | 15059 | 26489 | 17345 | 28775 | 19631 | 31061 | 21917 |
| 24204 | 15060 | 26490 | 17346 | 28776 | 19632 | 31062 | 21918 |
| 24205 | 15061 | 26491 | 17347 | 28777 | 19633 | 31063 | 21919 |
| 24206 | 15062 | 26492 | 17348 | 28778 | 19634 | 31064 | 21920 |
| 24207 | 15063 | 26493 | 17349 | 28779 | 19635 | 31065 | 21921 |
| 24208 | 15064 | 26494 | 17350 | 28780 | 19636 | 31066 | 21922 |
| 24209 | 15065 | 26495 | 17351 | 28781 | 19637 | 31067 | 21923 |
| 24210 | 15066 | 26496 | 17352 | 28782 | 19638 | 31068 | 21924 |
| 24211 | 15067 | 26497 | 17353 | 28783 | 19639 | 31069 | 21925 |
| 24212 | 15068 | 26498 | 17354 | 28784 | 19640 | 31070 | 21926 |
| 24213 | 15069 | 26499 | 17355 | 28785 | 19641 | 31071 | 21927 |
| 24214 | 15070 | 26500 | 17356 | 28786 | 19642 | 31072 | 21928 |
| 24215 | 15071 | 26501 | 17357 | 28787 | 19643 | 31073 | 21929 |
| 24216 | 15072 | 26502 | 17358 | 28788 | 19644 | 31074 | 21930 |
| 24217 | 15073 | 26503 | 17359 | 28789 | 19645 | 31075 | 21931 |
| 24218 | 15074 | 26504 | 17360 | 28790 | 19646 | 31076 | 21932 |
| 24219 | 15075 | 26505 | 17361 | 28791 | 19647 | 31077 | 21933 |
| 24220 | 15076 | 26506 | 17362 | 28792 | 19648 | 31078 | 21934 |
| 24221 | 15077 | 26507 | 17363 | 28793 | 19649 | 31079 | 21935 |
| 24222 | 15078 | 26508 | 17364 | 28794 | 19650 | 31080 | 21936 |
| 24223 | 15079 | 26509 | 17365 | 28795 | 19651 | 31081 | 21937 |
| 24224 | 15080 | 26510 | 17366 | 28796 | 19652 | 31082 | 21938 |
| 24225 | 15081 | 26511 | 17367 | 28797 | 19653 | 31083 | 21939 |
| 24226 | 15082 | 26512 | 17368 | 28798 | 19654 | 31084 | 21940 |
| 24227 | 15083 | 26513 | 17369 | 28799 | 19655 | 31085 | 21941 |
| 24228 | 15084 | 26514 | 17370 | 28800 | 19656 | 31086 | 21942 |
| 24229 | 15085 | 26515 | 17371 | 28801 | 19657 | 31087 | 21943 |
| 24230 | 15086 | 26516 | 17372 | 28802 | 19658 | 31088 | 21944 |
| 24231 | 15087 | 26517 | 17373 | 28803 | 19659 | 31089 | 21945 |
| 24232 | 15088 | 26518 | 17374 | 28804 | 19660 | 31090 | 21946 |
| 24233 | 15089 | 26519 | 17375 | 28805 | 19661 | 31091 | 21947 |
| 24234 | 15090 | 26520 | 17376 | 28806 | 19662 | 31092 | 21948 |
| 24235 | 15091 | 26521 | 17377 | 28807 | 19663 | 31093 | 21949 |
| 24236 | 15092 | 26522 | 17378 | 28808 | 19664 | 31094 | 21950 |
| 24237 | 15093 | 26523 | 17379 | 28809 | 19665 | 31095 | 21951 |
| 24238 | 15094 | 26524 | 17380 | 28810 | 19666 | 31096 | 21952 |
| 24239 | 15095 | 26525 | 17381 | 28811 | 19667 | 31097 | 21953 |
| 24240 | 15096 | 26526 | 17382 | 28812 | 19668 | 31098 | 21954 |
| 24241 | 15097 | 26527 | 17383 | 28813 | 19669 | 31099 | 21955 |
| 24242 | 15098 | 26528 | 17384 | 28814 | 19670 | 31100 | 21956 |
| 24243 | 15099 | 26529 | 17385 | 28815 | 19671 | 31101 | 21957 |
| 24244 | 15100 | 26530 | 17386 | 28816 | 19672 | 31102 | 21958 |
| 24245 | 15101 | 26531 | 17387 | 28817 | 19673 | 31103 | 21959 |
| 24246 | 15102 | 26532 | 17388 | 28818 | 19674 | 31104 | 21960 |
| 24247 | 15103 | 26533 | 17389 | 28819 | 19675 | 31105 | 21961 |
| 24248 | 15104 | 26534 | 17390 | 28820 | 19676 | 31106 | 21962 |
| 24249 | 15105 | 26535 | 17391 | 28821 | 19677 | 31107 | 21963 |
| 24250 | 15106 | 26536 | 17392 | 28822 | 19678 | 31108 | 21964 |
| 24251 | 15107 | 26537 | 17393 | 28823 | 19679 | 31109 | 21965 |
| 24252 | 15108 | 26538 | 17394 | 28824 | 19680 | 31110 | 21966 |
| 24253 | 15109 | 26539 | 17395 | 28825 | 19681 | 31111 | 21967 |
| 24254 | 15110 | 26540 | 17396 | 28826 | 19682 | 31112 | 21968 |
| 24255 | 15111 | 26541 | 17397 | 28827 | 19683 | 31113 | 21969 |
| 24256 | 15112 | 26542 | 17398 | 28828 | 19684 | 31114 | 21970 |
| 24257 | 15113 | 26543 | 17399 | 28829 | 19685 | 31115 | 21971 |
| 24258 | 15114 | 26544 | 17400 | 28830 | 19686 | 31116 | 21972 |
| 24259 | 15115 | 26545 | 17401 | 28831 | 19687 | 31117 | 21973 |
| 24260 | 15116 | 26546 | 17402 | 28832 | 19688 | 31118 | 21974 |
| 24261 | 15117 | 26547 | 17403 | 28833 | 19689 | 31119 | 21975 |
| 24262 | 15118 | 26548 | 17404 | 28834 | 19690 | 31120 | 21976 |
| 24263 | 15119 | 26549 | 17405 | 28835 | 19691 | 31121 | 21977 |
| 24264 | 15120 | 26550 | 17406 | 28836 | 19692 | 31122 | 21978 |
| 24265 | 15121 | 26551 | 17407 | 28837 | 19693 | 31123 | 21979 |
| 24266 | 15122 | 26552 | 17408 | 28838 | 19694 | 31124 | 21980 |
| 24267 | 15123 | 26553 | 17409 | 28839 | 19695 | 31125 | 21981 |
| 24268 | 15124 | 26554 | 17410 | 28840 | 19696 | 31126 | 21982 |
| 24269 | 15125 | 26555 | 17411 | 28841 | 19697 | 31127 | 21983 |
| 24270 | 15126 | 26556 | 17412 | 28842 | 19698 | 31128 | 21984 |
| 24271 | 15127 | 26557 | 17413 | 28843 | 19699 | 31129 | 21985 |
| 24272 | 15128 | 26558 | 17414 | 28844 | 19700 | 31130 | 21986 |
| 24273 | 15129 | 26559 | 17415 | 28845 | 19701 | 31131 | 21987 |
| 24274 | 15130 | 26560 | 17416 | 28846 | 19702 | 31132 | 21988 |
| 24275 | 15131 | 26561 | 17417 | 28847 | 19703 | 31133 | 21989 |
| 24276 | 15132 | 26562 | 17418 | 28848 | 19704 | 31134 | 21990 |
| 24277 | 15133 | 26563 | 17419 | 28849 | 19705 | 31135 | 21991 |
| 24278 | 15134 | 26564 | 17420 | 28850 | 19706 | 31136 | 21992 |
| 24279 | 15135 | 26565 | 17421 | 28851 | 19707 | 31137 | 21993 |
| 24280 | 15136 | 26566 | 17422 | 28852 | 19708 | 31138 | 21994 |
| 24281 | 15137 | 26567 | 17423 | 28853 | 19709 | 31139 | 21995 |
| 24282 | 15138 | 26568 | 17424 | 28854 | 19710 | 31140 | 21996 |
| 24283 | 15139 | 26569 | 17425 | 28855 | 19711 | 31141 | 21997 |
| 24284 | 15140 | 26570 | 17426 | 28856 | 19712 | 31142 | 21998 |
| 24285 | 15141 | 26571 | 17427 | 28857 | 19713 | 31143 | 21999 |
| 24286 | 15142 | 26572 | 17428 | 28858 | 19714 | 31144 | 22000 |
| 24287 | 15143 | 26573 | 17429 | 28859 | 19715 | 31145 | 22001 |
| 24288 | 15144 | 26574 | 17430 | 28860 | 19716 | 31146 | 22002 |
| 24289 | 15145 | 26575 | 17431 | 28861 | 19717 | 31147 | 22003 |
| 24290 | 15146 | 26576 | 17432 | 28862 | 19718 | 31148 | 22004 |
| 24291 | 15147 | 26577 | 17433 | 28863 | 19719 | 31149 | 22005 |
| 24292 | 15148 | 26578 | 17434 | 28864 | 19720 | 31150 | 22006 |
| 24293 | 15149 | 26579 | 17435 | 28865 | 19721 | 31151 | 22007 |
| 24294 | 15150 | 26580 | 17436 | 28866 | 19722 | 31152 | 22008 |
| 24295 | 15151 | 26581 | 17437 | 28867 | 19723 | 31153 | 22009 |
| 24296 | 15152 | 26582 | 17438 | 28868 | 19724 | 31154 | 22010 |
| 24297 | 15153 | 26583 | 17439 | 28869 | 19725 | 31155 | 22011 |
| 24298 | 15154 | 26584 | 17440 | 28870 | 19726 | 31156 | 22012 |
| 24299 | 15155 | 26585 | 17441 | 28871 | 19727 | 31157 | 22013 |
| 24300 | 15156 | 26586 | 17442 | 28872 | 19728 | 31158 | 22014 |
| 24301 | 15157 | 26587 | 17443 | 28873 | 19729 | 31159 | 22015 |
| 24302 | 15158 | 26588 | 17444 | 28874 | 19730 | 31160 | 22016 |
| 24303 | 15159 | 26589 | 17445 | 28875 | 19731 | 31161 | 22017 |
| 24304 | 15160 | 26590 | 17446 | 28876 | 19732 | 31162 | 22018 |
| 24305 | 15161 | 26591 | 17447 | 28877 | 19733 | 31163 | 22019 |
| 24306 | 15162 | 26592 | 17448 | 28878 | 19734 | 31164 | 22020 |
| 24307 | 15163 | 26593 | 17449 | 28879 | 19735 | 31165 | 22021 |
| 24308 | 15164 | 26594 | 17450 | 28880 | 19736 | 31166 | 22022 |
| 24309 | 15165 | 26595 | 17451 | 28881 | 19737 | 31167 | 22023 |
| 24310 | 15166 | 26596 | 17452 | 28882 | 19738 | 31168 | 22024 |
| 24311 | 15167 | 26597 | 17453 | 28883 | 19739 | 31169 | 22025 |
| 24312 | 15168 | 26598 | 17454 | 28884 | 19740 | 31170 | 22026 |
| 24313 | 15169 | 26599 | 17455 | 28885 | 19741 | 31171 | 22027 |
| 24314 | 15170 | 26600 | 17456 | 28886 | 19742 | 31172 | 22028 |
| 24315 | 15171 | 26601 | 17457 | 28887 | 19743 | 31173 | 22029 |
| 24316 | 15172 | 26602 | 17458 | 28888 | 19744 | 31174 | 22030 |
| 24317 | 15173 | 26603 | 17459 | 28889 | 19745 | 31175 | 22031 |
| 24318 | 15174 | 26604 | 17460 | 28890 | 19746 | 31176 | 22032 |
| 24319 | 15175 | 26605 | 17461 | 28891 | 19747 | 31177 | 22033 |
| 24320 | 15176 | 26606 | 17462 | 28892 | 19748 | 31178 | 22034 |
| 24321 | 15177 | 26607 | 17463 | 28893 | 19749 | 31179 | 22035 |
| 24322 | 15178 | 26608 | 17464 | 28894 | 19750 | 31180 | 22036 |
| 24323 | 15179 | 26609 | 17465 | 28895 | 19751 | 31181 | 22037 |
| 24324 | 15180 | 26610 | 17466 | 28896 | 19752 | 31182 | 22038 |
| 24325 | 15181 | 26611 | 17467 | 28897 | 19753 | 31183 | 22039 |
| 24326 | 15182 | 26612 | 17468 | 28898 | 19754 | 31184 | 22040 |
| 24327 | 15183 | 26613 | 17469 | 28899 | 19755 | 31185 | 22041 |
| 24328 | 15184 | 26614 | 17470 | 28900 | 19756 | 31186 | 22042 |
| 24329 | 15185 | 26615 | 17471 | 28901 | 19757 | 31187 | 22043 |
| 24330 | 15186 | 26616 | 17472 | 28902 | 19758 | 31188 | 22044 |
| 24331 | 15187 | 26617 | 17473 | 28903 | 19759 | 31189 | 22045 |
| 24332 | 15188 | 26618 | 17474 | 28904 | 19760 | 31190 | 22046 |
| 24333 | 15189 | 26619 | 17475 | 28905 | 19761 | 31191 | 22047 |
| 24334 | 15190 | 26620 | 17476 | 28906 | 19762 | 31192 | 22048 |
| 24335 | 15191 | 26621 | 17477 | 28907 | 19763 | 31193 | 22049 |
| 24336 | 15192 | 26622 | 17478 | 28908 | 19764 | 31194 | 22050 |
| 24337 | 15193 | 26623 | 17479 | 28909 | 19765 | 31195 | 22051 |
| 24338 | 15194 | 26624 | 17480 | 28910 | 19766 | 31196 | 22052 |
| 24339 | 15195 | 26625 | 17481 | 28911 | 19767 | 31197 | 22053 |
| 24340 | 15196 | 26626 | 17482 | 28912 | 19768 | 31198 | 22054 |
| 24341 | 15197 | 26627 | 17483 | 28913 | 19769 | 31199 | 22055 |
| 24342 | 15198 | 26628 | 17484 | 28914 | 19770 | 31200 | 22056 |
| 24343 | 15199 | 26629 | 17485 | 28915 | 19771 | 31201 | 22057 |
| 24344 | 15200 | 26630 | 17486 | 28916 | 19772 | 31202 | 22058 |
| 24345 | 15201 | 26631 | 17487 | 28917 | 19773 | 31203 | 22059 |
| 24346 | 15202 | 26632 | 17488 | 28918 | 19774 | 31204 | 22060 |
| 24347 | 15203 | 26633 | 17489 | 28919 | 19775 | 31205 | 22061 |
| 24348 | 15204 | 26634 | 17490 | 28920 | 19776 | 31206 | 22062 |
| 24349 | 15205 | 26635 | 17491 | 28921 | 19777 | 31207 | 22063 |
| 24350 | 15206 | 26636 | 17492 | 28922 | 19778 | 31208 | 22064 |
| 24351 | 15207 | 26637 | 17493 | 28923 | 19779 | 31209 | 22065 |
| 24352 | 15208 | 26638 | 17494 | 28924 | 19780 | 31210 | 22066 |
| 24353 | 15209 | 26639 | 17495 | 28925 | 19781 | 31211 | 22067 |
| 24354 | 15210 | 26640 | 17496 | 28926 | 19782 | 31212 | 22068 |
| 24355 | 15211 | 26641 | 17497 | 28927 | 19783 | 31213 | 22069 |
| 24356 | 15212 | 26642 | 17498 | 28928 | 19784 | 31214 | 22070 |
| 24357 | 15213 | 26643 | 17499 | 28929 | 19785 | 31215 | 22071 |
| 24358 | 15214 | 26644 | 17500 | 28930 | 19786 | 31216 | 22072 |
| 24359 | 15215 | 26645 | 17501 | 28931 | 19787 | 31217 | 22073 |
| 24360 | 15216 | 26646 | 17502 | 28932 | 19788 | 31218 | 22074 |
| 24361 | 15217 | 26647 | 17503 | 28933 | 19789 | 31219 | 22075 |
| 24362 | 15218 | 26648 | 17504 | 28934 | 19790 | 31220 | 22076 |
| 24363 | 15219 | 26649 | 17505 | 28935 | 19791 | 31221 | 22077 |
| 24364 | 15220 | 26650 | 17506 | 28936 | 19792 | 31222 | 22078 |
| 24365 | 15221 | 26651 | 17507 | 28937 | 19793 | 31223 | 22079 |
| 24366 | 15222 | 26652 | 17508 | 28938 | 19794 | 31224 | 22080 |
| 24367 | 15223 | 26653 | 17509 | 28939 | 19795 | 31225 | 22081 |
| 24368 | 15224 | 26654 | 17510 | 28940 | 19796 | 31226 | 22082 |
| 24369 | 15225 | 26655 | 17511 | 28941 | 19797 | 31227 | 22083 |
| 24370 | 15226 | 26656 | 17512 | 28942 | 19798 | 31228 | 22084 |
| 24371 | 15227 | 26657 | 17513 | 28943 | 19799 | 31229 | 22085 |
| 24372 | 15228 | 26658 | 17514 | 28944 | 19800 | 31230 | 22086 |
| 24373 | 15229 | 26659 | 17515 | 28945 | 19801 | 31231 | 22087 |
| 24374 | 15230 | 26660 | 17516 | 28946 | 19802 | 31232 | 22088 |
| 24375 | 15231 | 26661 | 17517 | 28947 | 19803 | 31233 | 22089 |
| 24376 | 15232 | 26662 | 17518 | 28948 | 19804 | 31234 | 22090 |
| 24377 | 15233 | 26663 | 17519 | 28949 | 19805 | 31235 | 22091 |
| 24378 | 15234 | 26664 | 17520 | 28950 | 19806 | 31236 | 22092 |
| 24379 | 15235 | 26665 | 17521 | 28951 | 19807 | 31237 | 22093 |
| 24380 | 15236 | 26666 | 17522 | 28952 | 19808 | 31238 | 22094 |
| 24381 | 15237 | 26667 | 17523 | 28953 | 19809 | 31239 | 22095 |
| 24382 | 15238 | 26668 | 17524 | 28954 | 19810 | 31240 | 22096 |
| 24383 | 15239 | 26669 | 17525 | 28955 | 19811 | 31241 | 22097 |
| 24384 | 15240 | 26670 | 17526 | 28956 | 19812 | 31242 | 22098 |
| 24385 | 15241 | 26671 | 17527 | 28957 | 19813 | 31243 | 22099 |
| 24386 | 15242 | 26672 | 17528 | 28958 | 19814 | 31244 | 22100 |
| 24387 | 15243 | 26673 | 17529 | 28959 | 19815 | 31245 | 22101 |
| 24388 | 15244 | 26674 | 17530 | 28960 | 19816 | 31246 | 22102 |
| 24389 | 15245 | 26675 | 17531 | 28961 | 19817 | 31247 | 22103 |
| 24390 | 15246 | 26676 | 17532 | 28962 | 19818 | 31248 | 22104 |
| 24391 | 15247 | 26677 | 17533 | 28963 | 19819 | 31249 | 22105 |
| 24392 | 15248 | 26678 | 17534 | 28964 | 19820 | 31250 | 22106 |
| 24393 | 15249 | 26679 | 17535 | 28965 | 19821 | 31251 | 22107 |
| 24394 | 15250 | 26680 | 17536 | 28966 | 19822 | 31252 | 22108 |
| 24395 | 15251 | 26681 | 17537 | 28967 | 19823 | 31253 | 22109 |
| 24396 | 15252 | 26682 | 17538 | 28968 | 19824 | 31254 | 22110 |
| 24397 | 15253 | 26683 | 17539 | 28969 | 19825 | 31255 | 22111 |
| 24398 | 15254 | 26684 | 17540 | 28970 | 19826 | 31256 | 22112 |
| 24399 | 15255 | 26685 | 17541 | 28971 | 19827 | 31257 | 22113 |
| 24400 | 15256 | 26686 | 17542 | 28972 | 19828 | 31258 | 22114 |
| 24401 | 15257 | 26687 | 17543 | 28973 | 19829 | 31259 | 22115 |
| 24402 | 15258 | 26688 | 17544 | 28974 | 19830 | 31260 | 22116 |
| 24403 | 15259 | 26689 | 17545 | 28975 | 19831 | 31261 | 22117 |
| 24404 | 15260 | 26690 | 17546 | 28976 | 19832 | 31262 | 22118 |
| 24405 | 15261 | 26691 | 17547 | 28977 | 19833 | 31263 | 22119 |
| 24406 | 15262 | 26692 | 17548 | 28978 | 19834 | 31264 | 22120 |
| 24407 | 15263 | 26693 | 17549 | 28979 | 19835 | 31265 | 22121 |
| 24408 | 15264 | 26694 | 17550 | 28980 | 19836 | 31266 | 22122 |
| 24409 | 15265 | 26695 | 17551 | 28981 | 19837 | 31267 | 22123 |
| 24410 | 15266 | 26696 | 17552 | 28982 | 19838 | 31268 | 22124 |
| 24411 | 15267 | 26697 | 17553 | 28983 | 19839 | 31269 | 22125 |
| 24412 | 15268 | 26698 | 17554 | 28984 | 19840 | 31270 | 22126 |
| 24413 | 15269 | 26699 | 17555 | 28985 | 19841 | 31271 | 22127 |
| 24414 | 15270 | 26700 | 17556 | 28986 | 19842 | 31272 | 22128 |
| 24415 | 15271 | 26701 | 17557 | 28987 | 19843 | 31273 | 22129 |
| 24416 | 15272 | 26702 | 17558 | 28988 | 19844 | 31274 | 22130 |
| 24417 | 15273 | 26703 | 17559 | 28989 | 19845 | 31275 | 22131 |
| 24418 | 15274 | 26704 | 17560 | 28990 | 19846 | 31276 | 22132 |
| 24419 | 15275 | 26705 | 17561 | 28991 | 19847 | 31277 | 22133 |
| 24420 | 15276 | 26706 | 17562 | 28992 | 19848 | 31278 | 22134 |
| 24421 | 15277 | 26707 | 17563 | 28993 | 19849 | 31279 | 22135 |
| 24422 | 15278 | 26708 | 17564 | 28994 | 19850 | 31280 | 22136 |
| 24423 | 15279 | 26709 | 17565 | 28995 | 19851 | 31281 | 22137 |
| 24424 | 15280 | 26710 | 17566 | 28996 | 19852 | 31282 | 22138 |
| 24425 | 15281 | 26711 | 17567 | 28997 | 19853 | 31283 | 22139 |
| 24426 | 15282 | 26712 | 17568 | 28998 | 19854 | 31284 | 22140 |
| 24427 | 15283 | 26713 | 17569 | 28999 | 19855 | 31285 | 22141 |
| 24428 | 15284 | 26714 | 17570 | 29000 | 19856 | 31286 | 22142 |
| 24429 | 15285 | 26715 | 17571 | 29001 | 19857 | 31287 | 22143 |
| 24430 | 15286 | 26716 | 17572 | 29002 | 19858 | 31288 | 22144 |
| 24431 | 15287 | 26717 | 17573 | 29003 | 19859 | 31289 | 22145 |
| 24432 | 15288 | 26718 | 17574 | 29004 | 19860 | 31290 | 22146 |
| 24433 | 15289 | 26719 | 17575 | 29005 | 19861 | 31291 | 22147 |
| 24434 | 15290 | 26720 | 17576 | 29006 | 19862 | 31292 | 22148 |
| 24435 | 15291 | 26721 | 17577 | 29007 | 19863 | 31293 | 22149 |
| 24436 | 15292 | 26722 | 17578 | 29008 | 19864 | 31294 | 22150 |
| 24437 | 15293 | 26723 | 17579 | 29009 | 19865 | 31295 | 22151 |
| 24438 | 15294 | 26724 | 17580 | 29010 | 19866 | 31296 | 22152 |
| 24439 | 15295 | 26725 | 17581 | 29011 | 19867 | 31297 | 22153 |
| 24440 | 15296 | 26726 | 17582 | 29012 | 19868 | 31298 | 22154 |
| 24441 | 15297 | 26727 | 17583 | 29013 | 19869 | 31299 | 22155 |
| 24442 | 15298 | 26728 | 17584 | 29014 | 19870 | 31300 | 22156 |
| 24443 | 15299 | 26729 | 17585 | 29015 | 19871 | 31301 | 22157 |
| 24444 | 15300 | 26730 | 17586 | 29016 | 19872 | 31302 | 22158 |
| 24445 | 15301 | 26731 | 17587 | 29017 | 19873 | 31303 | 22159 |
| 24446 | 15302 | 26732 | 17588 | 29018 | 19874 | 31304 | 22160 |
| 24447 | 15303 | 26733 | 17589 | 29019 | 19875 | 31305 | 22161 |
| 24448 | 15304 | 26734 | 17590 | 29020 | 19876 | 31306 | 22162 |
| 24449 | 15305 | 26735 | 17591 | 29021 | 19877 | 31307 | 22163 |
| 24450 | 15306 | 26736 | 17592 | 29022 | 19878 | 31308 | 22164 |
| 24451 | 15307 | 26737 | 17593 | 29023 | 19879 | 31309 | 22165 |
| 24452 | 15308 | 26738 | 17594 | 29024 | 19880 | 31310 | 22166 |
| 24453 | 15309 | 26739 | 17595 | 29025 | 19881 | 31311 | 22167 |
| 24454 | 15310 | 26740 | 17596 | 29026 | 19882 | 31312 | 22168 |
| 24455 | 15311 | 26741 | 17597 | 29027 | 19883 | 31313 | 22169 |
| 24456 | 15312 | 26742 | 17598 | 29028 | 19884 | 31314 | 22170 |
| 24457 | 15313 | 26743 | 17599 | 29029 | 19885 | 31315 | 22171 |
| 24458 | 15314 | 26744 | 17600 | 29030 | 19886 | 31316 | 22172 |
| 24459 | 15315 | 26745 | 17601 | 29031 | 19887 | 31317 | 22173 |
| 24460 | 15316 | 26746 | 17602 | 29032 | 19888 | 31318 | 22174 |
| 24461 | 15317 | 26747 | 17603 | 29033 | 19889 | 31319 | 22175 |
| 24462 | 15318 | 26748 | 17604 | 29034 | 19890 | | 22176 |
| 24463 | 15319 | 26749 | 17605 | 29035 | 19891 | | 22177 |
| 24464 | 15320 | 26750 | 17606 | 29036 | 19892 | | 22178 |
| 24465 | 15321 | 26751 | 17607 | 29037 | 19893 | | 22179 |
| 24466 | 15322 | 26752 | 17608 | 29038 | 19894 | | 22180 |
| 24467 | 15323 | 26753 | 17609 | 29039 | 19895 | | 22181 |
| 24468 | 15324 | 26754 | 17610 | 29040 | 19896 | | 22182 |
| 24469 | 15325 | 26755 | 17611 | 29041 | 19897 | | 22183 |
| 24470 | 15326 | 26756 | 17612 | 29042 | 19898 | | 22184 |
| 24471 | 15327 | 26757 | 17613 | 29043 | 19899 | | 22185 |
| 24472 | 15328 | 26758 | 17614 | 29044 | 19900 | | 22186 |
| 24473 | 15329 | 26759 | 17615 | 29045 | 19901 | | 22187 |
| 24474 | 15330 | 26760 | 17616 | 29046 | 19902 | | 22188 |
| 24475 | 15331 | 26761 | 17617 | 29047 | 19903 | | 22189 |
| 24476 | 15332 | 26762 | 17618 | 29048 | 19904 | | 22190 |
| 24477 | 15333 | 26763 | 17619 | 29049 | 19905 | | 22191 |
| 24478 | 15334 | 26764 | 17620 | 29050 | 19906 | | 22192 |
| 24479 | 15335 | 26765 | 17621 | 29051 | 19907 | | 22193 |
| 24480 | 15336 | 26766 | 17622 | 29052 | 19908 | | 22194 |
| 24481 | 15337 | 26767 | 17623 | 29053 | 19909 | | 22195 |
| 24482 | 15338 | 26768 | 17624 | 29054 | 19910 | | 22196 |
| 24483 | 15339 | 26769 | 17625 | 29055 | 19911 | | 22197 |
| 24484 | 15340 | 26770 | 17626 | 29056 | 19912 | | 22198 |
| 24485 | 15341 | 26771 | 17627 | 29057 | 19913 | | 22199 |
| 24486 | 15342 | 26772 | 17628 | 29058 | 19914 | | 22200 |
| 24487 | 15343 | 26773 | 17629 | 29059 | 19915 | | 22201 |
| 24488 | 15344 | 26774 | 17630 | 29060 | 19916 | | 22202 |
| 24489 | 15345 | 26775 | 17631 | 29061 | 19917 | | 22203 |
| 24490 | 15346 | 26776 | 17632 | 29062 | 19918 | | 22204 |
| 24491 | 15347 | 26777 | 17633 | 29063 | 19919 | | 22205 |
| 24492 | 15348 | 26778 | 17634 | 29064 | 19920 | | 22206 |
| 24493 | 15349 | 26779 | 17635 | 29065 | 19921 | | 22207 |
| 24494 | 15350 | 26780 | 17636 | 29066 | 19922 | | 22208 |
| 24495 | 15351 | 26781 | 17637 | 29067 | 19923 | | 22209 |
| 24496 | 15352 | 26782 | 17638 | 29068 | 19924 | | 22210 |
| 24497 | 15353 | 26783 | 17639 | 29069 | 19925 | | 22211 |
| 24498 | 15354 | 26784 | 17640 | 29070 | 19926 | | 22212 |
| 24499 | 15355 | 26785 | 17641 | 29071 | 19927 | | 22213 |
| 24500 | 15356 | 26786 | 17642 | 29072 | 19928 | | 22214 |
| 24501 | 15357 | 26787 | 17643 | 29073 | 19929 | | 22215 |
| 24502 | 15358 | 26788 | 17644 | 29074 | 19930 | | 22216 |
| 24503 | 15359 | 26789 | 17645 | 29075 | 19931 | | 22217 |
| 24504 | 15360 | 26790 | 17646 | 29076 | 19932 | | 22218 |
| 24505 | 15361 | 26791 | 17647 | 29077 | 19933 | | 22219 |
| 24506 | 15362 | 26792 | 17648 | 29078 | 19934 | | 22220 |
| 24507 | 15363 | 26793 | 17649 | 29079 | 19935 | | 22221 |
| 24508 | 15364 | 26794 | 17650 | 29080 | 19936 | | 22222 |
| 24509 | 15365 | 26795 | 17651 | 29081 | 19937 | | 22223 |
| 24510 | 15366 | 26796 | 17652 | 29082 | 19938 | | 22224 |
| 24511 | 15367 | 26797 | 17653 | 29083 | 19939 | | 22225 |
| 24512 | 15368 | 26798 | 17654 | 29084 | 19940 | | 22226 |
| 24513 | 15369 | 26799 | 17655 | 29085 | 19941 | | 22227 |
| 24514 | 15370 | 26800 | 17656 | 29086 | 19942 | | 22228 |
| 24515 | 15371 | 26801 | 17657 | 29087 | 19943 | | 22229 |
| 24516 | 15372 | 26802 | 17658 | 29088 | 19944 | | 22230 |
| 24517 | 15373 | 26803 | 17659 | 29089 | 19945 | | 22231 |
| 24518 | 15374 | 26804 | 17660 | 29090 | 19946 | | 22232 |
| 24519 | 15375 | 26805 | 17661 | 29091 | 19947 | | 22233 |
| 24520 | 15376 | 26806 | 17662 | 29092 | 19948 | | 22234 |
| 24521 | 15377 | 26807 | 17663 | 29093 | 19949 | | 22235 |
| 24522 | 15378 | 26808 | 17664 | 29094 | 19950 | | 22236 |
| 24523 | 15379 | 26809 | 17665 | 29095 | 19951 | | 22237 |
| 24524 | 15380 | 26810 | 17666 | 29096 | 19952 | | 22238 |
| 24525 | 15381 | 26811 | 17667 | 29097 | 19953 | | 22239 |
| 24526 | 15382 | 26812 | 17668 | 29098 | 19954 | | 22240 |
| 24527 | 15383 | 26813 | 17669 | 29099 | 19955 | | 22241 |
| 24528 | 15384 | 26814 | 17670 | 29100 | 19956 | | 22242 |
| 24529 | 15385 | 26815 | 17671 | 29101 | 19957 | | 22243 |
| 24530 | 15386 | 26816 | 17672 | 29102 | 19958 | | 22244 |
| 24531 | 15387 | 26817 | 17673 | 29103 | 19959 | | 22245 |
| 24532 | 15388 | 26818 | 17674 | 29104 | 19960 | | 22246 |
| 24533 | 15389 | 26819 | 17675 | 29105 | 19961 | | 22247 |
| 24534 | 15390 | 26820 | 17676 | 29106 | 19962 | | 22248 |
| 24535 | 15391 | 26821 | 17677 | 29107 | 19963 | | 22249 |
| 24536 | 15392 | 26822 | 17678 | 29108 | 19964 | | 22250 |
| 24537 | 15393 | 26823 | 17679 | 29109 | 19965 | | 22251 |
| 24538 | 15394 | 26824 | 17680 | 29110 | 19966 | | 22252 |
| 24539 | 15395 | 26825 | 17681 | 29111 | 19967 | | 22253 |
| 24540 | 15396 | 26826 | 17682 | 29112 | 19968 | | 22254 |
| 24541 | 15397 | 26827 | 17683 | 29113 | 19969 | | 22255 |
| 24542 | 15398 | 26828 | 17684 | 29114 | 19970 | | 22256 |
| 24543 | 15399 | 26829 | 17685 | 29115 | 19971 | | 22257 |
| 24544 | 15400 | 26830 | 17686 | 29116 | 19972 | | 22258 |
| 24545 | 15401 | 26831 | 17687 | 29117 | 19973 | | 22259 |
| 24546 | 15402 | 26832 | 17688 | 29118 | 19974 | | 22260 |
| 24547 | 15403 | 26833 | 17689 | 29119 | 19975 | | 22261 |
| 24548 | 15404 | 26834 | 17690 | 29120 | 19976 | | 22262 |
| 24549 | 15405 | 26835 | 17691 | 29121 | 19977 | | 22263 |
| 24550 | 15406 | 26836 | 17692 | 29122 | 19978 | | 22264 |
| 24551 | 15407 | 26837 | 17693 | 29123 | 19979 | | 22265 |
| 24552 | 15408 | 26838 | 17694 | 29124 | 19980 | | 22266 |
| 24553 | 15409 | 26839 | 17695 | 29125 | 19981 | | 22267 |
| 24554 | 15410 | 26840 | 17696 | 29126 | 19982 | | 22268 |
| 24555 | 15411 | 26841 | 17697 | 29127 | 19983 | | 22269 |
| 24556 | 15412 | 26842 | 17698 | 29128 | 19984 | | 22270 |
| 24557 | 15413 | 26843 | 17699 | 29129 | 19985 | | 22271 |
| 24558 | 15414 | 26844 | 17700 | 29130 | 19986 | | 22272 |
| 24559 | 15415 | 26845 | 17701 | 29131 | 19987 | | 22273 |
| 24560 | 15416 | 26846 | 17702 | 29132 | 19988 | | 22274 |
| 24561 | 15417 | 26847 | 17703 | 29133 | 19989 | | 22275 |
| 24562 | 15418 | 26848 | 17704 | 29134 | 19990 | | 22276 |
| 24563 | 15419 | 26849 | 17705 | 29135 | 19991 | | 22277 |
| 24564 | 15420 | 26850 | 17706 | 29136 | 19992 | | 22278 |
| 24565 | 15421 | 26851 | 17707 | 29137 | 19993 | | 22279 |
| 24566 | 15422 | 26852 | 17708 | 29138 | 19994 | | 22280 |
| 24567 | 15423 | 26853 | 17709 | 29139 | 19995 | | 22281 |
| 24568 | 15424 | 26854 | 17710 | 29140 | 19996 | | 22282 |
| 24569 | 15425 | 26855 | 17711 | 29141 | 19997 | | 22283 |
| 24570 | 15426 | 26856 | 17712 | 29142 | 19998 | | 22284 |
| 24571 | 15427 | 26857 | 17713 | 29143 | 19999 | | 22285 |
| 24572 | 15428 | 26858 | 17714 | 29144 | 20000 | | 22286 |
| 24573 | 15429 | 26859 | 17715 | 29145 | 20001 | | 22287 |
| 24574 | 15430 | 26860 | 17716 | 29146 | 20002 | | 22288 |
| 24575 | 15431 | 26861 | 17717 | 29147 | 20003 | | 22289 |
| 24576 | 15432 | 26862 | 17718 | 29148 | 20004 | | 22290 |
| 24577 | 15433 | 26863 | 17719 | 29149 | 20005 | | 22291 |
| 24578 | 15434 | 26864 | 17720 | 29150 | 20006 | | 22292 |
| 24579 | 15435 | 26865 | 17721 | 29151 | 20007 | | 22293 |
| 24580 | 15436 | 26866 | 17722 | 29152 | 20008 | | 22294 |
| 24581 | 15437 | 26867 | 17723 | 29153 | 20009 | | 22295 |
| 24582 | 15438 | 26868 | 17724 | 29154 | 20010 | | 22296 |
| 24583 | 15439 | 26869 | 17725 | 29155 | 20011 | | 22297 |
| 24584 | 15440 | 26870 | 17726 | 29156 | 20012 | | 22298 |
| 24585 | 15441 | 26871 | 17727 | 29157 | 20013 | | 22299 |
| 24586 | 15442 | 26872 | 17728 | 29158 | 20014 | | 22300 |
| 24587 | 15443 | 26873 | 17729 | 29159 | 20015 | | 22301 |
| 24588 | 15444 | 26874 | 17730 | 29160 | 20016 | | 22302 |
| 24589 | 15445 | 26875 | 17731 | 29161 | 20017 | | 22303 |
| 24590 | 15446 | 26876 | 17732 | 29162 | 20018 | | 22304 |
| 24591 | 15447 | 26877 | 17733 | 29163 | 20019 | | 22305 |
| 24592 | 15448 | 26878 | 17734 | 29164 | 20020 | | 22306 |
| 24593 | 15449 | 26879 | 17735 | 29165 | 20021 | | 22307 |
| 24594 | 15450 | 26880 | 17736 | 29166 | 20022 | | 22308 |
| 24595 | 15451 | 26881 | 17737 | 29167 | 20023 | | 22309 |
| 24596 | 15452 | 26882 | 17738 | 29168 | 20024 | | 22310 |
| 24597 | 15453 | 26883 | 17739 | 29169 | 20025 | | 22311 |
| 24598 | 15454 | 26884 | 17740 | 29170 | 20026 | | 22312 |
| 24599 | 15455 | 26885 | 17741 | 29171 | 20027 | | 22313 |
| 24600 | 15456 | 26886 | 17742 | 29172 | 20028 | | 22314 |
| 24601 | 15457 | 26887 | 17743 | 29173 | 20029 | | 22315 |
| 24602 | 15458 | 26888 | 17744 | 29174 | 20030 | | 22316 |
| 24603 | 15459 | 26889 | 17745 | 29175 | 20031 | | 22317 |
| 24604 | 15460 | 26890 | 17746 | 29176 | 20032 | | 22318 |
| 24605 | 15461 | 26891 | 17747 | 29177 | 20033 | | 22319 |
| 24606 | 15462 | 26892 | 17748 | 29178 | 20034 | | 22320 |
| 24607 | 15463 | 26893 | 17749 | 29179 | 20035 | | 22321 |
| 24608 | 15464 | 26894 | 17750 | 29180 | 20036 | | 22322 |
| 24609 | 15465 | 26895 | 17751 | 29181 | 20037 | | 22323 |
| 24610 | 15466 | 26896 | 17752 | 29182 | 20038 | | 22324 |
| 24611 | 15467 | 26897 | 17753 | 29183 | 20039 | | 22325 |
| 24612 | 15468 | 26898 | 17754 | 29184 | 20040 | | 22326 |
| 24613 | 15469 | 26899 | 17755 | 29185 | 20041 | | 22327 |
| 24614 | 15470 | 26900 | 17756 | 29186 | 20042 | | 22328 |
| 24615 | 15471 | 26901 | 17757 | 29187 | 20043 | | 22329 |
| 24616 | 15472 | 26902 | 17758 | 29188 | 20044 | | 22330 |
| 24617 | 15473 | 26903 | 17759 | 29189 | 20045 | | 22331 |
| 24618 | 15474 | 26904 | 17760 | 29190 | 20046 | | 22332 |
| 24619 | 15475 | 26905 | 17761 | 29191 | 20047 | | 22333 |
| 24620 | 15476 | 26906 | 17762 | 29192 | 20048 | | 22334 |
| 24621 | 15477 | 26907 | 17763 | 29193 | 20049 | | 22335 |
| 24622 | 15478 | 26908 | 17764 | 29194 | 20050 | | 22336 |
| 24623 | 15479 | 26909 | 17765 | 29195 | 20051 | | 22337 |
| 24624 | 15480 | 26910 | 17766 | 29196 | 20052 | | 22338 |
| 24625 | 15481 | 26911 | 17767 | 29197 | 20053 | | 22339 |
| 24626 | 15482 | 26912 | 17768 | 29198 | 20054 | | 22340 |
| 24627 | 15483 | 26913 | 17769 | 29199 | 20055 | | 22341 |
| 24628 | 15484 | 26914 | 17770 | 29200 | 20056 | | 22342 |
| 24629 | 15485 | 26915 | 17771 | 29201 | 20057 | | 22343 |
| 24630 | 15486 | 26916 | 17772 | 29202 | 20058 | | 22344 |
| 24631 | 15487 | 26917 | 17773 | 29203 | 20059 | | 22345 |
| 24632 | 15488 | 26918 | 17774 | 29204 | 20060 | | 22346 |
| 24633 | 15489 | 26919 | 17775 | 29205 | 20061 | | 22347 |
| 24634 | 15490 | 26920 | 17776 | 29206 | 20062 | | 22348 |
| 24635 | 15491 | 26921 | 17777 | 29207 | 20063 | | 22349 |
| 24636 | 15492 | 26922 | 17778 | 29208 | 20064 | | 22350 |
| 24637 | 15493 | 26923 | 17779 | 29209 | 20065 | | 22351 |
| 24638 | 15494 | 26924 | 17780 | 29210 | 20066 | | 22352 |
| 24639 | 15495 | 26925 | 17781 | 29211 | 20067 | | 22353 |
| 24640 | 15496 | 26926 | 17782 | 29212 | 20068 | | 22354 |
| 24641 | 15497 | 26927 | 17783 | 29213 | 20069 | | 22355 |
| 24642 | 15498 | 26928 | 17784 | 29214 | 20070 | | 22356 |
| 24643 | 15499 | 26929 | 17785 | 29215 | 20071 | | 22357 |
| 24644 | 15500 | 26930 | 17786 | 29216 | 20072 | | 22358 |
| 24645 | 15501 | 26931 | 17787 | 29217 | 20073 | | 22359 |
| 24646 | 15502 | 26932 | 17788 | 29218 | 20074 | | 22360 |
| 24647 | 15503 | 26933 | 17789 | 29219 | 20075 | | 22361 |
| 24648 | 15504 | 26934 | 17790 | 29220 | 20076 | | 22362 |
| 24649 | 15505 | 26935 | 17791 | 29221 | 20077 | | 22363 |
| 24650 | 15506 | 26936 | 17792 | 29222 | 20078 | | 22364 |
| 24651 | 15507 | 26937 | 17793 | 29223 | 20079 | | 22365 |
| 24652 | 15508 | 26938 | 17794 | 29224 | 20080 | | 22366 |
| 24653 | 15509 | 26939 | 17795 | 29225 | 20081 | | 22367 |
| 24654 | 15510 | 26940 | 17796 | 29226 | 20082 | | 22368 |
| 24655 | 15511 | 26941 | 17797 | 29227 | 20083 | | 22369 |
| 24656 | 15512 | 26942 | 17798 | 29228 | 20084 | | 22370 |
| 24657 | 15513 | 26943 | 17799 | 29229 | 20085 | | 22371 |
| 24658 | 15514 | 26944 | 17800 | 29230 | 20086 | | 22372 |
| 24659 | 15515 | 26945 | 17801 | 29231 | 20087 | | 22373 |
| 24660 | 15516 | 26946 | 17802 | 29232 | 20088 | | 22374 |
| 24661 | 15517 | 26947 | 17803 | 29233 | 20089 | | 22375 |
| 24662 | 15518 | 26948 | 17804 | 29234 | 20090 | | 22376 |
| 24663 | 15519 | 26949 | 17805 | 29235 | 20091 | | 22377 |
| 24664 | 15520 | 26950 | 17806 | 29236 | 20092 | | 22378 |
| 24665 | 15521 | 26951 | 17807 | 29237 | 20093 | | 22379 |
| 24666 | 15522 | 26952 | 17808 | 29238 | 20094 | | 22380 |
| 24667 | 15523 | 26953 | 17809 | 29239 | 20095 | | 22381 |
| 24668 | 15524 | 26954 | 17810 | 29240 | 20096 | | 22382 |
| 24669 | 15525 | 26955 | 17811 | 29241 | 20097 | | 22383 |
| 24670 | 15526 | 26956 | 17812 | 29242 | 20098 | | 22384 |
| 24671 | 15527 | 26957 | 17813 | 29243 | 20099 | | 22385 |
| 24672 | 15528 | 26958 | 17814 | 29244 | 20100 | | 22386 |
| 24673 | 15529 | 26959 | 17815 | 29245 | 20101 | | 22387 |
| 24674 | 15530 | 26960 | 17816 | 29246 | 20102 | | 22388 |
| 24675 | 15531 | 26961 | 17817 | 29247 | 20103 | | 22389 |
| 24676 | 15532 | 26962 | 17818 | 29248 | 20104 | | 22390 |
| 24677 | 15533 | 26963 | 17819 | 29249 | 20105 | | 22391 |
| 24678 | 15534 | 26964 | 17820 | 29250 | 20106 | | 22392 |
| 24679 | 15535 | 26965 | 17821 | 29251 | 20107 | | 22393 |
| 24680 | 15536 | 26966 | 17822 | 29252 | 20108 | | 22394 |
| 24681 | 15537 | 26967 | 17823 | 29253 | 20109 | | 22395 |
| 24682 | 15538 | 26968 | 17824 | 29254 | 20110 | | 22396 |
| 24683 | 15539 | 26969 | 17825 | 29255 | 20111 | | 22397 |
| 24684 | 15540 | 26970 | 17826 | 29256 | 20112 | | 22398 |
| 24685 | 15541 | 26971 | 17827 | 29257 | 20113 | | 22399 |
| 24686 | 15542 | 26972 | 17828 | 29258 | 20114 | | 22400 |
| 24687 | 15543 | 26973 | 17829 | 29259 | 20115 | | 22401 |
| 24688 | 15544 | 26974 | 17830 | 29260 | 20116 | | 22402 |
| 24689 | 15545 | 26975 | 17831 | 29261 | 20117 | | 22403 |
| 24690 | 15546 | 26976 | 17832 | 29262 | 20118 | | 22404 |
| 24691 | 15547 | 26977 | 17833 | 29263 | 20119 | | 22405 |
| 24692 | 15548 | 26978 | 17834 | 29264 | 20120 | | 22406 |
| 24693 | 15549 | 26979 | 17835 | 29265 | 20121 | | 22407 |
| 24694 | 15550 | 26980 | 17836 | 29266 | 20122 | | 22408 |
| 24695 | 15551 | 26981 | 17837 | 29267 | 20123 | | 22409 |
| 24696 | 15552 | 26982 | 17838 | 29268 | 20124 | | 22410 |
| 24697 | 15553 | 26983 | 17839 | 29269 | 20125 | | 22411 |
| 24698 | 15554 | 26984 | 17840 | 29270 | 20126 | | 22412 |
| 24699 | 15555 | 26985 | 17841 | 29271 | 20127 | | 22413 |
| 24700 | 15556 | 26986 | 17842 | 29272 | 20128 | | 22414 |
| 24701 | 15557 | 26987 | 17843 | 29273 | 20129 | | 22415 |
| 24702 | 15558 | 26988 | 17844 | 29274 | 20130 | | 22416 |
| 24703 | 15559 | 26989 | 17845 | 29275 | 20131 | | 22417 |
| 24704 | 15560 | 26990 | 17846 | 29276 | 20132 | | 22418 |
| 24705 | 15561 | 26991 | 17847 | 29277 | 20133 | | 22419 |
| 24706 | 15562 | 26992 | 17848 | 29278 | 20134 | | 22420 |
| 24707 | 15563 | 26993 | 17849 | 29279 | 20135 | | 22421 |
| 24708 | 15564 | 26994 | 17850 | 29280 | 20136 | | 22422 |
| 24709 | 15565 | 26995 | 17851 | 29281 | 20137 | | 22423 |
| 24710 | 15566 | 26996 | 17852 | 29282 | 20138 | | 22424 |
| 24711 | 15567 | 26997 | 17853 | 29283 | 20139 | | 22425 |
| 24712 | 15568 | 26998 | 17854 | 29284 | 20140 | | 22426 |
| 24713 | 15569 | 26999 | 17855 | 29285 | 20141 | | 22427 |
| 24714 | 15570 | 27000 | 17856 | 29286 | 20142 | | 22428 |
| 24715 | 15571 | 27001 | 17857 | 29287 | 20143 | | 22429 |
| 24716 | 15572 | 27002 | 17858 | 29288 | 20144 | | 22430 |
| 24717 | 15573 | 27003 | 17859 | 29289 | 20145 | | 22431 |
| 24718 | 15574 | 27004 | 17860 | 29290 | 20146 | | 22432 |
| 24719 | 15575 | 27005 | 17861 | 29291 | 20147 | | 22433 |
| 24720 | 15576 | 27006 | 17862 | 29292 | 20148 | | 22434 |
| 24721 | 15577 | 27007 | 17863 | 29293 | 20149 | | 22435 |
| 24722 | 15578 | 27008 | 17864 | 29294 | 20150 | | 22436 |
| 24723 | 15579 | 27009 | 17865 | 29295 | 20151 | | 22437 |
| 24724 | 15580 | 27010 | 17866 | 29296 | 20152 | | 22438 |
| 24725 | 15581 | 27011 | 17867 | 29297 | 20153 | | 22439 |
| 24726 | 15582 | 27012 | 17868 | 29298 | 20154 | | 22440 |
| 24727 | 15583 | 27013 | 17869 | 29299 | 20155 | | 22441 |
| 24728 | 15584 | 27014 | 17870 | 29300 | 20156 | | 22442 |
| 24729 | 15585 | 27015 | 17871 | 29301 | 20157 | | 22443 |
| 24730 | 15586 | 27016 | 17872 | 29302 | 20158 | | 22444 |
| 24731 | 15587 | 27017 | 17873 | 29303 | 20159 | | 22445 |
| 24732 | 15588 | 27018 | 17874 | 29304 | 20160 | | 22446 |
| 24733 | 15589 | 27019 | 17875 | 29305 | 20161 | | 22447 |
| 24734 | 15590 | 27020 | 17876 | 29306 | 20162 | | 22448 |
| 24735 | 15591 | 27021 | 17877 | 29307 | 20163 | | 22449 |
| 24736 | 15592 | 27022 | 17878 | 29308 | 20164 | | 22450 |
| 24737 | 15593 | 27023 | 17879 | 29309 | 20165 | | 22451 |
| 24738 | 15594 | 27024 | 17880 | 29310 | 20166 | | 22452 |
| 24739 | 15595 | 27025 | 17881 | 29311 | 20167 | | 22453 |
| 24740 | 15596 | 27026 | 17882 | 29312 | 20168 | | 22454 |
| 24741 | 15597 | 27027 | 17883 | 29313 | 20169 | | 22455 |
| 24742 | 15598 | 27028 | 17884 | 29314 | 20170 | | 22456 |
| 24743 | 15599 | 27029 | 17885 | 29315 | 20171 | | 22457 |
| 24744 | 15600 | 27030 | 17886 | 29316 | 20172 | | 22458 |
| 24745 | 15601 | 27031 | 17887 | 29317 | 20173 | | 22459 |
| 24746 | 15602 | 27032 | 17888 | 29318 | 20174 | | 22460 |
| 24747 | 15603 | 27033 | 17889 | 29319 | 20175 | | 22461 |
| 24748 | 15604 | 27034 | 17890 | 29320 | 20176 | | 22462 |
| 24749 | 15605 | 27035 | 17891 | 29321 | 20177 | | 22463 |
| 24750 | 15606 | 27036 | 17892 | 29322 | 20178 | | 22464 |
| 24751 | 15607 | 27037 | 17893 | 29323 | 20179 | | 22465 |
| 24752 | 15608 | 27038 | 17894 | 29324 | 20180 | | 22466 |
| 24753 | 15609 | 27039 | 17895 | 29325 | 20181 | | 22467 |
| 24754 | 15610 | 27040 | 17896 | 29326 | 20182 | | 22468 |
| 24755 | 15611 | 27041 | 17897 | 29327 | 20183 | | 22469 |
| 24756 | 15612 | 27042 | 17898 | 29328 | 20184 | | 22470 |
| 24757 | 15613 | 27043 | 17899 | 29329 | 20185 | | 22471 |
| 24758 | 15614 | 27044 | 17900 | 29330 | 20186 | | 22472 |
| 24759 | 15615 | 27045 | 17901 | 29331 | 20187 | | 22473 |
| 24760 | 15616 | 27046 | 17902 | 29332 | 20188 | | 22474 |
| 24761 | 15617 | 27047 | 17903 | 29333 | 20189 | | 22475 |
| 24762 | 15618 | 27048 | 17904 | 29334 | 20190 | | 22476 |
| 24763 | 15619 | 27049 | 17905 | 29335 | 20191 | | 22477 |
| 24764 | 15620 | 27050 | 17906 | 29336 | 20192 | | 22478 |
| 24765 | 15621 | 27051 | 17907 | 29337 | 20193 | | 22479 |
| 24766 | 15622 | 27052 | 17908 | 29338 | 20194 | | 22480 |
| 24767 | 15623 | 27053 | 17909 | 29339 | 20195 | | 22481 |
| 24768 | 15624 | 27054 | 17910 | 29340 | 20196 | | 22482 |
| 24769 | 15625 | 27055 | 17911 | 29341 | 20197 | | 22483 |
| 24770 | 15626 | 27056 | 17912 | 29342 | 20198 | | 22484 |
| 24771 | 15627 | 27057 | 17913 | 29343 | 20199 | | 22485 |
| 24772 | 15628 | 27058 | 17914 | 29344 | 20200 | | 22486 |
| 24773 | 15629 | 27059 | 17915 | 29345 | 20201 | | 22487 |
| 24774 | 15630 | 27060 | 17916 | 29346 | 20202 | | 22488 |
| 24775 | 15631 | 27061 | 17917 | 29347 | 20203 | | 22489 |
| 24776 | 15632 | 27062 | 17918 | 29348 | 20204 | | 22490 |
| 24777 | 15633 | 27063 | 17919 | 29349 | 20205 | | 22491 |
| 24778 | 15634 | 27064 | 17920 | 29350 | 20206 | | 22492 |
| 24779 | 15635 | 27065 | 17921 | 29351 | 20207 | | 22493 |
| 24780 | 15636 | 27066 | 17922 | 29352 | 20208 | | 22494 |
| 24781 | 15637 | 27067 | 17923 | 29353 | 20209 | | 22495 |
| 24782 | 15638 | 27068 | 17924 | 29354 | 20210 | | 22496 |
| 24783 | 15639 | 27069 | 17925 | 29355 | 20211 | | 22497 |
| 24784 | 15640 | 27070 | 17926 | 29356 | 20212 | | 22498 |
| 24785 | 15641 | 27071 | 17927 | 29357 | 20213 | | 22499 |
| 24786 | 15642 | 27072 | 17928 | 29358 | 20214 | | 22500 |
| 24787 | 15643 | 27073 | 17929 | 29359 | 20215 | | 22501 |
| 24788 | 15644 | 27074 | 17930 | 29360 | 20216 | | 22502 |
| 24789 | 15645 | 27075 | 17931 | 29361 | 20217 | | 22503 |
| 24790 | 15646 | 27076 | 17932 | 29362 | 20218 | | 22504 |
| 24791 | 15647 | 27077 | 17933 | 29363 | 20219 | | 22505 |
| 24792 | 15648 | 27078 | 17934 | 29364 | 20220 | | 22506 |
| 24793 | 15649 | 27079 | 17935 | 29365 | 20221 | | 22507 |
| 24794 | 15650 | 27080 | 17936 | 29366 | 20222 | | 22508 |
| 24795 | 15651 | 27081 | 17937 | 29367 | 20223 | | 22509 |
| 24796 | 15652 | 27082 | 17938 | 29368 | 20224 | | 22510 |
| 24797 | 15653 | 27083 | 17939 | 29369 | 20225 | | 22511 |
| 24798 | 15654 | 27084 | 17940 | 29370 | 20226 | | 22512 |
| 24799 | 15655 | 27085 | 17941 | 29371 | 20227 | | 22513 |
| 24800 | 15656 | 27086 | 17942 | 29372 | 20228 | | 22514 |
| 24801 | 15657 | 27087 | 17943 | 29373 | 20229 | | 22515 |
| 24802 | 15658 | 27088 | 17944 | 29374 | 20230 | | 22516 |
| 24803 | 15659 | 27089 | 17945 | 29375 | 20231 | | 22517 |
| 24804 | 15660 | 27090 | 17946 | 29376 | 20232 | | 22518 |
| 24805 | 15661 | 27091 | 17947 | 29377 | 20233 | | 22519 |
| 24806 | 15662 | 27092 | 17948 | 29378 | 20234 | | 22520 |
| 24807 | 15663 | 27093 | 17949 | 29379 | 20235 | | 22521 |
| 24808 | 15664 | 27094 | 17950 | 29380 | 20236 | | 22522 |
| 24809 | 15665 | 27095 | 17951 | 29381 | 20237 | | 22523 |
| 24810 | 15666 | 27096 | 17952 | 29382 | 20238 | | 22524 |
| 24811 | 15667 | 27097 | 17953 | 29383 | 20239 | | 22525 |
| 24812 | 15668 | 27098 | 17954 | 29384 | 20240 | | 22526 |
| 24813 | 15669 | 27099 | 17955 | 29385 | 20241 | | 22527 |
| 24814 | 15670 | 27100 | 17956 | 29386 | 20242 | | 22528 |
| 24815 | 15671 | 27101 | 17957 | 29387 | 20243 | | 22529 |
| 24816 | 15672 | 27102 | 17958 | 29388 | 20244 | | 22530 |
| 24817 | 15673 | 27103 | 17959 | 29389 | 20245 | | 22531 |
| 24818 | 15674 | 27104 | 17960 | 29390 | 20246 | | 22532 |
| 24819 | 15675 | 27105 | 17961 | 29391 | 20247 | | 22533 |
| 24820 | 15676 | 27106 | 17962 | 29392 | 20248 | | 22534 |
| 24821 | 15677 | 27107 | 17963 | 29393 | 20249 | | 22535 |
| 24822 | 15678 | 27108 | 17964 | 29394 | 20250 | | 22536 |
| 24823 | 15679 | 27109 | 17965 | 29395 | 20251 | | 22537 |
| 24824 | 15680 | 27110 | 17966 | 29396 | 20252 | | 22538 |
| 24825 | 15681 | 27111 | 17967 | 29397 | 20253 | | 22539 |
| 24826 | 15682 | 27112 | 17968 | 29398 | 20254 | | 22540 |
| 24827 | 15683 | 27113 | 17969 | 29399 | 20255 | | 22541 |
| 24828 | 15684 | 27114 | 17970 | 29400 | 20256 | | 22542 |
| 24829 | 15685 | 27115 | 17971 | 29401 | 20257 | | 22543 |
| 24830 | 15686 | 27116 | 17972 | 29402 | 20258 | | 22544 |
| 24831 | 15687 | 27117 | 17973 | 29403 | 20259 | | 22545 |
| 24832 | 15688 | 27118 | 17974 | 29404 | 20260 | | 22546 |
| 24833 | 15689 | 27119 | 17975 | 29405 | 20261 | | 22547 |
| 24834 | 15690 | 27120 | 17976 | 29406 | 20262 | | 22548 |
| 24835 | 15691 | 27121 | 17977 | 29407 | 20263 | | 22549 |
| 24836 | 15692 | 27122 | 17978 | 29408 | 20264 | | 22550 |
| 24837 | 15693 | 27123 | 17979 | 29409 | 20265 | | 22551 |
| 24838 | 15694 | 27124 | 17980 | 29410 | 20266 | | 22552 |
| 24839 | 15695 | 27125 | 17981 | 29411 | 20267 | | 22553 |
| 24840 | 15696 | 27126 | 17982 | 29412 | 20268 | | 22554 |
| 24841 | 15697 | 27127 | 17983 | 29413 | 20269 | | 22555 |
| 24842 | 15698 | 27128 | 17984 | 29414 | 20270 | | 22556 |
| 24843 | 15699 | 27129 | 17985 | 29415 | 20271 | | 22557 |
| 24844 | 15700 | 27130 | 17986 | 29416 | 20272 | | 22558 |
| 24845 | 15701 | 27131 | 17987 | 29417 | 20273 | | 22559 |
| 24846 | 15702 | 27132 | 17988 | 29418 | 20274 | | 22560 |
| 24847 | 15703 | 27133 | 17989 | 29419 | 20275 | | 22561 |
| 24848 | 15704 | 27134 | 17990 | 29420 | 20276 | | 22562 |
| 24849 | 15705 | 27135 | 17991 | 29421 | 20277 | | 22563 |
| 24850 | 15706 | 27136 | 17992 | 29422 | 20278 | | 22564 |
| 24851 | 15707 | 27137 | 17993 | 29423 | 20279 | | 22565 |
| 24852 | 15708 | 27138 | 17994 | 29424 | 20280 | | 22566 |
| 24853 | 15709 | 27139 | 17995 | 29425 | 20281 | | 22567 |
| 24854 | 15710 | 27140 | 17996 | 29426 | 20282 | | 22568 |
| 24855 | 15711 | 27141 | 17997 | 29427 | 20283 | | 22569 |
| 24856 | 15712 | 27142 | 17998 | 29428 | 20284 | | 22570 |
| 24857 | 15713 | 27143 | 17999 | 29429 | 20285 | | 22571 |
| 24858 | 15714 | 27144 | 18000 | 29430 | 20286 | | 22572 |
| 24859 | 15715 | 27145 | 18001 | 29431 | 20287 | | 22573 |
| 24860 | 15716 | 27146 | 18002 | 29432 | 20288 | | 22574 |
| 24861 | 15717 | 27147 | 18003 | 29433 | 20289 | | 22575 |
| 24862 | 15718 | 27148 | 18004 | 29434 | 20290 | | 22576 |
| 24863 | 15719 | 27149 | 18005 | 29435 | 20291 | | 22577 |
| 24864 | 15720 | 27150 | 18006 | 29436 | 20292 | | 22578 |
| 24865 | 15721 | 27151 | 18007 | 29437 | 20293 | | 22579 |
| 24866 | 15722 | 27152 | 18008 | 29438 | 20294 | | 22580 |
| 24867 | 15723 | 27153 | 18009 | 29439 | 20295 | | 22581 |
| 24868 | 15724 | 27154 | 18010 | 29440 | 20296 | | 22582 |
| 24869 | 15725 | 27155 | 18011 | 29441 | 20297 | | 22583 |
| 24870 | 15726 | 27156 | 18012 | 29442 | 20298 | | 22584 |
| 24871 | 15727 | 27157 | 18013 | 29443 | 20299 | | 22585 |
| 24872 | 15728 | 27158 | 18014 | 29444 | 20300 | | 22586 |
| 24873 | 15729 | 27159 | 18015 | 29445 | 20301 | | 22587 |
| 24874 | 15730 | 27160 | 18016 | 29446 | 20302 | | 22588 |
| 24875 | 15731 | 27161 | 18017 | 29447 | 20303 | | 22589 |
| 24876 | 15732 | 27162 | 18018 | 29448 | 20304 | | 22590 |
| 24877 | 15733 | 27163 | 18019 | 29449 | 20305 | | 22591 |
| 24878 | 15734 | 27164 | 18020 | 29450 | 20306 | | 22592 |
| 24879 | 15735 | 27165 | 18021 | 29451 | 20307 | | 22593 |
| 24880 | 15736 | 27166 | 18022 | 29452 | 20308 | | 22594 |
| 24881 | 15737 | 27167 | 18023 | 29453 | 20309 | | 22595 |
| 24882 | 15738 | 27168 | 18024 | 29454 | 20310 | | 22596 |
| 24883 | 15739 | 27169 | 18025 | 29455 | 20311 | | 22597 |
| 24884 | 15740 | 27170 | 18026 | 29456 | 20312 | | 22598 |
| 24885 | 15741 | 27171 | 18027 | 29457 | 20313 | | 22599 |
| 24886 | 15742 | 27172 | 18028 | 29458 | 20314 | | 22600 |
| 24887 | 15743 | 27173 | 18029 | 29459 | 20315 | | 22601 |
| 24888 | 15744 | 27174 | 18030 | 29460 | 20316 | | 22602 |
| 24889 | 15745 | 27175 | 18031 | 29461 | 20317 | | 22603 |
| 24890 | 15746 | 27176 | 18032 | 29462 | 20318 | | 22604 |
| 24891 | 15747 | 27177 | 18033 | 29463 | 20319 | | 22605 |
| 24892 | 15748 | 27178 | 18034 | 29464 | 20320 | | 22606 |
| 24893 | 15749 | 27179 | 18035 | 29465 | 20321 | | 22607 |
| 24894 | 15750 | 27180 | 18036 | 29466 | 20322 | | 22608 |
| 24895 | 15751 | 27181 | 18037 | 29467 | 20323 | | 22609 |
| 24896 | 15752 | 27182 | 18038 | 29468 | 20324 | | 22610 |
| 24897 | 15753 | 27183 | 18039 | 29469 | 20325 | | 22611 |
| 24898 | 15754 | 27184 | 18040 | 29470 | 20326 | | 22612 |
| 24899 | 15755 | 27185 | 18041 | 29471 | 20327 | | 22613 |
| 24900 | 15756 | 27186 | 18042 | 29472 | 20328 | | 22614 |
| 24901 | 15757 | 27187 | 18043 | 29473 | 20329 | | 22615 |
| 24902 | 15758 | 27188 | 18044 | 29474 | 20330 | | 22616 |
| 24903 | 15759 | 27189 | 18045 | 29475 | 20331 | | 22617 |
| 24904 | 15760 | 27190 | 18046 | 29476 | 20332 | | 22618 |
| 24905 | 15761 | 27191 | 18047 | 29477 | 20333 | | 22619 |
| 24906 | 15762 | 27192 | 18048 | 29478 | 20334 | | 22620 |
| 24907 | 15763 | 27193 | 18049 | 29479 | 20335 | | 22621 |
| 24908 | 15764 | 27194 | 18050 | 29480 | 20336 | | 22622 |
| 24909 | 15765 | 27195 | 18051 | 29481 | 20337 | | 22623 |
| 24910 | 15766 | 27196 | 18052 | 29482 | 20338 | | 22624 |
| 24911 | 15767 | 27197 | 18053 | 29483 | 20339 | | 22625 |
| 24912 | 15768 | 27198 | 18054 | 29484 | 20340 | | 22626 |
| 24913 | 15769 | 27199 | 18055 | 29485 | 20341 | | 22627 |
| 24914 | 15770 | 27200 | 18056 | 29486 | 20342 | | 22628 |
| 24915 | 15771 | 27201 | 18057 | 29487 | 20343 | | 22629 |
| 24916 | 15772 | 27202 | 18058 | 29488 | 20344 | | 22630 |
| 24917 | 15773 | 27203 | 18059 | 29489 | 20345 | | 22631 |
| 24918 | 15774 | 27204 | 18060 | 29490 | 20346 | | 22632 |
| 24919 | 15775 | 27205 | 18061 | 29491 | 20347 | | 22633 |
| 24920 | 15776 | 27206 | 18062 | 29492 | 20348 | | 22634 |
| 24921 | 15777 | 27207 | 18063 | 29493 | 20349 | | 22635 |
| 24922 | 15778 | 27208 | 18064 | 29494 | 20350 | | 22636 |
| 24923 | 15779 | 27209 | 18065 | 29495 | 20351 | | 22637 |
| 24924 | 15780 | 27210 | 18066 | 29496 | 20352 | | 22638 |
| 24925 | 15781 | 27211 | 18067 | 29497 | 20353 | | 22639 |
| 24926 | 15782 | 27212 | 18068 | 29498 | 20354 | | 22640 |
| 24927 | 15783 | 27213 | 18069 | 29499 | 20355 | | 22641 |
| 24928 | 15784 | 27214 | 18070 | 29500 | 20356 | | 22642 |
| 24929 | 15785 | 27215 | 18071 | 29501 | 20357 | | 22643 |
| 24930 | 15786 | 27216 | 18072 | 29502 | 20358 | | 22644 |
| 24931 | 15787 | 27217 | 18073 | 29503 | 20359 | | 22645 |
| 24932 | 15788 | 27218 | 18074 | 29504 | 20360 | | 22646 |
| 24933 | 15789 | 27219 | 18075 | 29505 | 20361 | | 22647 |
| 24934 | 15790 | 27220 | 18076 | 29506 | 20362 | | 22648 |
| 24935 | 15791 | 27221 | 18077 | 29507 | 20363 | | 22649 |
| 24936 | 15792 | 27222 | 18078 | 29508 | 20364 | | 22650 |
| 24937 | 15793 | 27223 | 18079 | 29509 | 20365 | | 22651 |
| 24938 | 15794 | 27224 | 18080 | 29510 | 20366 | | 22652 |
| 24939 | 15795 | 27225 | 18081 | 29511 | 20367 | | 22653 |
| 24940 | 15796 | 27226 | 18082 | 29512 | 20368 | | 22654 |
| 24941 | 15797 | 27227 | 18083 | 29513 | 20369 | | 22655 |
| 24942 | 15798 | 27228 | 18084 | 29514 | 20370 | | 22656 |
| 24943 | 15799 | 27229 | 18085 | 29515 | 20371 | | 22657 |
| 24944 | 15800 | 27230 | 18086 | 29516 | 20372 | | 22658 |
| 24945 | 15801 | 27231 | 18087 | 29517 | 20373 | | 22659 |
| 24946 | 15802 | 27232 | 18088 | 29518 | 20374 | | 22660 |
| 24947 | 15803 | 27233 | 18089 | 29519 | 20375 | | 22661 |
| 24948 | 15804 | 27234 | 18090 | 29520 | 20376 | | 22662 |
| 24949 | 15805 | 27235 | 18091 | 29521 | 20377 | | 22663 |
| 24950 | 15806 | 27236 | 18092 | 29522 | 20378 | | 22664 |
| 24951 | 15807 | 27237 | 18093 | 29523 | 20379 | | 22665 |
| 24952 | 15808 | 27238 | 18094 | 29524 | 20380 | | 22666 |
| 24953 | 15809 | 27239 | 18095 | 29525 | 20381 | | 22667 |
| 24954 | 15810 | 27240 | 18096 | 29526 | 20382 | | 22668 |
| 24955 | 15811 | 27241 | 18097 | 29527 | 20383 | | 22669 |
| 24956 | 15812 | 27242 | 18098 | 29528 | 20384 | | 22670 |
| 24957 | 15813 | 27243 | 18099 | 29529 | 20385 | | 22671 |
| 24958 | 15814 | 27244 | 18100 | 29530 | 20386 | | 22672 |
| 24959 | 15815 | 27245 | 18101 | 29531 | 20387 | | 22673 |
| 24960 | 15816 | 27246 | 18102 | 29532 | 20388 | | 22674 |
| 24961 | 15817 | 27247 | 18103 | 29533 | 20389 | | 22675 |
| 24962 | 15818 | 27248 | 18104 | 29534 | 20390 | | 22676 |
| 24963 | 15819 | 27249 | 18105 | 29535 | 20391 | | 22677 |
| 24964 | 15820 | 27250 | 18106 | 29536 | 20392 | | 22678 |
| 24965 | 15821 | 27251 | 18107 | 29537 | 20393 | | 22679 |
| 24966 | 15822 | 27252 | 18108 | 29538 | 20394 | | 22680 |
| 24967 | 15823 | 27253 | 18109 | 29539 | 20395 | | 22681 |
| 24968 | 15824 | 27254 | 18110 | 29540 | 20396 | | 22682 |
| 24969 | 15825 | 27255 | 18111 | 29541 | 20397 | | 22683 |
| 24970 | 15826 | 27256 | 18112 | 29542 | 20398 | | 22684 |
| 24971 | 15827 | 27257 | 18113 | 29543 | 20399 | | 22685 |
| 24972 | 15828 | 27258 | 18114 | 29544 | 20400 | | 22686 |
| 24973 | 15829 | 27259 | 18115 | 29545 | 20401 | | 22687 |
| 24974 | 15830 | 27260 | 18116 | 29546 | 20402 | | 22688 |
| 24975 | 15831 | 27261 | 18117 | 29547 | 20403 | | 22689 |
| 24976 | 15832 | 27262 | 18118 | 29548 | 20404 | | 22690 |
| 24977 | 15833 | 27263 | 18119 | 29549 | 20405 | | 22691 |
| 24978 | 15834 | 27264 | 18120 | 29550 | 20406 | | 22692 |
| 24979 | 15835 | 27265 | 18121 | 29551 | 20407 | | 22693 |
| 24980 | 15836 | 27266 | 18122 | 29552 | 20408 | | 22694 |
| 24981 | 15837 | 27267 | 18123 | 29553 | 20409 | | 22695 |
| 24982 | 15838 | 27268 | 18124 | 29554 | 20410 | | 22696 |
| 24983 | 15839 | 27269 | 18125 | 29555 | 20411 | | 22697 |
| 24984 | 15840 | 27270 | 18126 | 29556 | 20412 | | 22698 |
| 24985 | 15841 | 27271 | 18127 | 29557 | 20413 | | 22699 |
| 24986 | 15842 | 27272 | 18128 | 29558 | 20414 | | 22700 |
| 24987 | 15843 | 27273 | 18129 | 29559 | 20415 | | 22701 |
| 24988 | 15844 | 27274 | 18130 | 29560 | 20416 | | 22702 |
| 24989 | 15845 | 27275 | 18131 | 29561 | 20417 | | 22703 |
| 24990 | 15846 | 27276 | 18132 | 29562 | 20418 | | 22704 |
| 24991 | 15847 | 27277 | 18133 | 29563 | 20419 | | 22705 |
| 24992 | 15848 | 27278 | 18134 | 29564 | 20420 | | 22706 |
| 24993 | 15849 | 27279 | 18135 | 29565 | 20421 | | 22707 |
| 24994 | 15850 | 27280 | 18136 | 29566 | 20422 | | 22708 |
| 24995 | 15851 | 27281 | 18137 | 29567 | 20423 | | 22709 |
| 24996 | 15852 | 27282 | 18138 | 29568 | 20424 | | 22710 |
| 24997 | 15853 | 27283 | 18139 | 29569 | 20425 | | 22711 |
| 24998 | 15854 | 27284 | 18140 | 29570 | 20426 | | 22712 |
| 24999 | 15855 | 27285 | 18141 | 29571 | 20427 | | 22713 |
| 25000 | 15856 | 27286 | 18142 | 29572 | 20428 | | 22714 |
| 25001 | 15857 | 27287 | 18143 | 29573 | 20429 | | 22715 |
| 25002 | 15858 | 27288 | 18144 | 29574 | 20430 | | 22716 |
| 25003 | 15859 | 27289 | 18145 | 29575 | 20431 | | 22717 |
| 25004 | 15860 | 27290 | 18146 | 29576 | 20432 | | 22718 |
| 25005 | 15861 | 27291 | 18147 | 29577 | 20433 | | 22719 |
| 25006 | 15862 | 27292 | 18148 | 29578 | 20434 | | 22720 |
| 25007 | 15863 | 27293 | 18149 | 29579 | 20435 | | 22721 |
| 25008 | 15864 | 27294 | 18150 | 29580 | 20436 | | 22722 |
| 25009 | 15865 | 27295 | 18151 | 29581 | 20437 | | 22723 |
| 25010 | 15866 | 27296 | 18152 | 29582 | 20438 | | 22724 |
| 25011 | 15867 | 27297 | 18153 | 29583 | 20439 | | 22725 |
| 25012 | 15868 | 27298 | 18154 | 29584 | 20440 | | 22726 |
| 25013 | 15869 | 27299 | 18155 | 29585 | 20441 | | 22727 |
| 25014 | 15870 | 27300 | 18156 | 29586 | 20442 | | 22728 |
| 25015 | 15871 | 27301 | 18157 | 29587 | 20443 | | 22729 |
| 25016 | 15872 | 27302 | 18158 | 29588 | 20444 | | 22730 |
| 25017 | 15873 | 27303 | 18159 | 29589 | 20445 | | 22731 |
| 25018 | 15874 | 27304 | 18160 | 29590 | 20446 | | 22732 |
| 25019 | 15875 | 27305 | 18161 | 29591 | 20447 | | 22733 |
| 25020 | 15876 | 27306 | 18162 | 29592 | 20448 | | 22734 |
| 25021 | 15877 | 27307 | 18163 | 29593 | 20449 | | 22735 |
| 25022 | 15878 | 27308 | 18164 | 29594 | 20450 | | 22736 |
| 25023 | 15879 | 27309 | 18165 | 29595 | 20451 | | 22737 |
| 25024 | 15880 | 27310 | 18166 | 29596 | 20452 | | 22738 |
| 25025 | 15881 | 27311 | 18167 | 29597 | 20453 | | 22739 |
| 25026 | 15882 | 27312 | 18168 | 29598 | 20454 | | 22740 |
| 25027 | 15883 | 27313 | 18169 | 29599 | 20455 | | 22741 |
| 25028 | 15884 | 27314 | 18170 | 29600 | 20456 | | 22742 |
| 25029 | 15885 | 27315 | 18171 | 29601 | 20457 | | 22743 |
| 25030 | 15886 | 27316 | 18172 | 29602 | 20458 | | 22744 |
| 25031 | 15887 | 27317 | 18173 | 29603 | 20459 | | 22745 |
| 25032 | 15888 | 27318 | 18174 | 29604 | 20460 | | 22746 |
| 25033 | 15889 | 27319 | 18175 | 29605 | 20461 | | 22747 |
| 25034 | 15890 | 27320 | 18176 | 29606 | 20462 | | 22748 |
| 25035 | 15891 | 27321 | 18177 | 29607 | 20463 | | 22749 |
| 25036 | 15892 | 27322 | 18178 | 29608 | 20464 | | 22750 |
| 25037 | 15893 | 27323 | 18179 | 29609 | 20465 | | 22751 |
| 25038 | 15894 | 27324 | 18180 | 29610 | 20466 | | 22752 |
| 25039 | 15895 | 27325 | 18181 | 29611 | 20467 | | 22753 |
| 25040 | 15896 | 27326 | 18182 | 29612 | 20468 | | 22754 |
| 25041 | 15897 | 27327 | 18183 | 29613 | 20469 | | 22755 |
| 25042 | 15898 | 27328 | 18184 | 29614 | 20470 | | 22756 |
| 25043 | 15899 | 27329 | 18185 | 29615 | 20471 | | 22757 |
| 25044 | 15900 | 27330 | 18186 | 29616 | 20472 | | 22758 |
| 25045 | 15901 | 27331 | 18187 | 29617 | 20473 | | 22759 |
| 25046 | 15902 | 27332 | 18188 | 29618 | 20474 | | 22760 |
| 25047 | 15903 | 27333 | 18189 | 29619 | 20475 | | 22761 |
| 25048 | 15904 | 27334 | 18190 | 29620 | 20476 | | 22762 |
| 25049 | 15905 | 27335 | 18191 | 29621 | 20477 | | 22763 |
| 25050 | 15906 | 27336 | 18192 | 29622 | 20478 | | 22764 |
| 25051 | 15907 | 27337 | 18193 | 29623 | 20479 | | 22765 |
| 25052 | 15908 | 27338 | 18194 | 29624 | 20480 | | 22766 |
| 25053 | 15909 | 27339 | 18195 | 29625 | 20481 | | 22767 |
| 25054 | 15910 | 27340 | 18196 | 29626 | 20482 | | 22768 |
| 25055 | 15911 | 27341 | 18197 | 29627 | 20483 | | 22769 |
| 25056 | 15912 | 27342 | 18198 | 29628 | 20484 | | 22770 |
| 25057 | 15913 | 27343 | 18199 | 29629 | 20485 | | 22771 |
| 25058 | 15914 | 27344 | 18200 | 29630 | 20486 | | 22772 |
| 25059 | 15915 | 27345 | 18201 | 29631 | 20487 | | 22773 |
| 25060 | 15916 | 27346 | 18202 | 29632 | 20488 | | 22774 |
| 25061 | 15917 | 27347 | 18203 | 29633 | 20489 | | 22775 |
| 25062 | 15918 | 27348 | 18204 | 29634 | 20490 | | 22776 |
| 25063 | 15919 | 27349 | 18205 | 29635 | 20491 | | 22777 |
| 25064 | 15920 | 27350 | 18206 | 29636 | 20492 | | 22778 |
| 25065 | 15921 | 27351 | 18207 | 29637 | 20493 | | 22779 |
| 25066 | 15922 | 27352 | 18208 | 29638 | 20494 | | 22780 |
| 25067 | 15923 | 27353 | 18209 | 29639 | 20495 | | 22781 |
| 25068 | 15924 | 27354 | 18210 | 29640 | 20496 | | 22782 |
| 25069 | 15925 | 27355 | 18211 | 29641 | 20497 | | 22783 |
| 25070 | 15926 | 27356 | 18212 | 29642 | 20498 | | 22784 |
| 25071 | 15927 | 27357 | 18213 | 29643 | 20499 | | 22785 |
| 25072 | 15928 | 27358 | 18214 | 29644 | 20500 | | 22786 |
| 25073 | 15929 | 27359 | 18215 | 29645 | 20501 | | 22787 |
| 25074 | 15930 | 27360 | 18216 | 29646 | 20502 | | 22788 |
| 25075 | 15931 | 27361 | 18217 | 29647 | 20503 | | 22789 |
| 25076 | 15932 | 27362 | 18218 | 29648 | 20504 | | 22790 |
| 25077 | 15933 | 27363 | 18219 | 29649 | 20505 | | 22791 |
| 25078 | 15934 | 27364 | 18220 | 29650 | 20506 | | 22792 |
| 25079 | 15935 | 27365 | 18221 | 29651 | 20507 | | 22793 |
| 25080 | 15936 | 27366 | 18222 | 29652 | 20508 | | 22794 |
| 25081 | 15937 | 27367 | 18223 | 29653 | 20509 | | 22795 |
| 25082 | 15938 | 27368 | 18224 | 29654 | 20510 | | 22796 |
| 25083 | 15939 | 27369 | 18225 | 29655 | 20511 | | 22797 |
| 25084 | 15940 | 27370 | 18226 | 29656 | 20512 | | 22798 |
| 25085 | 15941 | 27371 | 18227 | 29657 | 20513 | | 22799 |
| 25086 | 15942 | 27372 | 18228 | 29658 | 20514 | | 22800 |
| 25087 | 15943 | 27373 | 18229 | 29659 | 20515 | | 22801 |
| 25088 | 15944 | 27374 | 18230 | 29660 | 20516 | | 22802 |
| 25089 | 15945 | 27375 | 18231 | 29661 | 20517 | | 22803 |
| 25090 | 15946 | 27376 | 18232 | 29662 | 20518 | | 22804 |
| 25091 | 15947 | 27377 | 18233 | 29663 | 20519 | | 22805 |
| 25092 | 15948 | 27378 | 18234 | 29664 | 20520 | | 22806 |
| 25093 | 15949 | 27379 | 18235 | 29665 | 20521 | | 22807 |
| 25094 | 15950 | 27380 | 18236 | 29666 | 20522 | | 22808 |
| 25095 | 15951 | 27381 | 18237 | 29667 | 20523 | | 22809 |
| 25096 | 15952 | 27382 | 18238 | 29668 | 20524 | | 22810 |
| 25097 | 15953 | 27383 | 18239 | 29669 | 20525 | | 22811 |
| 25098 | 15954 | 27384 | 18240 | 29670 | 20526 | | 22812 |
| 25099 | 15955 | 27385 | 18241 | 29671 | 20527 | | 22813 |
| 25100 | 15956 | 27386 | 18242 | 29672 | 20528 | | 22814 |
| 25101 | 15957 | 27387 | 18243 | 29673 | 20529 | | 22815 |
| 25102 | 15958 | 27388 | 18244 | 29674 | 20530 | | 22816 |
| 25103 | 15959 | 27389 | 18245 | 29675 | 20531 | | 22817 |
| 25104 | 15960 | 27390 | 18246 | 29676 | 20532 | | 22818 |
| 25105 | 15961 | 27391 | 18247 | 29677 | 20533 | | 22819 |
| 25106 | 15962 | 27392 | 18248 | 29678 | 20534 | | 22820 |
| 25107 | 15963 | 27393 | 18249 | 29679 | 20535 | | 22821 |
| 25108 | 15964 | 27394 | 18250 | 29680 | 20536 | | 22822 |
| 25109 | 15965 | 27395 | 18251 | 29681 | 20537 | | 22823 |
| 25110 | 15966 | 27396 | 18252 | 29682 | 20538 | | 22824 |
| 25111 | 15967 | 27397 | 18253 | 29683 | 20539 | | 22825 |
| 25112 | 15968 | 27398 | 18254 | 29684 | 20540 | | 22826 |
| 25113 | 15969 | 27399 | 18255 | 29685 | 20541 | | 22827 |
| 25114 | 15970 | 27400 | 18256 | 29686 | 20542 | | 22828 |
| 25115 | 15971 | 27401 | 18257 | 29687 | 20543 | | 22829 |
| 25116 | 15972 | 27402 | 18258 | 29688 | 20544 | | 22830 |
| 25117 | 15973 | 27403 | 18259 | 29689 | 20545 | | 22831 |
| 25118 | 15974 | 27404 | 18260 | 29690 | 20546 | | 22832 |
| 25119 | 15975 | 27405 | 18261 | 29691 | 20547 | | 22833 |
| 25120 | 15976 | 27406 | 18262 | 29692 | 20548 | | 22834 |
| 25121 | 15977 | 27407 | 18263 | 29693 | 20549 | | 22835 |
| 25122 | 15978 | 27408 | 18264 | 29694 | 20550 | | 22836 |
| 25123 | 15979 | 27409 | 18265 | 29695 | 20551 | | 22837 |
| 25124 | 15980 | 27410 | 18266 | 29696 | 20552 | | 22838 |
| 25125 | 15981 | 27411 | 18267 | 29697 | 20553 | | 22839 |
| 25126 | 15982 | 27412 | 18268 | 29698 | 20554 | | 22840 |
| 25127 | 15983 | 27413 | 18269 | 29699 | 20555 | | 22841 |
| 25128 | 15984 | 27414 | 18270 | 29700 | 20556 | | 22842 |
| 25129 | 15985 | 27415 | 18271 | 29701 | 20557 | | 22843 |
| 25130 | 15986 | 27416 | 18272 | 29702 | 20558 | | 22844 |
| 25131 | 15987 | 27417 | 18273 | 29703 | 20559 | | 22845 |
| 25132 | 15988 | 27418 | 18274 | 29704 | 20560 | | 22846 |
| 25133 | 15989 | 27419 | 18275 | 29705 | 20561 | | 22847 |
| 25134 | 15990 | 27420 | 18276 | 29706 | 20562 | | 22848 |
| 25135 | 15991 | 27421 | 18277 | 29707 | 20563 | | 22849 |
| 25136 | 15992 | 27422 | 18278 | 29708 | 20564 | | 22850 |
| 25137 | 15993 | 27423 | 18279 | 29709 | 20565 | | 22851 |
| 25138 | 15994 | 27424 | 18280 | 29710 | 20566 | | 22852 |
| 25139 | 15995 | 27425 | 18281 | 29711 | 20567 | | 22853 |
| 25140 | 15996 | 27426 | 18282 | 29712 | 20568 | | 22854 |
| 25141 | 15997 | 27427 | 18283 | 29713 | 20569 | | 22855 |
| 25142 | 15998 | 27428 | 18284 | 29714 | 20570 | | 22856 |
| 25143 | 15999 | 27429 | 18285 | 29715 | 20571 | | 22857 |
| 25144 | 16000 | 27430 | 18286 | 29716 | 20572 | | 22858 |
| 25145 | 16001 | 27431 | 18287 | 29717 | 20573 | | 22859 |
| 25146 | 16002 | 27432 | 18288 | 29718 | 20574 | | 22860 |
| 25147 | 16003 | 27433 | 18289 | 29719 | 20575 | | 22861 |
| 25148 | 16004 | 27434 | 18290 | 29720 | 20576 | | 22862 |
| 25149 | 16005 | 27435 | 18291 | 29721 | 20577 | | 22863 |
| 25150 | 16006 | 27436 | 18292 | 29722 | 20578 | | 22864 |
| 25151 | 16007 | 27437 | 18293 | 29723 | 20579 | | 22865 |
| 25152 | 16008 | 27438 | 18294 | 29724 | 20580 | | 22866 |
| 25153 | 16009 | 27439 | 18295 | 29725 | 20581 | | 22867 |
| 25154 | 16010 | 27440 | 18296 | 29726 | 20582 | | 22868 |
| 25155 | 16011 | 27441 | 18297 | 29727 | 20583 | | 22869 |
| 25156 | 16012 | 27442 | 18298 | 29728 | 20584 | | 22870 |
| 25157 | 16013 | 27443 | 18299 | 29729 | 20585 | | 22871 |
| 25158 | 16014 | 27444 | 18300 | 29730 | 20586 | | 22872 |
| 25159 | 16015 | 27445 | 18301 | 29731 | 20587 | | 22873 |
| 25160 | 16016 | 27446 | 18302 | 29732 | 20588 | | 22874 |
| 25161 | 16017 | 27447 | 18303 | 29733 | 20589 | | 22875 |
| 25162 | 16018 | 27448 | 18304 | 29734 | 20590 | | 22876 |
| 25163 | 16019 | 27449 | 18305 | 29735 | 20591 | | 22877 |
| 25164 | 16020 | 27450 | 18306 | 29736 | 20592 | | 22878 |
| 25165 | 16021 | 27451 | 18307 | 29737 | 20593 | | 22879 |
| 25166 | 16022 | 27452 | 18308 | 29738 | 20594 | | 22880 |
| 25167 | 16023 | 27453 | 18309 | 29739 | 20595 | | 22881 |
| 25168 | 16024 | 27454 | 18310 | 29740 | 20596 | | 22882 |
| 25169 | 16025 | 27455 | 18311 | 29741 | 20597 | | 22883 |
| 25170 | 16026 | 27456 | 18312 | 29742 | 20598 | | 22884 |
| 25171 | 16027 | 27457 | 18313 | 29743 | 20599 | | 22885 |
| 25172 | 16028 | 27458 | 18314 | 29744 | 20600 | | 22886 |
| 25173 | 16029 | 27459 | 18315 | 29745 | 20601 | | 22887 |
| 25174 | 16030 | 27460 | 18316 | 29746 | 20602 | | 22888 |
| 25175 | 16031 | 27461 | 18317 | 29747 | 20603 | | 22889 |
| 25176 | 16032 | 27462 | 18318 | 29748 | 20604 | | 22890 |
| 25177 | 16033 | 27463 | 18319 | 29749 | 20605 | | 22891 |
| 25178 | 16034 | 27464 | 18320 | 29750 | 20606 | | 22892 |
| 25179 | 16035 | 27465 | 18321 | 29751 | 20607 | | 22893 |
| 25180 | 16036 | 27466 | 18322 | 29752 | 20608 | | 22894 |
| 25181 | 16037 | 27467 | 18323 | 29753 | 20609 | | 22895 |
| 25182 | 16038 | 27468 | 18324 | 29754 | 20610 | | 22896 |
| 25183 | 16039 | 27469 | 18325 | 29755 | 20611 | | 22897 |
| 25184 | 16040 | 27470 | 18326 | 29756 | 20612 | | 22898 |
| 25185 | 16041 | 27471 | 18327 | 29757 | 20613 | | 22899 |
| 25186 | 16042 | 27472 | 18328 | 29758 | 20614 | | 22900 |
| 25187 | 16043 | 27473 | 18329 | 29759 | 20615 | | 22901 |
| 25188 | 16044 | 27474 | 18330 | 29760 | 20616 | | 22902 |
| 25189 | 16045 | 27475 | 18331 | 29761 | 20617 | | 22903 |
| 25190 | 16046 | 27476 | 18332 | 29762 | 20618 | | 22904 |
| 25191 | 16047 | 27477 | 18333 | 29763 | 20619 | | 22905 |
| 25192 | 16048 | 27478 | 18334 | 29764 | 20620 | | 22906 |
| 25193 | 16049 | 27479 | 18335 | 29765 | 20621 | | 22907 |
| 25194 | 16050 | 27480 | 18336 | 29766 | 20622 | | 22908 |
| 25195 | 16051 | 27481 | 18337 | 29767 | 20623 | | 22909 |
| 25196 | 16052 | 27482 | 18338 | 29768 | 20624 | | 22910 |
| 25197 | 16053 | 27483 | 18339 | 29769 | 20625 | | 22911 |
| 25198 | 16054 | 27484 | 18340 | 29770 | 20626 | | 22912 |
| 25199 | 16055 | 27485 | 18341 | 29771 | 20627 | | 22913 |
| 25200 | 16056 | 27486 | 18342 | 29772 | 20628 | | 22914 |
| 25201 | 16057 | 27487 | 18343 | 29773 | 20629 | | 22915 |
| 25202 | 16058 | 27488 | 18344 | 29774 | 20630 | | 22916 |
| 25203 | 16059 | 27489 | 18345 | 29775 | 20631 | | 22917 |
| 25204 | 16060 | 27490 | 18346 | 29776 | 20632 | | 22918 |
| 25205 | 16061 | 27491 | 18347 | 29777 | 20633 | | 22919 |
| 25206 | 16062 | 27492 | 18348 | 29778 | 20634 | | 22920 |
| 25207 | 16063 | 27493 | 18349 | 29779 | 20635 | | 22921 |
| 25208 | 16064 | 27494 | 18350 | 29780 | 20636 | | 22922 |
| 25209 | 16065 | 27495 | 18351 | 29781 | 20637 | | 22923 |
| 25210 | 16066 | 27496 | 18352 | 29782 | 20638 | | 22924 |
| 25211 | 16067 | 27497 | 18353 | 29783 | 20639 | | 22925 |
| 25212 | 16068 | 27498 | 18354 | 29784 | 20640 | | 22926 |
| 25213 | 16069 | 27499 | 18355 | 29785 | 20641 | | 22927 |
| 25214 | 16070 | 27500 | 18356 | 29786 | 20642 | | 22928 |
| 25215 | 16071 | 27501 | 18357 | 29787 | 20643 | | 22929 |
| 25216 | 16072 | 27502 | 18358 | 29788 | 20644 | | 22930 |
| 25217 | 16073 | 27503 | 18359 | 29789 | 20645 | | 22931 |
| 25218 | 16074 | 27504 | 18360 | 29790 | 20646 | | 22932 |
| 25219 | 16075 | 27505 | 18361 | 29791 | 20647 | | 22933 |
| 25220 | 16076 | 27506 | 18362 | 29792 | 20648 | | 22934 |
| 25221 | 16077 | 27507 | 18363 | 29793 | 20649 | | 22935 |
| 25222 | 16078 | 27508 | 18364 | 29794 | 20650 | | 22936 |
| 25223 | 16079 | 27509 | 18365 | 29795 | 20651 | | 22937 |
| 25224 | 16080 | 27510 | 18366 | 29796 | 20652 | | 22938 |
| 25225 | 16081 | 27511 | 18367 | 29797 | 20653 | | 22939 |
| 25226 | 16082 | 27512 | 18368 | 29798 | 20654 | | 22940 |
| 25227 | 16083 | 27513 | 18369 | 29799 | 20655 | | 22941 |
| 25228 | 16084 | 27514 | 18370 | 29800 | 20656 | | 22942 |
| 25229 | 16085 | 27515 | 18371 | 29801 | 20657 | | 22943 |
| 25230 | 16086 | 27516 | 18372 | 29802 | 20658 | | 22944 |
| 25231 | 16087 | 27517 | 18373 | 29803 | 20659 | | 22945 |
| 25232 | 16088 | 27518 | 18374 | 29804 | 20660 | | 22946 |
| 25233 | 16089 | 27519 | 18375 | 29805 | 20661 | | 22947 |
| 25234 | 16090 | 27520 | 18376 | 29806 | 20662 | | 22948 |
| 25235 | 16091 | 27521 | 18377 | 29807 | 20663 | | 22949 |
| 25236 | 16092 | 27522 | 18378 | 29808 | 20664 | | 22950 |
| 25237 | 16093 | 27523 | 18379 | 29809 | 20665 | | 22951 |
| 25238 | 16094 | 27524 | 18380 | 29810 | 20666 | | 22952 |
| 25239 | 16095 | 27525 | 18381 | 29811 | 20667 | | 22953 |
| 25240 | 16096 | 27526 | 18382 | 29812 | 20668 | | 22954 |
| 25241 | 16097 | 27527 | 18383 | 29813 | 20669 | | 22955 |
| 25242 | 16098 | 27528 | 18384 | 29814 | 20670 | | 22956 |
| 25243 | 16099 | 27529 | 18385 | 29815 | 20671 | | 22957 |
| 25244 | 16100 | 27530 | 18386 | 29816 | 20672 | | 22958 |
| 25245 | 16101 | 27531 | 18387 | 29817 | 20673 | | 22959 |
| 25246 | 16102 | 27532 | 18388 | 29818 | 20674 | | 22960 |
| 25247 | 16103 | 27533 | 18389 | 29819 | 20675 | | 22961 |
| 25248 | 16104 | 27534 | 18390 | 29820 | 20676 | | 22962 |
| 25249 | 16105 | 27535 | 18391 | 29821 | 20677 | | 22963 |
| 25250 | 16106 | 27536 | 18392 | 29822 | 20678 | | 22964 |
| 25251 | 16107 | 27537 | 18393 | 29823 | 20679 | | 22965 |
| 25252 | 16108 | 27538 | 18394 | 29824 | 20680 | | 22966 |
| 25253 | 16109 | 27539 | 18395 | 29825 | 20681 | | 22967 |
| 25254 | 16110 | 27540 | 18396 | 29826 | 20682 | | 22968 |
| 25255 | 16111 | 27541 | 18397 | 29827 | 20683 | | 22969 |
| 25256 | 16112 | 27542 | 18398 | 29828 | 20684 | | 22970 |
| 25257 | 16113 | 27543 | 18399 | 29829 | 20685 | | 22971 |
| 25258 | 16114 | 27544 | 18400 | 29830 | 20686 | | 22972 |
| 25259 | 16115 | 27545 | 18401 | 29831 | 20687 | | 22973 |
| 25260 | 16116 | 27546 | 18402 | 29832 | 20688 | | 22974 |
| 25261 | 16117 | 27547 | 18403 | 29833 | 20689 | | 22975 |
| 25262 | 16118 | 27548 | 18404 | 29834 | 20690 | | 22976 |
| 25263 | 16119 | 27549 | 18405 | 29835 | 20691 | | 22977 |
| 25264 | 16120 | 27550 | 18406 | 29836 | 20692 | | 22978 |
| 25265 | 16121 | 27551 | 18407 | 29837 | 20693 | | 22979 |
| 25266 | 16122 | 27552 | 18408 | 29838 | 20694 | | 22980 |
| 25267 | 16123 | 27553 | 18409 | 29839 | 20695 | | 22981 |
| 25268 | 16124 | 27554 | 18410 | 29840 | 20696 | | 22982 |
| 25269 | 16125 | 27555 | 18411 | 29841 | 20697 | | 22983 |
| 25270 | 16126 | 27556 | 18412 | 29842 | 20698 | | 22984 |
| 25271 | 16127 | 27557 | 18413 | 29843 | 20699 | | 22985 |
| 25272 | 16128 | 27558 | 18414 | 29844 | 20700 | | 22986 |
| 25273 | 16129 | 27559 | 18415 | 29845 | 20701 | | 22987 |
| 25274 | 16130 | 27560 | 18416 | 29846 | 20702 | | 22988 |
| 25275 | 16131 | 27561 | 18417 | 29847 | 20703 | | 22989 |
| 25276 | 16132 | 27562 | 18418 | 29848 | 20704 | | 22990 |
| 25277 | 16133 | 27563 | 18419 | 29849 | 20705 | | 22991 |
| 25278 | 16134 | 27564 | 18420 | 29850 | 20706 | | 22992 |
| 25279 | 16135 | 27565 | 18421 | 29851 | 20707 | | 22993 |
| 25280 | 16136 | 27566 | 18422 | 29852 | 20708 | | 22994 |
| 25281 | 16137 | 27567 | 18423 | 29853 | 20709 | | 22995 |
| 25282 | 16138 | 27568 | 18424 | 29854 | 20710 | | 22996 |
| 25283 | 16139 | 27569 | 18425 | 29855 | 20711 | | 22997 |
| 25284 | 16140 | 27570 | 18426 | 29856 | 20712 | | 22998 |
| 25285 | 16141 | 27571 | 18427 | 29857 | 20713 | | 22999 |
| 25286 | 16142 | 27572 | 18428 | 29858 | 20714 | | 23000 |
| 25287 | 16143 | 27573 | 18429 | 29859 | 20715 | | 23001 |
| 25288 | 16144 | 27574 | 18430 | 29860 | 20716 | | 23002 |
| 25289 | 16145 | 27575 | 18431 | 29861 | 20717 | | 23003 |
| 25290 | 16146 | 27576 | 18432 | 29862 | 20718 | | 23004 |
| 25291 | 16147 | 27577 | 18433 | 29863 | 20719 | | 23005 |
| 25292 | 16148 | 27578 | 18434 | 29864 | 20720 | | 23006 |
| 25293 | 16149 | 27579 | 18435 | 29865 | 20721 | | 23007 |
| 25294 | 16150 | 27580 | 18436 | 29866 | 20722 | | 23008 |
| 25295 | 16151 | 27581 | 18437 | 29867 | 20723 | | 23009 |
| 25296 | 16152 | 27582 | 18438 | 29868 | 20724 | | 23010 |
| 25297 | 16153 | 27583 | 18439 | 29869 | 20725 | | 23011 |
| 25298 | 16154 | 27584 | 18440 | 29870 | 20726 | | 23012 |
| 25299 | 16155 | 27585 | 18441 | 29871 | 20727 | | 23013 |
| 25300 | 16156 | 27586 | 18442 | 29872 | 20728 | | 23014 |
| 25301 | 16157 | 27587 | 18443 | 29873 | 20729 | | 23015 |
| 25302 | 16158 | 27588 | 18444 | 29874 | 20730 | | 23016 |
| 25303 | 16159 | 27589 | 18445 | 29875 | 20731 | | 23017 |
| 25304 | 16160 | 27590 | 18446 | 29876 | 20732 | | 23018 |
| 25305 | 16161 | 27591 | 18447 | 29877 | 20733 | | 23019 |
| 25306 | 16162 | 27592 | 18448 | 29878 | 20734 | | 23020 |
| 25307 | 16163 | 27593 | 18449 | 29879 | 20735 | | 23021 |
| 25308 | 16164 | 27594 | 18450 | 29880 | 20736 | | 23022 |
| 25309 | 16165 | 27595 | 18451 | 29881 | 20737 | | 23023 |
| 25310 | 16166 | 27596 | 18452 | 29882 | 20738 | | 23024 |
| 25311 | 16167 | 27597 | 18453 | 29883 | 20739 | | 23025 |
| 25312 | 16168 | 27598 | 18454 | 29884 | 20740 | | 23026 |
| 25313 | 16169 | 27599 | 18455 | 29885 | 20741 | | 23027 |
| 25314 | 16170 | 27600 | 18456 | 29886 | 20742 | | 23028 |
| 25315 | 16171 | 27601 | 18457 | 29887 | 20743 | | 23029 |
| 25316 | 16172 | 27602 | 18458 | 29888 | 20744 | | 23030 |
| 25317 | 16173 | 27603 | 18459 | 29889 | 20745 | | 23031 |
| 25318 | 16174 | 27604 | 18460 | 29890 | 20746 | | 23032 |
| 25319 | 16175 | 27605 | 18461 | 29891 | 20747 | | 23033 |
| 25320 | 16176 | 27606 | 18462 | 29892 | 20748 | | 23034 |
| 25321 | 16177 | 27607 | 18463 | 29893 | 20749 | | 23035 |
| 25322 | 16178 | 27608 | 18464 | 29894 | 20750 | | 23036 |
| 25323 | 16179 | 27609 | 18465 | 29895 | 20751 | | 23037 |
| 25324 | 16180 | 27610 | 18466 | 29896 | 20752 | | 23038 |
| 25325 | 16181 | 27611 | 18467 | 29897 | 20753 | | 23039 |
| 25326 | 16182 | 27612 | 18468 | 29898 | 20754 | | 23040 |
| 25327 | 16183 | 27613 | 18469 | 29899 | 20755 | | 23041 |
| 25328 | 16184 | 27614 | 18470 | 29900 | 20756 | | 23042 |
| 25329 | 16185 | 27615 | 18471 | 29901 | 20757 | | 23043 |
| 25330 | 16186 | 27616 | 18472 | 29902 | 20758 | | 23044 |
| 25331 | 16187 | 27617 | 18473 | 29903 | 20759 | | 23045 |
| 25332 | 16188 | 27618 | 18474 | 29904 | 20760 | | 23046 |
| 25333 | 16189 | 27619 | 18475 | 29905 | 20761 | | 23047 |
| 25334 | 16190 | 27620 | 18476 | 29906 | 20762 | | 23048 |
| 25335 | 16191 | 27621 | 18477 | 29907 | 20763 | | 23049 |
| 25336 | 16192 | 27622 | 18478 | 29908 | 20764 | | 23050 |
| 25337 | 16193 | 27623 | 18479 | 29909 | 20765 | | 23051 |
| 25338 | 16194 | 27624 | 18480 | 29910 | 20766 | | 23052 |
| 25339 | 16195 | 27625 | 18481 | 29911 | 20767 | | 23053 |
| 25340 | 16196 | 27626 | 18482 | 29912 | 20768 | | 23054 |
| 25341 | 16197 | 27627 | 18483 | 29913 | 20769 | | 23055 |
| 25342 | 16198 | 27628 | 18484 | 29914 | 20770 | | 23056 |
| 25343 | 16199 | 27629 | 18485 | 29915 | 20771 | | 23057 |
| 25344 | 16200 | 27630 | 18486 | 29916 | 20772 | | 23058 |
| 25345 | 16201 | 27631 | 18487 | 29917 | 20773 | | 23059 |
| 25346 | 16202 | 27632 | 18488 | 29918 | 20774 | | 23060 |
| 25347 | 16203 | 27633 | 18489 | 29919 | 20775 | | 23061 |
| 25348 | 16204 | 27634 | 18490 | 29920 | 20776 | | 23062 |
| 25349 | 16205 | 27635 | 18491 | 29921 | 20777 | | 23063 |
| 25350 | 16206 | 27636 | 18492 | 29922 | 20778 | | 23064 |
| 25351 | 16207 | 27637 | 18493 | 29923 | 20779 | | 23065 |
| 25352 | 16208 | 27638 | 18494 | 29924 | 20780 | | 23066 |
| 25353 | 16209 | 27639 | 18495 | 29925 | 20781 | | 23067 |
| 25354 | 16210 | 27640 | 18496 | 29926 | 20782 | | 23068 |
| 25355 | 16211 | 27641 | 18497 | 29927 | 20783 | | 23069 |
| 25356 | 16212 | 27642 | 18498 | 29928 | 20784 | | 23070 |
| 25357 | 16213 | 27643 | 18499 | 29929 | 20785 | | 23071 |
| 25358 | 16214 | 27644 | 18500 | 29930 | 20786 | | 23072 |
| 25359 | 16215 | 27645 | 18501 | 29931 | 20787 | | 23073 |
| 25360 | 16216 | 27646 | 18502 | 29932 | 20788 | | 23074 |
| 25361 | 16217 | 27647 | 18503 | 29933 | 20789 | | 23075 |
| 25362 | 16218 | 27648 | 18504 | 29934 | 20790 | | 23076 |
| 25363 | 16219 | 27649 | 18505 | 29935 | 20791 | | 23077 |
| 25364 | 16220 | 27650 | 18506 | 29936 | 20792 | | 23078 |
| 25365 | 16221 | 27651 | 18507 | 29937 | 20793 | | 23079 |
| 25366 | 16222 | 27652 | 18508 | 29938 | 20794 | | 23080 |
| 25367 | 16223 | 27653 | 18509 | 29939 | 20795 | | 23081 |
| 25368 | 16224 | 27654 | 18510 | 29940 | 20796 | | 23082 |
| 25369 | 16225 | 27655 | 18511 | 29941 | 20797 | | 23083 |
| 25370 | 16226 | 27656 | 18512 | 29942 | 20798 | | 23084 |
| 25371 | 16227 | 27657 | 18513 | 29943 | 20799 | | 23085 |
| 25372 | 16228 | 27658 | 18514 | 29944 | 20800 | | 23086 |
| 25373 | 16229 | 27659 | 18515 | 29945 | 20801 | | 23087 |
| 25374 | 16230 | 27660 | 18516 | 29946 | 20802 | | 23088 |
| 25375 | 16231 | 27661 | 18517 | 29947 | 20803 | | 23089 |
| 25376 | 16232 | 27662 | 18518 | 29948 | 20804 | | 23090 |
| 25377 | 16233 | 27663 | 18519 | 29949 | 20805 | | 23091 |
| 25378 | 16234 | 27664 | 18520 | 29950 | 20806 | | 23092 |
| 25379 | 16235 | 27665 | 18521 | 29951 | 20807 | | 23093 |
| 25380 | 16236 | 27666 | 18522 | 29952 | 20808 | | 23094 |
| 25381 | 16237 | 27667 | 18523 | 29953 | 20809 | | 23095 |
| 25382 | 16238 | 27668 | 18524 | 29954 | 20810 | | 23096 |
| 25383 | 16239 | 27669 | 18525 | 29955 | 20811 | | 23097 |
| 25384 | 16240 | 27670 | 18526 | 29956 | 20812 | | 23098 |
| 25385 | 16241 | 27671 | 18527 | 29957 | 20813 | | 23099 |
| 25386 | 16242 | 27672 | 18528 | 29958 | 20814 | | 23100 |
| 25387 | 16243 | 27673 | 18529 | 29959 | 20815 | | 23101 |
| 25388 | 16244 | 27674 | 18530 | 29960 | 20816 | | 23102 |
| 25389 | 16245 | 27675 | 18531 | 29961 | 20817 | | 23103 |
| 25390 | 16246 | 27676 | 18532 | 29962 | 20818 | | 23104 |
| 25391 | 16247 | 27677 | 18533 | 29963 | 20819 | | 23105 |
| 25392 | 16248 | 27678 | 18534 | 29964 | 20820 | | 23106 |
| 25393 | 16249 | 27679 | 18535 | 29965 | 20821 | | 23107 |
| 25394 | 16250 | 27680 | 18536 | 29966 | 20822 | | 23108 |
| 25395 | 16251 | 27681 | 18537 | 29967 | 20823 | | 23109 |
| 25396 | 16252 | 27682 | 18538 | 29968 | 20824 | | 23110 |
| 25397 | 16253 | 27683 | 18539 | 29969 | 20825 | | 23111 |
| 25398 | 16254 | 27684 | 18540 | 29970 | 20826 | | 23112 |
| 25399 | 16255 | 27685 | 18541 | 29971 | 20827 | | 23113 |
| 25400 | 16256 | 27686 | 18542 | 29972 | 20828 | | 23114 |
| 25401 | 16257 | 27687 | 18543 | 29973 | 20829 | | 23115 |
| 25402 | 16258 | 27688 | 18544 | 29974 | 20830 | | 23116 |
| 25403 | 16259 | 27689 | 18545 | 29975 | 20831 | | 23117 |
| 25404 | 16260 | 27690 | 18546 | 29976 | 20832 | | 23118 |
| 25405 | 16261 | 27691 | 18547 | 29977 | 20833 | | 23119 |
| 25406 | 16262 | 27692 | 18548 | 29978 | 20834 | | 23120 |
| 25407 | 16263 | 27693 | 18549 | 29979 | 20835 | | 23121 |
| 25408 | 16264 | 27694 | 18550 | 29980 | 20836 | | 23122 |
| 25409 | 16265 | 27695 | 18551 | 29981 | 20837 | | 23123 |
| 25410 | 16266 | 27696 | 18552 | 29982 | 20838 | | 23124 |
| 25411 | 16267 | 27697 | 18553 | 29983 | 20839 | | 23125 |
| 25412 | 16268 | 27698 | 18554 | 29984 | 20840 | | 23126 |
| 25413 | 16269 | 27699 | 18555 | 29985 | 20841 | | 23127 |
| 25414 | 16270 | 27700 | 18556 | 29986 | 20842 | | 23128 |
| 25415 | 16271 | 27701 | 18557 | 29987 | 20843 | | 23129 |
| 25416 | 16272 | 27702 | 18558 | 29988 | 20844 | | 23130 |
| 25417 | 16273 | 27703 | 18559 | 29989 | 20845 | | 23131 |
| 25418 | 16274 | 27704 | 18560 | 29990 | 20846 | | 23132 |
| 25419 | 16275 | 27705 | 18561 | 29991 | 20847 | | 23133 |
| 25420 | 16276 | 27706 | 18562 | 29992 | 20848 | | 23134 |
| 25421 | 16277 | 27707 | 18563 | 29993 | 20849 | | 23135 |
| 25422 | 16278 | 27708 | 18564 | 29994 | 20850 | | 23136 |
| 25423 | 16279 | 27709 | 18565 | 29995 | 20851 | | 23137 |
| 25424 | 16280 | 27710 | 18566 | 29996 | 20852 | | 23138 |
| 25425 | 16281 | 27711 | 18567 | 29997 | 20853 | | 23139 |
| 25426 | 16282 | 27712 | 18568 | 29998 | 20854 | | 23140 |
| 25427 | 16283 | 27713 | 18569 | 29999 | 20855 | | 23141 |
| 25428 | 16284 | 27714 | 18570 | 30000 | 20856 | | 23142 |
| 25429 | 16285 | 27715 | 18571 | 30001 | 20857 | | 23143 |
| 25430 | 16286 | 27716 | 18572 | 30002 | 20858 | | 23144 |
| 25431 | 16287 | 27717 | 18573 | 30003 | 20859 | | 23145 |
| 25432 | 16288 | 27718 | 18574 | 30004 | 20860 | | 23146 |
| 25433 | 16289 | 27719 | 18575 | 30005 | 20861 | | 23147 |
| 25434 | 16290 | 27720 | 18576 | 30006 | 20862 | | 23148 |
| 25435 | 16291 | 27721 | 18577 | 30007 | 20863 | | 23149 |
| 25436 | 16292 | 27722 | 18578 | 30008 | 20864 | | 23150 |
| 25437 | 16293 | 27723 | 18579 | 30009 | 20865 | | 23151 |
| 25438 | 16294 | 27724 | 18580 | 30010 | 20866 | | 23152 |
| 25439 | 16295 | 27725 | 18581 | 30011 | 20867 | | 23153 |
| 25440 | 16296 | 27726 | 18582 | 30012 | 20868 | | 23154 |
| 25441 | 16297 | 27727 | 18583 | 30013 | 20869 | | 23155 |
| 25442 | 16298 | 27728 | 18584 | 30014 | 20870 | | 23156 |
| 25443 | 16299 | 27729 | 18585 | 30015 | 20871 | | 23157 |
| 25444 | 16300 | 27730 | 18586 | 30016 | 20872 | | 23158 |
| 25445 | 16301 | 27731 | 18587 | 30017 | 20873 | | 23159 |
| 25446 | 16302 | 27732 | 18588 | 30018 | 20874 | | 23160 |
| 25447 | 16303 | 27733 | 18589 | 30019 | 20875 | | 23161 |
| 25448 | 16304 | 27734 | 18590 | 30020 | 20876 | | 23162 |
| 25449 | 16305 | 27735 | 18591 | 30021 | 20877 | | 23163 |
| 25450 | 16306 | 27736 | 18592 | 30022 | 20878 | | 23164 |
| 25451 | 16307 | 27737 | 18593 | 30023 | 20879 | | 23165 |
| 25452 | 16308 | 27738 | 18594 | 30024 | 20880 | | 23166 |
| 25453 | 16309 | 27739 | 18595 | 30025 | 20881 | | 23167 |
| 25454 | 16310 | 27740 | 18596 | 30026 | 20882 | | 23168 |
| 25455 | 16311 | 27741 | 18597 | 30027 | 20883 | | 23169 |
| 25456 | 16312 | 27742 | 18598 | 30028 | 20884 | | 23170 |
| 25457 | 16313 | 27743 | 18599 | 30029 | 20885 | | 23171 |
| 25458 | 16314 | 27744 | 18600 | 30030 | 20886 | | 23172 |
| 25459 | 16315 | 27745 | 18601 | 30031 | 20887 | | 23173 |
| 25460 | 16316 | 27746 | 18602 | 30032 | 20888 | | 23174 |
| 25461 | 16317 | 27747 | 18603 | 30033 | 20889 | | 23175 |
| 25462 | 16318 | 27748 | 18604 | 30034 | 20890 | | 23176 |
| 25463 | 16319 | 27749 | 18605 | 30035 | 20891 | | 23177 |
| 25464 | 16320 | 27750 | 18606 | 30036 | 20892 | | 23178 |
| 25465 | 16321 | 27751 | 18607 | 30037 | 20893 | | 23179 |
| 25466 | 16322 | 27752 | 18608 | 30038 | 20894 | | 23180 |
| 25467 | 16323 | 27753 | 18609 | 30039 | 20895 | | 23181 |
| 25468 | 16324 | 27754 | 18610 | 30040 | 20896 | | 23182 |
| 25469 | 16325 | 27755 | 18611 | 30041 | 20897 | | 23183 |
| 25470 | 16326 | 27756 | 18612 | 30042 | 20898 | | 23184 |
| 25471 | 16327 | 27757 | 18613 | 30043 | 20899 | | 23185 |
| 25472 | 16328 | 27758 | 18614 | 30044 | 20900 | | 23186 |
| 25473 | 16329 | 27759 | 18615 | 30045 | 20901 | | 23187 |
| 25474 | 16330 | 27760 | 18616 | 30046 | 20902 | | 23188 |
| 25475 | 16331 | 27761 | 18617 | 30047 | 20903 | | 23189 |
| 25476 | 16332 | 27762 | 18618 | 30048 | 20904 | | 23190 |
| 25477 | 16333 | 27763 | 18619 | 30049 | 20905 | | 23191 |
| 25478 | 16334 | 27764 | 18620 | 30050 | 20906 | | 23192 |
| 25479 | 16335 | 27765 | 18621 | 30051 | 20907 | | 23193 |
| 25480 | 16336 | 27766 | 18622 | 30052 | 20908 | | 23194 |
| 25481 | 16337 | 27767 | 18623 | 30053 | 20909 | | 23195 |
| 25482 | 16338 | 27768 | 18624 | 30054 | 20910 | | 23196 |
| 25483 | 16339 | 27769 | 18625 | 30055 | 20911 | | 23197 |
| 25484 | 16340 | 27770 | 18626 | 30056 | 20912 | | 23198 |
| 25485 | 16341 | 27771 | 18627 | 30057 | 20913 | | 23199 |
| 25486 | 16342 | 27772 | 18628 | 30058 | 20914 | | 23200 |
| 25487 | 16343 | 27773 | 18629 | 30059 | 20915 | | 23201 |
| 25488 | 16344 | 27774 | 18630 | 30060 | 20916 | | 23202 |
| 25489 | 16345 | 27775 | 18631 | 30061 | 20917 | | 23203 |
| 25490 | 16346 | 27776 | 18632 | 30062 | 20918 | | 23204 |
| 25491 | 16347 | 27777 | 18633 | 30063 | 20919 | | 23205 |
| 25492 | 16348 | 27778 | 18634 | 30064 | 20920 | | 23206 |
| 25493 | 16349 | 27779 | 18635 | 30065 | 20921 | | 23207 |
| 25494 | 16350 | 27780 | 18636 | 30066 | 20922 | | 23208 |
| 25495 | 16351 | 27781 | 18637 | 30067 | 20923 | | 23209 |
| 25496 | 16352 | 27782 | 18638 | 30068 | 20924 | | 23210 |
| 25497 | 16353 | 27783 | 18639 | 30069 | 20925 | | 23211 |
| 25498 | 16354 | 27784 | 18640 | 30070 | 20926 | | 23212 |
| 25499 | 16355 | 27785 | 18641 | 30071 | 20927 | | 23213 |
| 25500 | 16356 | 27786 | 18642 | 30072 | 20928 | | 23214 |
| 25501 | 16357 | 27787 | 18643 | 30073 | 20929 | | 23215 |
| 25502 | 16358 | 27788 | 18644 | 30074 | 20930 | | 23216 |
| 25503 | 16359 | 27789 | 18645 | 30075 | 20931 | | 23217 |
| 25504 | 16360 | 27790 | 18646 | 30076 | 20932 | | 23218 |
| 25505 | 16361 | 27791 | 18647 | 30077 | 20933 | | 23219 |
| 25506 | 16362 | 27792 | 18648 | 30078 | 20934 | | 23220 |
| 25507 | 16363 | 27793 | 18649 | 30079 | 20935 | | 23221 |
| 25508 | 16364 | 27794 | 18650 | 30080 | 20936 | | 23222 |
| 25509 | 16365 | 27795 | 18651 | 30081 | 20937 | | 23223 |
| 25510 | 16366 | 27796 | 18652 | 30082 | 20938 | | 23224 |
| 25511 | 16367 | 27797 | 18653 | 30083 | 20939 | | 23225 |
| 25512 | 16368 | 27798 | 18654 | 30084 | 20940 | | 23226 |
| 25513 | 16369 | 27799 | 18655 | 30085 | 20941 | | 23227 |
| 25514 | 16370 | 27800 | 18656 | 30086 | 20942 | | 23228 |
| 25515 | 16371 | 27801 | 18657 | 30087 | 20943 | | 23229 |
| 25516 | 16372 | 27802 | 18658 | 30088 | 20944 | | 23230 |
| 25517 | 16373 | 27803 | 18659 | 30089 | 20945 | | 23231 |
| 25518 | 16374 | 27804 | 18660 | 30090 | 20946 | | 23232 |
| 25519 | 16375 | 27805 | 18661 | 30091 | 20947 | | 23233 |
| 25520 | 16376 | 27806 | 18662 | 30092 | 20948 | | 23234 |
| 25521 | 16377 | 27807 | 18663 | 30093 | 20949 | | 23235 |
| 25522 | 16378 | 27808 | 18664 | 30094 | 20950 | | 23236 |
| 25523 | 16379 | 27809 | 18665 | 30095 | 20951 | | 23237 |
| 25524 | 16380 | 27810 | 18666 | 30096 | 20952 | | 23238 |
| 25525 | 16381 | 27811 | 18667 | 30097 | 20953 | | 23239 |
| 25526 | 16382 | 27812 | 18668 | 30098 | 20954 | | 23240 |
| 25527 | 16383 | 27813 | 18669 | 30099 | 20955 | | 23241 |
| 25528 | 16384 | 27814 | 18670 | 30100 | 20956 | | 23242 |
| 25529 | 16385 | 27815 | 18671 | 30101 | 20957 | | 23243 |
| 25530 | 16386 | 27816 | 18672 | 30102 | 20958 | | 23244 |
| 25531 | 16387 | 27817 | 18673 | 30103 | 20959 | | 23245 |
| 25532 | 16388 | 27818 | 18674 | 30104 | 20960 | | 23246 |
| 25533 | 16389 | 27819 | 18675 | 30105 | 20961 | | 23247 |
| 25534 | 16390 | 27820 | 18676 | 30106 | 20962 | | 23248 |
| 25535 | 16391 | 27821 | 18677 | 30107 | 20963 | | 23249 |
| 25536 | 16392 | 27822 | 18678 | 30108 | 20964 | | 23250 |
| 25537 | 16393 | 27823 | 18679 | 30109 | 20965 | | 23251 |
| 25538 | 16394 | 27824 | 18680 | 30110 | 20966 | | 23252 |
| 25539 | 16395 | 27825 | 18681 | 30111 | 20967 | | 23253 |
| 25540 | 16396 | 27826 | 18682 | 30112 | 20968 | | 23254 |
| 25541 | 16397 | 27827 | 18683 | 30113 | 20969 | | 23255 |
| 25542 | 16398 | 27828 | 18684 | 30114 | 20970 | | 23256 |
| 25543 | 16399 | 27829 | 18685 | 30115 | 20971 | | 23257 |
| 25544 | 16400 | 27830 | 18686 | 30116 | 20972 | | 23258 |
| 25545 | 16401 | 27831 | 18687 | 30117 | 20973 | | 23259 |
| 25546 | 16402 | 27832 | 18688 | 30118 | 20974 | | 23260 |
| 25547 | 16403 | 27833 | 18689 | 30119 | 20975 | | 23261 |
| 25548 | 16404 | 27834 | 18690 | 30120 | 20976 | | 23262 |
| 25549 | 16405 | 27835 | 18691 | 30121 | 20977 | | 23263 |
| 25550 | 16406 | 27836 | 18692 | 30122 | 20978 | | 23264 |
| 25551 | 16407 | 27837 | 18693 | 30123 | 20979 | | 23265 |
| 25552 | 16408 | 27838 | 18694 | 30124 | 20980 | | 23266 |
| 25553 | 16409 | 27839 | 18695 | 30125 | 20981 | | 23267 |
| 25554 | 16410 | 27840 | 18696 | 30126 | 20982 | | 23268 |
| 25555 | 16411 | 27841 | 18697 | 30127 | 20983 | | 23269 |
| 25556 | 16412 | 27842 | 18698 | 30128 | 20984 | | 23270 |
| 25557 | 16413 | 27843 | 18699 | 30129 | 20985 | | 23271 |
| 25558 | 16414 | 27844 | 18700 | 30130 | 20986 | | 23272 |
| 25559 | 16415 | 27845 | 18701 | 30131 | 20987 | | 23273 |
| 25560 | 16416 | 27846 | 18702 | 30132 | 20988 | | 23274 |
| 25561 | 16417 | 27847 | 18703 | 30133 | 20989 | | 23275 |
| 25562 | 16418 | 27848 | 18704 | 30134 | 20990 | | 23276 |
| 25563 | 16419 | 27849 | 18705 | 30135 | 20991 | | 23277 |
| 25564 | 16420 | 27850 | 18706 | 30136 | 20992 | | 23278 |
| 25565 | 16421 | 27851 | 18707 | 30137 | 20993 | | 23279 |
| 25566 | 16422 | 27852 | 18708 | 30138 | 20994 | | 23280 |
| 25567 | 16423 | 27853 | 18709 | 30139 | 20995 | | 23281 |
| 25568 | 16424 | 27854 | 18710 | 30140 | 20996 | | 23282 |
| 25569 | 16425 | 27855 | 18711 | 30141 | 20997 | | 23283 |
| 25570 | 16426 | 27856 | 18712 | 30142 | 20998 | | 23284 |
| 25571 | 16427 | 27857 | 18713 | 30143 | 20999 | | 23285 |
| 25572 | 16428 | 27858 | 18714 | 30144 | 21000 | | 23286 |
| 25573 | 16429 | 27859 | 18715 | 30145 | 21001 | | 23287 |
| 25574 | 16430 | 27860 | 18716 | 30146 | 21002 | | 23288 |
| 25575 | 16431 | 27861 | 18717 | 30147 | 21003 | | 23289 |
| 25576 | 16432 | 27862 | 18718 | 30148 | 21004 | | 23290 |
| 25577 | 16433 | 27863 | 18719 | 30149 | 21005 | | 23291 |
| 25578 | 16434 | 27864 | 18720 | 30150 | 21006 | | 23292 |
| 25579 | 16435 | 27865 | 18721 | 30151 | 21007 | | 23293 |
| 25580 | 16436 | 27866 | 18722 | 30152 | 21008 | | 23294 |
| 25581 | 16437 | 27867 | 18723 | 30153 | 21009 | | 23295 |
| 25582 | 16438 | 27868 | 18724 | 30154 | 21010 | | 23296 |
| 25583 | 16439 | 27869 | 18725 | 30155 | 21011 | | 23297 |
| 25584 | 16440 | 27870 | 18726 | 30156 | 21012 | | 23298 |
| 25585 | 16441 | 27871 | 18727 | 30157 | 21013 | | 23299 |
| 25586 | 16442 | 27872 | 18728 | 30158 | 21014 | | 23300 |
| 25587 | 16443 | 27873 | 18729 | 30159 | 21015 | | 23301 |
| 25588 | 16444 | 27874 | 18730 | 30160 | 21016 | | 23302 |
| 25589 | 16445 | 27875 | 18731 | 30161 | 21017 | | 23303 |
| 25590 | 16446 | 27876 | 18732 | 30162 | 21018 | | 23304 |
| 25591 | 16447 | 27877 | 18733 | 30163 | 21019 | | 23305 |
| 25592 | 16448 | 27878 | 18734 | 30164 | 21020 | | 23306 |
| 25593 | 16449 | 27879 | 18735 | 30165 | 21021 | | 23307 |
| 25594 | 16450 | 27880 | 18736 | 30166 | 21022 | | 23308 |
| 25595 | 16451 | 27881 | 18737 | 30167 | 21023 | | 23309 |
| 25596 | 16452 | 27882 | 18738 | 30168 | 21024 | | 23310 |
| 25597 | 16453 | 27883 | 18739 | 30169 | 21025 | | 23311 |
| 25598 | 16454 | 27884 | 18740 | 30170 | 21026 | | 23312 |
| 25599 | 16455 | 27885 | 18741 | 30171 | 21027 | | 23313 |
| 25600 | 16456 | 27886 | 18742 | 30172 | 21028 | | 23314 |
| 25601 | 16457 | 27887 | 18743 | 30173 | 21029 | | 23315 |
| 25602 | 16458 | 27888 | 18744 | 30174 | 21030 | | 23316 |
| 25603 | 16459 | 27889 | 18745 | 30175 | 21031 | | 23317 |
| 25604 | 16460 | 27890 | 18746 | 30176 | 21032 | | 23318 |
| 25605 | 16461 | 27891 | 18747 | 30177 | 21033 | | 23319 |
| 25606 | 16462 | 27892 | 18748 | 30178 | 21034 | | 23320 |
| 25607 | 16463 | 27893 | 18749 | 30179 | 21035 | | 23321 |
| 25608 | 16464 | 27894 | 18750 | 30180 | 21036 | | 23322 |
| 25609 | 16465 | 27895 | 18751 | 30181 | 21037 | | 23323 |
| 25610 | 16466 | 27896 | 18752 | 30182 | 21038 | | 23324 |
| 25611 | 16467 | 27897 | 18753 | 30183 | 21039 | | 23325 |
| 25612 | 16468 | 27898 | 18754 | 30184 | 21040 | | 23326 |
| 25613 | 16469 | 27899 | 18755 | 30185 | 21041 | | 23327 |
| 25614 | 16470 | 27900 | 18756 | 30186 | 21042 | | 23328 |
| 25615 | 16471 | 27901 | 18757 | 30187 | 21043 | | 23329 |
| 25616 | 16472 | 27902 | 18758 | 30188 | 21044 | | 23330 |
| 25617 | 16473 | 27903 | 18759 | 30189 | 21045 | | 23331 |
| 25618 | 16474 | 27904 | 18760 | 30190 | 21046 | | 23332 |
| 25619 | 16475 | 27905 | 18761 | 30191 | 21047 | | 23333 |
| 25620 | 16476 | 27906 | 18762 | 30192 | 21048 | | 23334 |
| 25621 | 16477 | 27907 | 18763 | 30193 | 21049 | | 23335 |
| 25622 | 16478 | 27908 | 18764 | 30194 | 21050 | | 23336 |
| 25623 | 16479 | 27909 | 18765 | 30195 | 21051 | | 23337 |
| 25624 | 16480 | 27910 | 18766 | 30196 | 21052 | | 23338 |
| 25625 | 16481 | 27911 | 18767 | 30197 | 21053 | | 23339 |
| 25626 | 16482 | 27912 | 18768 | 30198 | 21054 | | 23340 |
| 25627 | 16483 | 27913 | 18769 | 30199 | 21055 | | 23341 |
| 25628 | 16484 | 27914 | 18770 | 30200 | 21056 | | 23342 |
| 25629 | 16485 | 27915 | 18771 | 30201 | 21057 | | 23343 |
| 25630 | 16486 | 27916 | 18772 | 30202 | 21058 | | 23344 |
| 25631 | 16487 | 27917 | 18773 | 30203 | 21059 | | 23345 |
| 25632 | 16488 | 27918 | 18774 | 30204 | 21060 | | 23346 |
| 25633 | 16489 | 27919 | 18775 | 30205 | 21061 | | 23347 |
| 25634 | 16490 | 27920 | 18776 | 30206 | 21062 | | 23348 |
| 25635 | 16491 | 27921 | 18777 | 30207 | 21063 | | 23349 |
| 25636 | 16492 | 27922 | 18778 | 30208 | 21064 | | 23350 |
| 25637 | 16493 | 27923 | 18779 | 30209 | 21065 | | 23351 |
| 25638 | 16494 | 27924 | 18780 | 30210 | 21066 | | 23352 |
| 25639 | 16495 | 27925 | 18781 | 30211 | 21067 | | 23353 |
| 25640 | 16496 | 27926 | 18782 | 30212 | 21068 | | 23354 |
| 25641 | 16497 | 27927 | 18783 | 30213 | 21069 | | 23355 |
| 25642 | 16498 | 27928 | 18784 | 30214 | 21070 | | 23356 |
| 25643 | 16499 | 27929 | 18785 | 30215 | 21071 | | 23357 |
| 25644 | 16500 | 27930 | 18786 | 30216 | 21072 | | 23358 |
| 25645 | 16501 | 27931 | 18787 | 30217 | 21073 | | 23359 |
| 25646 | 16502 | 27932 | 18788 | 30218 | 21074 | | 23360 |
| 25647 | 16503 | 27933 | 18789 | 30219 | 21075 | | 23361 |
| 25648 | 16504 | 27934 | 18790 | 30220 | 21076 | | 23362 |
| 25649 | 16505 | 27935 | 18791 | 30221 | 21077 | | 23363 |
| 25650 | 16506 | 27936 | 18792 | 30222 | 21078 | | 23364 |
| 25651 | 16507 | 27937 | 18793 | 30223 | 21079 | | 23365 |
| 25652 | 16508 | 27938 | 18794 | 30224 | 21080 | | 23366 |
| 25653 | 16509 | 27939 | 18795 | 30225 | 21081 | | 23367 |
| 25654 | 16510 | 27940 | 18796 | 30226 | 21082 | | 23368 |
| 25655 | 16511 | 27941 | 18797 | 30227 | 21083 | | 23369 |
| 25656 | 16512 | 27942 | 18798 | 30228 | 21084 | | 23370 |
| 25657 | 16513 | 27943 | 18799 | 30229 | 21085 | | 23371 |
| 25658 | 16514 | 27944 | 18800 | 30230 | 21086 | | 23372 |
| 25659 | 16515 | 27945 | 18801 | 30231 | 21087 | | 23373 |
| 25660 | 16516 | 27946 | 18802 | 30232 | 21088 | | 23374 |
| 25661 | 16517 | 27947 | 18803 | 30233 | 21089 | | 23375 |
| 25662 | 16518 | 27948 | 18804 | 30234 | 21090 | | 23376 |
| 25663 | 16519 | 27949 | 18805 | 30235 | 21091 | | 23377 |
| 25664 | 16520 | 27950 | 18806 | 30236 | 21092 | | 23378 |
| 25665 | 16521 | 27951 | 18807 | 30237 | 21093 | | 23379 |
| 25666 | 16522 | 27952 | 18808 | 30238 | 21094 | | 23380 |
| 25667 | 16523 | 27953 | 18809 | 30239 | 21095 | | 23381 |
| 25668 | 16524 | 27954 | 18810 | 30240 | 21096 | | 23382 |
| 25669 | 16525 | 27955 | 18811 | 30241 | 21097 | | 23383 |
| 25670 | 16526 | 27956 | 18812 | 30242 | 21098 | | 23384 |
| 25671 | 16527 | 27957 | 18813 | 30243 | 21099 | | 23385 |
| 25672 | 16528 | 27958 | 18814 | 30244 | 21100 | | 23386 |
| 25673 | 16529 | 27959 | 18815 | 30245 | 21101 | | 23387 |
| 25674 | 16530 | 27960 | 18816 | 30246 | 21102 | | 23388 |
| 25675 | 16531 | 27961 | 18817 | 30247 | 21103 | | 23389 |
| 25676 | 16532 | 27962 | 18818 | 30248 | 21104 | | 23390 |
| 25677 | 16533 | 27963 | 18819 | 30249 | 21105 | | 23391 |
| 25678 | 16534 | 27964 | 18820 | 30250 | 21106 | | 23392 |
| 25679 | 16535 | 27965 | 18821 | 30251 | 21107 | | 23393 |
| 25680 | 16536 | 27966 | 18822 | 30252 | 21108 | | 23394 |
| 25681 | 16537 | 27967 | 18823 | 30253 | 21109 | | 23395 |
| 25682 | 16538 | 27968 | 18824 | 30254 | 21110 | | 23396 |
| 25683 | 16539 | 27969 | 18825 | 30255 | 21111 | | 23397 |
| 25684 | 16540 | 27970 | 18826 | 30256 | 21112 | | 23398 |
| 25685 | 16541 | 27971 | 18827 | 30257 | 21113 | | 23399 |
| 25686 | 16542 | 27972 | 18828 | 30258 | 21114 | | 23400 |
| 25687 | 16543 | 27973 | 18829 | 30259 | 21115 | | 23401 |
| 25688 | 16544 | 27974 | 18830 | 30260 | 21116 | | 23402 |
| 25689 | 16545 | 27975 | 18831 | 30261 | 21117 | | 23403 |
| 25690 | 16546 | 27976 | 18832 | 30262 | 21118 | | 23404 |
| 25691 | 16547 | 27977 | 18833 | 30263 | 21119 | | 23405 |
| 25692 | 16548 | 27978 | 18834 | 30264 | 21120 | | 23406 |
| 25693 | 16549 | 27979 | 18835 | 30265 | 21121 | | 23407 |
| 25694 | 16550 | 27980 | 18836 | 30266 | 21122 | | 23408 |
| 25695 | 16551 | 27981 | 18837 | 30267 | 21123 | | 23409 |
| 25696 | 16552 | 27982 | 18838 | 30268 | 21124 | | 23410 |
| 25697 | 16553 | 27983 | 18839 | 30269 | 21125 | | 23411 |
| 25698 | 16554 | 27984 | 18840 | 30270 | 21126 | | 23412 |
| 25699 | 16555 | 27985 | 18841 | 30271 | 21127 | | 23413 |
| 25700 | 16556 | 27986 | 18842 | 30272 | 21128 | | 23414 |
| 25701 | 16557 | 27987 | 18843 | 30273 | 21129 | | 23415 |
| 25702 | 16558 | 27988 | 18844 | 30274 | 21130 | | 23416 |
| 25703 | 16559 | 27989 | 18845 | 30275 | 21131 | | 23417 |
| 25704 | 16560 | 27990 | 18846 | 30276 | 21132 | | 23418 |
| 25705 | 16561 | 27991 | 18847 | 30277 | 21133 | | 23419 |
| 25706 | 16562 | 27992 | 18848 | 30278 | 21134 | | 23420 |
| 25707 | 16563 | 27993 | 18849 | 30279 | 21135 | | 23421 |
| 25708 | 16564 | 27994 | 18850 | 30280 | 21136 | | 23422 |
| 25709 | 16565 | 27995 | 18851 | 30281 | 21137 | | 23423 |
| 25710 | 16566 | 27996 | 18852 | 30282 | 21138 | | 23424 |
| 25711 | 16567 | 27997 | 18853 | 30283 | 21139 | | 23425 |
| 25712 | 16568 | 27998 | 18854 | 30284 | 21140 | | 23426 |
| 25713 | 16569 | 27999 | 18855 | 30285 | 21141 | | 23427 |
| 25714 | 16570 | 28000 | 18856 | 30286 | 21142 | | 23428 |
| 25715 | 16571 | 28001 | 18857 | 30287 | 21143 | | 23429 |
| 25716 | 16572 | 28002 | 18858 | 30288 | 21144 | | 23430 |
| 25717 | 16573 | 28003 | 18859 | 30289 | 21145 | | 23431 |
| 25718 | 16574 | 28004 | 18860 | 30290 | 21146 | | 23432 |
| 25719 | 16575 | 28005 | 18861 | 30291 | 21147 | | 23433 |
| 25720 | 16576 | 28006 | 18862 | 30292 | 21148 | | 23434 |
| 25721 | 16577 | 28007 | 18863 | 30293 | 21149 | | 23435 |
| 25722 | 16578 | 28008 | 18864 | 30294 | 21150 | | 23436 |
| 25723 | 16579 | 28009 | 18865 | 30295 | 21151 | | 23437 |
| 25724 | 16580 | 28010 | 18866 | 30296 | 21152 | | 23438 |
| 25725 | 16581 | 28011 | 18867 | 30297 | 21153 | | 23439 |
| 25726 | 16582 | 28012 | 18868 | 30298 | 21154 | | 23440 |
| 25727 | 16583 | 28013 | 18869 | 30299 | 21155 | | 23441 |
| 25728 | 16584 | 28014 | 18870 | 30300 | 21156 | | 23442 |
| 25729 | 16585 | 28015 | 18871 | 30301 | 21157 | | 23443 |
| 25730 | 16586 | 28016 | 18872 | 30302 | 21158 | | 23444 |
| 25731 | 16587 | 28017 | 18873 | 30303 | 21159 | | 23445 |
| 25732 | 16588 | 28018 | 18874 | 30304 | 21160 | | 23446 |
| 25733 | 16589 | 28019 | 18875 | 30305 | 21161 | | 23447 |
| 25734 | 16590 | 28020 | 18876 | 30306 | 21162 | | 23448 |
| 25735 | 16591 | 28021 | 18877 | 30307 | 21163 | | 23449 |
| 25736 | 16592 | 28022 | 18878 | 30308 | 21164 | | 23450 |
| 25737 | 16593 | 28023 | 18879 | 30309 | 21165 | | 23451 |
| 25738 | 16594 | 28024 | 18880 | 30310 | 21166 | | 23452 |
| 25739 | 16595 | 28025 | 18881 | 30311 | 21167 | | 23453 |
| 25740 | 16596 | 28026 | 18882 | 30312 | 21168 | | 23454 |
| 25741 | 16597 | 28027 | 18883 | 30313 | 21169 | | 23455 |
| 25742 | 16598 | 28028 | 18884 | 30314 | 21170 | | 23456 |
| 25743 | 16599 | 28029 | 18885 | 30315 | 21171 | | 23457 |
| 25744 | 16600 | 28030 | 18886 | 30316 | 21172 | | 23458 |
| 25745 | 16601 | 28031 | 18887 | 30317 | 21173 | | 23459 |
| 25746 | 16602 | 28032 | 18888 | 30318 | 21174 | | 23460 |
| 25747 | 16603 | 28033 | 18889 | 30319 | 21175 | | 23461 |
| 25748 | 16604 | 28034 | 18890 | 30320 | 21176 | | 23462 |
| 25749 | 16605 | 28035 | 18891 | 30321 | 21177 | | 23463 |
| 25750 | 16606 | 28036 | 18892 | 30322 | 21178 | | 23464 |
| 25751 | 16607 | 28037 | 18893 | 30323 | 21179 | | 23465 |
| 25752 | 16608 | 28038 | 18894 | 30324 | 21180 | | 23466 |
| 25753 | 16609 | 28039 | 18895 | 30325 | 21181 | | 23467 |
| 25754 | 16610 | 28040 | 18896 | 30326 | 21182 | | 23468 |
| 25755 | 16611 | 28041 | 18897 | 30327 | 21183 | | 23469 |
| 25756 | 16612 | 28042 | 18898 | 30328 | 21184 | | 23470 |
| 25757 | 16613 | 28043 | 18899 | 30329 | 21185 | | 23471 |
| 25758 | 16614 | 28044 | 18900 | 30330 | 21186 | | 23472 |
| 25759 | 16615 | 28045 | 18901 | 30331 | 21187 | | 23473 |
| 25760 | 16616 | 28046 | 18902 | 30332 | 21188 | | 23474 |
| 25761 | 16617 | 28047 | 18903 | 30333 | 21189 | | 23475 |
| 25762 | 16618 | 28048 | 18904 | 30334 | 21190 | | 23476 |
| 25763 | 16619 | 28049 | 18905 | 30335 | 21191 | | 23477 |
| 25764 | 16620 | 28050 | 18906 | 30336 | 21192 | | 23478 |
| 25765 | 16621 | 28051 | 18907 | 30337 | 21193 | | 23479 |
| 25766 | 16622 | 28052 | 18908 | 30338 | 21194 | | 23480 |
| 25767 | 16623 | 28053 | 18909 | 30339 | 21195 | | 23481 |
| 25768 | 16624 | 28054 | 18910 | 30340 | 21196 | | 23482 |
| 25769 | 16625 | 28055 | 18911 | 30341 | 21197 | | 23483 |
| 25770 | 16626 | 28056 | 18912 | 30342 | 21198 | | 23484 |
| 25771 | 16627 | 28057 | 18913 | 30343 | 21199 | | 23485 |
| 25772 | 16628 | 28058 | 18914 | 30344 | 21200 | | 23486 |
| 25773 | 16629 | 28059 | 18915 | 30345 | 21201 | | 23487 |
| 25774 | 16630 | 28060 | 18916 | 30346 | 21202 | | 23488 |
| 25775 | 16631 | 28061 | 18917 | 30347 | 21203 | | 23489 |
| 25776 | 16632 | 28062 | 18918 | 30348 | 21204 | | 23490 |
| 25777 | 16633 | 28063 | 18919 | 30349 | 21205 | | 23491 |
| 25778 | 16634 | 28064 | 18920 | 30350 | 21206 | | 23492 |
| 25779 | 16635 | 28065 | 18921 | 30351 | 21207 | | 23493 |
| 25780 | 16636 | 28066 | 18922 | 30352 | 21208 | | 23494 |
| 25781 | 16637 | 28067 | 18923 | 30353 | 21209 | | 23495 |
| 25782 | 16638 | 28068 | 18924 | 30354 | 21210 | | 23496 |
| 25783 | 16639 | 28069 | 18925 | 30355 | 21211 | | 23497 |
| 25784 | 16640 | 28070 | 18926 | 30356 | 21212 | | 23498 |
| 25785 | 16641 | 28071 | 18927 | 30357 | 21213 | | 23499 |
| 25786 | 16642 | 28072 | 18928 | 30358 | 21214 | | 23500 |
| 25787 | 16643 | 28073 | 18929 | 30359 | 21215 | | 23501 |
| 25788 | 16644 | 28074 | 18930 | 30360 | 21216 | | 23502 |
| 25789 | 16645 | 28075 | 18931 | 30361 | 21217 | | 23503 |
| 25790 | 16646 | 28076 | 18932 | 30362 | 21218 | | 23504 |
| 25791 | 16647 | 28077 | 18933 | 30363 | 21219 | | 23505 |
| 25792 | 16648 | 28078 | 18934 | 30364 | 21220 | | 23506 |
| 25793 | 16649 | 28079 | 18935 | 30365 | 21221 | | 23507 |
| 25794 | 16650 | 28080 | 18936 | 30366 | 21222 | | 23508 |
| 25795 | 16651 | 28081 | 18937 | 30367 | 21223 | | 23509 |
| 25796 | 16652 | 28082 | 18938 | 30368 | 21224 | | 23510 |
| 25797 | 16653 | 28083 | 18939 | 30369 | 21225 | | 23511 |
| 25798 | 16654 | 28084 | 18940 | 30370 | 21226 | | 23512 |
| 25799 | 16655 | 28085 | 18941 | 30371 | 21227 | | 23513 |
| 25800 | 16656 | 28086 | 18942 | 30372 | 21228 | | 23514 |
| 25801 | 16657 | 28087 | 18943 | 30373 | 21229 | | 23515 |
| 25802 | 16658 | 28088 | 18944 | 30374 | 21230 | | 23516 |
| 25803 | 16659 | 28089 | 18945 | 30375 | 21231 | | 23517 |
| 25804 | 16660 | 28090 | 18946 | 30376 | 21232 | | 23518 |
| 25805 | 16661 | 28091 | 18947 | 30377 | 21233 | | 23519 |
| 25806 | 16662 | 28092 | 18948 | 30378 | 21234 | | 23520 |
| 25807 | 16663 | 28093 | 18949 | 30379 | 21235 | | 23521 |
| 25808 | 16664 | 28094 | 18950 | 30380 | 21236 | | 23522 |
| 25809 | 16665 | 28095 | 18951 | 30381 | 21237 | | 23523 |
| 25810 | 16666 | 28096 | 18952 | 30382 | 21238 | | 23524 |
| 25811 | 16667 | 28097 | 18953 | 30383 | 21239 | | 23525 |
| 25812 | 16668 | 28098 | 18954 | 30384 | 21240 | | 23526 |
| 25813 | 16669 | 28099 | 18955 | 30385 | 21241 | | 23527 |
| 25814 | 16670 | 28100 | 18956 | 30386 | 21242 | | 23528 |
| 25815 | 16671 | 28101 | 18957 | 30387 | 21243 | | 23529 |
| 25816 | 16672 | 28102 | 18958 | 30388 | 21244 | | 23530 |
| 25817 | 16673 | 28103 | 18959 | 30389 | 21245 | | 23531 |
| 25818 | 16674 | 28104 | 18960 | 30390 | 21246 | | 23532 |
| 25819 | 16675 | 28105 | 18961 | 30391 | 21247 | | 23533 |
| 25820 | 16676 | 28106 | 18962 | 30392 | 21248 | | 23534 |
| 25821 | 16677 | 28107 | 18963 | 30393 | 21249 | | 23535 |
| 25822 | 16678 | 28108 | 18964 | 30394 | 21250 | | 23536 |
| 25823 | 16679 | 28109 | 18965 | 30395 | 21251 | | 23537 |
| 25824 | 16680 | 28110 | 18966 | 30396 | 21252 | | 23538 |
| 25825 | 16681 | 28111 | 18967 | 30397 | 21253 | | 23539 |
| 25826 | 16682 | 28112 | 18968 | 30398 | 21254 | | 23540 |
| 25827 | 16683 | 28113 | 18969 | 30399 | 21255 | | 23541 |
| 25828 | 16684 | 28114 | 18970 | 30400 | 21256 | | 23542 |
| 25829 | 16685 | 28115 | 18971 | 30401 | 21257 | | 23543 |
| 25830 | 16686 | 28116 | 18972 | 30402 | 21258 | | 23544 |
| 25831 | 16687 | 28117 | 18973 | 30403 | 21259 | | 23545 |
| 25832 | 16688 | 28118 | 18974 | 30404 | 21260 | | 23546 |
| 25833 | 16689 | 28119 | 18975 | 30405 | 21261 | | 23547 |
| 25834 | 16690 | 28120 | 18976 | 30406 | 21262 | | 23548 |
| 25835 | 16691 | 28121 | 18977 | 30407 | 21263 | | 23549 |
| 25836 | 16692 | 28122 | 18978 | 30408 | 21264 | | 23550 |
| 25837 | 16693 | 28123 | 18979 | 30409 | 21265 | | 23551 |
| 25838 | 16694 | 28124 | 18980 | 30410 | 21266 | | 23552 |
| 25839 | 16695 | 28125 | 18981 | 30411 | 21267 | | 23553 |
| 25840 | 16696 | 28126 | 18982 | 30412 | 21268 | | 23554 |
| 25841 | 16697 | 28127 | 18983 | 30413 | 21269 | | 23555 |
| 25842 | 16698 | 28128 | 18984 | 30414 | 21270 | | 23556 |
| 25843 | 16699 | 28129 | 18985 | 30415 | 21271 | | 23557 |
| 25844 | 16700 | 28130 | 18986 | 30416 | 21272 | | 23558 |
| 25845 | 16701 | 28131 | 18987 | 30417 | 21273 | | 23559 |
| 25846 | 16702 | 28132 | 18988 | 30418 | 21274 | | 23560 |
| 25847 | 16703 | 28133 | 18989 | 30419 | 21275 | | 23561 |
| 25848 | 16704 | 28134 | 18990 | 30420 | 21276 | | 23562 |
| 25849 | 16705 | 28135 | 18991 | 30421 | 21277 | | 23563 |
| 25850 | 16706 | 28136 | 18992 | 30422 | 21278 | | 23564 |
| 25851 | 16707 | 28137 | 18993 | 30423 | 21279 | | 23565 |
| 25852 | 16708 | 28138 | 18994 | 30424 | 21280 | | 23566 |
| 25853 | 16709 | 28139 | 18995 | 30425 | 21281 | | 23567 |
| 25854 | 16710 | 28140 | 18996 | 30426 | 21282 | | 23568 |
| 25855 | 16711 | 28141 | 18997 | 30427 | 21283 | | 23569 |
| 25856 | 16712 | 28142 | 18998 | 30428 | 21284 | | 23570 |
| 25857 | 16713 | 28143 | 18999 | 30429 | 21285 | | 23571 |
| 25858 | 16714 | 28144 | 19000 | 30430 | 21286 | | 23572 |
| 25859 | 16715 | 28145 | 19001 | 30431 | 21287 | | 23573 |
| 25860 | 16716 | 28146 | 19002 | 30432 | 21288 | | 23574 |
| 25861 | 16717 | 28147 | 19003 | 30433 | 21289 | | 23575 |
| 25862 | 16718 | 28148 | 19004 | 30434 | 21290 | | 23576 |
| 25863 | 16719 | 28149 | 19005 | 30435 | 21291 | | 23577 |
| 25864 | 16720 | 28150 | 19006 | 30436 | 21292 | | 23578 |
| 25865 | 16721 | 28151 | 19007 | 30437 | 21293 | | 23579 |
| 25866 | 16722 | 28152 | 19008 | 30438 | 21294 | | 23580 |
| 25867 | 16723 | 28153 | 19009 | 30439 | 21295 | | 23581 |
| 25868 | 16724 | 28154 | 19010 | 30440 | 21296 | | 23582 |
| 25869 | 16725 | 28155 | 19011 | 30441 | 21297 | | 23583 |
| 25870 | 16726 | 28156 | 19012 | 30442 | 21298 | | 23584 |
| 25871 | 16727 | 28157 | 19013 | 30443 | 21299 | | 23585 |
| 25872 | 16728 | 28158 | 19014 | 30444 | 21300 | | 23586 |
| 25873 | 16729 | 28159 | 19015 | 30445 | 21301 | | 23587 |
| 25874 | 16730 | 28160 | 19016 | 30446 | 21302 | | 23588 |
| 25875 | 16731 | 28161 | 19017 | 30447 | 21303 | | 23589 |
| 25876 | 16732 | 28162 | 19018 | 30448 | 21304 | | 23590 |
| 25877 | 16733 | 28163 | 19019 | 30449 | 21305 | | 23591 |
| 25878 | 16734 | 28164 | 19020 | 30450 | 21306 | | 23592 |
| 25879 | 16735 | 28165 | 19021 | 30451 | 21307 | | 23593 |
| 25880 | 16736 | 28166 | 19022 | 30452 | 21308 | | 23594 |
| 25881 | 16737 | 28167 | 19023 | 30453 | 21309 | | 23595 |
| 25882 | 16738 | 28168 | 19024 | 30454 | 21310 | | 23596 |
| 25883 | 16739 | 28169 | 19025 | 30455 | 21311 | | 23597 |
| 25884 | 16740 | 28170 | 19026 | 30456 | 21312 | | 23598 |
| 25885 | 16741 | 28171 | 19027 | 30457 | 21313 | | 23599 |
| 25886 | 16742 | 28172 | 19028 | 30458 | 21314 | | 23600 |
| 25887 | 16743 | 28173 | 19029 | 30459 | 21315 | | 23601 |
| 25888 | 16744 | 28174 | 19030 | 30460 | 21316 | | 23602 |
| 25889 | 16745 | 28175 | 19031 | 30461 | 21317 | | 23603 |
| 25890 | 16746 | 28176 | 19032 | 30462 | 21318 | | 23604 |
| 25891 | 16747 | 28177 | 19033 | 30463 | 21319 | | 23605 |
| 25892 | 16748 | 28178 | 19034 | 30464 | 21320 | | 23606 |
| 25893 | 16749 | 28179 | 19035 | 30465 | 21321 | | 23607 |
| 25894 | 16750 | 28180 | 19036 | 30466 | 21322 | | 23608 |
| 25895 | 16751 | 28181 | 19037 | 30467 | 21323 | | 23609 |
| 25896 | 16752 | 28182 | 19038 | 30468 | 21324 | | 23610 |
| 25897 | 16753 | 28183 | 19039 | 30469 | 21325 | | 23611 |
| 25898 | 16754 | 28184 | 19040 | 30470 | 21326 | | 23612 |
| 25899 | 16755 | 28185 | 19041 | 30471 | 21327 | | 23613 |
| 25900 | 16756 | 28186 | 19042 | 30472 | 21328 | | 23614 |
| 25901 | 16757 | 28187 | 19043 | 30473 | 21329 | | 23615 |
| 25902 | 16758 | 28188 | 19044 | 30474 | 21330 | | 23616 |
| 25903 | 16759 | 28189 | 19045 | 30475 | 21331 | | 23617 |
| 25904 | 16760 | 28190 | 19046 | 30476 | 21332 | | 23618 |
| 25905 | 16761 | 28191 | 19047 | 30477 | 21333 | | 23619 |
| 25906 | 16762 | 28192 | 19048 | 30478 | 21334 | | 23620 |
| 25907 | 16763 | 28193 | 19049 | 30479 | 21335 | | 23621 |
| 25908 | 16764 | 28194 | 19050 | 30480 | 21336 | | 23622 |
| 25909 | 16765 | 28195 | 19051 | 30481 | 21337 | | 23623 |
| 25910 | 16766 | 28196 | 19052 | 30482 | 21338 | | 23624 |
| 25911 | 16767 | 28197 | 19053 | 30483 | 21339 | | 23625 |
| 25912 | 16768 | 28198 | 19054 | 30484 | 21340 | | 23626 |
| 25913 | 16769 | 28199 | 19055 | 30485 | 21341 | | 23627 |
| 25914 | 16770 | 28200 | 19056 | 30486 | 21342 | | 23628 |
| 25915 | 16771 | 28201 | 19057 | 30487 | 21343 | | 23629 |
| 25916 | 16772 | 28202 | 19058 | 30488 | 21344 | | 23630 |
| 25917 | 16773 | 28203 | 19059 | 30489 | 21345 | | 23631 |
| 25918 | 16774 | 28204 | 19060 | 30490 | 21346 | | 23632 |
| 25919 | 16775 | 28205 | 19061 | 30491 | 21347 | | 23633 |
| 25920 | 16776 | 28206 | 19062 | 30492 | 21348 | | 23634 |
| 25921 | 16777 | 28207 | 19063 | 30493 | 21349 | | 23635 |
| 25922 | 16778 | 28208 | 19064 | 30494 | 21350 | | 23636 |
| 25923 | 16779 | 28209 | 19065 | 30495 | 21351 | | 23637 |
| 25924 | 16780 | 28210 | 19066 | 30496 | 21352 | | 23638 |
| 25925 | 16781 | 28211 | 19067 | 30497 | 21353 | | 23639 |
| 25926 | 16782 | 28212 | 19068 | 30498 | 21354 | | 23640 |
| 25927 | 16783 | 28213 | 19069 | 30499 | 21355 | | 23641 |
| 25928 | 16784 | 28214 | 19070 | 30500 | 21356 | | 23642 |
| 25929 | 16785 | 28215 | 19071 | 30501 | 21357 | | 23643 |
| 25930 | 16786 | 28216 | 19072 | 30502 | 21358 | | 23644 |
| 25931 | 16787 | 28217 | 19073 | 30503 | 21359 | | 23645 |
| 25932 | 16788 | 28218 | 19074 | 30504 | 21360 | | 23646 |
| 25933 | 16789 | 28219 | 19075 | 30505 | 21361 | | 23647 |
| 25934 | 16790 | 28220 | 19076 | 30506 | 21362 | | 23648 |
| 25935 | 16791 | 28221 | 19077 | 30507 | 21363 | | 23649 |
| 25936 | 16792 | 28222 | 19078 | 30508 | 21364 | | 23650 |
| 25937 | 16793 | 28223 | 19079 | 30509 | 21365 | | 23651 |
| 25938 | 16794 | 28224 | 19080 | 30510 | 21366 | | 23652 |
| 25939 | 16795 | 28225 | 19081 | 30511 | 21367 | | 23653 |
| 25940 | 16796 | 28226 | 19082 | 30512 | 21368 | | 23654 |
| 25941 | 16797 | 28227 | 19083 | 30513 | 21369 | | 23655 |
| 25942 | 16798 | 28228 | 19084 | 30514 | 21370 | | 23656 |
| 25943 | 16799 | 28229 | 19085 | 30515 | 21371 | | 23657 |
| 25944 | 16800 | 28230 | 19086 | 30516 | 21372 | | 23658 |
| 25945 | 16801 | 28231 | 19087 | 30517 | 21373 | | 23659 |
| 25946 | 16802 | 28232 | 19088 | 30518 | 21374 | | 23660 |
| 25947 | 16803 | 28233 | 19089 | 30519 | 21375 | | 23661 |
| 25948 | 16804 | 28234 | 19090 | 30520 | 21376 | | 23662 |
| 25949 | 16805 | 28235 | 19091 | 30521 | 21377 | | 23663 |
| 25950 | 16806 | 28236 | 19092 | 30522 | 21378 | | 23664 |
| 25951 | 16807 | 28237 | 19093 | 30523 | 21379 | | 23665 |
| 25952 | 16808 | 28238 | 19094 | 30524 | 21380 | | 23666 |
| 25953 | 16809 | 28239 | 19095 | 30525 | 21381 | | 23667 |
| 25954 | 16810 | 28240 | 19096 | 30526 | 21382 | | 23668 |
| 25955 | 16811 | 28241 | 19097 | 30527 | 21383 | | 23669 |
| 25956 | 16812 | 28242 | 19098 | 30528 | 21384 | | 23670 |
| 25957 | 16813 | 28243 | 19099 | 30529 | 21385 | | 23671 |
| 25958 | 16814 | 28244 | 19100 | 30530 | 21386 | | 23672 |
| 25959 | 16815 | 28245 | 19101 | 30531 | 21387 | | 23673 |
| 25960 | 16816 | 28246 | 19102 | 30532 | 21388 | | 23674 |
| 25961 | 16817 | 28247 | 19103 | 30533 | 21389 | | 23675 |
| 25962 | 16818 | 28248 | 19104 | 30534 | 21390 | | 23676 |
| 25963 | 16819 | 28249 | 19105 | 30535 | 21391 | | 23677 |
| 25964 | 16820 | 28250 | 19106 | 30536 | 21392 | | 23678 |
| 25965 | 16821 | 28251 | 19107 | 30537 | 21393 | | 23679 |
| 25966 | 16822 | 28252 | 19108 | 30538 | 21394 | | 23680 |
| 25967 | 16823 | 28253 | 19109 | 30539 | 21395 | | 23681 |
| 25968 | 16824 | 28254 | 19110 | 30540 | 21396 | | 23682 |
| 25969 | 16825 | 28255 | 19111 | 30541 | 21397 | | 23683 |
| 25970 | 16826 | 28256 | 19112 | 30542 | 21398 | | 23684 |
| 25971 | 16827 | 28257 | 19113 | 30543 | 21399 | | 23685 |
| 25972 | 16828 | 28258 | 19114 | 30544 | 21400 | | 23686 |
| 25973 | 16829 | 28259 | 19115 | 30545 | 21401 | | 23687 |
| 25974 | 16830 | 28260 | 19116 | 30546 | 21402 | | 23688 |
| 25975 | 16831 | 28261 | 19117 | 30547 | 21403 | | 23689 |
| 25976 | 16832 | 28262 | 19118 | 30548 | 21404 | | 23690 |
| 25977 | 16833 | 28263 | 19119 | 30549 | 21405 | | 23691 |
| 25978 | 16834 | 28264 | 19120 | 30550 | 21406 | | 23692 |
| 25979 | 16835 | 28265 | 19121 | 30551 | 21407 | | 23693 |
| 25980 | 16836 | 28266 | 19122 | 30552 | 21408 | | 23694 |
| 25981 | 16837 | 28267 | 19123 | 30553 | 21409 | | 23695 |
| 25982 | 16838 | 28268 | 19124 | 30554 | 21410 | | 23696 |
| 25983 | 16839 | 28269 | 19125 | 30555 | 21411 | | 23697 |
| 25984 | 16840 | 28270 | 19126 | 30556 | 21412 | | 23698 |
| 25985 | 16841 | 28271 | 19127 | 30557 | 21413 | | 23699 |
| 25986 | 16842 | 28272 | 19128 | 30558 | 21414 | | 23700 |
| 25987 | 16843 | 28273 | 19129 | 30559 | 21415 | | 23701 |
| 25988 | 16844 | 28274 | 19130 | 30560 | 21416 | | 23702 |
| 25989 | 16845 | 28275 | 19131 | 30561 | 21417 | | 23703 |
| 25990 | 16846 | 28276 | 19132 | 30562 | 21418 | | 23704 |
| 25991 | 16847 | 28277 | 19133 | 30563 | 21419 | | 23705 |
| 25992 | 16848 | 28278 | 19134 | 30564 | 21420 | | 23706 |
| 25993 | 16849 | 28279 | 19135 | 30565 | 21421 | | 23707 |
| 25994 | 16850 | 28280 | 19136 | 30566 | 21422 | | 23708 |
| 25995 | 16851 | 28281 | 19137 | 30567 | 21423 | | 23709 |
| 25996 | 16852 | 28282 | 19138 | 30568 | 21424 | | 23710 |
| 25997 | 16853 | 28283 | 19139 | 30569 | 21425 | | 23711 |
| 25998 | 16854 | 28284 | 19140 | 30570 | 21426 | | 23712 |
| 25999 | 16855 | 28285 | 19141 | 30571 | 21427 | | 23713 |
| 26000 | 16856 | 28286 | 19142 | 30572 | 21428 | | 23714 |
| 26001 | 16857 | 28287 | 19143 | 30573 | 21429 | | 23715 |
| 26002 | 16858 | 28288 | 19144 | 30574 | 21430 | | 23716 |
| 26003 | 16859 | 28289 | 19145 | 30575 | 21431 | | 23717 |
| 26004 | 16860 | 28290 | 19146 | 30576 | 21432 | | 23718 |
| 26005 | 16861 | 28291 | 19147 | 30577 | 21433 | | 23719 |
| 26006 | 16862 | 28292 | 19148 | 30578 | 21434 | | 23720 |
| 26007 | 16863 | 28293 | 19149 | 30579 | 21435 | | 23721 |
| 26008 | 16864 | 28294 | 19150 | 30580 | 21436 | | 23722 |
| 26009 | 16865 | 28295 | 19151 | 30581 | 21437 | | 23723 |
| 26010 | 16866 | 28296 | 19152 | 30582 | 21438 | | 23724 |
| 26011 | 16867 | 28297 | 19153 | 30583 | 21439 | | 23725 |
| 26012 | 16868 | 28298 | 19154 | 30584 | 21440 | | 23726 |
| 26013 | 16869 | 28299 | 19155 | 30585 | 21441 | | 23727 |
| 26014 | 16870 | 28300 | 19156 | 30586 | 21442 | | 23728 |
| 26015 | 16871 | 28301 | 19157 | 30587 | 21443 | | 23729 |
| 26016 | 16872 | 28302 | 19158 | 30588 | 21444 | | 23730 |
| 26017 | 16873 | 28303 | 19159 | 30589 | 21445 | | 23731 |
| 26018 | 16874 | 28304 | 19160 | 30590 | 21446 | | 23732 |
| 26019 | 16875 | 28305 | 19161 | 30591 | 21447 | | 23733 |
| 26020 | 16876 | 28306 | 19162 | 30592 | 21448 | | 23734 |
| 26021 | 16877 | 28307 | 19163 | 30593 | 21449 | | 23735 |
| 26022 | 16878 | 28308 | 19164 | 30594 | 21450 | | 23736 |
| 26023 | 16879 | 28309 | 19165 | 30595 | 21451 | | 23737 |
| 26024 | 16880 | 28310 | 19166 | 30596 | 21452 | | 23738 |
| 26025 | 16881 | 28311 | 19167 | 30597 | 21453 | | 23739 |
| 26026 | 16882 | 28312 | 19168 | 30598 | 21454 | | 23740 |
| 26027 | 16883 | 28313 | 19169 | 30599 | 21455 | | 23741 |
| 26028 | 16884 | 28314 | 19170 | 30600 | 21456 | | 23742 |
| 26029 | 16885 | 28315 | 19171 | 30601 | 21457 | | 23743 |
| 26030 | 16886 | 28316 | 19172 | 30602 | 21458 | | 23744 |
| 26031 | 16887 | 28317 | 19173 | 30603 | 21459 | | 23745 |
| 26032 | 16888 | 28318 | 19174 | 30604 | 21460 | | 23746 |
| 26033 | 16889 | 28319 | 19175 | 30605 | 21461 | | 23747 |
| 26034 | 16890 | 28320 | 19176 | 30606 | 21462 | | 23748 |
| 26035 | 16891 | 28321 | 19177 | 30607 | 21463 | | 23749 |
| 26036 | 16892 | 28322 | 19178 | 30608 | 21464 | | 23750 |
| 26037 | 16893 | 28323 | 19179 | 30609 | 21465 | | 23751 |
| 26038 | 16894 | 28324 | 19180 | 30610 | 21466 | | 23752 |
| 26039 | 16895 | 28325 | 19181 | 30611 | 21467 | | 23753 |
| 26040 | 16896 | 28326 | 19182 | 30612 | 21468 | | 23754 |
| 26041 | 16897 | 28327 | 19183 | 30613 | 21469 | | 23755 |
| 26042 | 16898 | 28328 | 19184 | 30614 | 21470 | | 23756 |
| 26043 | 16899 | 28329 | 19185 | 30615 | 21471 | | 23757 |
| 26044 | 16900 | 28330 | 19186 | 30616 | 21472 | | 23758 |
| 26045 | 16901 | 28331 | 19187 | 30617 | 21473 | | 23759 |
| 26046 | 16902 | 28332 | 19188 | 30618 | 21474 | | 23760 |
| 26047 | 16903 | 28333 | 19189 | 30619 | 21475 | | 23761 |
| 26048 | 16904 | 28334 | 19190 | 30620 | 21476 | | 23762 |
| 26049 | 16905 | 28335 | 19191 | 30621 | 21477 | | 23763 |
| 26050 | 16906 | 28336 | 19192 | 30622 | 21478 | | 23764 |
| 26051 | 16907 | 28337 | 19193 | 30623 | 21479 | | 23765 |
| 26052 | 16908 | 28338 | 19194 | 30624 | 21480 | | 23766 |
| 26053 | 16909 | 28339 | 19195 | 30625 | 21481 | | 23767 |
| 26054 | 16910 | 28340 | 19196 | 30626 | 21482 | | 23768 |
| 26055 | 16911 | 28341 | 19197 | 30627 | 21483 | | 23769 |
| 26056 | 16912 | 28342 | 19198 | 30628 | 21484 | | 23770 |
| 26057 | 16913 | 28343 | 19199 | 30629 | 21485 | | 23771 |
| 26058 | 16914 | 28344 | 19200 | 30630 | 21486 | | 23772 |
| 26059 | 16915 | 28345 | 19201 | 30631 | 21487 | | 23773 |
| 26060 | 16916 | 28346 | 19202 | 30632 | 21488 | | 23774 |
| 26061 | 16917 | 28347 | 19203 | 30633 | 21489 | | 23775 |
| 26062 | 16918 | 28348 | 19204 | 30634 | 21490 | | 23776 |
| 26063 | 16919 | 28349 | 19205 | 30635 | 21491 | | 23777 |
| 26064 | 16920 | 28350 | 19206 | 30636 | 21492 | | 23778 |
| 26065 | 16921 | 28351 | 19207 | 30637 | 21493 | | 23779 |
| 26066 | 16922 | 28352 | 19208 | 30638 | 21494 | | 23780 |
| 26067 | 16923 | 28353 | 19209 | 30639 | 21495 | | 23781 |
| 26068 | 16924 | 28354 | 19210 | 30640 | 21496 | | 23782 |
| 26069 | 16925 | 28355 | 19211 | 30641 | 21497 | | 23783 |
| 26070 | 16926 | 28356 | 19212 | 30642 | 21498 | | 23784 |
| 26071 | 16927 | 28357 | 19213 | 30643 | 21499 | | 23785 |
| 26072 | 16928 | 28358 | 19214 | 30644 | 21500 | | 23786 |
| 26073 | 16929 | 28359 | 19215 | 30645 | 21501 | | 23787 |
| 26074 | 16930 | 28360 | 19216 | 30646 | 21502 | | 23788 |
| 26075 | 16931 | 28361 | 19217 | 30647 | 21503 | | 23789 |
| 26076 | 16932 | 28362 | 19218 | 30648 | 21504 | | 23790 |
| 26077 | 16933 | 28363 | 19219 | 30649 | 21505 | | 23791 |
| 26078 | 16934 | 28364 | 19220 | 30650 | 21506 | | 23792 |
| 26079 | 16935 | 28365 | 19221 | 30651 | 21507 | | 23793 |
| 26080 | 16936 | 28366 | 19222 | 30652 | 21508 | | 23794 |
| 26081 | 16937 | 28367 | 19223 | 30653 | 21509 | | 23795 |
| 26082 | 16938 | 28368 | 19224 | 30654 | 21510 | | 23796 |
| 26083 | 16939 | 28369 | 19225 | 30655 | 21511 | | 23797 |
| 26084 | 16940 | 28370 | 19226 | 30656 | 21512 | | 23798 |
| 26085 | 16941 | 28371 | 19227 | 30657 | 21513 | | 23799 |
| 26086 | 16942 | 28372 | 19228 | 30658 | 21514 | | 23800 |
| 26087 | 16943 | 28373 | 19229 | 30659 | 21515 | | 23801 |
| 26088 | 16944 | 28374 | 19230 | 30660 | 21516 | | 23802 |
| 26089 | 16945 | 28375 | 19231 | 30661 | 21517 | | 23803 |
| 26090 | 16946 | 28376 | 19232 | 30662 | 21518 | | 23804 |
| 26091 | 16947 | 28377 | 19233 | 30663 | 21519 | | 23805 |
| 26092 | 16948 | 28378 | 19234 | 30664 | 21520 | | 23806 |
| 26093 | 16949 | 28379 | 19235 | 30665 | 21521 | | 23807 |
| 26094 | 16950 | 28380 | 19236 | 30666 | 21522 | | 23808 |
| 26095 | 16951 | 28381 | 19237 | 30667 | 21523 | | 23809 |
| 26096 | 16952 | 28382 | 19238 | 30668 | 21524 | | 23810 |
| 26097 | 16953 | 28383 | 19239 | 30669 | 21525 | | 23811 |
| 26098 | 16954 | 28384 | 19240 | 30670 | 21526 | | 23812 |
| 26099 | 16955 | 28385 | 19241 | 30671 | 21527 | | 23813 |
| 26100 | 16956 | 28386 | 19242 | 30672 | 21528 | | 23814 |
| 26101 | 16957 | 28387 | 19243 | 30673 | 21529 | | 23815 |
| 26102 | 16958 | 28388 | 19244 | 30674 | 21530 | | 23816 |
| 26103 | 16959 | 28389 | 19245 | 30675 | 21531 | | 23817 |
| 26104 | 16960 | 28390 | 19246 | 30676 | 21532 | | 23818 |
| 26105 | 16961 | 28391 | 19247 | 30677 | 21533 | | 23819 |
| 26106 | 16962 | 28392 | 19248 | 30678 | 21534 | | 23820 |
| 26107 | 16963 | 28393 | 19249 | 30679 | 21535 | | 23821 |
| 26108 | 16964 | 28394 | 19250 | 30680 | 21536 | | 23822 |
| 26109 | 16965 | 28395 | 19251 | 30681 | 21537 | | 23823 |
| 26110 | 16966 | 28396 | 19252 | 30682 | 21538 | | 23824 |
| 26111 | 16967 | 28397 | 19253 | 30683 | 21539 | | 23825 |
| 26112 | 16968 | 28398 | 19254 | 30684 | 21540 | | 23826 |
| 26113 | 16969 | 28399 | 19255 | 30685 | 21541 | | 23827 |
| 26114 | 16970 | 28400 | 19256 | 30686 | 21542 | | 23828 |
| 26115 | 16971 | 28401 | 19257 | 30687 | 21543 | | 23829 |
| 26116 | 16972 | 28402 | 19258 | 30688 | 21544 | | 23830 |
| 26117 | 16973 | 28403 | 19259 | 30689 | 21545 | | 23831 |
| 26118 | 16974 | 28404 | 19260 | 30690 | 21546 | | 23832 |
| 26119 | 16975 | 28405 | 19261 | 30691 | 21547 | | 23833 |
| 26120 | 16976 | 28406 | 19262 | 30692 | 21548 | | 23834 |
| 26121 | 16977 | 28407 | 19263 | 30693 | 21549 | | 23835 |
| 26122 | 16978 | 28408 | 19264 | 30694 | 21550 | | 23836 |
| 26123 | 16979 | 28409 | 19265 | 30695 | 21551 | | 23837 |
| 26124 | 16980 | 28410 | 19266 | 30696 | 21552 | | 23838 |
| 26125 | 16981 | 28411 | 19267 | 30697 | 21553 | | 23839 |
| 26126 | 16982 | 28412 | 19268 | 30698 | 21554 | | 23840 |
| 26127 | 16983 | 28413 | 19269 | 30699 | 21555 | | 23841 |
| 26128 | 16984 | 28414 | 19270 | 30700 | 21556 | | 23842 |
| 26129 | 16985 | 28415 | 19271 | 30701 | 21557 | | 23843 |
| 26130 | 16986 | 28416 | 19272 | 30702 | 21558 | | 23844 |
| 26131 | 16987 | 28417 | 19273 | 30703 | 21559 | | 23845 |
| 26132 | 16988 | 28418 | 19274 | 30704 | 21560 | | 23846 |
| 26133 | 16989 | 28419 | 19275 | 30705 | 21561 | | 23847 |
| 26134 | 16990 | 28420 | 19276 | 30706 | 21562 | | 23848 |
| 26135 | 16991 | 28421 | 19277 | 30707 | 21563 | | 23849 |
| 26136 | 16992 | 28422 | 19278 | 30708 | 21564 | | 23850 |
| 26137 | 16993 | 28423 | 19279 | 30709 | 21565 | | 23851 |
| 26138 | 16994 | 28424 | 19280 | 30710 | 21566 | | 23852 |
| 26139 | 16995 | 28425 | 19281 | 30711 | 21567 | | 23853 |
| 26140 | 16996 | 28426 | 19282 | 30712 | 21568 | | 23854 |
| 26141 | 16997 | 28427 | 19283 | 30713 | 21569 | | 23855 |
| 26142 | 16998 | 28428 | 19284 | 30714 | 21570 | | 23856 |
| 26143 | 16999 | 28429 | 19285 | 30715 | 21571 | | 23857 |
| 26144 | 17000 | 28430 | 19286 | 30716 | 21572 | | 23858 |
| 26145 | 17001 | 28431 | 19287 | 30717 | 21573 | | 23859 |
| 26146 | 17002 | 28432 | 19288 | 30718 | 21574 | | 23860 |
| 26147 | 17003 | 28433 | 19289 | 30719 | 21575 | | 23861 |
| 26148 | 17004 | 28434 | 19290 | 30720 | 21576 | | 23862 |
| 26149 | 17005 | 28435 | 19291 | 30721 | 21577 | | 23863 |
| 26150 | 17006 | 28436 | 19292 | 30722 | 21578 | | 23864 |
| 26151 | 17007 | 28437 | 19293 | 30723 | 21579 | | 23865 |
| 26152 | 17008 | 28438 | 19294 | 30724 | 21580 | | 23866 |
| 26153 | 17009 | 28439 | 19295 | 30725 | 21581 | | 23867 |
| 26154 | 17010 | 28440 | 19296 | 30726 | 21582 | | 23868 |
| 26155 | 17011 | 28441 | 19297 | 30727 | 21583 | | 23869 |
| 26156 | 17012 | 28442 | 19298 | 30728 | 21584 | | 23870 |
| 26157 | 17013 | 28443 | 19299 | 30729 | 21585 | | 23871 |
| 26158 | 17014 | 28444 | 19300 | 30730 | 21586 | | 23872 |
| 26159 | 17015 | 28445 | 19301 | 30731 | 21587 | | 23873 |
| 26160 | 17016 | 28446 | 19302 | 30732 | 21588 | | 23874 |
| 26161 | 17017 | 28447 | 19303 | 30733 | 21589 | | 23875 |
| 26162 | 17018 | 28448 | 19304 | 30734 | 21590 | | 23876 |
| 26163 | 17019 | 28449 | 19305 | 30735 | 21591 | | 23877 |
| 26164 | 17020 | 28450 | 19306 | 30736 | 21592 | | 23878 |
| 26165 | 17021 | 28451 | 19307 | 30737 | 21593 | | 23879 |
| 26166 | 17022 | 28452 | 19308 | 30738 | 21594 | | 23880 |
| 26167 | 17023 | 28453 | 19309 | 30739 | 21595 | | 23881 |
| 26168 | 17024 | 28454 | 19310 | 30740 | 21596 | | 23882 |
| 26169 | 17025 | 28455 | 19311 | 30741 | 21597 | | 23883 |
| 26170 | 17026 | 28456 | 19312 | 30742 | 21598 | | 23884 |
| 26171 | 17027 | 28457 | 19313 | 30743 | 21599 | | 23885 |
| 26172 | 17028 | 28458 | 19314 | 30744 | 21600 | | 23886 |
| 26173 | 17029 | 28459 | 19315 | 30745 | 21601 | | 23887 |
| 26174 | 17030 | 28460 | 19316 | 30746 | 21602 | | 23888 |
| 26175 | 17031 | 28461 | 19317 | 30747 | 21603 | | 23889 |
| 26176 | 17032 | 28462 | 19318 | 30748 | 21604 | | 23890 |
| 26177 | 17033 | 28463 | 19319 | 30749 | 21605 | | 23891 |
| 26178 | 17034 | 28464 | 19320 | 30750 | 21606 | | 23892 |
| 26179 | 17035 | 28465 | 19321 | 30751 | 21607 | | 23893 |
| 26180 | 17036 | 28466 | 19322 | 30752 | 21608 | | 23894 |
| 26181 | 17037 | 28467 | 19323 | 30753 | 21609 | | 23895 |
| 26182 | 17038 | 28468 | 19324 | 30754 | 21610 | | 23896 |
| 26183 | 17039 | 28469 | 19325 | 30755 | 21611 | | 23897 |
| 26184 | 17040 | 28470 | 19326 | 30756 | 21612 | | 23898 |
| 26185 | 17041 | 28471 | 19327 | 30757 | 21613 | | 23899 |
| 26186 | 17042 | 28472 | 19328 | 30758 | 21614 | | 23900 |
| 26187 | 17043 | 28473 | 19329 | 30759 | 21615 | | 23901 |
| 26188 | 17044 | 28474 | 19330 | 30760 | 21616 | | 23902 |
| 26189 | 17045 | 28475 | 19331 | 30761 | 21617 | | 23903 |
| 26190 | 17046 | 28476 | 19332 | 30762 | 21618 | | 23904 |
| 26191 | 17047 | 28477 | 19333 | 30763 | 21619 | | 23905 |
| 26192 | 17048 | 28478 | 19334 | 30764 | 21620 | | 23906 |
| 26193 | 17049 | 28479 | 19335 | 30765 | 21621 | | 23907 |
| 26194 | 17050 | 28480 | 19336 | 30766 | 21622 | | 23908 |
| 26195 | 17051 | 28481 | 19337 | 30767 | 21623 | | 23909 |
| 26196 | 17052 | 28482 | 19338 | 30768 | 21624 | | 23910 |
| 26197 | 17053 | 28483 | 19339 | 30769 | 21625 | | 23911 |
| 26198 | 17054 | 28484 | 19340 | 30770 | 21626 | | 23912 |
| 26199 | 17055 | 28485 | 19341 | 30771 | 21627 | | 23913 |
| 26200 | 17056 | 28486 | 19342 | 30772 | 21628 | | 23914 |
| 26201 | 17057 | 28487 | 19343 | 30773 | 21629 | | 23915 |
| 26202 | 17058 | 28488 | 19344 | 30774 | 21630 | | 23916 |
| 26203 | 17059 | 28489 | 19345 | 30775 | 21631 | | 23917 |
| 26204 | 17060 | 28490 | 19346 | 30776 | 21632 | | 23918 |
| 26205 | 17061 | 28491 | 19347 | 30777 | 21633 | | 23919 |
| 26206 | 17062 | 28492 | 19348 | 30778 | 21634 | | 23920 |
| 26207 | 17063 | 28493 | 19349 | 30779 | 21635 | | 23921 |
| 26208 | 17064 | 28494 | 19350 | 30780 | 21636 | | 23922 |
| 26209 | 17065 | 28495 | 19351 | 30781 | 21637 | | 23923 |
| 26210 | 17066 | 28496 | 19352 | 30782 | 21638 | | 23924 |
| 26211 | 17067 | 28497 | 19353 | 30783 | 21639 | | 23925 |
| 26212 | 17068 | 28498 | 19354 | 30784 | 21640 | | 23926 |
| 26213 | 17069 | 28499 | 19355 | 30785 | 21641 | | 23927 |
| 26214 | 17070 | 28500 | 19356 | 30786 | 21642 | | 23928 |
| 26215 | 17071 | 28501 | 19357 | 30787 | 21643 | | 23929 |
| 26216 | 17072 | 28502 | 19358 | 30788 | 21644 | | 23930 |
| 26217 | 17073 | 28503 | 19359 | 30789 | 21645 | | 23931 |
| 26218 | 17074 | 28504 | 19360 | 30790 | 21646 | | 23932 |
| 26219 | 17075 | 28505 | 19361 | 30791 | 21647 | | 23933 |
| 26220 | 17076 | 28506 | 19362 | 30792 | 21648 | | 23934 |
| 26221 | 17077 | 28507 | 19363 | 30793 | 21649 | | 23935 |
| 26222 | 17078 | 28508 | 19364 | 30794 | 21650 | | 23936 |
| 26223 | 17079 | 28509 | 19365 | 30795 | 21651 | | 23937 |
| 26224 | 17080 | 28510 | 19366 | 30796 | 21652 | | 23938 |
| 26225 | 17081 | 28511 | 19367 | 30797 | 21653 | | 23939 |
| 26226 | 17082 | 28512 | 19368 | 30798 | 21654 | | 23940 |
| 26227 | 17083 | 28513 | 19369 | 30799 | 21655 | | 23941 |
| 26228 | 17084 | 28514 | 19370 | 30800 | 21656 | | 23942 |
| 26229 | 17085 | 28515 | 19371 | 30801 | 21657 | | 23943 |
| 26230 | 17086 | 28516 | 19372 | 30802 | 21658 | | 23944 |
| 26231 | 17087 | 28517 | 19373 | 30803 | 21659 | | 23945 |
| 26232 | 17088 | 28518 | 19374 | 30804 | 21660 | | 23946 |
| 26233 | 17089 | 28519 | 19375 | 30805 | 21661 | | 23947 |
| 26234 | 17090 | 28520 | 19376 | 30806 | 21662 | | 23948 |
| 26235 | 17091 | 28521 | 19377 | 30807 | 21663 | | 23949 |
| 26236 | 17092 | 28522 | 19378 | 30808 | 21664 | | 23950 |
| 26237 | 17093 | 28523 | 19379 | 30809 | 21665 | | 23951 |
| 26238 | 17094 | 28524 | 19380 | 30810 | 21666 | | 23952 |
| 26239 | 17095 | 28525 | 19381 | 30811 | 21667 | | 23953 |
| 26240 | 17096 | 28526 | 19382 | 30812 | 21668 | | 23954 |
| 26241 | 17097 | 28527 | 19383 | 30813 | 21669 | | 23955 |
| 26242 | 17098 | 28528 | 19384 | 30814 | 21670 | | 23956 |
| 26243 | 17099 | 28529 | 19385 | 30815 | 21671 | | 23957 |
| 26244 | 17100 | 28530 | 19386 | 30816 | 21672 | | 23958 |
| 26245 | 17101 | 28531 | 19387 | 30817 | 21673 | | 23959 |
| 26246 | 17102 | 28532 | 19388 | 30818 | 21674 | | 23960 |
| 26247 | 17103 | 28533 | 19389 | 30819 | 21675 | | 23961 |
| 26248 | 17104 | 28534 | 19390 | 30820 | 21676 | | 23962 |
| 26249 | 17105 | 28535 | 19391 | 30821 | 21677 | | 23963 |
| 26250 | 17106 | 28536 | 19392 | 30822 | 21678 | | 23964 |
| 26251 | 17107 | 28537 | 19393 | 30823 | 21679 | | 23965 |
| 26252 | 17108 | 28538 | 19394 | 30824 | 21680 | | 23966 |
| 26253 | 17109 | 28539 | 19395 | 30825 | 21681 | | 23967 |
| 26254 | 17110 | 28540 | 19396 | 30826 | 21682 | | 23968 |
| 26255 | 17111 | 28541 | 19397 | 30827 | 21683 | | 23969 |
| 26256 | 17112 | 28542 | 19398 | 30828 | 21684 | | 23970 |
| 26257 | 17113 | 28543 | 19399 | 30829 | 21685 | | 23971 |
| 26258 | 17114 | 28544 | 19400 | 30830 | 21686 | | 23972 |
| 26259 | 17115 | 28545 | 19401 | 30831 | 21687 | | 23973 |
| 26260 | 17116 | 28546 | 19402 | 30832 | 21688 | | 23974 |
| 26261 | 17117 | 28547 | 19403 | 30833 | 21689 | | 23975 |
| 26262 | 17118 | 28548 | 19404 | 30834 | 21690 | | 23976 |
| 26263 | 17119 | 28549 | 19405 | 30835 | 21691 | | 23977 |
| 26264 | 17120 | 28550 | 19406 | 30836 | 21692 | | 23978 |
| 26265 | 17121 | 28551 | 19407 | 30837 | 21693 | | 23979 |
| 26266 | 17122 | 28552 | 19408 | 30838 | 21694 | | 23980 |
| 26267 | 17123 | 28553 | 19409 | 30839 | 21695 | | 23981 |
| 26268 | 17124 | 28554 | 19410 | 30840 | 21696 | | 23982 |
| 26269 | 17125 | 28555 | 19411 | 30841 | 21697 | | 23983 |
| 26270 | 17126 | 28556 | 19412 | 30842 | 21698 | | 23984 |
| 26271 | 17127 | 28557 | 19413 | 30843 | 21699 | | 23985 |
| 26272 | 17128 | 28558 | 19414 | 30844 | 21700 | | 23986 |
| 26273 | 17129 | 28559 | 19415 | 30845 | 21701 | | 23987 |
| 26274 | 17130 | 28560 | 19416 | 30846 | 21702 | | 23988 |
| 26275 | 17131 | 28561 | 19417 | 30847 | 21703 | | 23989 |
| 26276 | 17132 | 28562 | 19418 | 30848 | 21704 | | 23990 |
| 26277 | 17133 | 28563 | 19419 | 30849 | 21705 | | 23991 |
| 26278 | 17134 | 28564 | 19420 | 30850 | 21706 | | 23992 |
| 26279 | 17135 | 28565 | 19421 | 30851 | 21707 | | 23993 |
| 26280 | 17136 | 28566 | 19422 | 30852 | 21708 | | 23994 |
| 26281 | 17137 | 28567 | 19423 | 30853 | 21709 | | 23995 |
| 26282 | 17138 | 28568 | 19424 | 30854 | 21710 | | 23996 |
| 26283 | 17139 | 28569 | 19425 | 30855 | 21711 | | 23997 |
| 26284 | 17140 | 28570 | 19426 | 30856 | 21712 | | 23998 |
| 26285 | 17141 | 28571 | 19427 | 30857 | 21713 | | 23999 |
| 26286 | 17142 | 28572 | 19428 | 30858 | 21714 | | 24000 |
| 26287 | 17143 | 28573 | 19429 | 30859 | 21715 | | 24001 |
| 26288 | 17144 | 28574 | 19430 | 30860 | 21716 | | 24002 |
| 26289 | 17145 | 28575 | 19431 | 30861 | 21717 | | 24003 |
| 26290 | 17146 | 28576 | 19432 | 30862 | 21718 | | 24004 |
| 26291 | 17147 | 28577 | 19433 | 30863 | 21719 | | 24005 |
| 26292 | 17148 | 28578 | 19434 | 30864 | 21720 | | 24006 |
| 26293 | 17149 | 28579 | 19435 | 30865 | 21721 | | 24007 |
| 26294 | 17150 | 28580 | 19436 | 30866 | 21722 | | 24008 |
| 26295 | 17151 | 28581 | 19437 | 30867 | 21723 | | 24009 |
| 26296 | 17152 | 28582 | 19438 | 30868 | 21724 | | 24010 |
| 26297 | 17153 | 28583 | 19439 | 30869 | 21725 | | 24011 |
| 26298 | 17154 | 28584 | 19440 | 30870 | 21726 | | 24012 |
| 26299 | 17155 | 28585 | 19441 | 30871 | 21727 | | 24013 |
| 26300 | 17156 | 28586 | 19442 | 30872 | 21728 | | 24014 |
| 26301 | 17157 | 28587 | 19443 | 30873 | 21729 | | 24015 |
| 26302 | 17158 | 28588 | 19444 | 30874 | 21730 | | 24016 |
| 26303 | 17159 | 28589 | 19445 | 30875 | 21731 | | 24017 |
| 26304 | 17160 | 28590 | 19446 | 30876 | 21732 | | 24018 |
| 26305 | 17161 | 28591 | 19447 | 30877 | 21733 | | 24019 |
| 26306 | 17162 | 28592 | 19448 | 30878 | 21734 | | 24020 |
| 26307 | 17163 | 28593 | 19449 | 30879 | 21735 | | 24021 |
| 26308 | 17164 | 28594 | 19450 | 30880 | 21736 | | 24022 |
| 26309 | 17165 | 28595 | 19451 | 30881 | 21737 | | 24023 |
| 26310 | 17166 | 28596 | 19452 | 30882 | 21738 | | 24024 |
| 26311 | 17167 | 28597 | 19453 | 30883 | 21739 | | 24025 |
| 26312 | 17168 | 28598 | 19454 | 30884 | 21740 | | 24026 |
| 26313 | 17169 | 28599 | 19455 | 30885 | 21741 | | 24027 |
| 26314 | 17170 | 28600 | 19456 | 30886 | 21742 | | 24028 |
| 26315 | 17171 | 28601 | 19457 | 30887 | 21743 | | 24029 |
| 26316 | 17172 | 28602 | 19458 | 30888 | 21744 | | 24030 |
| 26317 | 17173 | 28603 | 19459 | 30889 | 21745 | | 24031 |
| 26318 | 17174 | 28604 | 19460 | 30890 | 21746 | | 24032 |
| 26319 | 17175 | 28605 | 19461 | 30891 | 21747 | | 24033 |
| 26320 | 17176 | 28606 | 19462 | 30892 | 21748 | | 24034 |
| 26321 | 17177 | 28607 | 19463 | 30893 | 21749 | | 24035 |
| 26322 | 17178 | 28608 | 19464 | 30894 | 21750 | | 24036 |
| 26323 | 17179 | 28609 | 19465 | 30895 | 21751 | | 24037 |
| 26324 | 17180 | 28610 | 19466 | 30896 | 21752 | | 24038 |
| 26325 | 17181 | 28611 | 19467 | 30897 | 21753 | | 24039 |
| 26326 | 17182 | 28612 | 19468 | 30898 | 21754 | | 24040 |
| 26327 | 17183 | 28613 | 19469 | 30899 | 21755 | | 24041 |
| 26328 | 17184 | 28614 | 19470 | 30900 | 21756 | | 24042 |
| 26329 | 17185 | 28615 | 19471 | 30901 | 21757 | | 24043 |
| 26330 | 17186 | 28616 | 19472 | 30902 | 21758 | | 24044 |
| 26331 | 17187 | 28617 | 19473 | 30903 | 21759 | | 24045 |
| 26332 | 17188 | 28618 | 19474 | 30904 | 21760 | | 24046 |
| 26333 | 17189 | 28619 | 19475 | 30905 | 21761 | | 24047 |
| 26334 | 17190 | 28620 | 19476 | 30906 | 21762 | | 24048 |
| 26335 | 17191 | 28621 | 19477 | 30907 | 21763 | | 24049 |
| 26336 | 17192 | 28622 | 19478 | 30908 | 21764 | | 24050 |
| 26337 | 17193 | 28623 | 19479 | 30909 | 21765 | | 24051 |
| 26338 | 17194 | 28624 | 19480 | 30910 | 21766 | | 24052 |
| 26339 | 17195 | 28625 | 19481 | 30911 | 21767 | | 24053 |
| 26340 | 17196 | 28626 | 19482 | 30912 | 21768 | | 24054 |
| 26341 | 17197 | 28627 | 19483 | 30913 | 21769 | | 24055 |
| 26342 | 17198 | 28628 | 19484 | 30914 | 21770 | | 24056 |
| 26343 | 17199 | 28629 | 19485 | 30915 | 21771 | | 24057 |
| 26344 | 17200 | 28630 | 19486 | 30916 | 21772 | | 24058 |
| 26345 | 17201 | 28631 | 19487 | 30917 | 21773 | | 24059 |
| 26346 | 17202 | 28632 | 19488 | 30918 | 21774 | | 24060 |
| 26347 | 17203 | 28633 | 19489 | 30919 | 21775 | | 24061 |
| 26348 | 17204 | 28634 | 19490 | 30920 | 21776 | | 24062 |
| 26349 | 17205 | 28635 | 19491 | 30921 | 21777 | | 24063 |
| 26350 | 17206 | 28636 | 19492 | 30922 | 21778 | | 24064 |
| 26351 | 17207 | 28637 | 19493 | 30923 | 21779 | | 24065 |
| 26352 | 17208 | 28638 | 19494 | 30924 | 21780 | | 24066 |
| 26353 | 17209 | 28639 | 19495 | 30925 | 21781 | | 24067 |
| 26354 | 17210 | 28640 | 19496 | 30926 | 21782 | | 24068 |
| 26355 | 17211 | 28641 | 19497 | 30927 | 21783 | | 24069 |
| 26356 | 17212 | 28642 | 19498 | 30928 | 21784 | | 24070 |
| 26357 | 17213 | 28643 | 19499 | 30929 | 21785 | | 24071 |
| 26358 | 17214 | 28644 | 19500 | 30930 | 21786 | | 24072 |
| 26359 | 17215 | 28645 | 19501 | 30931 | 21787 | | 24073 |
| 26360 | 17216 | 28646 | 19502 | 30932 | 21788 | | 24074 |
| 26361 | 17217 | 28647 | 19503 | 30933 | 21789 | | 24075 |
| 26362 | 17218 | 28648 | 19504 | 30934 | 21790 | | 24076 |
| 26363 | 17219 | 28649 | 19505 | 30935 | 21791 | | 24077 |
| 26364 | 17220 | 28650 | 19506 | 30936 | 21792 | | 24078 |
| 26365 | 17221 | 28651 | 19507 | 30937 | 21793 | | 24079 |
| 26366 | 17222 | 28652 | 19508 | 30938 | 21794 | | 24080 |
| 26367 | 17223 | 28653 | 19509 | 30939 | 21795 | | 24081 |
| 26368 | 17224 | 28654 | 19510 | 30940 | 21796 | | 24082 |
| 26369 | 17225 | 28655 | 19511 | 30941 | 21797 | | 24083 |
| 26370 | 17226 | 28656 | 19512 | 30942 | 21798 | | 24084 |
| 26371 | 17227 | 28657 | 19513 | 30943 | 21799 | | 24085 |
| 26372 | 17228 | 28658 | 19514 | 30944 | 21800 | | 24086 |
| 26373 | 17229 | 28659 | 19515 | 30945 | 21801 | | 24087 |
| 26374 | 17230 | 28660 | 19516 | 30946 | 21802 | | 24088 |
| 26375 | 17231 | 28661 | 19517 | 30947 | 21803 | | 24089 |
| 26376 | 17232 | 28662 | 19518 | 30948 | 21804 | | 24090 |
| 26377 | 17233 | 28663 | 19519 | 30949 | 21805 | | 24091 |
| 26378 | 17234 | 28664 | 19520 | 30950 | 21806 | | 24092 |
| 26379 | 17235 | 28665 | 19521 | 30951 | 21807 | | 24093 |
| 26380 | 17236 | 28666 | 19522 | 30952 | 21808 | | 24094 |
| 26381 | 17237 | 28667 | 19523 | 30953 | 21809 | | 24095 |
| 26382 | 17238 | 28668 | 19524 | 30954 | 21810 | | 24096 |
| 26383 | 17239 | 28669 | 19525 | 30955 | 21811 | | 24097 |
| 26384 | 17240 | 28670 | 19526 | 30956 | 21812 | | 24098 |
| 26385 | 17241 | 28671 | 19527 | 30957 | 21813 | | 24099 |
| 26386 | 17242 | 28672 | 19528 | 30958 | 21814 | | 24100 |
| 26387 | 17243 | 28673 | 19529 | 30959 | 21815 | | 24101 |
| 26388 | 17244 | 28674 | 19530 | 30960 | 21816 | | 24102 |
| 26389 | 17245 | 28675 | 19531 | 30961 | 21817 | | 24103 |
| 26390 | 17246 | 28676 | 19532 | 30962 | 21818 | | 24104 |
| 26391 | 17247 | 28677 | 19533 | 30963 | 21819 | | 24105 |
| 26392 | 17248 | 28678 | 19534 | 30964 | 21820 | | 24106 |
| 26393 | 17249 | 28679 | 19535 | 30965 | 21821 | | 24107 |
| 26394 | 17250 | 28680 | 19536 | 30966 | 21822 | | 24108 |
| 26395 | 17251 | 28681 | 19537 | 30967 | 21823 | | 24109 |
| 26396 | 17252 | 28682 | 19538 | 30968 | 21824 | | 24110 |
| 26397 | 17253 | 28683 | 19539 | 30969 | 21825 | | 24111 |
| 26398 | 17254 | 28684 | 19540 | 30970 | 21826 | | 24112 |
| 26399 | 17255 | 28685 | 19541 | 30971 | 21827 | | 24113 |
| 26400 | 17256 | 28686 | 19542 | 30972 | 21828 | | 24114 |
| 26401 | 17257 | 28687 | 19543 | 30973 | 21829 | | 24115 |
| 26402 | 17258 | 28688 | 19544 | 30974 | 21830 | | 24116 |
| 26403 | 17259 | 28689 | 19545 | 30975 | 21831 | | 24117 |
| 26404 | 17260 | 28690 | 19546 | 30976 | 21832 | | 24118 |
| 26405 | 17261 | 28691 | 19547 | 30977 | 21833 | | 24119 |
| 26406 | 17262 | 28692 | 19548 | 30978 | 21834 | | 24120 |
| 26407 | 17263 | 28693 | 19549 | 30979 | 21835 | | 24121 |
| 26408 | 17264 | 28694 | 19550 | 30980 | 21836 | | 24122 |
| 26409 | 17265 | 28695 | 19551 | 30981 | 21837 | | 24123 |
| 26410 | 17266 | 28696 | 19552 | 30982 | 21838 | | 24124 |
| 26411 | 17267 | 28697 | 19553 | 30983 | 21839 | | 24125 |
| 26412 | 17268 | 28698 | 19554 | 30984 | 21840 | | 24126 |
| 26413 | 17269 | 28699 | 19555 | 30985 | 21841 | | 24127 |
| 26414 | 17270 | 28700 | 19556 | 30986 | 21842 | | 24128 |
| 26415 | 17271 | 28701 | 19557 | 30987 | 21843 | | 24129 |
| 26416 | 17272 | 28702 | 19558 | 30988 | 21844 | | 24130 |
| 26417 | 17273 | 28703 | 19559 | 30989 | 21845 | | 24131 |
| 26418 | 17274 | 28704 | 19560 | 30990 | 21846 | | 24132 |
| 26419 | 17275 | 28705 | 19561 | 30991 | 21847 | | 24133 |
| 26420 | 17276 | 28706 | 19562 | 30992 | 21848 | | 24134 |
| 26421 | 17277 | 28707 | 19563 | 30993 | 21849 | | 24135 |
| 26422 | 17278 | 28708 | 19564 | 30994 | 21850 | | 24136 |
| 26423 | 17279 | 28709 | 19565 | 30995 | 21851 | | 24137 |
| 26424 | 17280 | 28710 | 19566 | 30996 | 21852 | | 24138 |
| 26425 | 17281 | 28711 | 19567 | 30997 | 21853 | | 24139 |
| 26426 | 17282 | 28712 | 19568 | 30998 | 21854 | | 24140 |
| 26427 | 17283 | 28713 | 19569 | 30999 | 21855 | | 24141 |
| 26428 | 17284 | 28714 | 19570 | 31000 | 21856 | | 24142 |
| 26429 | 17285 | 28715 | 19571 | 31001 | 21857 | | 24143 |
| 26430 | 17286 | 28716 | 19572 | 31002 | 21858 | | 24144 |
| 26431 | 17287 | 28717 | 19573 | 31003 | 21859 | | 24145 |
| 26432 | 17288 | 28718 | 19574 | 31004 | 21860 | | 24146 |
| 26433 | 17289 | 28719 | 19575 | 31005 | 21861 | | 24147 |
| 26434 | 17290 | 28720 | 19576 | 31006 | 21862 | | 24148 |
| 26435 | 17291 | 28721 | 19577 | 31007 | 21863 | | 24149 |
| 26436 | 17292 | 28722 | 19578 | 31008 | 21864 | | 24150 |
| 26437 | 17293 | 28723 | 19579 | 31009 | 21865 | | 24151 |
| 26438 | 17294 | 28724 | 19580 | 31010 | 21866 | | 24152 |
| 26439 | 17295 | 28725 | 19581 | 31011 | 21867 | | 24153 |
| 26440 | 17296 | 28726 | 19582 | 31012 | 21868 | | 24154 |
| 26441 | 17297 | 28727 | 19583 | 31013 | 21869 | | 24155 |
| 26442 | 17298 | 28728 | 19584 | 31014 | 21870 | | 24156 |
| 26443 | 17299 | 28729 | 19585 | 31015 | 21871 | | 24157 |
| 26444 | 17300 | 28730 | 19586 | 31016 | 21872 | | 24158 |
| 26445 | 17301 | 28731 | 19587 | 31017 | 21873 | | 24159 |
| 26446 | 17302 | 28732 | 19588 | 31018 | 21874 | | 24160 |
| 26447 | 17303 | 28733 | 19589 | 31019 | 21875 | | 24161 |
| 26448 | 17304 | 28734 | 19590 | 31020 | 21876 | | 24162 |
| 26449 | 17305 | 28735 | 19591 | 31021 | 21877 | | 24163 |
| 26450 | 17306 | 28736 | 19592 | 31022 | 21878 | | 24164 |
| 26451 | 17307 | 28737 | 19593 | 31023 | 21879 | | 24165 |

In some embodiments, compositions comprise an effector protein wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, about 1400, about 1420, about 1440, about 1460, about 1480, about 1490, about 1500, about 1520, about 1540, about 1560, about 1580, about 1600, about 1620, about 1640, about 1660, about 1680, about 1700, about 1720, about 1740, about 1760, about 1780, about 1800, about 1820, about 1840, about 1860, about 1880, about 1900, or about 1920 contiguous amino acids of a sequence selected from SEQ ID NOS: 1-10,484 or 15,022-24,165.

In some embodiments, compositions comprise an effector protein wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, 1250-1350, 1301-1400, 1350-1450, 1401-1500, 1450-1550, 1501-1600, 1550-1650, 1601-1700, 1650-1750, 1701-1800, 1850-1950, 1801-1900, or 1850-1950 of a sequence selected from SEQ ID NOS: 1-10,484 or 15,022-24,165.

In some embodiments, compositions comprise an effector protein wherein the effector protein comprises an amino acid sequence that is at least 90%, at least 95%, or 100% identical to a portion of a sequence selected from SEQ ID NOS: 1-10,484 or 15,022-24,165, and wherein the portion of the sequence is about 30%, about 40% about 50%, about 60%, about 70%, about 80%, or about 90% of a sequence selected from SEQ ID NOS: 1-10,484 or 15,022-24,165.

In some embodiments, compositions comprise an effector protein, wherein portion of the amino acid sequence of the effector protein is at least 65%, at least 70%, at least 75%, at least 80%, at least 85%, at least 90%, at least 95%, at least 97%, or at least 98%, at least 99%, or 100% identical to an equal length portion of a sequence selected from SEQ ID NOs: 1-21. In some embodiments, the length of the portion is selected from: 20 to 40, 40 to 60, 60 to 80, 80 to 100, 100 to 120, 120 to 140, 140 to 160, 160 to 180, 180 to 200, 200 to 220, 220 to 240, 240 to 260, 260 to 280, 280 to 300, 320 to 340, 340 to 360, 360 to 380, and 380 to 400 linked amino acids. In some embodiments, the length of the portion is selected from: 400 to 420, 420 to 440, 440 to 460, 460 to 480, 480 to 500, 520 to 540, 540 to 560, 560 to 580, 580 to 600, 600 to 620, 620 to 640, 640 to 660, 660 to 680, and 680 to 700, 700 to 720, 720 to 740, 740 to 760, 760 to 780, 780 to 800, 820 to 840, 840 to 860, 860 to 880, 880 to 900, 900 to 920, 920 to 940, 940 to 960, 960 to 980, 980 to 1000 linked amino acids. In some embodiments, the length of the portion is selected from: 1000 to 1020, 1020 to 1040, 1040 to 1060, 1060 to 1080, 1080 to 1100, 1100 to 1120, 1120 to 1140, 1140 to 1160, 1160 to 1180, 1180 to 1200, 1220 to 1240, 1240 to 1260, 1260 to 1280, 1280 to 1300, 1300 to 1320, 1320 to 1340, 1340 to 1360, 1360 to 1380, 1380 to 1400, 1420 to 1440, 1440 to 1460, 1460 to 1480, 1480 to 1500, 1500 to 1520, 1520 to 1540, 1540 to 1560, 1560 to 1580, 1580 to 1600 linked amino acids.

In some embodiments, effector proteins comprise a functional domain. The functional domain may comprise nucleic acid binding activity. The functional domain may comprise catalytic activity, also referred to as enzymatic activity. The catalytic activity may be nuclease activity. The nuclease activity may comprise cleaving a strand of a nucleic acid. The nuclease activity may comprise cleaving only one strand of a double stranded nucleic acid, also referred to as nicking. In some embodiments, the functional domain is an HNH domain. In some embodiments, the functional domain is a RuvC domain. In some embodiments, the RuvC domain comprises multiple subdomains. In some embodiments, the functional domain is a zinc finger binding domain. In some embodiments, the functional domain is a HEPN domain. In some embodiments, effector proteins lack a certain functional domain. In some embodiments, the effector protein lacks an HNH domain. In some embodiments, effector proteins lack a zinc finger binding domain.

In some embodiments, effector proteins catalyze cleavage of a target nucleic acid in a cell or a sample. In some embodiments, the target nucleic acid is single stranded (ss). In some embodiments, the target nucleic acid is double stranded (ds). In some embodiments, the target nucleic acid is dsDNA. In some embodiments, the target nucleic acid is ssDNA. In some embodiments, the target nucleic acid is RNA. In some embodiments, effector proteins cleave the target nucleic acid within a target sequence of the target nucleic acid.

In some embodiments, effector proteins catalyze cis cleavage activity. In some embodiments, effector proteins cleave both strands of dsDNA.

In some embodiments, effector proteins cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleosides of a 5′ or 3′ terminus of a PAM sequence. A target nucleic acid may comprise a PAM sequence adjacent to a sequence that is complementary to a guide nucleic acid spacer sequence. In some embodiments, effector proteins do not require a PAM sequence to cleave or a nick a target nucleic acid.

### Engineered Proteins

In some embodiments, effector proteins disclosed herein are engineered proteins. Engineered proteins are not identical to a naturally-occurring protein. Engineered proteins may not comprise an amino acid sequence that is identical to that of a naturally-occurring protein. In some embodiments, the amino acid sequence of an engineered protein is not identical to that of a naturally occurring protein. Engineered proteins may provide an increased activity relative to a naturally occurring protein. Engineered proteins may provide a reduced activity relative to a naturally occurring protein. The activity may be nuclease activity. The activity may be nickase activity. The activity may be nucleic acid binding activity. In some embodiments, a modification of the effector proteins may include addition of one or more amino acids, deletion of one or more amino acids, substitution of one or more amino acids, or combinations thereof. In some embodiments, effector proteins disclosed herein are engineered proteins. Unless otherwise indicated, reference to effector proteins throughout the present disclosure include engineered proteins thereof.

Engineered proteins may provide an increased or reduced activity relative to a naturally occurring protein under a given condition of a cell or sample in which the activity occurs. The condition may be temperature. The temperature may be greater than 20° C., greater than 25° C., greater than 30° C., greater than 35° C., greater than 40° C., greater than 45° C., greater than 50° C., greater than 55° C., greater than 60° C., greater than 65° C., or greater than 70° C., but not greater than 80° C. The condition may be the presence of a salt. The salt may be a magnesium salt, a zinc salt, a potassium salt, a calcium salt or a sodium salt. The condition may be the concentration of one or more salts.

In some embodiments, the amino acid sequence of an engineered protein comprises at least one residue that is different from that of a naturally occurring protein. In some embodiments, the amino acid sequence of an engineered protein comprises at least 2, at least 3, at least 4, at least 5, at least 6, at least 7, at least 8, at least 9 or at least 10 residues that are different from that of a naturally occurring protein. The residues in the engineered protein that differ from those at corresponding positions of the naturally occurring protein (when the engineered and naturally occurring proteins are aligned for maximal identity) may be referred to as substituted residues or amino acid substitutions. In some embodiments, the substituted residues are non-conserved residues relative to the residues at corresponding positions of the naturally occurring protein. A non-conserved residue has a different physicochemical property from the amino acid for which it substitutes. Physicochemical properties include aliphatic, cyclic, aromatic, basic, acidic and hydroxyl-containing. Glycine, alanine, valine, leucine and isoleucine are aliphatic. Serine, Cysteine, threonine and methionine are hydroxyl-containing. Proline is cyclic. Phenylalanine, tyrosine, tryptophan are basic. Aspartate, Glutamate, Asparagine and glutamine are acidic.

In some embodiments, engineered proteins are designed to be catalytically inactive or to have reduced catalytic activity relative to a naturally occurring protein. A catalytically inactive effector protein may be generated by substituting an amino acid that confers a catalytic activity (also referred to as a “catalytic residue”) with a substituted residue that does not support the catalytic activity. In some embodiments, the substituted residue has an aliphatic side chain. In some embodiments, the substituted residue is glycine. In some embodiments, the substituted residue is valine. In some embodiments, the substituted residue is leucine. In some embodiments, the substituted residue is alanine. In some embodiments, the amino acid is aspartate and it is substituted with asparagine. In some embodiments, the amino acid is glutamate and it is substituted with glutamine. An amino acid that confers catalytic activity may be identified by performing sequence alignment of an unmodified effector protein with a similar enzyme having at least one identified catalytic residue; selecting at least one putative catalytic residue in the unmodified effector protein within the portion of the unmodified effector protein that aligns with a portion of the similar enzyme that comprises the identified catalytic residue; substituting the at least one putative catalytic residue of the unmodified effector protein with the different amino acid; and comparing the catalytic activity of the unmodified effector protein to the modified effector protein. A similar enzyme may be an enzyme that is at least 50%, at least 60%, at least 70%, at least 80%, or at least 90% identical to the unmodified effector protein. A similar enzyme may be an enzyme that is not greater than 99.9% identical to the unmodified effector protein. In some embodiments, the portion of the unmodified effector protein that aligns with a portion of the similar enzyme is at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids in length. In some embodiments, the portion of the unmodified effector protein that aligns with a portion of the similar enzyme is not greater than 200 amino acids. In some embodiments, the portion of the unmodified effector protein that aligns with a portion of the similar enzyme comprises a functional domain (e.g., HEPN, HNH, RuvC, zinc finger binding). In some embodiments, comparing the catalytic activity comprises performing a cleavage assay. An example of generating a catalytically inactive effector protein is provided in Example 7.

In some embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the sequences as set forth in TABLE 1. In some embodiments, an effector protein provided herein comprises an amino acid sequence that is at least 65% identical to any one of the sequences as set forth in TABLE 1. In some embodiments, an effector protein provided herein comprises an amino acid sequence that is at least 70% identical to any one of the sequences as set forth in TABLE 1. In some embodiments, an effector protein provided herein comprises an amino acid sequence that is at least 75% identical to any one of the sequences as set forth in TABLE 1. In some embodiments, an effector protein provided herein comprises an amino acid sequence that is at least 80% identical to any one of the sequences as set forth in TABLE 1. In some embodiments, an effector protein provided herein comprises an amino acid sequence that is at least 85% identical to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% identical to any one of the sequences as set forth in TABLE 1. In some embodiments, an effector protein provided herein comprises an amino acid sequence that is at least 97% identical to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% identical to any one of the sequences as set forth in TABLE 1. In some embodiments, an effector protein provided herein comprises an amino acid sequence that is identical to any one of the sequences as set forth in TABLE 1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. The term “variant” refers to a form or version of a protein that differs from the wild-type protein. A variant may have a different function or activity relative to the wild-type protein.

In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

The term “conservative substitution” refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, the one or more amino acid alterations may result in a change in activity of the effector protein relative to a naturally-occurring counterpart. For example, and as described in further detail below, the one or more amino acid alteration increases or decreases catalytic activity of the effector protein relative to a naturally-occurring counterpart. In some embodiments, the one or more amino acid alterations results in a catalytically inactive effector protein variant.

In some embodiments, effector proteins described herein are encoded by a codon optimized nucleic acid. In some embodiments, a nucleic acid sequence encoding an effector protein described herein, is codon optimized. In some embodiments, effector proteins described herein may be codon optimized for expression in a specific cell, for example, a bacterial cell, a plant cell, a eukaryotic cell, an animal cell, a mammalian cell, or a human cell. In some embodiments, the effector protein is codon optimized for a human cell.

In some embodiments, effector proteins may comprise one or more modifications that may provide altered activity as compared to a naturally-occurring counterpart (e.g., a naturally-occurring nuclease or nickase, etc. activity which may be a naturally-occurring effector protein). In some embodiments, activity (e.g., nickase, nuclease, binding, etc, activity) of effector proteins described herein can be measured relative to a naturally-occurring effector protein or compositions containing the same in a cleavage assay. For example, effector proteins may comprise one or more modifications that may provide increased activity as compared to a naturally-occurring counterpart. As another example, effector proteins may provide increased catalytic activity (e.g., nickase, nuclease, binding, etc. activity) as compared to a naturally-occurring counterpart. Effector proteins may provide enhanced nucleic acid binding activity (e.g., enhanced binding of a guide nucleic acid, and/or target nucleic acid) as compared to a naturally-occurring counterpart. An effector protein may have a 1%, 5%, 10%, 20%, 30%, 40%, 50%, 60%, 70%, 80%, 90%, 100%, 120%, 140%, 160%, 180%, 200%, or more, increase of the activity of a naturally-occurring counterpart.

Alternatively, effector proteins may comprise one or more modifications that reduce the activity of the effector proteins relative to a naturally occurring nuclease, or nickase etc. . . . An effector protein may have a 100%, 90%, 80%, 70%, 60%, 50%, 40%, 30%, 20%, 10%, 5%, 1%, or less, decrease of the activity of a naturally occurring counterpart. Decreased activity may be decreased catalytic activity (e.g., nickase, nuclease, binding, etc. activity) as compared to a naturally-occurring counterpart.

An effector protein that has decreased catalytic activity may be referred to as catalytically or enzymatically inactive, catalytically or enzymatically dead, as a dead protein or a dCas protein. In some embodiments, such a protein may comprise an enzymatically inactive domain (e.g. inactive nuclease domain). For example, a nuclease domain (e.g., RuvC domain) of an effector protein may be deleted or mutated relative to a wildtype counterpart so that it is no longer functional or comprises reduced nuclease activity. In some embodiments, a catalytically inactive effector protein may bind to a guide nucleic acid and/or a target nucleic acid but does not cleave the target nucleic acid. In some embodiments, a catalytically inactive effector protein may associate with a guide nucleic acid to activate or repress transcription of a target nucleic acid. In some embodiments, a catalytically inactive effector protein is fused to a fusion partner protein that confers an alternative activity to an effector protein activity. Such fusion proteins are described herein and throughout. The term, “fused,” as used herein, refers to at least two sequences that are connected together, such as by a linker, or by conjugation (e.g., chemical conjugation or enzymatic conjugation). The term “fused” includes a linker.

### Fusion Proteins

In some embodiments, compositions, systems, and methods comprise a fusion protein or uses thereof. A fusion protein generally comprises an effector protein and a fusion partner protein (also referred to as a “fusion partner”). In some embodiments, the fusion partner. In general, the effector protein and the fusion partner are heterologous proteins. In some embodiments, the fusion protein comprises a polypeptide or peptide that is fused or linked to the effector protein. In some embodiments, the fusion protein is a heterologous peptide or polypeptide as described herein. In some embodiments, the amino terminus of the fusion partner is linked/fused to the carboxy terminus of the effector protein. In some embodiments, the carboxy terminus of the fusion partner protein is linked/fused to the amino terminus of the effector protein by the linker. In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In some embodiments, effector proteins described herein can be modified with the addition of one or more heterologous peptides or heterologous polypeptides (referred to collectively herein as a heterologous polypeptide). In some embodiments, an effector protein modified with the addition of one or more heterologous peptides or heterologous polypeptides may be referred to herein as a fusion protein. Such fusion proteins are described herein and throughout.

In some embodiments, a heterologous peptide or heterologous polypeptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. In some embodiments, the subcellular localization signal is a nuclear export signal (NES), a sequence to keep an effector protein retained in the cytoplasm, a mitochondrial localization signal for targeting to the mitochondria, a chloroplast localization signal for targeting to a chloroplast, an ER retention signal, and the like. In some embodiments, an effector protein described herein is not modified with a subcellular localization signal so that the polypeptide is not targeted to the nucleus, which can be advantageous depending on the circumstance (e.g., when the target nucleic acid is an RNA that is present in the cytosol).

In some embodiments, a heterologous peptide or heterologous polypeptide comprises a chloroplast transit peptide (CTP), also referred to as a chloroplast localization signal or a plastid transit peptide, which targets the effector protein to a chloroplast. Chromosomal transgenes from bacterial sources may require a sequence encoding a CTP sequence fused to a sequence encoding an expressed protein (e.g., the effector protein) if the expressed protein is to be compartmentalized in the plant plastid (e.g., chloroplast). The CTP may be removed in a processing step during translocation into the plastid. Accordingly, localization of an effector protein to a chloroplast is often accomplished by means of operably linking a polynucleotide sequence encoding a CTP sequence to the 5′ region of a polynucleotide encoding the exogenous protein.

In some embodiments, the heterologous polypeptide is an endosomal escape peptide (EEP). An EEP is an agent that quickly disrupts the endosome in order to minimize the amount of time that a delivered molecule, such an effector protein, spends in the endosome-like environment, and to avoid getting trapped in the endosomal vesicles and degraded in the lysosomal compartment.

In some embodiments, the heterologous polypeptide is a cell penetrating peptide (CPP), also known as a Protein Transduction Domain (PTD). A CPP or PTD is a polypeptide, polynucleotide, carbohydrate, or organic or inorganic compound that facilitates traversing a lipid bilayer, micelle, cell membrane, organelle membrane, or vesicle membrane.

Further suitable heterologous polypeptides include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a heterologous peptide or heterologous polypeptide comprises a protein tag. In some embodiments, the protein tag is referred to as purification tag or a fluorescent protein. The protein tag may be detectable for use in detection of the effector protein and/or purification of the effector protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6λHis tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the effector protein disclosed herein. A heterologous polypeptide may be located at or near the carboxy terminus (C-terminus) of the effector proteins disclosed herein. In some embodiments, a heterologous polypeptide is located internally in an effector protein described herein (i.e., is not at the N- or C-terminus of an effector protein described herein) at a suitable insertion site.

In some embodiments, an effector protein described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

In some embodiments, a fusion partner imparts some function or activity to a fusion protein that is not provided by an effector protein. Such activities may include but are not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., when a fusion sequence is used that modifies the target nucleic acid or modifies a protein associated with the target nucleic acid). The guide RNA may bind to a target nucleic acid (e.g., a single strand of a target nucleic acid) or a portion thereof, an amplicon thereof, or a portion thereof. By way of non-limiting example, a guide nucleic acid may bind to a target nucleic acid, such as DNA or RNA, from a cancer gene or gene associated with a genetic disorder, or an amplicon thereof, as described herein.

In some embodiments, a fusion partner may provide signaling activity. In some embodiments, a fusion partner may inhibit or promote the formation of multimeric complex of an effector protein. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

In some cases, fusion effector proteins modify a target nucleic acid or the expression thereof. In some cases, the modifications are transient (e.g., transcription repression or activation). In some cases, the modifications are inheritable. For instance, epigenetic modifications made to a target nucleic acid, or to proteins associated with the target nucleic acid, e.g., nucleosomal histones, in a cell, are observed in cells produced by proliferation of the cell.

In some embodiments, fusion partners inhibit or reduce expression of a target nucleic acid. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof. Non-limiting examples of fusion partners that decrease or inhibit transcription include, but are not limited to: histone lysine methyltransferases; histone lysine demethylases; histone lysine deacetylases; and DNA methylases; and functional domains thereof.

In some embodiments, fusion partners activate or increase expression of a target nucleic acid. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. Non-limiting examples of fusion partners that activate or increase transcription include, but are not limited to: histone lysine methyltransferases; histone lysine demethylases; histone acetyltransferases; and DNA demethylases; and functional domains thereof.

In some embodiments, fusion partners comprise an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

In some cases, fusion effector proteins modify a target nucleic acid or the expression thereof, wherein the target nucleic acid comprises a deoxyribonucleoside, a ribonucleoside or a combination thereof. The target nucleic acid may comprise or consist of a single stranded RNA (ssRNA), a double-stranded RNA (dsRNA), a single-stranded DNA (ssDNA), or a double stranded DNA (dsDNA). Non-limiting examples of fusion partners for modifying ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

### Multimeric Complex Formation Modification Activity

In some embodiments, a fusion partner may inhibit the formation of a multimeric complex of an effector protein. Alternatively, the fusion partner promotes the formation of a multimeric complex of the effector protein. By way of non-limiting example, the fusion protein may comprise an effector protein described herein and a fusion partner comprising a Calcineurin A tag, wherein the fusion protein dimerizes in the presence of Tacrolimus (FK506). Also, by way of non-limiting example, the fusion protein may comprise an effector protein described herein and a SpyTag configured to dimerize or associate with another effector protein in a multimeric complex. Multimeric complex formation is further described herein.

### Nucleic Acid Modification Activity

In some embodiments, fusion partners have enzymatic activity that modifies a nucleic acid, such as a target nucleic acid. In some embodiments, the target nucleic acid may comprise or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

The term “transposase activity” refers to catalytic activity that results in the transposition of a first nucleic acid into a second nucleic acid.

In some embodiments, fusion partners target a ssRNA, dsRNA, ssDNA, or a dsDNA. In some embodiments, fusion partners target ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); and other suitable domains that affect nucleic acid modifications.

In some embodiments, an effector protein is a fusion protein, wherein the effector protein is fused to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme fused or linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any function variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as fused to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C·G-to-G·C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U·G in DNA is initiated by a UNG, which recognizes a U·G mismatch and cleaves the glyosidic bond between a uracil and a deoxyribose backbone of DNA. In some embodiments, BER results in the reversion of the U⋅G intermediate created by the first CBE back to a C⋅G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U⋅G mismatch to favor a U⋅A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A⋅T base pair to a G⋅C base pair. In some embodiments, the ABE converts a target A⋅T base pair to G⋅C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, an adenine base editing enzyme of an ABE is an adenosine deaminase. Non-limiting exemplary adenosine base editors suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety).

In some embodiments, the base editor comprises a base editing enzyme fused to TadA by a linker (e.g., wherein the base editing enzyme is fused to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA fused to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is fused to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

### Prime Editing

In some embodiments, a fusion protein and/or a fusion partner can comprise a prime editing enzyme. In some embodiments, a prime editing enzyme comprises a reverse transcriptase. A non-limiting example of a reverse transcriptase is an M-MLV RT enzyme and variants thereof having polymerase activity. In some embodiments, the M-MLV RT enzyme comprises at least one mutation selected from D200N, L603W, T330P, T306K, and W313F relative to wildtype M-MLV RT enzyme.

In some embodiments, a prime editing enzyme may require a prime editing guide RNA (pegRNA) to catalyze an editing. Such a pegRNA may be capable of identifying a target nucleotide or target sequence in a target nucleic acid to be edited and encoding a new genetic information that replaces the target nucleotide or target sequence in the target nucleic acid. A prime editing enzyme may require a pegRNA and a single guide RNA to catalyze the editing. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the dsDNA molecule; a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### Protein Modification Activity

In some embodiments, a fusion partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion partners activate or increase expression of a target nucleic acid. Such fusion proteins comprising the described fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In some embodiments, the transcriptional activators may promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. In some embodiments, the fusion partner is a reverse transcriptase.

Non-limiting examples of fusion partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. Such fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, fusion partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP A1 binds to exonic splicing silencers (ESSs) through its RRM domains and inhibits exon inclusion through a C-terminal Glycine-rich domain. In some embodiments, the RNA splicing factors may regulate alternative use of splice site (ss) by binding to regulatory sequences between two alternative sites. For example, in some embodiments, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, fusion partners comprise a recombinase. In some embodiments, effector proteins described herein are fused with the recombinase. In some embodiments, the effector proteins have reduced nuclease activity or no nuclease activity. In some embodiments, the recombinase is a site-specific recombinase.

In some embodiments, a catalytically inactive effector protein is fused with a recombinase, wherein the recombinase can be a site-specific recombinase. Such polypeptides can be used for site-directed transgene insertion. The term “transgene” refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include: Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

In some embodiments, the fusion partner protein is fused to the 3′ end of the effector protein. In some embodiments, the effector protein is located at an internal location of the fusion partner protein. In some embodiments, the fusion partner protein is located at an internal location of the Cas effector protein. For example, a base editing enzyme (e.g., a deaminase enzyme) is inserted at an internal location of a Cas effector protein. The effector protein may be fused directly or indirectly (e.g., via a linker) to the fusion partner protein. Exemplary linkers are described herein.

In some embodiments, the fusion effector protein or the guide nucleic acid comprises a chemical modification that allows for direct crosslinking between the guide nucleic acid or the effector protein and the fusion partner. By way of non-limiting example, the chemical modification may comprise any one of a SNAP-tag, CLIP-tag, ACP-tag, Halo-tag, and an MCP-tag. In some embodiments, modifications are introduced with a Click Reaction, also known as Click Chemistry. The Click reaction may be copper dependent or copper independent.

In some embodiments, guide nucleic acids comprise an aptamer. The aptamer may serve as a linker between the effector protein and the fusion partner by interacting non-covalently with both. In some embodiments, the aptamer binds a fusion partner, wherein the fusion partner is a transcriptional activator. In some embodiments, the aptamer binds a fusion partner, wherein the fusion partner is a transcriptional inhibitor. In some embodiments, the aptamer binds a fusion partner, wherein the fusion partner comprises a base editor. In some embodiments, the aptamer binds the fusion partner directly. In some embodiments, the aptamer binds the fusion partner indirectly. Aptamers may bind the fusion partner indirectly through an aptamer binding protein. By way of non-limiting example, the aptamer binding protein may be MS2 and the aptamer sequence may be ACATGAGGATCACCCATGT (SEQ ID NO: 15,016); the aptamer binding protein may be PP7 and the aptamer sequence may be GGAGCAGACGATATGGCGTCGCTCC (SEQ ID NO: 15,017); or the aptamer binding protein may be BoxB and the aptamer sequence may be GCCCTGAAGAAGGGC (SEQ ID NO: 15,018).

In some embodiments, the fusion partner is located within effector protein. For example, the fusion partner may be a domain of a fusion partner protein that is internally integrated into the effector protein. In other words, the fusion partner may be located between the 5′ and 3′ ends of the effector protein without disrupting the ability of the fusion effector protein to recognize/bind a target nucleic acid. In some embodiments, the fusion partner replaces a portion of the effector protein. In some embodiments, the fusion partner replaces a domain of the effector protein. In some embodiments, the fusion partner does not replace a portion of the effector protein.

An effector protein disclosed herein or fusion effector protein may comprise a nuclear localization signal (NLS). In some cases, the NLS may comprise a sequence of KRPAATKKAGQAKKKK (SEQ ID NO: 15,019). In some cases, the NLS comprises or consists of a sequence of PKKKRKV (SEQ ID NO: 15,020). In some cases, the NLS comprises or consists of a sequence of LPPLERLTL (SEQ ID NO: 15,021). An effector protein may be codon optimized for expression in a specific cell, for example, a bacterial cell, a plant cell, a eukaryotic cell, an animal cell, a mammalian cell, or a human cell. In some embodiments, the effector protein is codon optimized for a human cell. The NLS may be located at a variety of locations, including, but not limited to 5′ of the effector protein, 5′ of the fusion partner, 3′ of the effector protein, 3′ of the fusion partner, between the effector protein and the fusion partner, within the fusion partner, within the effector protein.

### Linkers for Peptides

In general, effector proteins and fusion partners of a fusion effector protein are connected by a linker. In some embodiments, a linker comprises a bond or molecule that links a first polypeptide to a second polypeptide. The linker may comprise or consist of a covalent bond. The linker may comprise or consist of a chemical group. In some embodiments, the linker comprises an amino acid. In some embodiments, a peptide linker comprises at least two amino acids linked by an amide bond. In general, the linker connects a terminus of the effector protein to a terminus of the fusion partner. In some embodiments, carboxy terminus of the effector protein is linked to the amino terminus of the fusion partner. In some embodiments, carboxy terminus of the fusion partner is linked to the amino terminus of the effector protein. In some embodiments, the effector protein and the fusion partner are directly linked by a covalent bond.

In some embodiments, linkers comprise one or more amino acids. In some embodiments, linker is a protein. In some embodiments, a terminus of the effector protein is linked to a terminus of the fusion partner through an amide bond. In some embodiments, a terminus of the effector protein is linked to a terminus of the fusion partner through a peptide bond. In some embodiments, linkers comprise an amino acid. In some embodiments, linkers comprise a peptide. In some embodiments, an effector protein is coupled to a fusion partner by a linker protein. In some embodiments, the linker may have any of a variety of amino acid sequences. In some embodiments, the linker may comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

Linkers may be produced by using synthetic, linker-encoding oligonucleotides to couple proteins, or may be encoded by a nucleic acid sequence encoding a fusion protein (e.g., an effector protein coupled to a fusion partner). In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)n, glycine-serine polymers (including, for example, (GS)n, GSGGSn, GGSGGSn, and GGGSn, where n is an integer of at least one), glycine-alanine polymers, and alanine-serine polymers. In some embodiments, linkers may comprise amino acid sequences including, but not limited to, GGSG, GGSGG, GSGSG, GSGGG, GGGSG, and GSSSG. In some embodiments, the linker comprises one or more repeats a tri-peptide GGS. In some embodiments, the linker is an XTEN linker.

In some embodiments, linkers do not comprise an amino acid. In some embodiments, linkers do not comprise a peptide. In some embodiments, linkers comprise a nucleotide, a polynucleotide, a polymer, or a lipid. In some embodiments, linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, an alkyl linker, or a combination thereof. In some embodiments, linkers comprise or consist of a nucleic acid. In some embodiments, the nucleic acid comprises DNA. In some embodiments, the nucleic acid comprises RNA. In some embodiments, the effector protein and the fusion partner each interact with the nucleic acid, the nucleic acid thereby linking the effector protein and the fusion partner. In some embodiments, the nucleic acid serves as a scaffold for both the effector protein and the fusion partner to interact with, thereby linking the effector protein and the fusion partner. Such nucleic acids include those described by Tadakuma et al., (2016), Progress in Molecular Biology and Translational Science, Volume 139, pp. 121-163, incorporated herein by reference.

### Multimeric Complexes

Compositions, systems, and methods of the present disclosure may comprise a multimeric complex or uses thereof, wherein the multimeric complex comprises one or more effector proteins that non-covalently interact with one another. A multimeric complex may comprise enhanced activity relative to the activity of any one of its effector proteins alone. For example, a multimeric complex comprising two effector proteins (e.g., in dimeric form) may comprise greater nucleic acid binding affinity and/or nuclease activity than that of either of the effector proteins provided in monomeric form. In another example, a multimeric complex comprising an effector protein and an effector partner may comprise greater nucleic acid binding affinity and/or nuclease activity than that of either of the effector protein or effector partner provided in monomeric form.

The terms “effector partner” and “partner polypeptide” refer to a polypeptide that does not have 100% sequence identity with an effector protein described herein. In some instances, an effector partner described herein may be found in a homologous genome as an effector protein described herein.

A multimeric complex may have an affinity for a target sequence of a target nucleic acid and is capable of catalytic activity (e.g., cleaving, nicking, inserting or otherwise editing the nucleic acid) at or near the target sequence. A multimeric complex may have an affinity for a donor nucleic acid and is capable of catalytic activity (e.g., cleaving, nicking, editing or otherwise modifying the nucleic acid by creating cuts) at or near one or more ends of the donor nucleic acid. Multimeric complexes may be activated when complexed with a guide nucleic acid. Multimeric complexes may be activated when complexed with a target nucleic acid. Multimeric complexes may be activated when complexed with a guide nucleic acid, a target nucleic acid, and/or a donor nucleic acid. In some embodiments, the multimeric complex cleaves the target nucleic acid. In some embodiments, the multimeric complex nicks the target nucleic acid.

Various aspects of the present disclosure include compositions and methods comprising multiple effector proteins, and uses thereof, respectively. An effector protein comprising at least 65%, at least 70%, at least 75%, at least 80%, at least 85%, at least 90%, at least 95% or 100% sequence identity to any one of the sequences of TABLE 1 may be provided with a second effector protein. Two effector proteins may target different nucleic acid sequences. Two effector proteins may target different types of nucleic acids (e.g., a first effector protein may target double- and single-stranded nucleic acids, and a second effector protein may only target single-stranded nucleic acids). It is understood that when discussing the use of more than one effector protein in compositions, systems, and methods provided herein, the multimeric complex form is also described.

In some embodiments, multimeric complexes comprise at least one effector protein comprising an amino acid sequence with at least 65%, at least 70%, at least 75%, at least 80%, at least 85%, at least 90%, at least 95% or 100% identity to any one of the sequences of TABLE 1. In some embodiments, the multimeric complex is a dimer comprising two effector proteins of identical amino acid sequences. In some embodiments, the multimeric complex comprises a first effector protein and a second effector protein, wherein the amino acid sequence of the first effector protein is at least 90%, at least 92%, at least 94%, at least 96%, at least 98% identical, or at least 99% identical to the amino acid sequence of the second effector protein.

In some embodiments, the multimeric complex is a heterodimeric complex comprising at least two effector proteins of different amino acid sequences. In some embodiments, the multimeric complex is a heterodimeric complex comprising a first effector protein and a second effector protein, wherein the amino acid sequence of the first effector protein is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% identical to the amino acid sequence of the second effector protein.

In some embodiments, a multimeric complex comprises at least two effector proteins. In some embodiments, a multimeric complex comprises more than two effector proteins. In some embodiments, a multimeric complex comprises two, three or four effector proteins. In some embodiments, at least one effector protein of the multimeric complex comprises an amino acid sequence with at least 65%, at least 70%, at least 75%, at least 80%, at least 85%, at least 90%, at least 95% or 100% identity to any one of the sequences of TABLE 1. In some embodiments, each effector protein of the multimeric complex independently comprises an amino acid sequence with at least 65%, at least 70%, at least 75%, at least 80%, at least 85%, at least 90%, at least 95% or 100% identity to any one of the sequences of TABLE 1.

### Synthesis, Isolation and Assaying

Effector proteins of the present disclosure may be synthesized, using any suitable method. In some embodiments, the effector proteins may be produced in vitro or by eukaryotic cells or by prokaryotic cells. In some embodiments, the effector proteins may be further processed by unfolding (e.g. heat denaturation, dithiothreitol reduction, etc.) and may be further refolded, using any suitable method.

Any suitable method of generating and assaying the effector proteins described herein may be used. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, M D (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art. Exemplary methods are also described in the Examples provided herein.

In some embodiments, an effector protein provided herein is an isolated effector protein. In some embodiments, the effector proteins may be isolated and purified for use in compositions, systems, and/or methods described herein. In some embodiments, methods described here may include the step of isolating effector proteins described herein. Any suitable method to provide isolated effector proteins described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, compositions, systems, and methods described herein may further comprise a purification tag that can be attached to an effector protein, or a nucleic acid encoding the purification tag that can be attached to a nucleic acid encoding the effector protein as described herein. In some embodiments, the purification tag may be an amino acid sequence which can attach or bind with high affinity to a separation substrate and assist in isolating the protein of interest from its environment, which may be its biological source, such as a cell lysate. Attachment of the purification tag may be at the N or C terminus of the effector protein. Furthermore, an amino acid sequence recognized by a protease or a nucleic acid encoding for an amino acid sequence recognized by a protease, such as TEV protease or the HRV3C protease may be inserted between the purification tag and the effector protein, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, effector proteins described herein are isolated from cell lysate. In some embodiments, the compositions described herein may comprise 20% or more by weight, 75% or more by weight, 95% or more by weight, or 99.5% or more by weight of an effector protein, related to the method of preparation of compositions described herein and its purification thereof, wherein percentages may be upon total protein content in relation to contaminants. Thus, in some embodiments, the effector protein is at least 80% pure, at least 85% pure, at least 90% pure, at least 95% pure, at least 98% pure, or at least 99% pure (e.g., free of contaminants, non-engineered proteins or other macromolecules, etc.).

### Protospacer Adjacent Motif (PAM) Sequences

Effector proteins of the present disclosure may cleave or nick a target nucleic acid within or near a protospacer adjacent motif (PAM) sequence of the target nucleic acid. In some embodiments, the target nucleic acid is a double stranded nucleic acid comprising a target strand and a non-target strand.

In some embodiments, cleavage occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the effector protein does not require a PAM to bind and/or cleave a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence. In some embodiments, an RNP cleaves the single stranded target nucleic acid.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, such a PAM described herein is directly adjacent to the target sequence on the target strand or the non-target strand. In some embodiments, an RNP cleaves the target strand or the non-target strand. In some embodiments, the RNP cleaves both, the target strand and the non-target strand. In some embodiments, an RNP recognizes the PAM sequence, and hybridizes to a target sequence of the target nucleic acid. In some embodiments, the RNP cleaves the target nucleic acid, wherein the RNP has recognized the PAM sequence and is hybridized to the target sequence.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

An effector protein of the present disclosure, or a multimeric complex thereof, may cleave or nick a target nucleic acid within or near a protospacer adjacent motif (PAM) sequence of the target nucleic acid. In some embodiments, cleavage occurs within 1, 2, 3, 4, 5, 6, 7, 8, 9 or 10 nucleotides of a 5′ or 3′ terminus of a PAM sequence.

In some embodiments, compositions, methods and systems described herein do not comprise a PAM sequence. In some embodiments, effector proteins do not recognize a PAM sequence. In some embodiments, compositions, methods and systems described herein comprise a protospacer-flanking site (PFS) sequence. A PFS sequence may be useful for the detection and/or modification of RNA.

### V. Nucleic Acid Systems

### Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In some embodiments, the guide nucleic acid comprises a nucleotide sequence. Such nucleotide sequence may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid. Similarly, disclosure of the nucleotide sequences described herein also discloses a complementary nucleotide sequence, a reverse nucleotide sequence, and the reverse complement nucleotide sequence, any one of which can be a nucleotide sequence for use in a guide nucleic acid. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid may comprise a sequence that is bound by an effector protein. In general, the guide nucleic acid comprises a CRISPR RNA (crRNA), at least a portion of which is complementary to a target sequence of a target nucleic acid. In some embodiments, the guide nucleic acid comprises a trans-activating CRISPR RNA (tracrRNA) that interacts with the effector protein. In some embodiments, the crRNA and the tracrRNA are covalently linked, also referred to herein as a single guide RNA (sgRNA). In some embodiments, the crRNA and tracrRNA are linked by a phosphodiester bond. In some embodiments, the crRNA and tracrRNA are linked by one or more linked nucleotides. In some embodiments, a crRNA and tracrRNA function as two separate, unlinked molecules. In some embodiments, the composition does not comprise a tracrRNA. In some embodiments, the crRNA comprises a sequence that is bound by an effector protein.

The terms, “length” and “linked nucleosides,” as used herein, refer to a nucleic acid (polynucleotide) or polypeptide, may be expressed as “kilobases” (kb) or “base pairs (bp),”. Thus, a length of 1 kb refers to a length of 1000 linked nucleosides, and a length of 500 bp refers to a length of 500 linked nucleosides. Similarly, a protein having a length of 500 linked amino acids may also be simply described as having a length of 500 amino acids.

Guide nucleic acids may comprise DNA, RNA, or a combination thereof (e.g., RNA with a thymine base). Guide nucleic acids may include a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a naturally occurring guide nucleic acid. A guide nucleic acid may comprise a non-naturally occurring guide nucleic acid, including a guide nucleic acid that is designed to contain a chemical or biochemical modification. The sequence of a guide nucleic acid may comprise two or more heterologous sequences. Guide RNAs may be chemically synthesized or recombinantly produced.

Guide nucleic acids, when complexed with an effector protein, may bring the effector protein into proximity of a target nucleic acid. Sufficient conditions for hybridization of a guide nucleic acid to a target nucleic acid and/or for binding of a guide nucleic acid to an effector protein include in vivo physiological conditions of a desired cell type or in vitro conditions sufficient for assaying catalytic activity of a protein, polypeptide or peptide described herein, such as the nuclease activity of an effector protein.

The compositions, systems, and methods of the present disclosure may comprise a guide nucleic acid, a nucleic acid encoding the guide nucleic acid, or a use thereof. Unless otherwise indicated, compositions, systems and methods comprising guide nucleic acids or uses thereof, as described herein and throughout, include DNA molecules, such as expression vectors, that encode a guide nucleic acid. Guide nucleic acids are also referred to herein as “guide RNA.” A guide nucleic acid, as well as any components thereof (e.g., spacer sequence, repeat sequence, linker nucleotide sequence, etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

In general, a guide nucleic acid comprises a first region that is not complementary to a target nucleic acid (FR) and a second region is complementary to the target nucleic acid (SR). In some embodiments, FR is located 5′ to SR (FR-SR). In some embodiments, SR is located 5′ to FR (SR-FR).

In some embodiments, the FR comprises one or more repeat sequences. In some embodiments, an effector protein binds to at least a portion of the FR. In some embodiments, the SR comprises a spacer sequence, wherein the spacer sequence can interact in a sequence-specific manner with (e.g., has complementarity with, or can hybridize to a target sequence in) a target nucleic acid.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene. In some embodiments, the first loci and/or the second loci of the target nucleic acid are located in an exon of a gene. In some embodiments, the first loci and/or the second loci of the target nucleic acid span an exon-intron junction of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides. In some embodiments, a guide nucleic acid comprises at least 25 linked nucleosides. A guide nucleic acid may comprise 10 to 50 linked nucleosides. In some cases, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some cases, the guide nucleic acid has about 10 to about 60, about 20 to about 50, or about 30 to about 40 linked nucleosides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, guide nucleic acids comprise one or more nucleotide sequences as described herein. Such nucleotide sequences described herein may be described as a nucleotide sequence of either DNA or RNA, however, no matter the form the sequence is described, it is readily understood that such nucleotide sequences may be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the sequences described herein. Alternative nucleotides may be any one or more of A, C, G, T or U, or a deletion, or an insertion.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is linked to a spacer sequence. In some embodiments, a guide nucleic acid comprises a repeat sequence linked to a spacer sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

### Spacer Sequence

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides. In some cases, the spacer sequence is 18-24 linked nucleosides in length. In some cases, the spacer sequence is at least 15 linked nucleosides in length. In some cases, the spacer sequence is at least 16, 18, 20, or 22 linked nucleosides in length. In some cases, the spacer sequence is at least 17 linked nucleosides in length. In some cases, the spacer sequence is at least 18 linked nucleosides in length. In some cases, the spacer sequence is at least 20 linked nucleosides in length. In some cases, the spacer sequence is at least 80%, at least 85%, at least 90%, at least 95% or 100% complementary to a target sequence of the target nucleic acid. In some cases, the spacer sequence is 100% complementary to the target sequence of the target nucleic acid. In some cases, the spacer sequence comprises at least 15 contiguous nucleobases that are complementary to the target nucleic acid.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

It is understood that the sequence of a spacer sequence need not be 100% complementary to that of a target sequence of a target nucleic acid to hybridize or hybridize specifically to the target sequence. The guide nucleic acid may comprise at least one uracil between nucleic acid residues 5 to 20 of the spacer sequence that is not complementary to the corresponding nucleoside of the target sequence. The guide nucleic acid may comprise at least one uracil between nucleic acid residues 5 to 9, 10 to 14, or 15 to 20 of the spacer sequence that is not complementary to the corresponding nucleoside of the target sequence. In some cases, the region of the target nucleic acid that is complementary to the spacer sequence comprises an epigenetic modification or a post-transcriptional modification. In some cases, the epigenetic modification comprises acetylation, methylation, or thiol modification.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a target nucleic acid, such as DNA or RNA, may be a cancer gene or gene associated with a genetic disorder, or an amplicon thereof, as described herein. In some embodiments, a target nucleic acid is a gene selected from TABLE 3. In some embodiments, a spacer sequence comprises a sequence that is at least 65%, at least 70%, at least 75%, at least 80%, at least 85%, at least 90%, at least 95% or 100% complementary to a target sequence of a gene selected from TABLE 3. In some embodiments, a target nucleic acid is a nucleic acid associated with a disease or syndrome set forth in TABLE 4. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome set forth in TABLE 4. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the spacer sequence of a spacer sequence need not be 100% complementary to that of a target sequence of a target nucleic acid to hybridize or hybridize specifically to the target sequence. For example, the spacer sequence may comprise at least one alteration, such as a substituted or modified nucleotide, that is not complementary to the corresponding nucleotide of the target sequence.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

tracrRNA

In some embodiments, the guide RNA comprises a tracrRNA. The tracrRNA may be linked to a crRNA to form a composite gRNA. In some cases, the crRNA and the tracrRNA are provided as a single nucleic acid (e.g., covalently linked). In some embodiments, compositions comprise a tracrRNA that is separate from, but forms a complex with a crRNA to form a gRNA system. In some embodiments, the crRNA and the tracrRNA are separate polynucleotides.

In general, a tracrRNA comprises a nucleotide sequence that is bound by an effector protein. A tracrRNA may comprise at least one secondary structure (e.g., hairpin loop) that facilitates the binding of an effector protein. A tracrRNA may include a repeat hybridization sequence and a hairpin region. The term “repeat hybridization sequence” refers to a sequence of nucleotides of a tracrRNA that is capable of hybridizing to a repeat sequence of a guide nucleic acid. The repeat hybridization sequence may hybridize to all or part of the repeat sequence of a crRNA. The repeat hybridization sequence may be positioned 3′ of the hairpin region. The repeat hybridization sequence may be positioned 5′ of the hairpin region. The hairpin region may include a first sequence, a second sequence that is reverse complementary to the first sequence, and a stem-loop linking the first sequence and the second sequence.

In some embodiments, tracrRNAs comprise a stem-loop structure comprising a stem region and a loop region. In some cases, the stem region is 4 to 8 linked nucleosides in length. In some cases, the stem region is 5 to 6 linked nucleosides in length. In some cases, the stem region is 4 to 5 linked nucleosides in length. In some cases, the tracrRNA comprises a pseudoknot (e.g., a secondary structure comprising a stem at least partially hybridized to a second stem or half-stem secondary structure). An effector protein may recognize a tracrRNA comprising multiple stem regions. In some embodiments, the amino acid sequences of the multiple stem regions are identical to one another. In some embodiments, the amino acid sequences of at least one of the multiple stem regions is not identical to those of the others. In some cases, the tracrRNA comprises at least 2, at least 3, at least 4, or at least 5 stem regions. In some embodiments, the length of a tracrRNA is about 50 to about 105, about 50 to about 95, about 50 to about 73, about 50 to about 71, about 50 to about 68, or about 50 to about 56 linked nucleosides. In some embodiments, the length of a tracrRNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of a tracrRNA is 40 to 60 nucleotides. In some embodiments, the length of a tracrRNA is 50, 56, 68, 71, 73, 95, or 105 linked nucleosides. In some embodiments, the length of a tracrRNA is 50 nucleotides.

An exemplary tracrRNA may comprise, from 5′ to 3′, a 5′ region, a hairpin region, a repeat hybridization sequence, and a 3′ region. In some cases, the 5′ region may hybridize to the 3′ region. In some embodiments, the 5′ region does not hybridize to the 3′ region. In some cases, the 3′ region is covalently linked to the crRNA (e.g., through a phosphodiester bond). In some embodiments, a tracrRNA may comprise an unhybridized region at the 3′ end of the tracrRNA. The unhybridized region may have a length of about 1, about 2, about 3, about 4, about 5, about 6, about 7, about 8, about 9, about 10, about 12, about 14, about 16, about 18, or about 20 linked nucleosides. In some embodiments, the length of the un-hybridized region is 0 to 20 linked nucleosides.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

Guide nucleic acids and portions thereof may be found in or identified from a CRISPR array present in the genome of a host organism. A crRNA may be the product of processing of a longer precursor CRISPR RNA (pre-crRNA) transcribed from the CRISPR array by cleavage of the pre-crRNA within each direct repeat sequence to afford shorter, mature crRNAs. A crRNA may be generated by a variety of mechanisms, including the use of dedicated endonucleases (e.g., Cas6 or Cas5d in Type I and III systems), coupling of a host endonuclease (e.g., RNase III) with tracrRNA (Type II systems), or a ribonuclease activity endogenous to the effector protein itself (e.g., Cpf1 from Type V systems). A crRNA may also be specifically generated outside of processing of a pre-crRNA and individually contacted to an effector protein in vivo or in vitro.

In general, a crRNA comprises a spacer sequence that hybridizes to a target sequence of a target nucleic acid, and a repeat sequence that interacts with a tracrRNA or an effector protein. Typically, the repeat sequence is adjacent to the spacer sequence. For example, a guide RNA that interacts with an effector protein comprises a repeat sequence that is 5′ of the spacer sequence.

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

In some cases, an effector protein cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some cases, a repeat sequence of a guide RNA comprises mutations or truncations relative to respective regions in a corresponding pre-crRNA.

sgRNA

In some embodiments, a guide nucleic acid comprises a sgRNA. The terms “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components

In some embodiments, a sgRNA comprises one or more of one or more of a crRNA, a repeat sequence, a spacer sequence, a linker, or combinations thereof. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

### A Dual Nucleic Acid System

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule.

The terms, “transactivating”, “trans-activating”, “trans-activated”, “transactivated” and grammatical equivalents thereof, as used herein, in the context of a dual nucleic acid system refers to an outcome of the system, wherein a polypeptide is enabled to have a binding and/or nuclease activity on a target nucleic acid, by a tracrRNA or a tracrRNA-crRNA duplex.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### VI. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

The term “engineered modification” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a base editing, a base modification, a backbone modification, a sugar modification, or combinations thereof. In some embodiments, the modifications can be of one or more nucleotides, nucleosides, or nucleobases in a nucleic acid.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides, 2′ fluoro modified nucleotides; locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH₂ component parts; and combinations thereof.

### VII. Vectors and Multiplexed Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein, a donor nucleic acid, and a guide nucleic acid or a nucleic acid encoding the guide nucleic acid. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

The terms “promoter” and “promoter sequence” refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. The term “regulatory element” refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence. In some embodiments, the vector is a non-viral vector, and a physical method or a chemical method is employed for delivery into the somatic cell.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode an effector protein and a guide nucleic acid. A vector may encode an effector protein, a guide nucleic acid, and a donor nucleic acid.

In some embodiments, a vector comprises one or more guide nucleic acids, or a nucleotide sequence encoding the one or more guide nucleic acids as described herein. In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector comprises one or more donor nucleic acids as described herein. In some embodiments, the one or more donor nucleic acids comprise at least two donor nucleic acids. In some embodiments, the at least two donor nucleic acids are the same. In some embodiments, the at least two donor nucleic acids are different from each other. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more donor nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, CAG, TRE, UAS, Ac5, Polyhedrin, CaMKIIa, GAL1-10, H1, TEF1, GDS, ADH1, CaMV35S, HSV TK, Ubi, U6, MNDU3, MSCV, MND, and CAG.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or an engineered guide nucleic acid (or a nucleic acid that, when transcribed, produces same) are not co-administered with donor nucleic acid in a single vehicle. In some embodiments, an effector protein (or a nucleic acid encoding same), an engineered guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3): 146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the effector protein, and/or the donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, the viral vector comprises a nucleic acid to be delivered into a host cell by a recombinantly produced virus or viral particle. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more effector proteins, a nucleic acid encoding one or more fusion proteins (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells may comprise baculovirus. In some embodiments, production of rAAV virus particles in insect cells may comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### VIII. Target Nucleic Acids

Disclosed herein are compositions, systems and methods for detecting and/or editing a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, arRNA, a tRNA, a non-coding RNA, a long non-coding RNA, a microRNA (miRNA), and a single-stranded RNA (ssRNA). In some embodiments, the target nucleic acid is complementary DNA (cDNA) synthesized from a single-stranded RNA template in a reaction catalyzed by a reverse transcriptase. Exemplary chemical methods include delivery of the recombinant polynucleotide via liposomes such as, cationic lipids or neutral lipids; dendrimers; nanoparticles; or cell-penetrating peptides.

In some embodiments, the target nucleic acid is an mRNA. In some embodiments, the target nucleic acid is from a virus, a parasite, or a bacterium described herein.

In some embodiments, a target nucleic acid comprising a target sequence comprises a PAM sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification (e.g., an editing) after contacting with an RNP. In some embodiments, the editing is a change in the sequence of the target nucleic acid. In some embodiments, the change comprises an insertion, deletion, or substitution of one or more nucleotides compared to the target nucleic acid that has not undergone any modification.

Nucleic acids, such as DNA and pre-mRNA, described herein can contain at least one intron and at least one exon, wherein as read in the 5′ to the 3′ direction of a nucleic acid strand, the 3′ end of an intron can be adjacent to the 5′ end of an exon, and wherein said intron and exon correspond for transcription purposes. If a nucleic acid strand contains more than one intron and exon, the 5′ end of the second intron is adjacent to the 3′ end of the first exon, and 5′ end of the second exon is adjacent to the 3′ end of the second intron. The junction between an intron and an exon can be referred to herein as a splice junction, wherein a 5′ splice site (SS) can refer to the +1/+2 position at the 5′ end of intron and a 3′SS can refer to the last two positions at the 3′ end of an intron. Alternatively, a 5′ SS can refer to the 5′ end of an exon and a 3′SS can refer to the 3′ end of an exon. In some embodiments, nucleic acids can contain one or more elements that act as a signal during transcription, splicing, and/or translation. In some embodiments, signaling elements include a 5′SS, a 3′SS, a premature stop codon, U1 and/or U2 binding sequences, and cis acting elements such as branch site (BS), polypyridine tract (PYT), exonic and intronic splicing enhancers (ESEs and ISEs) or silencers (ESSs and ISSs). In some embodiments, nucleic acids may also comprise a untranslated region (UTR), such as a 5′ UTR or a 3′ UTR. In some embodiments, the start of an exon or intron is referred to interchangeably herein as the 5′ end of an exon or intron, respectively. Likewise, in some embodiments, the end of an exon or intron is referred to interchangeably herein as the 3′ end of an exon or intron, respectively.

In some embodiments, at least a portion of at least one target sequence is within about 1, about 5 or more, about 10 or more, about 15 or more, about 20 or more, about 25 or more, about 30 or more, about 35 or more, about 40 or more, about 45 or more, about 50 or more, about 55 or more, about 60 or more, about 65 or more, about 70 or more, about 75 or more, about 80 or more, about 85 or more, about 90 or more, about 95 or more, about 100 or more, about 105 or more, about 110 or more, about 115 or more, about 120 or more, about 125 or more, about 130 or more, about 135 or more, about 140 or more, about 145 or more, or about 150 to about 300 nucleotides adjacent to: the 5′ end of an exon; the 3′ end of an exon; the 5′ end of an intron; the 3′ end of an intron; one or more signaling element comprising a 5′SS, a 3′SS, a premature stop codon, U1 binding sequence, U2 binding sequence, a BS, a PYT, ESE, an ISE, an ESS, an ISS; a 5′ UTR; a 3′ UTR; more than one of the foregoing, or any combination thereof. In some embodiments, the target nucleic acid comprises a target locus. In some embodiments, the target nucleic acid comprises more than one target loci. In some embodiments, the target nucleic acid comprises two target loci. Accordingly, in some embodiments, the target nucleic acid can comprise one or more target sequences.

In some embodiments, compositions, systems, and methods described herein comprise an edited target nucleic acid which can describe a target nucleic acid wherein the target nucleic acid has undergone a change, for example, after contact with an effector protein. In some embodiments, the editing is an alteration in the sequence of the target nucleic acid. In some embodiments, the edited target nucleic acid comprises an insertion, deletion, or replacement of one or more nucleotides compared to the unedited target nucleic acid. In some embodiments, the editing is a mutation.

### Mutations

In some embodiments, target nucleic acids described herein comprise a mutation. In some embodiments, a composition, system or method described herein can be used to edit a target nucleic acid comprising a mutation such that the mutation is edited to be the wild-type nucleotide or nucleotide sequence. In some embodiments, a composition, system or method described herein can be used to detect a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. The term, “indel” refers to an insertion-deletion or indel mutation, which is a type of genetic mutation that results from the insertion and/or deletion of one or more nucleotide in a target nucleic acid. An indel can vary in length (e.g., 1 to 1,000 nucleotides in length) and be detected by any suitable method, including sequencing. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, may be encoded in the sequence of a target nucleic acid from the germline of an organism or may be encoded in a target nucleic acid from a diseased cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids set forth in TABLE 3. In some embodiments, a target nucleic acid described herein comprises a mutation associated with a disease, wherein the disease is any one of the diseases set forth in TABLE 4.

### Detection and Identification of Target Nucleic Acid

In some embodiments, a target nucleic acid is in a cell. In some embodiments, the cell is a single-cell eukaryotic organism; a plant cell an algal cell; a fungal cell; an animal cell; a cell of an invertebrate animal; a cell of a vertebrate animal such as fish, amphibian, reptile, bird, and mammal; or a cell of a mammal such as a human, a non-human primate, an ungulate, a feline, a bovine, an ovine, and a caprine. In some embodiments, the cell is a eukaryotic cell. In some embodiments, the cell is a mammalian cell, a human cell, or a plant cell. In some embodiments, the cell is a human cell. In some embodiments, the human cell is a: muscle cell, liver cell, lung cell, cardiac cell, visceral cell, cardiac muscle cell, smooth muscle cell, cardiomyocyte, nodal cardiac muscle cell, smooth muscle cell, visceral muscle cell, skeletal muscle cell, myocyte, red (or slow) skeletal muscle cell, white (fast) skeletal muscle cell, intermediate skeletal muscle, muscle satellite cell, muscle stem cell, myoblast, muscle progenitor cell, induced pluripotent stem cell (iPS), or a cell derived from an iPS cell, modified to have its gene edited and differentiated into myoblasts, muscle progenitor cells, muscle satellite cells, muscle stem cells, skeletal muscle cells, cardiac muscle cells or smooth muscle cells.

In some embodiments, an effector protein-guide nucleic acid complex may comprise high selectivity for a target sequence. In some embodiments, an RNP comprise a selectivity of at least 200:1, 100:1, 50:1, 20:1, 10:1, or 5:1 for a target nucleic acid over a single nucleotide variant of the target nucleic acid. In some embodiments, an RNP may comprise a selectivity of at least 5:1 for a target nucleic acid over a single nucleotide variant of the target nucleic acid.

By leveraging such effector protein selectivity, some methods described herein may detect a target nucleic acid present in the sample in various concentrations or amounts as a target nucleic acid population. In some embodiments, the method detects at least 2 target nucleic acid populations. In some embodiments, the method detects at least 3, 4, 5, 6, 7, 8, 9, 10, 20, 30, 40, or 50 target nucleic acid populations. In some embodiments, the method detects 3 to 50, 5 to 40, or 10 to 25 target nucleic acid populations. In some embodiments, the method detects at least 2 individual target nucleic acids. In some embodiments, the method detects at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids. In some embodiments, the method detects 1 to 10,000, 100 to 8000, 400 to 6000, 500 to 5000, 1000 to 4000, or 2000 to 3000 individual target nucleic acids. In some embodiments, the method detects target nucleic acid present at least at one copy per 10 non-target nucleic acids, 10² non-target nucleic acids, 10³ non-target nucleic acids, 10⁴ non-target nucleic acids, 105 non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, compositions described herein exhibit indiscriminate trans-cleavage of ssRNA, enabling their use for detection of RNA in samples. In some embodiments, target ssRNA are generated from many nucleic acid templates (RNA) in order to achieve cleavage of the FQ reporter in the DETECTR platform. Certain effector proteins may be activated by ssRNA, upon which they may exhibit trans-cleavage of ssRNA and may, thereby, be used to cleave ssRNA FQ reporter molecules in the DETECTR system. These effector proteins may target ssRNA present in the sample or ssRNA generated and/or amplified from any number of nucleic acid templates (RNA). Described herein are reagents comprising a single stranded reporter nucleic acid comprising a detection moiety, wherein the reporter nucleic acid (e.g., the ssDNA-FQ reporter described above) is capable of being cleaved by the Effector protein, upon generation and amplification of ssRNA from a nucleic acid template using the methods disclosed herein, thereby generating a first detectable signal.

In some embodiments, a target nucleic acid is an amplified nucleic acid of interest. In some embodiments, the nucleic acid of interest is any nucleic acid disclosed herein or from any sample as disclosed herein. In some embodiments, the nucleic acid of interest is an RNA that is reverse transcribed before amplification. In some embodiments, the nucleic acid of interest is amplified then the amplicons is transcribed into RNA.

In some embodiments, target nucleic acids may activate an effector protein to initiate sequence-independent cleavage of a nucleic acid-based reporter (e.g., a reporter comprising an RNA sequence, or a reporter comprising DNA and RNA). For example, an effector protein of the present disclosure is activated by a target nucleic acid to cleave reporters having an RNA (also referred to herein as an “RNA reporter”). Alternatively, an effector protein of the present disclosure is activated by a target nucleic acid to cleave reporters having an RNA. Alternatively, an effector protein of the present disclosure is activated by a target RNA to cleave reporters having an RNA (also referred to herein as a “RNA reporter”). The RNA reporter may comprise a single-stranded RNA labelled with a detection moiety or may be any RNA reporter as disclosed herein.

Further description of editing or detecting a target nucleic acid in a gene of interest can be found in more detail in Kim et al., “Enhancement of target specificity of CRISPR-Cas 12a by using a chimeric DNA-RNA guide”, Nucleic Acids Res. 2020 Sep. 4; 48(15):8601-8616; Wang et al., “Specificity profiling of CRISPR system reveals greatly enhanced off-target gene editing”, Scientific Reports volume 10, Article number: 2269 (2020); Tuladhar et al., “CRISPR-Cas9-based mutagenesis frequently provokes on-target mRNA misregulation”, Nature Communications volume 10, Article number: 4056 (2019); Dong et al., “Genome-Wide Off-Target Analysis in CRISPR-Cas9 Modified Mice and Their Offspring”, G3, Volume 9, Issue 11, 1 Nov. 2019, Pages 3645-3651; Winter et al., “Genome-wide CRISPR screen reveals novel host factors required for Staphylococcus aureus α-hemolysin-mediated toxicity”, Scientific Reports volume 6, Article number: 24242 (2016); and Ma et al., “A CRISPR-Based Screen Identifies Genes Essential for West-Nile-Virus-Induced Cell Death”, Cell Rep. 2015 Jul. 28; 12(4):673-83, which are hereby incorporated by reference in their entirety.

### Certain Samples

Various sample types comprising a target nucleic acid of interest are consistent with the present disclosure. These samples may comprise a target nucleic acid for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample may be obtained to test for presence of a disease, cancer, genetic disorder, or any mutation of interest.

In some embodiments, a sample comprises a target nucleic acid from 0.05% to 20% of total nucleic acids in the sample. In some embodiments, the target nucleic acid is 0.1% to 10% of the total nucleic acids in the sample. In some embodiments, the target nucleic acid is 0.1% to 5% of the total nucleic acids in the sample. In some embodiments, the target nucleic acid is 0.1% to 1% of the total nucleic acids in the sample. In some embodiments, the target nucleic acid is in any amount less than 100% of the total nucleic acids in the sample. In some embodiments, the target nucleic acid is 100% of the total nucleic acids in the sample. In some embodiments, the sample comprises a portion of the target nucleic acid and at least one nucleic acid comprising less than 100% sequence identity to the portion of the target nucleic acid but no less than 50% sequence identity to the portion of the target nucleic acid. For example, the portion of the target nucleic acid comprises a mutation as compared to at least one nucleic acid comprising less than 100% sequence identity to the portion of the target nucleic acid but no less than 50% sequence identity to the portion of the target nucleic acid. In some embodiments, the portion of the target nucleic acid comprises a single nucleotide mutation as compared to at least one nucleic acid comprising less than 100% sequence identity to the portion of the target nucleic acid but no less than 50% sequence identity to the portion of the target nucleic acid.

In some embodiments, a sample comprises target nucleic acid populations at different concentrations or amounts. In some embodiments, the sample has at least 2 target nucleic acid populations. In some embodiments, the sample has at least 3, 4, 5, 6, 7, 8, 9, 10, 20, 30, 40, or 50 target nucleic acid populations. In some embodiments, the sample has 3 to 50, 5 to 40, or 10 to 25 target nucleic acid populations.

In some embodiments, a sample has at least 2 individual target nucleic acids. In some embodiments, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 individual target nucleic acids.

In some embodiments, the sample comprises 1 to 10,000, 100 to 8000, 400 to 6000, 500 to 5000, 1000 to 4000, or 2000 to 3000 individual target nucleic acids.

In some embodiments, a sample comprises one copy of target nucleic acid per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

In some embodiments, samples comprise a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 UM to 100 μM, 100 μM to 1 mM, 1 nM to 10 nM, 1 nM to 100 nM, 1 nM to 1 μM, 1 nM to 10 μM, 1 nM to 10 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 UM, 100 nM to 100 μM, 100 nM to 1 mM, 1 μM to 10 μM, 1 μM to 100 μM, 1 μM to 1 mM, 1 μM to 100 μM, 10 μM to 1 mM, or 100 μM to 1 mM. In some embodiments, the sample comprises a target nucleic acid at a concentration of 20 nM to 200 μM, 50 nM to 100 μM, 200 nM to 50 μM, 500 nM to 20 μM, or 2 μM to 10 μM. In some embodiments, the target nucleic acid is not present in the sample.

In some embodiments, samples comprise fewer than 10 copies, fewer than 100 copies, fewer than 1000 copies, fewer than 10,000 copies, fewer than 100,000 copies, or fewer than 1,000,000 copies of a target nucleic acid. In some embodiments, the sample comprises 10 copies to 100 copies, 100 copies to 1000 copies, 1000 copies to 10,000 copies, 10,000 copies to 100,000 copies, 100,000 copies to 1,000,000 copies, 10 copies to 1000 copies, 10 copies to 10,000 copies, 10 copies to 100,000 copies, 10 copies to 1,000,000 copies, 100 copies to 10,000 copies, 100 copies to 100,000 copies, 100 copies to 1,000,000 copies, 1,000 copies to 100,000 copies, or 1,000 copies to 1,000,000 copies of a target nucleic acid. In some embodiments, the sample comprises 10 copies to 500,000 copies, 200 copies to 200,000 copies, 500 copies to 100,000 copies, 1000 copies to 50,000 copies, 2000 copies to 20,000 copies, 3000 copies to 10,000 copies, or 4000 copies to 8000 copies. In some embodiments, the target nucleic acid is not present in the sample.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). A tissue sample from a subject may be dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unedited, unmodified) sample. Raw samples may be applied to a system for detecting or editing a target nucleic acid, such as those described herein. In some embodiments, the sample is diluted with a buffer or a fluid or concentrated prior to its application to the system or be applied neat to the detection system. Sometimes, the sample contains no more 20 μl of buffer or fluid. The sample, in some embodiments, is contained in no more than 1, 5, 10, 15, 20, 25, 30, 35 40, 45, 50, 55, 60, 65, 70, 75, 80, 90, 100, 200, 300, 400, 500 μl, or any of value 1 μl to 500 μl, preferably 10 μL to 200 μL, or more preferably 50 μL to 100 μL of buffer or fluid. Sometimes, the sample is contained in more than 500 μl.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

In some embodiments, samples are used for diagnosing a disease. In some embodiments the disease is cancer. The sample used for cancer testing may comprise at least one target nucleic acid that may hybridize to a guide nucleic acid of the reagents described herein. The target nucleic acid, in some embodiments, comprises a portion of a gene comprising a mutation associated with a disease, such as cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker. In some embodiments, the assay may be used to detect “hotspots” in target nucleic acids that may be predictive of a cancer. In some embodiments, the target nucleic acid comprises a portion of a nucleic acid that is associated with a cancer. In some embodiments, the target nucleic acid is a portion of a nucleic acid from a genomic locus, any DNA amplicon of, a reverse transcribed mRNA, or a cDNA from a locus of at least one of a gene set forth in TABLE 3. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, samples are used to diagnose a genetic disorder, also referred to as genetic disorder testing. The sample used for genetic disorder testing may comprise at least one target nucleic acid that may hybridize to a guide nucleic acid of the reagents described herein. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is a nucleic acid from a genomic locus, a transcribed mRNA, or a reverse transcribed mRNA, a DNA amplicon of or a cDNA from a locus of at least one of a gene set forth in TABLE 3.

A sample used for phenotyping testing may comprise at least one target nucleic acid that may hybridize to a guide nucleic acid of the reagents described herein. The target nucleic acid, in some embodiments, is a nucleic acid encoding a sequence associated with a phenotypic trait. A sample used for genotyping testing may comprise at least one target nucleic acid that may hybridize to a guide nucleic acid of the reagents described herein. A target nucleic acid, in some embodiments, is a nucleic acid encoding a sequence associated with a genotype of interest. A sample used for ancestral testing may comprise at least one target nucleic acid that may hybridize to a guide nucleic acid of the reagents described herein. A target nucleic acid, in some embodiments, is a nucleic acid encoding a sequence associated with a geographic region of origin or ethnic group. A sample may be used for identifying a disease status. For example, a sample is any sample described herein, and is obtained from a subject for use in identifying a disease status of a subject. In some embodiments, the disease is cancer. In some embodiments, the disease is a genetic disorder. In some embodiments, a method comprises obtaining a serum sample from a subject; and identifying a disease status of the subject.

### Certain Target Nucleic Acids

Disclosed herein are compositions, systems and methods for modifying and detecting target nucleic acids. In some embodiments, the target nucleic acid is a double stranded nucleic acid. In some embodiments, the target nucleic acid is a single stranded nucleic acid. In some embodiments, the target nucleic acid is a double stranded nucleic acid that is prepared into single stranded nucleic acids before or upon contacting a reagent or sample. In some embodiments, the target nucleic acid comprises DNA. In some embodiments, the target nucleic acid comprises RNA. The target nucleic acids include but are not limited to mRNA, rRNA, tRNA, non-coding RNA, long non-coding RNA, and microRNA (miRNA). In some cases, the target nucleic acid is single-stranded RNA (ssRNA) or mRNA.

In some embodiments, target nucleic acids comprise a mutation. The mutation may be a mutation of 1, 2, 3, 4, 5, 6, 7, 8, 9, 10, 11, 12, 13, 14, 15, 16, 17, 18, 19, 20 or more nucleotides. The mutation may result in the insertion of at least one amino acid in a polypeptide encoded by the target nucleic acid. The mutation may result in the deletion of at least one amino acid in a polypeptide encoded by the target nucleic acid. The mutation may result in the substitution of at least one amino acid in a polypeptide encoded by the target nucleic acid. The mutation may result in misfolding of the polypeptide. The mutation may result in a premature stop codon. The mutation may result in a truncation of the protein.

In some embodiments, at least a portion of a guide nucleic acid of a composition described herein hybridizes to a region of the target nucleic acid comprising the mutation. In some embodiments, at least a portion of a guide nucleic acid of a composition described herein hybridizes to a region of the target nucleic acid that is within 10 nucleotides, within 50 nucleotides, within 100 nucleotides, or within 200 nucleotides of the mutation. The mutation may be located in a non-coding region or a coding region of a gene.

In some embodiments, the mutation is an autosomal dominant mutation. In some embodiments, the mutation is a dominant negative mutation. In some embodiments, the mutation is a loss of function mutation. In some embodiments, the mutation is a single nucleotide polymorphism (SNP). In some embodiments, the SNP is associated with a phenotype of the sample or a phenotype of the organism from which the sample was taken. The SNP, in some cases, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, the target nucleic acid comprises a mutation associated with a disease. In some examples, a mutation associated with a disease refers to a mutation which causes the disease, contributes to the development of the disease, or indicates the existence of the disease. In some embodiments, the mutation causes the disease.

Non-limiting examples of diseases associated with genetic mutations are cystic fibrosis, Duchenne muscular dystrophy, β-thalassemia, hemophilia, sickle cell anemia, amyotrophic lateral sclerosis (ALS), severe combined immunodeficiency, Huntington's disease, Alzheimer's Disease, alpha-1 antitrypsin deficiency, myotonic dystrophy Type 1, and Usher syndrome. The disease may comprise, at least in part, a cancer, an inherited disorder, an ophthalmological disorder, a neurological disorder, a blood disorder, a metabolic disorder, or a combination thereof.

The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay may be used to detect “hotspots” in target nucleic acids that may be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. In some cases, the target nucleic acid comprises a portion of a nucleic acid from a genomic locus, any DNA amplicon of, a reverse transcribed mRNA, or a cDNA from a locus of at least one of: TRAC, B2M, PD1, PCSK9, DNMT1, HPRT1, RPL32P3, CCR5, FANCF, GRIN2B, EMX1, AAVS1, ALKBH5, CLTA, CDK11, CTNNB1, AXIN1, LRP6, TBK1, BAP1, TLE3, PPM1A, BCL2L2, SUFU, RICTOR, VPS35, TOP1, SIRT1, PTEN, MMD, PAQR8, H2AX, POU5F1, OCT4, SYS1, ARFRP1, TSPAN14, EMC2, EMC3, SEL1L, DERL2, UBE2G2, UBE2J1, and HRD1.

In some cases, the target nucleic acid is a portion of a nucleic acid from a genomic locus, any DNA amplicon of, a reverse transcribed mRNA, or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USHIC, USH2A, VPS13A, VPS13B, VPS45, VRK1, VSX2, WNT10A, XPA, XPC, and ZFYVE26.

The target nucleic acid may be from any organism, including, but not limited to, a bacterium, a virus, a parasite, a protozoon, a fungus, a mammal, a plant, and an insect. As another non-limiting example, the target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides).

In some embodiments, the target nucleic acid is selected from those listed in TABLE 3.

TABLE 3
EXEMPLARY TARGETS
Exemplary targets

AAVS1, ABCA4, ABCB11, ABCC8, ABCD1, ABCG5, ABCG8, ACAD9, ACADM, ACADVL, ACAT1,
ACOX1, ACSF3, ACTA1, ADA, ADAMTS2, ADGRG1, AGA, AGL, AGPS, AGXT, AHI1, AIRE,
ALDH3A2, ALDOB, ALG6, ALK, ALKBH5, ALMS1, ALPL, AMRC9, AMT, ANAPC10, ANAPC11,
ANGPTL3, ANGPTLA, APC, Apo(a), APOCIII, APOEε4, APOL1, APP, AQP2, AR, ARFRP1, ARG1,
ARH, ARL13B, ARL6, ARSA, ARSB, ASL, ASNS, ASPA, ASS1, ATM, ATP6V1B1, ATP7A, ATP7B, ATRX,
ATXN1, ATXN10, ATXN2, ATXN3, ATXN7, ATXN8OS, AXIN1, AXIN2, B2M, BACE-1, BAK1, BAP1,
BARD1, BAX2, BBS1, BBS10, BBS12, BBS2, BCKDHA, BCKDHB, BCL2L2, BCS1L, BEST1, Betaglobin
gene, BLM, BMPR1A, BRAF, BRAFV600E, BRCA1, BRCA2, BRIP1, BSND, C9orf72, CA4, CACNA1A,
CAH1, CAPN3, CASR, CBS, CCNB1 CC2D2A, CCR5, CD1, CD2, CD3, CD3D, CD3Z, CD4, CD5,
CD6, CD7, CD8A, CD8B, CD9, CD14, CD18, CD19, CD21, CD22, CD23, CD27, CD28, CD30, CD33,
CD34, CD36, CD38, CD40, CD40L, CD44, CD46, CD47, CD48, CD52, CD55, CD57, CD58, CD59,
CD68, CD69, CD72, CD73, CD74, CD79A, CD80, CD81, CD83, CD84, CD86, CD90, CD93, CD96,
CD99, CD100, CD123, CD160, CD163, CD164, CD164L2, CD166, CD200, CD204, CD207, CD209,
CD226, CD244, CD247, CD274, CD276, CD300, CD320, CDC73, CDH1, CDH23, CDK11, CDK4,
CDKN1A, CDKN1B, CDKN1C, CDKN2A, CDKN2B, CEBPA, CELA3B, CEP290, CERKL, CFB, CFTR,
CHCHD10, CHEK2, CHM, CHRNE, CIDEB, CIITA, CLN3, CLN5, CLN6, CLN8, CLRN1, CLTA,
CMT1A, CNBP, CNGB1, CNGB3, COL1A1, COL1A2, COL27A1, COL4A3, COL4A4, COL4A5,
COL6A1, COL6A2, COL6A3, COL7A1, CPSI, CPTIA, CPT2, CRB1, CREBBP, CRX, CRYAA, CTNNA1,
CTNNB1, CTNND2, CTNS, CTSK, CXCL12, CYBA, CYBB, CYP11B1, CYP11B2, CYP17A1, CYP19A1,
CYP21A2, CYP27A1, DBT, DCC, DCLRE1C, DERL2, DFNA36, DFNB31, DGAT2, DHCR7, DHDDS,
DICER1, DIS3L2, DLD, DMD, DMPK, DNAH5, DNAI1, DNAI2, DNM2, DNMT1, DPC4, DYSF, EDA,
EDN3, EDNRB, EGFR, EIF2B5, EMC2, EMC3, EMD, EMX1, EN1, EPCAM, ERCC6, ERCC8, ESCO2,
ETFA, ETFDH, ETHE1, EVC, EVC2, EYS, F5, F9, FXI, FAH, FAM161A, FANCA, FANCB, FANCC,
FANCD1, FANCD2, FANCE, FANCF, FANCG, FANCI, FANCJ, FANCL, FANCM, FANCN, FANCP,
FANCS, FBN1, FGF14, FGFR2, FGFR3, FGA, FGB, FGG, FH, FHL1, FIX, FKRP, FKTN, FLCN,
FMR1, FOXP3, FSCN2, FSHD1, FUS, FUT8, FVIII, FXII, FXN, G6PC, GAA, GALC, GALK1, GALT,
GAMT, GATA2, GATA-4, GBA, GBEI, GCDH, GCGR, GDNF, GFAP, GFMI, GHR, GJB1, GJB2, GLA,
GLB1, GLDC, GLE1, GNE, GNPTAB, GNPTG, GNS, GPAM, GPC3, GPR98, GREM1, GRHPR,
GRIN2B, H2AFX, H2AX, HADHA, HAX1, HBA1, HBA2, HBB, HBV cccDNA, HER2, HEXA, HEXB,
HFE, HGSNAT, HLCS, HMGCL, HAO1, HOGA1, HOXB13, HPRPF3, HPRT1, HPS1, HPS3, HRAS,
HRD1, HSD3B2, HSD17B4, HSD17B13, HTT, HUSI, HYALI, HYLSI, IDS, IDUA, IFITM5, IKBKAP,
IL2RG, IL7R, IMPDH1, INPP5E, IRF4, ITGB2, ITPRI, IVD, JAGI, JAKI, JAK3, KCNC3, KCND3,
KCNJ11, KLKB1, KLHL7, KRAS, LAMA1, LAMA2, LAMA3, LAMB3, LAMC2, LCA5, LDHA, LDLR,
LDLRAP1, LHX3, LIFR, LIPA, LMNA, LMOD3, LOR, LOXHD1, LPA, LPL, LRAT, LRP6, LRPPRC,
LRRK2, MADR2, MAN2B1, MAPT, MARCI, MAX, MCM6, MCOLNI, MECP2, MED17, MEFV, MENI,
MERTK, MESP2, MET, METex14, MFN2, MFSD8, MIA3, MITF, MKL2, MKS1, MLC1, MLH1, MLH3,
MMAA, MMAB, MMACHC, MMADHC, MMD, MPI, MPL, MPV17, MSH2, MSH3, MSH6, MTHFD1L,
MTHFR, MTM1, MTRR, MTTP, MUT, MUTYH, MYC, MYH7, MYO7A, NAGLU, NAGS, NBN, NDRG1,
NDUFAF5, NDUFS6, NEB, NF1, NF2, NKX2-5, NOG, NOTCH1, NOTCH2, NPC1, NPC2, NPHP1,
NPHS1, NPHS2, NRAS, NR2E3, NTHL1, NTRK, NTRK1, OAT, OCT4, OFD1, OPA3, OTC, PAH,
PALB2, PAQR8, PAX3, PC, PCCA, PCCB, PCDH15, PCSK9, PD1, PDCD1, PDE6B, PDGFRA,
PDHA1, PDHB, PEX1, PEX10, PEX12, PEX13, PEX14, PEX16, PEX19, PEX2, PEX26, PEX3, PEX5,
PEX6, PEX7, PFKM, PHGDH, PHOX2B, PKD1, PKD2, PKHDI, PKK, PLEKHG4, PMM2, PMP22,
PMS1, PMS2, PNPLA3, POLD1, POLE, POMGNT1, POT1, POU5F1, PPM1A, PPP2R2B, PPT1,
PRCD, PRKAG2, PRKAR1A, PRKCG, PRNP, PROM1, PROP1, PRPF31, PRPF8, PRPH2, PRPS1,
PSAP, PSD3, PSD95, PSEN1, PSEN2, PSRC1, PTCH1, PTEN, PTS, PUS1, PYGM, RAB23, RAD50,
RAD51C, RAD51D, RAG1, RAG2, RAPSN, RARS2, RB1, RDH12, RECQL4, RET, RHO, RICTOR,
RMRP, ROS1, RP1, RP2, RPE65, RPGR, RPGRIP1L, RPL32P3, RS1, RTCA, RTEL1, RUNX1, SACS,
SAMHD1, SCN1A, SCN2A, SDHA, SDHAF2, SDHB, SDHC, SDHD, SEL1L, SEPSEC5, SERPINA1,
SERPINC1, SERPING1, SGCA, SGCB, SGCG, SGSH, SIRT1, SLC12A3, SLC12A6, SLC17A5, SLC22A5,
SLC25A13, SLC25A15, SLC26A2, SLC26A4, SLC35A3, SLC35B4, SLC37A4, SLC39A4, SLC4A11,
SLC6A8, SLC7A7, SMAD3, SMAD4, SMARCA4, SMARCAL1, SMARCB1, SMARCE1, SMN1, SMPD1,
SNAI2, SNCA, SNRNP200, SOD1, SOX10, SPARA7, SPTBN2, STAR, STAT3, STK11, SUFU, SUMF1,
SYNE1, SYNE2, SYS1, TARDBP, TAT, TBK1, TBP, TCIRG1, TCTN3, TECPR2, TERC, TERT, TFR2,
TGFBR2, TGM1, TH, TLE3, TMEM127, TMEM138, TMEM216, TMEM43, TMEM67, TMPRSS6, TOP1,
TOPORS, TP53, TPM2, TNNT1, TNN3, TNNI2, TPP1, TRAC, TRMU, TSC1, TSC2, TSFM, TSPAN14,
TTBK2, TTC8, TTPA, TTR, TULP1, TYMP, UBE2G2, UBE2J1, UBE3A, USH1C, USH1G, USH2A,
VEGF, VHL, VPS13A, VPS13B, VPS35, VPS45, VRK1, VSX2, VWF, WAS, WDR19, WDR48, WNT10A,
WRN, WS2B, WS2C, WT1, XPA, XPC, XPF, XRCC3, YAP1, ZAC1, ZEB1, ZFYVE26, and ZNF423

### IX. Compositions

Disclosed herein are compositions comprising one or more effector proteins described herein or nucleic acids encoding the one or more effector proteins, one or more guide nucleic acids described herein or nucleic acids encoding the one or more guide nucleic acids described herein, or combinations thereof. In some embodiments, a repeat sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions comprise one or more donor nucleic acids described herein. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

### Pharmaceutical Compositions

In some embodiments, compositions described herein are pharmaceutical compositions. In some embodiments, the pharmaceutical compositions comprise compositions described herein and a pharmaceutically acceptable carrier or diluent. “Pharmaceutically acceptable excipient, carrier or diluent” refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### X. Systems

Disclosed herein, in some aspects, are systems for detecting a target nucleic acid, comprising any one of the effector proteins described herein. In some embodiments, systems comprise a guide nucleic acid. Systems may be used to detect a target nucleic acid. In some embodiments, systems comprise an effector protein described herein, a reagent, support medium, or a combination thereof. In some embodiments, systems comprise a fusion protein described herein. In some embodiments, effector proteins comprise an amino acid sequence that is at least 70%, at least 75%, at least 80%, at least 85%, at least 90%, at least 95%, or 100% identical to any one of the amino acid sequences selected from SEQ ID NOS: 1-10,484 or 15,022-24,165. In some embodiments, the amino acid sequence of the effector protein is at least 70%, at least 75%, at least 80%, at least 85%, at least 90%, at least 95%, or 100% identical to any one of the amino acid sequences selected from SEQ ID NOS: 1-10,484 or 15,022-24,165. In some embodiments, systems comprise an effector protein that is at least 90% identical to an effector protein sequence provide in TABLE 1, and a guide nucleic acid that is at least 90% identical to a corresponding guide nucleic from TABLE 1, wherein corresponding means the effector protein sequence and guide nucleic acid sequence are selected from the same column number (e.g., A1 and B1) and same row.

Systems may be used for detecting the presence of a target nucleic acid associated with or causative of a disease, such as cancer, a genetic disorder, or an infection. In some embodiments, systems are useful for phenotyping, genotyping, or determining ancestry. Unless specified otherwise, systems include kits and may be referred to as kits. Unless specified otherwise, systems include devices and may also be referred to as devices. Systems described herein may be provided in the form of a companion diagnostic assay or device, a point-of-care assay or device, or an over-the-counter diagnostic assay/device.

Reagents and effector proteins of various systems may be provided in a reagent chamber or on a support medium. Alternatively, the reagent and/or effector protein may be contacted with the reagent chamber or the support medium by the individual using the system. An exemplary reagent chamber is a test well or container. The opening of the reagent chamber may be large enough to accommodate the support medium. Optionally, the system comprises a buffer and a dropper. The buffer may be provided in a dropper bottle for ease of dispensing. The dropper may be disposable and transfer a fixed volume. The dropper may be used to place a sample into the reagent chamber or on the support medium.

Disclosed herein are systems for detecting and/or editing target nucleic acid. In some embodiments, systems comprise components comprising one or more of: compositions described herein; a solution or buffer; a reagent; a support medium; other components or appurtenances as described herein; or combinations thereof.

### System Solutions

In general, system components comprise a solution in which the activity of an effector protein occurs. Often, the solution comprises or consists essentially of a buffer. The solution or buffer may comprise a buffering agent, a salt, a crowding agent, a detergent, a reducing agent, a competitor, or a combination thereof. Often the buffer is the primary component or the basis for the solution in which the activity occurs. Thus, concentrations for components of buffers described herein (e.g., buffering agents, salts, crowding agents, detergents, reducing agents, and competitors) are the same or essentially the same as the concentration of these components in the solution in which the activity occurs. In some embodiments, a buffer is required for cell lysis activity or viral lysis activity.

In some embodiments, systems comprise a buffer, wherein the buffer comprise at least one buffering agent. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. In some embodiments, the concentration of the buffering agent in the buffer is 1 mM to 200 mM. A buffer compatible with an effector protein may comprise a buffering agent at a concentration of 10 mM to 30 mM. A buffer compatible with an effector protein may comprise a buffering agent at a concentration of about 20 mM. A buffering agent may provide a pH for the buffer or the solution in which the activity of the effector protein occurs. The pH may be 3 to 4, 3.5 to 4.5, 4 to 5, 4.5 to 5.5, 5 to 6, 5.5 to 6.5, 6 to 7, 6.5 to 7.5, 7 to 8, 7.5 to 8.5, 8 to 9, 8.5 to 9.5, 9 to 10, or 9.5 to 10.5.

In some embodiments, systems comprise a solution, wherein the solution comprises at least one salt. In some embodiments, the at least one salt is selected from potassium acetate, magnesium acetate, sodium chloride, potassium chloride, magnesium chloride, calcium chloride, and any combination thereof. In some embodiments, the concentration of the at least one salt in the solution is 5 mM to 100 mM, 5 mM to 10 mM, 1 mM to 60 mM, or 1 mM to 10 mM. In some embodiments, the concentration of the at least one salt is about 105 mM. In some embodiments, the concentration of the at least one salt is about 55 mM. In some embodiments, the concentration of the at least one salt is about 7 mM. In some embodiments, the solution comprises potassium acetate and magnesium acetate. In some embodiments, the solution comprises sodium chloride and magnesium chloride. In some embodiments, the solution comprises potassium chloride and magnesium chloride. In some embodiments, the salt is a magnesium salt and the concentration of magnesium in the solution is at least 5 mM, 7 mM, at least 9 mM, at least 11 mM, at least 13 mM, or at least 15 mM. In some embodiments, the concentration of magnesium is less than 20 mM, less than 18 mM, or less than 16 mM.

In some embodiments, systems comprise a solution, wherein the solution comprises at least one crowding agent. A crowding agent may reduce the volume of solvent available for other molecules in the solution, thereby increasing the effective concentrations of said molecules. Exemplary crowding agents include glycerol and bovine serum albumin. In some embodiments, the crowding agent is glycerol. In some embodiments, the concentration of the crowding agent in the solution is 0.01% (v/v) to 10% (v/v). In some embodiments, the concentration of the crowding agent in the solution is 0.5% (v/v) to 10% (v/v).

In some embodiments, systems comprise a solution, wherein the solution comprises at least one detergent. Exemplary detergents include Tween, Triton-X, and IGEPAL. A solution may comprise Tween, Triton-X, or any combination thereof. A solution may comprise Triton-X. A solution may comprise IGEPAL CA-630. In some embodiments, the concentration of the detergent in the solution is 2% (v/v) or less. In some embodiments, the concentration of the detergent in the solution is 1% (v/v) or less. In some embodiments, the concentration of the detergent in the solution is 0.00001% (v/v) to 0.01% (v/v). In some embodiments, the concentration of the detergent in the solution is about 0.01% (v/v).

In some embodiments, systems comprise a solution, wherein the solution comprises at least one reducing agent. Exemplary reducing agents comprise dithiothreitol (DTT), B-mercaptoethanol (BME), or tris(2-carboxyethyl) phosphine (TCEP). In some embodiments, the reducing agent is DTT. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is 0.5 mM to 2 mM. In some embodiments, the concentration of the reducing agent in the solution is 0.01 mM to 100 mM. In some embodiments, the concentration of the reducing agent in the solution is 0.1 mM to 10 mM. In some embodiments, the concentration of the reducing agent in the solution is about 1 mM.

In some embodiments, systems comprise a solution, wherein the solution comprises a competitor. In general, competitors compete with the target nucleic acid or the reporter nucleic acid for cleavage by the effector protein or a dimer thereof. Exemplary competitors include heparin, and imidazole, and salmon sperm DNA. In some embodiments, the concentration of the competitor in the solution is 1 μg/mL to 100 μg/mL. In some embodiments, the concentration of the competitor in the solution is 40 μg/mL to 60 μg/mL.

In some embodiments, systems comprise a solution, wherein the solution comprises a co-factor. In some embodiments, the co-factor allows an effector protein or a multimeric complex thereof to perform a function, including pre-crRNA processing and/or target nucleic acid cleavage. The suitability of a cofactor for an effector protein or a multimeric complex thereof may be assessed, such as by methods based on those described by Sundaresan et al. (Cell Rep. 2017 Dec. 26; 21(13): 3728-3739). In some embodiments, an effector or a multimeric complex thereof forms a complex with a co-factor. In some embodiments, the co-factor is a divalent metal ion. In some embodiments, the divalent metal ion is selected from Mg²⁺, Mn²⁺, Zn²⁺, Ca²⁺, Cu²⁺. In some embodiments, the divalent metal ion is Mg²⁺. In some embodiments, the co-factor is Mg²⁺.

### Reporters

In some embodiments, systems disclosed herein comprise a reporter. By way of non-limiting and illustrative example, a reporter may comprise a single stranded nucleic acid and a detection moiety (e.g., a labeled single stranded RNA reporter), wherein the nucleic acid is capable of being cleaved by an effector protein (e.g., a CRISPR/Cas protein as disclosed herein) or a multimeric complex thereof, releasing the detection moiety, and generating a detectable signal. As used herein, “reporter” is used interchangeably with “reporter nucleic acid” or “reporter molecule”. The effector proteins disclosed herein, activated upon hybridization of a guide nucleic acid to a target nucleic acid, may cleave the reporter. Cleaving the “reporter” may be referred to herein as cleaving the “reporter nucleic acid,” the “reporter molecule,” or the “nucleic acid of the reporter.” Reporters may comprise RNA. Reporters may comprise DNA. Reporters may be double-stranded. Reporters may be single-stranded.

In some embodiments, reporters comprise a protein capable of generating a signal. A signal may be a calorimetric, potentiometric, amperometric, optical (e.g., fluorescent, colorimetric, etc.), or piezo-electric signal. In some embodiments, the reporter comprises a detection moiety. Suitable detectable labels and/or moieties that may provide a signal include, but are not limited to, an enzyme, a radioisotope, a member of a specific binding pair; a fluorophore; a fluorescent protein; a quantum dot; and the like.

In some embodiments, the reporter comprises a detection moiety and a quenching moiety. In some embodiments, the reporter comprises a cleavage site, wherein the detection moiety is located at a first site on the reporter and the quenching moiety is located at a second site on the reporter, wherein the first site and the second site are separated by the cleavage site. Sometimes the quenching moiety is a fluorescence quenching moiety. In some embodiments, the quenching moiety is 5′ to the cleavage site and the detection moiety is 3′ to the cleavage site. In some embodiments, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some embodiments, the detection moiety is at the 5′ terminus of the nucleic acid of a reporter. In some embodiments, the quenching moiety is at the 3′ terminus of the nucleic acid of a reporter.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, and glucose oxidase (GO).

In some embodiments, the detection moiety comprises an invertase. The substrate of the invertase may be sucrose. A DNS reagent may be included in the system to produce a colorimetric change when the invertase converts sucrose to glucose. In some embodiments, the reporter nucleic acid and invertase are conjugated using a heterobifunctional linker by sulfo-SMCC chemistry.

Suitable fluorophores may provide a detectable fluorescence signal in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Non-limiting examples of fluorophores are fluorescein amidite, 6-Fluorescein, IRDye 700, TYE 665, Alex Fluor 594, or ATTO TM 633 (NHS Ester). The fluorophore may be an infrared fluorophore. The fluorophore may emit fluorescence in the range of 500 nm and 720 nm. In some embodiments, the fluorophore emits fluorescence at a wavelength of 700 nm or higher. In other embodiments, the fluorophore emits fluorescence at about 665 nm. In some embodiments, the fluorophore emits fluorescence in the range of 500 nm to 520 nm, 500 nm to 540 nm, 500 nm to 590 nm, 590 nm to 600 nm, 600 nm to 610 nm, 610 nm to 620 nm, 620 nm to 630 nm, 630 nm to 640 nm, 640 nm to 650 nm, 650 nm to 660 nm, 660 nm to 670 nm, 670 nm to 680 nm, 690 nm to 690 nm, 690 nm to 700 nm, 700 nm to 710 nm, 710 nm to 720 nm, or 720 nm to 730 nm. In some embodiments, the fluorophore emits fluorescence in the range 450 nm to 750 nm, 500 nm to 650 nm, or 550 to 650 nm.

Systems may comprise a quenching moiety. A quenching moiety may be chosen based on its ability to quench the detection moiety. A quenching moiety may be a non-fluorescent fluorescence quencher. A quenching moiety may quench a detection moiety that emits fluorescence in the range of 500 nm and 720 nm. A quenching moiety may quench a detection moiety that emits fluorescence in the range of 500 nm and 720 nm. In some embodiments, the quenching moiety quenches a detection moiety that emits fluorescence at a wavelength of 700 nm or higher. In other embodiments, the quenching moiety quenches a detection moiety that emits fluorescence at about 660 nm or about 670 nm. In some embodiments, the quenching moiety quenches a detection moiety that emits fluorescence in the range of 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some embodiments, the quenching moiety quenches a detection moiety that emits fluorescence in the range 450 nm to 750 nm, 500 nm to 650 nm, or 550 to 650 nm. A quenching moiety may quench fluorescein amidite, 6-Fluorescein, IRDye 700, TYE 665, Alex Fluor 594, or ATTO TM 633 (NHS Ester). A quenching moiety may be Iowa Black RQ, Iowa Black FQ or IRDye QC-1 Quencher. A quenching moiety may quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety may be Iowa Black RQ (Integrated DNA Technologies), Iowa Black FQ (Integrated DNA Technologies) or IRDye QC-1 Quencher (LiCor). Any of the quenching moieties described herein may be from any commercially available source, may be an alternative with a similar function, a generic, or a non-tradename of the quenching moieties listed.

The generation of the detectable signal from the release of the detection moiety may indicate that cleavage by the effector protein has occurred and that the sample contains the target nucleic acid. In some embodiments, the detection moiety comprises a fluorescent dye. Sometimes the detection moiety comprises a fluorescence resonance energy transfer (FRET) pair. In some embodiments, the detection moiety comprises an infrared (IR) dye. In some embodiments, the detection moiety comprises an ultraviolet (UV) dye. Alternatively, or in combination, the detection moiety comprises a protein. Sometimes the detection moiety comprises a biotin. Sometimes the detection moiety comprises at least one of avidin or streptavidin. In some embodiments, the detection moiety comprises a polysaccharide, a polymer, or a nanoparticle. In some embodiments, the detection moiety comprises a gold nanoparticle or a latex nanoparticle.

A detection moiety may be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal may be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal may be a colorimetric signal or a signal visible by eye. In some embodiments, the detectable signal may be fluorescent, electrical, chemical, electrochemical, or magnetic. In some embodiments, the first detection signal may be generated by interaction of the detection moiety to the capture molecule in the detection region, where the first detection signal indicates that the sample contained the target nucleic acid. Sometimes systems are capable of detecting more than one type of target nucleic acid, wherein the system comprises more than one type of guide nucleic acid and more than one type of reporter nucleic acid. In some embodiments, the detectable signal may be generated directly by the cleavage event. Alternatively, or in combination, the detectable signal may be generated indirectly by the signal event. Sometimes the detectable signal is not a fluorescent signal. In some embodiments, the detectable signal may be a colorimetric or color-based signal. In some embodiments, the detected target nucleic acid may be identified based on its spatial location on the detection region of the support medium. In some embodiments, the second detectable signal may be generated in a spatially distinct location than the first generated signal.

In some embodiments, the reporter nucleic acid is a single-stranded nucleic acid sequence comprising ribonucleotides. The nucleic acid of a reporter may be a single-stranded nucleic acid sequence comprising at least one ribonucleotide. In some embodiments, the nucleic acid of a reporter is a single-stranded nucleic acid comprising at least one ribonucleotide residue at an internal position that functions as a cleavage site. In some embodiments, the nucleic acid of a reporter comprises at least 2, at least 3, at least 4, at least 5, at least 6, at least 7, at least 8, at least 9, or at least 10 ribonucleotide residues at an internal position. In some embodiments, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some embodiments, the nucleic acid of a reporter has only ribonucleotide residues. In some embodiments, the nucleic acid of a reporter has only DNA residues. In some embodiments, the nucleic acid comprises nucleotides resistant to cleavage by the effector protein described herein. In some embodiments, the nucleic acid of a reporter comprises synthetic nucleotides. In some embodiments, the nucleic acid of a reporter comprises at least one ribonucleotide residue and at least one non-ribonucleotide residue.

In some embodiments, the nucleic acid of a reporter comprises at least one uracil ribonucleotide. In some embodiments, the nucleic acid of a reporter comprises at least two uracil ribonucleotides. Sometimes the nucleic acid of a reporter has only uracil ribonucleotides. In some embodiments, the nucleic acid of a reporter comprises at least one adenine ribonucleotide. In some embodiments, the nucleic acid of a reporter comprises at least two adenine ribonucleotides. In some embodiments, the nucleic acid of a reporter has only adenine ribonucleotides. In some embodiments, the nucleic acid of a reporter comprises at least one cytosine ribonucleotide. In some embodiments, the nucleic acid of a reporter comprises at least two cytosine ribonucleotides. In some embodiments, the nucleic acid of a reporter comprises at least one guanine ribonucleotide. In some embodiments, the nucleic acid of a reporter comprises at least two guanine ribonucleotides. In some embodiments, a nucleic acid of a reporter comprises a single unmodified ribonucleotide. In some embodiments, a nucleic acid of a reporter comprises only unmodified DNAs.

In some embodiments, the nucleic acid of a reporter is 5 to 20, 5 to 15, 5 to 10, 7 to 20, 7 to 15, or 7 to 10 nucleotides in length. In some embodiments, the nucleic acid of a reporter is 3 to 20, 4 to 10, 5 to 10, or 5 to 8 nucleotides in length. In some embodiments, the nucleic acid of a reporter is 5 to 12 nucleotides in length. In some embodiments, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some embodiments, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, systems comprise a plurality of reporters. The plurality of reporters may comprise a plurality of signals. In some embodiments, systems comprise at least 2, at least 3, at least 4, at least 5, at least 6, at least 7, at least 8, at least 9, at least 10, at least 11, at least 12, at least 13, at least 14, at least 15, at least 20, at least 30, at least 40, or at least 50 reporters. In some embodiments, there are 2 to 50, 3 to 40, 4 to 30, 5 to 20, or 6 to 10 different reporters.

Herein, detection of reporter cleavage to determine the presence of a target nucleic acid may be referred to as ‘DETECTR’. In some embodiments described herein is a method of assaying for a target nucleic acid in a sample comprising contacting the target nucleic acid with an effector protein, a non-naturally occurring guide nucleic acid that hybridizes to a segment of the target nucleic acid, and a reporter nucleic acid, and assaying for a change in a signal, wherein the change in the signal is produced by cleavage of the reporter nucleic acid.

In the presence of a large amount of non-target nucleic acids, an activity of an effector protein (e.g., an effector protein as disclosed herein) may be inhibited. This is because the activated effector proteins collaterally cleave any nucleic acids. If total nucleic acids are present in large amounts, they may outcompete reporters for the effector proteins. In some embodiments, systems comprise an excess of reporter(s), such that when the system is operated and a solution of the system comprising the reporter is combined with a sample comprising a target nucleic acid, the concentration of the reporter in the combined solution-sample is greater than the concentration of the target nucleic acid. In some embodiments, the sample comprises amplified target nucleic acid. In some embodiments, the sample comprises an unamplified target nucleic acid. In some embodiments, the concentration of the reporter is greater than the concentration of target nucleic acids and non-target nucleic acids. The non-target nucleic acids may be from the original sample, either lysed or unlysed. The non-target nucleic acids may comprise byproducts of amplification. In some embodiments, systems comprise a reporter wherein the concentration of the reporter in a solution is 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold excess of total nucleic acids. In some embodiments, systems comprise a reporter wherein the concentration of the reporter in a solution is 1.5 fold to 100 fold, 2 fold to 10 fold, 10 fold to 20 fold, 20 fold to 30 fold, 30 fold to 40 fold, 40 fold to 50 fold, 50 fold to 60 fold, 60 fold to 70 fold, 70 fold to 80 fold, 80 fold to 90 fold, 90 fold to 100 fold, 1.5 fold to 10 fold, 1.5 fold to 20 fold, 10 fold to 40 fold, 20 fold to 60 fold, or 10 fold to 80 fold excess of total nucleic acids.

### Amplification Reagents/Components

In some embodiments, systems described herein comprise a reagent or component for amplifying a nucleic acid. Non-limiting examples of reagents for amplifying a nucleic acid include polymerases, primers, and nucleotides. In some embodiments, systems comprise reagents for nucleic acid amplification of a target nucleic acid in a sample. Nucleic acid amplification of the target nucleic acid may improve at least one of sensitivity, specificity, or accuracy of the assay in detecting the target nucleic acid. In some embodiments, nucleic acid amplification is isothermal nucleic acid amplification, providing for the use of the system or system in remote regions or low resource settings without specialized equipment for amplification. In some embodiments, amplification of the target nucleic acid increases the concentration of the target nucleic acid in the sample relative to the concentration of nucleic acids that do not correspond to the target nucleic acid.

The reagents for nucleic acid amplification may comprise a recombinase, an oligonucleotide primer, a single-stranded DNA binding (SSB) protein, a polymerase, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, systems described herein comprise a PCR tube, a PCR well or a PCR plate. In some embodiments, the wells of the PCR plate may be pre-aliquoted with the reagent for amplifying a nucleic acid, as well as a guide nucleic acid, an effector protein, a multimeric complex, or any combination thereof.

In some embodiments, systems comprise a PCR plate; a guide nucleic acid targeting a target sequence; an effector protein capable of being activated when complexed with the guide nucleic acid and the target sequence; and a single stranded reporter nucleic acid comprising a detection moiety, wherein the reporter nucleic acid is capable of being cleaved by the activated nuclease, thereby generating a detectable signal.

In some embodiments, systems described herein comprise a support medium; a guide nucleic acid targeting a target sequence; and an effector protein capable of being activated when complexed with the guide nucleic acid and the target sequence. In some embodiments, nucleic acid amplification is performed in a nucleic acid amplification region on the support medium. Alternatively, or in combination, the nucleic acid amplification is performed in a reagent chamber, and the resulting sample is applied to the support medium.

In some embodiments, a system described herein for editing a target nucleic acid comprises a PCR plate; a guide nucleic acid targeting a target sequence; and an effector protein capable of being activated when complexed with the guide nucleic acid and the target sequence. In some embodiments, the wells of the PCR plate may be pre-aliquoted with the guide nucleic acid targeting a target sequence, and an effector protein capable of being activated when complexed with the guide nucleic acid and the target sequence. A user may thus add the biological sample of interest to a well of the pre-aliquoted PCR plate.

In some embodiments, wells of the PCR plate may be pre-aliquoted with a guide nucleic acid targeting a target sequence, an effector protein capable of being activated when complexed with the guide nucleic acid and the target sequence, and at least one population of a single stranded reporter nucleic acid comprising a detection moiety. In some embodiments, the reporter nucleic acid is capable of being cleaved by the activated nuclease, thereby generating a detectable signal. A user may thus add the biological sample of interest to a well of the pre-aliquoted PCR plate and measure for the detectable signal with a fluorescent light reader or a visible light reader.

In some embodiments, amplification reaction of nucleic acid as described herein is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the amplification reaction is performed for 1 to 60, 5 to 55, 10 to 50, 15 to 45, 20 to 40, or 25 to 35 minutes. In some embodiments, the amplification reaction is performed at a temperature of around 20-45° C. In some embodiments, the amplification reaction is performed at a temperature no greater than 20° C., 25° C., 30° C., 35° C., 37° C., 40° C., 45° C., or any value 20° C. to 45° C. In some embodiments, the amplification reaction is performed at a temperature of at least 20° C., 25° C., 30° C., 35° C., 37° C., 40° C., or 45° C., or any value 20° C. to 45° C.

In some embodiments, the amplification reaction is performed at a temperature of 20° C. to 45° C., 25° C. to 40° C., 30° C. to 40° C., or 35° C. to 40° C.

In some embodiments, systems comprise primers for amplifying a target nucleic acid to produce an amplification product comprising the target nucleic acid and a PAM. In some embodiments, at least one of the primers may comprise the PAM that is incorporated into the amplification product during amplification. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid, methods of assaying for a target nucleic acid that lacks a PAM by amplifying the target nucleic acid to introduce a PAM, and compositions used in introducing a PAM by amplification into the target nucleic acid.

### Additional System Components

In some embodiments, systems include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In some embodiments, the containers are formed from a variety of materials such as glass, plastic, or polymers. In some embodiments, the system or systems described herein contain packaging materials. Examples of packaging materials include, but are not limited to, pouches, blister packs, bottles, tubes, bags, containers, bottles, and any packaging material suitable for intended mode of use.

In some embodiments, systems described herein include labels listing contents and/or instructions for use, or package inserts with instructions for use. In some embodiments, the systems include a set of instructions and/or a label is on or associated with the container. In some embodiments, the label is on a container when letters, numbers or other characters forming the label are attached, molded, or etched into the container itself; a label is associated with a container when it is present within a receptacle or carrier that also holds the container (e.g., as a package insert). In some embodiments, the label is used to indicate that the contents are to be used for a specific therapeutic application. In some embodiments, the label indicates directions for use of the contents, such as in the methods described herein. In some embodiments, after packaging the formed product and wrapping or boxing to maintain a sterile barrier, the product is terminally sterilized by heat sterilization, gas sterilization, gamma irradiation, or by electron beam sterilization. Alternatively, in some embodiments, the product is prepared and packaged by aseptic processing.

In some embodiments, systems comprise a solid support. An RNP or effector protein may be attached to a solid support. The solid support may be an electrode or a bead. The bead may be a magnetic bead. Upon cleavage, the RNP is liberated from the solid support and interacts with other mixtures. For example, upon cleavage of the nucleic acid of the RNP, the effector protein of the RNP flows through a chamber into a mixture comprising a substrate. When the effector protein meets the substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

### Certain System Conditions

In some embodiments, systems and methods are employed under certain conditions that enhance an activity of the effector protein relative to alternative conditions, as measured by a detectable signal released from cleavage of a reporter in the presence of the target nucleic acid. In some embodiments, the reporter nucleic acid is a homopolymeric reporter nucleic acid comprising 5 to 20 consecutive adenines, 5 to 20 consecutive thymines, 5 to 20 consecutive cytosines, or 5 to 20 consecutive guanines. In some embodiments, the reporter is an RNA-FQ reporter.

In some embodiments, effector proteins disclosed herein recognize, bind, or are activated by, different target nucleic acids having different sequences, but are active toward the same reporter nucleic acid, allowing for facile multiplexing in a single assay having a single ssRNA-FQ reporter.

In some embodiments, systems and methods are employed under certain conditions that enhance cis-cleavage activity of the effector protein.

Certain conditions that may enhance the activity of an effector protein include a certain salt presence or salt concentration of the solution in which the activity occurs. For example, cis-cleavage activity of an effector protein may be inhibited or halted by a high salt concentration. The salt may be a sodium salt, a potassium salt, or a magnesium salt. In some embodiments, the salt is NaCl. In some embodiments, the salt is KNO₃. In some embodiments, the salt concentration is less than 150 mM, less than 125 mM, less than 100 mM, less than 75 mM, less than 50 mM, or less than 25 mM.

Certain conditions that may enhance the activity of an effector protein include the pH of a solution in which the activity. In some embodiments, the pH is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH is less than 7. In some embodiments, the pH is greater than 7.

Certain conditions that may enhance the activity of an effector protein includes the temperature at which the activity is performed. In some embodiments, the temperature is about 25° C. to about 50° C. In some embodiments, the temperature is about 20° C. to about 40° C., about 30° C. to about 50° C., or about 40° C. to about 60° C. In some embodiments, the temperature is about 25° C., about 30° C., about 35° C., about 40° C., about 45° C., or about 50° C.

### XI. Methods and Formulations for Introducing System Components and Compositions into a Target Cell

A guide nucleic acid (or a nucleic acid comprising a nucleotide sequence encoding same) and/or an effector protein described herein may be introduced into a host cell by any of a variety of well-known methods. As a non-limiting example, a guide nucleic acid and/or effector protein may be combined with a lipid. As another non-limiting example, a guide nucleic acid and/or effector protein may be combined with a particle or formulated into a particle.

### Methods for Introducing System Components and Compositions to a Host

Described herein are methods of introducing various components described herein to a host. A host may be any suitable host, such as a host cell. When described herein, a host cell may be an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells may be, or have been, used as recipients for methods of introduction described herein, and include the progeny of the original cell which has been transformed by the methods of introduction described herein. It is understood that the progeny of a single cell may not necessarily be completely identical in morphology or in genomic or total DNA complement as the original parent, due to natural, accidental, or deliberate mutation. A host cell may be a recombinant host cell or a genetically modified host cell, if a heterologous nucleic acid, e.g., an expression vector, has been introduced into the cell.

Methods of introducing a nucleic acid and/or protein into a host cell are known in the art, and any convenient method may be used to introduce a subject nucleic acid (e.g., an expression construct/vector) into a target cell (e.g., a human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like. In some embodiments, the nucleic acid and/or protein are introduced into a disease cell comprised in a pharmaceutical composition comprising the guide nucleic acid and/or effector protein and a pharmaceutically acceptable excipient.

In some embodiments, molecules of interest, such as nucleic acids of interest, are introduced to a host. In some embodiments, polypeptides, such as an effector protein are introduced to a host. In some embodiments, vectors, such as lipid particles and/or viral vectors may be introduced to a host. Introduction may be for contact with a host or for assimilation into the host, for example, introduction into a host cell.

In some embodiments, described herein are methods of introducing one or more nucleic acids, such as a nucleic acid encoding an effector protein, a nucleic acid that, when transcribed, produces an engineered guide nucleic acid, and/or a donor nucleic acid, or combinations thereof, into a host cell. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout.

Introducing one or more nucleic acids into a host cell may occur in any culture media and under any culture conditions that promote the survival of the cells. Introducing one or more nucleic acids into a host cell may be carried out in vivo or ex vivo. Introducing one or more nucleic acids into a host cell may be carried out in vitro.

In some embodiments, an effector protein may be provided as RNA. The RNA may be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.). In some embodiments, introduction of one or more nucleic acid may be through the use of a vector and/or a vector system, accordingly, in some embodiments, compositions and system described herein comprise a vector and/or a vector system.

Vectors may be introduced directly to a host. In some embodiments, host cells may be contacted with one or more vectors as described herein, and in some embodiments, said vectors are taken up by the cells. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest.

Components described herein may also be introduced directly to a host. For example, an engineered guide nucleic acid may be introduced to a host, specifically introduced into a host cell. Methods of introducing nucleic acids, such as RNA into cells include, but are not limited to direct injection, transfection, or any other method used for the introduction of nucleic acids.

Polypeptides (e.g., effector proteins) described herein may also be introduced directly to a host. In some embodiments, polypeptides described herein may be modified to promote introduction to a host. For example, polypeptides described herein may be modified to increase the solubility of the polypeptide. Such a polypeptide may optionally be fused to a polypeptide domain that increases solubility. The domain may be linked to the polypeptide through a defined protease cleavage site, such as TEV sequence which is cleaved by TEV protease. The linker may also include one or more flexible sequences, e.g. from 1 to 10 glycine residues. In some embodiments, the cleavage of the polypeptide is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. In another example, the polypeptide may be modified to improve stability. For example, the polypeptides may be PEGylated, where the polyethyleneoxy group provides for enhanced lifetime in the blood stream. Polypeptides may also be modified to promote uptake by a host, such as a host cell. For example, a polypeptide described herein may be fused to a polypeptide permeant domain to promote uptake by a host cell. Any suitable permeant domains may be used in the non-integrating polypeptides of the present disclosure, including peptides, peptidomimetics, and non-peptide carriers. Examples include penetratin, a permeant peptide may be derived from the third alpha helix of Drosophila melanogaster transcription factor Antennapaedia; the HIV-1 tat basic region amino acid sequence, e.g., amino acids 49-57 of a naturally-occurring tat protein; and poly-arginine motifs, for example, the region of amino acids 34-56 of HIV-1 rev protein, nonaarginine, octa-arginine, and the like. The site at which the fusion is made may be selected in order to optimize the biological activity, secretion or binding characteristics of the polypeptide. The optimal site may be determined by suitable methods.

### Formulations for Introducing System Components and Compositions to a Host

Described herein are formulations of introducing compositions or components of a system described herein to a host. In some embodiments, such formulations, systems and compositions described herein comprise an effector protein and a carrier (e.g., excipient, diluent, vehicle, or filling agent). In some aspects of the present disclosure, the effector protein is provided in a pharmaceutical composition comprising the effector protein and any pharmaceutically acceptable excipient, carrier, or diluent.

### XII. Methods of Modifying a Nucleic Acid

Provided herein are compositions, methods, and systems for modifying (e.g., editing) target nucleic acids. In general, modifying refers to changing the physical composition of a target nucleic acid. However, compositions, methods, and systems disclosed herein may also be capable of modifying target nucleic acids, such as making epigenetic modifications of target nucleic acids, which does not change the nucleotide sequence of the target nucleic acids per se. Effector proteins, compositions and systems described herein may be used for modifying a target nucleic acid, which includes editing a target nucleic acid sequence. Modifying a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or otherwise changing one or more nucleotides of the target nucleic acid. Modifying a target nucleic acid may comprise one or more of: methylating, demethylating, deaminating, or oxidizing one or more nucleotides of the target nucleic acid.

Compositions, methods, and systems described herein may modify a coding portion of a gene, a non-coding portion of a gene, or a combination thereof. Modifying at least one gene using the compositions, methods or systems described herein may reduce or increase expression of one or more genes. In some embodiments, the compositions, methods or systems reduce expression of one or more genes by at least 10%, at least 20%, at least 30%, at least 40%, at least 50%, at least 60%, at least 70%, at least 80%, at least 90%, or at least 95%. In some embodiments, the compositions, methods or systems remove all expression of a gene, also referred to as genetic knock out. In some embodiments, the compositions, methods or systems increase expression of one or more genes by at least 10%, at least 20%, at least 30%, at least 40%, at least 50%, at least 60%, at least 70%, at least 80%, at least 90%, or at least 100%.

In some embodiments, the compositions, methods or systems comprise a nucleic acid expression vector, or use thereof, to introduce an effector protein, guide nucleic acid, donor template or any combination thereof to a cell. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector. In some embodiments, compositions and methods comprise a lipid, polymer, nanoparticle, or a combination thereof, or use thereof, to introduce a Cas protein, guide nucleic acid, donor template or any combination thereof to a cell. Non-limiting examples of lipids and polymers are cationic polymers, cationic lipids, or bio-responsive polymers. In some embodiments, the bio-responsive polymer exploits chemical-physical properties of the endosomal environment (e.g., pH) to preferentially release the genetic material in the intracellular space.

Methods of modifying may comprise contacting a target nucleic acid with one or more components, compositions or systems described herein. In some embodiments, a method of modifying comprises contacting a target nucleic acid with at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a system described herein wherein the system comprises components comprising at least one of: a) one or more effector proteins, or one or more nucleic acids encoding one or more effector proteins; or b) one or more guide nucleic acids, or one or more nucleic acids encoding one or more guide nucleic acids. In some embodiments, a method of modifying comprises contacting a target nucleic acid with a composition described herein comprising at least one of: a) one or more effector proteins, or one or more nucleic acids encoding one or more effector proteins; or b) one or more guide nucleic acids, or one or more nucleic acids encoding one or more guide nucleic acids; in a composition.

Editing a target nucleic acid sequence may introduce a mutation (e.g., point mutations, deletions) in a target nucleic acid relative to a corresponding wildtype nucleotide sequence. Editing may remove or correct a disease-causing mutation in a nucleic acid sequence to produce a corresponding wildtype nucleotide sequence. Editing a target nucleic acid sequence may remove/correct point mutations, deletions, null mutations, or tissue-specific mutations in a target nucleic acid. Editing a target nucleic acid sequence may be used to generate gene knock-out, gene knock-in, gene editing, gene tagging, or a combination thereof. Methods of the disclosure may be targeted to any locus in a genome of a cell.

Modifying may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer sequence. In some embodiments, the effector proteins introduce a single-stranded break in a target nucleic acid to produce a cleaved nucleic acid. In some embodiments, the effector protein is capable of introducing a break in a single stranded RNA (ssRNA). The effector protein may be coupled to a guide nucleic acid that targets a particular region of interest in the ssRNA. In some embodiments, the target nucleic acid, and the resulting cleaved nucleic acid is contacted with a nucleic acid for homologous recombination (e.g., homology directed repair (HDR)) or non-homologous end joining (NHEJ). In some embodiments, a double-stranded break in the target nucleic acid may be repaired (e.g., by NHEJ or HDR) without insertion of a donor template, such that the repair results in an indel in the target nucleic acid at or near the site of the double-stranded break. In some embodiments, an indel, sometimes referred to as an insertion-deletion or indel mutation, is a type of genetic mutation that results from the insertion and/or deletion of one or more nucleotide in a target nucleic acid. An indel may vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation. Indel percentage is the percentage of sequencing reads that show at least one nucleotide has been mutation that results from the insertion and/or deletion of nucleotides regardless of the size of insertion or deletion, or number of nucleotides mutated. For example, if there is at least one nucleotide deletion detected in a given target nucleic acid, it counts towards the percent indel value. As another example, if one copy of the target nucleic acid has one nucleotide deleted, and another copy of the target nucleic acid has 10 nucleotides deleted, they are counted the same. This number reflects the percentage of target nucleic acids that are edited by a given effector protein.

In some embodiments, methods of modifying described herein cleave a target nucleic acid at one or more locations to generate a cleaved target nucleic acid. In some embodiments, the cleaved target nucleic acid undergoes recombination (e.g., NHEJ or HDR). In some embodiments, cleavage in the target nucleic acid may be repaired (e.g., by NHEJ or HDR) without insertion of a donor nucleic acid, such that the repair results in an indel in the target nucleic acid at or near the site of the cleavage site. In some embodiments, cleavage in the target nucleic acid may be repaired (e.g., by NHEJ or HDR) with insertion of a donor nucleic acid, such that the repair results in an indel in the target nucleic acid at or near the site of the cleavage site.

In some embodiments, wherein the compositions, systems, and methods of the present disclosure comprise an additional guide nucleic acid or a use thereof, and such dual-guided compositions, systems, and methods described herein may modify the target nucleic acid in two locations. In some embodiments, dual-guided modifying may comprise cleavage of the target nucleic acid in the two locations targeted by the guide nucleic acids. In some embodiments, upon removal of the sequence between the guide nucleic acids, the wild-type reading frame is restored. A wild-type reading frame may be a reading frame that produces at least a partially, or fully, functional protein. A non-wild-type reading frame may be a reading frame that produces a non-functional or partially non-functional protein. The term “functional protein” refers to protein that retains at least some if not all activity relative to the wildtype protein. A functional protein can also include a protein having enhanced activity relative to the wildtype protein. Assays are known and available for detecting and quantifying protein activity, e.g., colorimetric and fluorescent assays. In some instances, a functional protein is a wildtype protein. In some instances, a functional protein is a functional portion of a wildtype protein.

Accordingly, in some embodiments, compositions, systems, and methods described herein may edit 1 to 1,000 nucleotides or any integer in between, in a target nucleic acid. In some embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, may be edited by the compositions, systems, and methods described herein. In some embodiments, 1, 2, 3, 4, 5, 6, 7, 8, 9, 10, or more nucleotides may be edited by the compositions, systems, and methods described herein. In some embodiments, 10, 20, 30, 40, 50, 60, 70, 80 90, 100 or more nucleotides, or any integer in between, may be edited by the compositions, systems, and methods described herein. In some embodiments, 100, 200, 300, 400, 500, 600, 700, 800, 900 or more nucleotides, or any integer in between, may be edited by the compositions, systems, and methods described herein.

Methods may comprise use of two or more effector proteins. An illustrative method for introducing a break in a target nucleic acid comprises contacting the target nucleic acid with: (a) a first engineered guide nucleic acid comprising a region that binds to a first effector protein described herein; and (b) a second engineered guide nucleic acid comprising a region that binds to a second effector protein described herein, wherein the first engineered guide nucleic acid comprises an additional region that hybridizes to the target nucleic acid and wherein the second engineered guide nucleic acid comprises an additional region that hybridizes to the target nucleic acid. In some embodiments, the first and second effector protein are identical. In some embodiments, the first and second effector protein are not identical.

In some embodiments, editing a target nucleic acid comprises genome editing. Genome editing may comprise editing a genome, chromosome, plasmid, or other genetic material of a cell or organism. In some embodiments, the genome, chromosome, plasmid, or other genetic material of the cell or organism is modified in vivo. In some embodiments, the genome, chromosome, plasmid, or other genetic material of the cell or organism is modified in a cell. In some embodiments, the genome, chromosome, plasmid, or other genetic material of the cell or organism is modified in vitro. For example, a plasmid may be edited in vitro using a composition described herein and introduced into a cell or organism.

In some embodiments, editing a target nucleic acid may comprise deleting a sequence from a target nucleic acid. For example, a mutated sequence or a sequence associated with a disease may be removed from a target nucleic acid. In some embodiments, editing a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, editing a target nucleic acid may comprise introducing a sequence into a target nucleic acid. For example, a beneficial sequence or a sequence that may reduce or eliminate a disease may be inserted into the target nucleic acid.

In some embodiments, methods comprise inserting a donor nucleic acid into a cleaved target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

In some embodiments, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, editing a target nucleic acid may comprise introducing a first break in a first region of the target nucleic acid and a second break in a second region of the target nucleic acid. For example, editing a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby editing the target nucleic acid. In some embodiments, a segment of the target nucleic acid between the first break and the second break may be replaced (e.g., with donor nucleic acid), thereby editing the target nucleic acid.

Methods, systems and compositions described herein may edit a target nucleic acid wherein such editing may effect one or more indels. In some embodiments, where compositions, systems, and/or methods described herein effect one or more indels, the impact on the transcription and/or translation of the target nucleic acid may be predicted depending on: 1) the amount of indels generated; and 2) the location of the indel on the target nucleic acid. For example, as described herein, in some embodiments, if the amount of indels is not divisible by three, and the indels occur within or along a protein coding region, then the edit or mutation may be a frameshift mutation. In some embodiments, if the amount of indels is divisible by three, then a frameshift mutation may not be effected, but a splicing disruption mutation and/or sequence skip mutation may be effected, such as an exon skip mutation. In some embodiments, if the amount of indels is not evenly divisible by three, then a frameshift mutation may be effected.

Methods, systems and compositions described herein may edit a target nucleic acid wherein such editing may be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity may be detected by next generation sequencing of one or more target loci of a target nucleic acid where indel percentage is calculated as the fraction of sequencing reads containing insertions or deletions relative to an unedited reference sequence. In some embodiments, methods, systems, and compositions comprising an effector protein and guide nucleic acid described herein may exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein may exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In some embodiments, editing of a target nucleic acid as described herein effects one or more mutations comprising splicing disruption mutations, frameshift mutations (e.g., 1+ or 2+frameshift mutation), sequence deletion, sequence skipping, sequence reframing, sequence knock-in, or any combination thereof. In some embodiments, the splicing disruption can be an editing that disrupts a splicing of a target nucleic acid or a splicing of a sequence that is transcribed from a target nucleic acid relative to a target nucleic acid without the splicing disruption. In some embodiments, the frameshift mutation can be an editing that alters the reading frame of a target nucleic acid relative to a target nucleic acid without the frameshift mutation. In some embodiments, the frameshift mutation can be a +2 frameshift mutation, wherein a reading frame is edited by 2 bases. In some embodiments, the frameshift mutation can be a +1 frameshift mutation, wherein a reading frame is edited by 1 base. In some embodiments, the frameshift mutation is an editing that alters the number of bases in a target nucleic acid so that it is not divisible by three. In some embodiments, the frameshift mutation can be an editing that is not a splicing disruption. In some embodiments a sequence as described in reference to the sequence deletion, sequence skipping, sequence reframing, and sequence knock-in can be a DNA sequence, a RNA sequence, an edited DNA or RNA sequence, a mutated sequence, a wild-type sequence, a coding sequence, a non-coding sequence, an exonic sequence (exon), an intronic sequence (intron), or any combination thereof. In some embodiments, the sequence deletion is an editing where one or more sequences in a target nucleic acid are deleted relative to a target nucleic acid without the sequence deletion. In some embodiments, the sequence deletion can result in or effect a splicing disruption or a frameshift mutation. In some embodiments, the sequence deletion result in or effect a splicing disruption. In some embodiments, the sequence skipping is an editing where one or more sequences in a target nucleic acid are skipped upon transcription or translation of the target nucleic acid relative to a target nucleic acid without the sequence skipping. In some embodiments, the sequence skipping can result in or effect a splicing disruption or a frameshift mutation. In some embodiments, the sequence skipping can result in or effect a splicing disruption. In some embodiments, the sequence reframing is an editing where one or more bases in a target are edited so that the reading frame of the sequence is reframed relative to a target nucleic acid without the sequence reframing. In some embodiments, the sequence reframing can result in or effect a splicing disruption or a frameshift mutation. In some embodiments, the sequence reframing can result in or effect a frameshift mutation. In some embodiments, the sequence knock-in is an editing where one or more sequences is inserted into a target nucleic acid relative to a target nucleic acid without the sequence knock-in. In some embodiments, the sequence knock-in can result in or effect a splicing disruption or a frameshift mutation. In some embodiments, the sequence knock-in can result in or effect a splicing disruption.

In some embodiments, editing of a target nucleic acid can be locus specific, wherein compositions, systems, and methods described herein can edit a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing of a specific locus can affect any one of a splicing disruption, frameshift (e.g., 1+ or 2+ frameshift), sequence deletion, sequence skipping, sequence reframing, sequence knock-in, or any combination thereof. In some embodiments, editing of a target nucleic acid can be locus specific, modification specific, or both. In some embodiments, editing of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be edited in vitro using a composition described herein and introduced into a cell or organism. Methods of editing a target nucleic acid or modulating the expression of a target nucleic acid may be performed ex vivo. For example, methods may comprise obtaining a cell from a subject, editing a target nucleic acid in the cell with methods described herein, and returning the cell to the subject.

In some embodiments, methods of modifying described herein comprise contacting a target nucleic acid with one or more components, compositions or systems described herein. In some embodiments, the one or more components, compositions or systems described herein comprise at least one of: a) one or more effector proteins, or one or more nucleic acids encoding one or more effector proteins; and b) one or more guide nucleic acids, or one or more nucleic acids encoding one or more guide nucleic acids. In some embodiments, the one or more effector proteins introduce a single-stranded break or a double-stranded break in the target nucleic acid.

In some embodiments, methods of modifying described herein comprise using one or more guide nucleic acids or uses thereof, wherein the methods modify a target nucleic acid at a single location. In some embodiments, the methods comprise contacting an RNP comprising an effector protein and a guide nucleic acid to the target nucleic acid. In some embodiments, the methods introduce a mutation (e.g., point mutations, deletions) in the target nucleic acid relative to a corresponding wildtype nucleotide sequence. In some embodiments, the methods remove or correct a disease-causing mutation in a nucleic acid sequence to produce a corresponding wildtype nucleotide sequence. In some embodiments, the methods remove/correct point mutations, deletions, null mutations, or tissue-specific mutations in a target nucleic acid. In some embodiments, the methods introduce a single stranded cleavage, a nick, a deletion of one or two nucleotides, an insertion of one or two nucleotides, a substitution of one or two nucleotides, an epigenetic modification (e.g., methylation, demethylation, acetylation, or deacetylation), or a combination thereof to the target nucleic acid. In some embodiments, the methods comprise using an effector protein and two guide nucleic acids, wherein two RNPs cleave the target nucleic acid at the same location, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of modifying described herein comprise using one or more guide nucleic acids or uses thereof, wherein the methods modify a target nucleic acid at two different locations. In some embodiments, the methods introduce two cleavage sites in the target nucleic acid, wherein a first cleavage site and a second cleavage site comprise one or more nucleotides therebetween. In some embodiments, the methods cause deletion of the one or more nucleotides. In some embodiments, the deletion restores a wild-type reading frame. In some embodiments, the wild-type reading frame produces at least a partially functional protein. In some embodiments, the deletion causes a non-wild-type reading frame. In some embodiments, a non-wild-type reading frame produces a partially functional protein or non-functional protein. In some embodiments, the at least partially functional protein has at least 50%, at least 60%, at least 70%, at least 80%, at least 90%, at least 100%, at least 110%, at least 120%, at least 130%, at least 140%, at least 150%, at least 180%, at least 200%, at least 300%, at least 400% activity compared to a corresponding wildtype protein. In some embodiments, the methods comprise using an effector protein and two guide nucleic acids, wherein two RNPs cleave the target nucleic acid at different locations, wherein a first RNP comprises the effector protein and a first guide nucleic acid, and wherein a second RNP comprises the effector protein and a second guide nucleic acid. In some embodiments, methods comprising using two effector protein and two guide nucleic acids, wherein both RNPs cleave the target nucleic acid at the same location, wherein a first RNP comprises a first effector protein and a first target nucleic acid, and wherein a second RNP comprises a second effector protein and a second target nucleic acid.

In some embodiments, methods of editing described herein comprise inserting a donor nucleic acid into a cleaved target nucleic acid. In some embodiments, the cleaved target nucleic acid formed by introducing a single-stranded break into a target nucleic acid. The donor nucleic acid may be inserted at a specified (e.g., effector protein targeted) point within the target nucleic acid. In some embodiments, the cleaved target nucleic acid is cleaved at a single location. In such embodiments, the methods comprise contacting a target nucleic acid with an effector protein described herein, thereby introducing a single-stranded break in the target nucleic acid; and contacting the target nucleic acid with a donor nucleic acid for homologous recombination, optionally by HDR or NHEJ, thereby introducing a new sequence into the target nucleic acid (e.g., at a cleavage site). In some embodiments, the cleaved target nucleic acid is cleaved at two locations. In such embodiments, the methods comprise contacting a target nucleic acid with an effector protein described herein, thereby introducing a single-stranded break in the target nucleic acid; contacting the target nucleic acid with a second effector protein described herein, to generate a second cleavage site in the target nucleic acid, ligating the regions flanking the first and second cleavage site, optionally through NHEJ or single-strand annealing, thereby resulting in the excision of a portion of the target nucleic acid between the first and second cleavage sites from the target nucleic acid; and contacting the target nucleic acid with a donor nucleic acid for homologous recombination, optionally by HDR or NHEJ, thereby introducing a new sequence into the target nucleic acid (e.g., in between two cleavage sites).

Provided herein are methods of modifying target nucleic acids or the expression thereof. In some embodiments, methods comprise editing a target nucleic acid. In general, editing refers to modifying the nucleobase sequence of a target nucleic acid. Also provided herein are methods of modulating the expression of a target nucleic acid. Fusion effector proteins and systems described herein may be used for such methods. Methods of editing a target nucleic acid may comprise one or more of cleaving the target nucleic acid, deleting one or more nucleotides of the target nucleic acid, inserting one or more nucleotides into the target nucleic acid, modifying one or more nucleotides of the target nucleic acid. Methods of modulating expression of target nucleic acids may comprise modifying the target nucleic acid or a protein associated with the target nucleic acid, e.g., a histone.

In some embodiments, methods of modifying a target nucleic acid comprise contacting a target nucleic acid with a composition described herein. In some embodiments, methods comprise contacting a target nucleic acid with an effector protein described herein. In some embodiments, methods comprise contacting a target nucleic acid with a fusion effector protein described herein. The effector protein may be an effector protein described herein, including catalytically inactive effector proteins. The effector protein may comprise an amino acid sequence that is at least 70%, at least 75%, at least 80%, at least 85%, at least 90%, at least 95%, at least 97%, at least 98%, at least 99% or 100% identical to a sequence selected from SEQ ID NOS: 1-10,484 or 15,022-24,165. In some embodiments, the amino acid sequence of the effector protein is at least 70%, at least 75%, at least 80%, at least 85%, at least 90%, at least 95%, at least 97%, at least 98%, at least 99% or 100% identical to a sequence selected from SEQ ID NOS: 1-10,484 or 15,022-24,165. In some embodiments, methods comprise contacting a target nucleic acid with an effector protein that is at least 90% identical to an effector protein sequence provide in TABLE 1, and a guide nucleic acid that is at least 90% identical to a corresponding guide nucleic from TABLE 1, wherein corresponding means the effector protein sequence and guide nucleic acid sequence are selected from the same column number (e.g., A1 and B1) and same row.

In some embodiments, methods comprise contacting a target nucleic acid with a donor nucleic acid. In some embodiments, composition described herein comprise a donor nucleic acid. Methods may comprise contacting a target nucleic acid, including but not limited to a cell comprising the target nucleic acid, with such compositions. In some embodiments, the donor nucleic acid is inserted at a site that has been cleaved by a composition disclosed herein. In some embodiments, the donor nucleic acid comprises a sequence that serves as a template in the process of homologous recombination. The sequence may carry one or more nucleobase modifications that are to be introduced into the target nucleic acid. By using this donor nucleic acid as a template, the genetic information, including the modification(s), is copied into the target nucleic acid by way of homologous recombination. In reference to a viral vector, the term donor nucleic acid refers to a sequence of nucleotides that will be or has been introduced into a cell following transfection of the viral vector. The donor nucleic acid may be introduced into the cell by any mechanism of the transfecting viral vector, including, but not limited to, integration into the genome of the cell or introduction of an episomal plasmid or viral genome.

In some embodiments, methods comprise base editing. In some embodiments, base editing comprises contacting a target nucleic acid with a fusion effector protein comprising an effector protein fused to a base editing enzyme, such as a deaminase, thereby changing a nucleobase of the target nucleic acid to an alternative nucleobase. In some embodiments, the nucleobase of the target nucleic acid is adenine (A) and the method comprises changing A to guanine (G). In some embodiments, the nucleobase of the target nucleic acid is cytosine (C) and the method comprises changing C to thymine (T). In some embodiments, the nucleobase of the target nucleic acid is C and the method comprises changing C to G. In some embodiments, the nucleobase of the target nucleic acid is A and the method comprises changing A to G.

In some embodiments, methods introduce a nucleobase change in a target nucleic acid relative to a corresponding wildtype or mutant nucleobase sequence. In some embodiments, methods remove or correct a disease-causing mutation in a nucleic acid sequence, e.g., to produce a corresponding wildtype nucleobase sequence. In some embodiments, methods remove/correct point mutations, deletions, null mutations, or tissue-specific mutations in a target nucleic acid. In some embodiments, methods generate gene knock-out, gene knock-in, gene editing, gene tagging, or a combination thereof. Methods of the disclosure may be targeted to a locus in a genome of a cell.

In some embodiments, methods of editing a target nucleic acid or modulating the expression of a target nucleic acid are performed in vivo. In some embodiments, methods of editing a target nucleic acid or modulating the expression of a target nucleic acid are performed in vitro. For example, a plasmid may be modified in vitro using a composition described herein and introduced into a cell or organism. In some embodiments, methods of editing a target nucleic acid or modulating the expression of a target nucleic acid are performed ex vivo. For example, methods may comprise obtaining a cell from a subject, modifying a target nucleic acid in the cell with methods and compositions described herein, and returning the cell to the subject. Methods of editing performed ex vivo may be particularly advantageous to produce CAR T-cells. In some embodiments, methods comprise editing a target nucleic acid or modulating the expression of the target nucleic acid in a cell or a subject. The cell may be a dividing cell. The cell may be a terminally differentiated cell. In some embodiments, the target nucleic acid is a gene.

Methods of editing a target nucleic acid or modulating the expression of a target nucleic acid described herein may be employed to generate a genetically modified cell. The cell may be a prokaryotic cell. The cell may be an archaeal cell. The cell may be a eukaryotic cell. The cell may be a mammalian cell. The cell may be a human cell. The cell may be a T cell. The cell may be a hematopoietic stem cell. The cell may be a bone marrow derived cell, a white blood cell, a blood cell progenitor, or a combination thereof. Generating a genetically modified cell may comprise contacting a target cell with an effector protein or a fusion effector protein described herein and a guide nucleic acid. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some cases, the nanoparticle delivery comprises lipid nanoparticle delivery or gold nanoparticle delivery. In some cases, the nanoparticle delivery comprises lipid nanoparticle delivery. In some cases, the nanoparticle delivery comprises gold nanoparticle delivery.

Methods may comprise cell line engineering. Generally, cell line engineering comprises modifying a pre-existing cell (e.g., naturally-occurring or engineered) or pre-existing cell line to produce a novel cell line or modified cell line. In some embodiments, modifying the pre-existing cell or cell line comprises contacting the pre-existing cell or cell line with an effector protein or fusion effector protein described herein and a guide nucleic acid. The resulting modified cell line may be useful for production of a protein of interest. Non-limiting examples of cell lines includes: 132-d5 human fetal fibroblasts, 10.1 mouse fibroblasts, 293-T, 3T3, 3T3 Swiss, 3T3-L1, 721, 9L, A-549, A10, A172, A20, A253, A2780, A2780ADR, A2780cis, A375, A431, ALC, ARH-77, B16, B35, BALB/3T3 mouse embryo fibroblast, BC-3, BCP-1 cells, BEAS-2B, BHK-21, BR 293, BS-C-1 monkey kidney epithelial, Bcl-1, bEnd.3, BxPC3, C3H-10T1/2, C6/36, C8161, CCRF-CEM, CHO, CHO Dhfr-/-, CHO-7, CHO-IR, CHO-K1, CHO-K2, CHO-T, CIR, CML T1, CMT, COR-L23, COR-L23/5010, COR-L23/CPR, COR-L23/R23, COS, COS-1, COS-6, COS-7, COS-M6A, COV-434, CT26, CTLL-2, CV1, CaCo2, Cal-27, Calu1, D17, DH82, DLD2, DU145, DuCaP, EL4, EM2, EM3, EMT6/AR1, EMT6/AR10.0, FM3, H1299, H69, HASMC, HB54, HB55, HB56, HCA2, HEK-293, HEKa, HEKn, HL-60, HMEC, HT-29, HUVEC, HeLa, HeLa B, HeLa T4, HeLa-S3, Hep G2, Hepa1c1c7, Huh1, Huh4, Huh7, IC21, J45.01, J82, JY cells, Jurkat, Jurkat, K562 cells, KCL22, KG1, KYO1, Ku812, LNCap, LRMB, MC-38, MCF-10A, MCF-7, MDA-MB-231, MDA-MB-435, MDA-MB-468, MDCK II, MDCK II, MEF, mIMCD-3C8161, MOLT, MONO-MAC 6, MOR/0.2R, MRC5, MTD-1A, Ma-Mel 1-48, MiaPaCell, MyEnd, NALM-1, NCI-H69/CPR, NCI-H69/LX10, NCI-H69/LX20, NCI-H69/LX4, NHDF, NIH-3T3, NRK, NRK-52E, NW-145, OPCN/OPCT cell lines, P388D1, PC-3, PNT-1A/PNT 2, Panc1, Peer, RIN-5F, RMA/RMAS, RPTE, Rat6, Raw264.7, RenCa, SEM-K2, SK-UT, SKOV3, SW480, SW620, Saos-2 cells, Sf-9, SkBr3, T-47D, T2, T24, T84, TF1, THP1 cell line, TIB55, U373, U87, U937, VCaP, Vero cells, WEHI-231, WM39, WT-49, X63, YAC-1, and YAR.

### Donor Nucleic Acids

In some embodiments, a donor nucleic acid comprises a nucleic acid that is incorporated into a target nucleic acid or genome. In some embodiments, a donor nucleic acid comprises a sequence that is derived from a plant, bacteria, fungi, virus, or an animal. In some embodiments, the animal is a non-human animal, such as, by way of non-limiting example, a mouse, rat, hamster, rabbit, pig, bovine, deer, sheep, goat, chicken, cat, dog, ferret, a bird, non-human primate (e.g., marmoset, rhesus monkey). In some embodiments, the non-human animal is a domesticated mammal or an agricultural mammal. In some embodiments, the animal is a human. In some embodiments, the sequence comprises a human wild-type (WT) gene or a portion thereof. In some embodiments, the human WT gene or the portion thereof comprises a nucleotide sequence that is at least 70%, at least 80%, at least 90%, at least 92%, at least 95%, at least 97%, at least 99%, or 100% identical to an equal length portion of the WT sequence of any one of the genes recited in TABLE 3. In some embodiments, the donor nucleic acid is incorporated into an insertion site of a target nucleic acid.

In some embodiments, a donor nucleic acid of any suitable size is integrated into a target nucleic acid or a genome. In some embodiments, the donor nucleic acid integrated into the target nucleic acid or the genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 kilobases in length. In some embodiments, the donor nucleic acid is more than 500 kilobases (kb) in length.

In some embodiments, a viral vector comprising a donor nucleic acid introduces the donor nucleic acid into a cell following transfection. In some embodiments, the donor nucleic acid is introduced into the cell by any mechanism of the transfecting viral vector, including, but not limited to, integration into the genome of the cell or introduction of an episomal plasmid or viral genome.

In some embodiments, an effector protein as described herein facilitates insertion of a donor nucleic acid at a site of cleavage or between two cleavage sites by cleaving (hydrolysis of a phosphodiester bond) of a nucleic acid resulting in a nick or double strand break-nuclease activity.

In some embodiments, a donor nucleic acid serves as a template in the process of homologous recombination, which may carry an alteration that is to be or has been introduced into a target nucleic acid. By using the donor nucleic acid as a template, the genetic information, including the alteration, is copied into the target nucleic acid by way of homologous recombination.

### Genetically Modified Cells and Organisms

Methods of editing described herein may be employed to generate a genetically modified cell. In some embodiments, the cell is a eukaryotic cell (e.g., a mammalian cell) or a prokaryotic cell (e.g., an archaeal cell). In some embodiments, the cell is derived from a multicellular organism and cultured as a unicellular entity. In some embodiments, the cell comprises a heritable genetic modification, such that progeny cells derived therefrom comprise the heritable genetic mutation. In some embodiments, the cell is progeny of a genetically modified cell comprising a genetic modification of the genetically modified parent cell. In some embodiments, the genetically modified cell comprises a deletion, insertion, mutation, or non-native sequence relative to a wild-type version of the cell or the organism from which the cell was derived.

Methods of editing described herein may be performed in a cell. In some embodiments, the cell is in vitro. In some embodiments, the cell is in vivo. In some embodiments, the cell is ex vivo. In some embodiments, the cell is an isolated cell. In some embodiments, the cell is inside of an organism. In some embodiments, the cell is an organism. In some embodiments, the cell is in a cell culture. In some embodiments, the cell is one of a collection of cells. In some embodiments, the cell is a mammalian cell or derived there from. In some embodiments, the cell is a rodent cell or derived there from. In some embodiments, the cell is a human cell or derived there from. In some embodiments, the cell is a eukaryotic cell or derived there from. In some embodiments, the cell is a progenitor cell or derived there from. In some embodiments, the cell is a pluripotent stem cell or derived there from. In some embodiments, the cell is an animal cell or derived there from. In some embodiments, the cell is an invertebrate cell or derived there from. In some embodiments, the cell is a vertebrate cell or derived there from. In some embodiments, the cell is from a specific organ or tissue. In some embodiments, the cell is a hepatocyte. In some embodiments, the tissue is a subject's blood, bone marrow, or cord blood. In some embodiments, the tissue is a heterologous donor blood, cord blood, or bone marrow. In some embodiments, the tissue is an allogenic blood, cord blood, or bone marrow. In some embodiments, the tissue may be muscle. In some embodiments, the muscle may be a skeletal muscle. In some embodiments, skeletal muscles include the following: abductor digiti minimi (foot), abductor digiti minimi (hand), abductor hallucis, abductor pollicis brevis, abductor pollicis longus, adductor brevis, adductor hallucis, adductor longus, adductor magnus, adductor pollicis, anconeus, articularis cubiti, articularis genu, aryepiglotticus, auricularis, biceps brachii, biceps femoris, brachialis, brachioradialis, buccinator, bulbospongiosus, constrictor of pharynx-inferior, constrictor of pharynx-middle, constrictor of pharynx-superior, coracobrachialis, corrugator supercilii, cremaster, cricothyroid, dartos, deep transverse perinei, deltoid, depressor anguli oris, depressor labii inferioris, diaphragm, digastric, digastric (anterior view), erector spinae—spinalis, erector spinae—iliocostalis, erector spinae—longissimus, extensor carpi radialis brevis, extensor carpi radialis longus, extensor carpi ulnaris, extensor digiti minimi (hand), extensor digitorum (hand), extensor digitorum brevis (foot), extensor digitorum longus (foot), extensor hallucis brevis, extensor hallucis longus, extensor indicis, extensor pollicis brevis, extensor pollicis longus, external oblique abdominis, flexor carpi radialis, flexor carpi ulnaris, flexor digiti minimi brevis (foot), flexor digiti minimi brevis (hand), flexor digitorum brevis, flexor digitorum longus (foot), flexor digitorum profundus, flexor digitorum superficialis, flexor hallucis brevis, flexor hallucis longus, flexor pollicis brevis, flexor pollicis longus, frontalis, gastrocnemius, gemellus inferior, gemellus superior, genioglossus, geniohyoid, gluteus maximus, gluteus medius, gluteus minimus, gracilis, hyoglossus, iliacus, inferior oblique, inferior rectus, infraspinatus, intercostals external, intercostals innermost, intercostals internal, internal oblique abdominis, interossei-dorsal of hand, interossei-dorsal of foot, interossei-palmar of hand, interossei—plantar of foot, interspinales, intertransversarii, intrinsic muscles of tongue, ishiocavernosus, lateral cricoarytenoid, lateral pterygoid, lateral rectus, latissimus dorsi, levator anguli oris, levator ani-coccygeus, levator ani-iliococcygeus, levator ani-pubococcygeus, levator ani-puborectalis, levator ani-pubovaginalis, levator labii superioris, levator labii superioris, alaeque nasi, levator palpebrae superioris, levator scapulae, levator veli palatini, levatores costarum, longus capitis, longus colli, lumbricals of foot, lumbricals of hand, masseter, medial pterygoid, medial rectus, mentalis, m. uvulae, mylohyoid, nasalis, oblique arytenoid, obliquus capitis inferior, obliquus capitis superior, obturator externus, obturator internus (A), obturator internus (B), omohyoid, opponens digiti minimi (hand), opponens pollicis, orbicularis oculi, orbicularis oris, palatoglossus, palatopharyngeus, palmaris brevis, palmaris longus, pectineus, pectoralis major, pectoralis minor, peroneus brevis, peroneus longus, peroneus tertius, piriformis (A), piriformis (B), plantaris, platysma, popliteus, posterior cricoarytenoid, procerus, pronator quadratus, pronator teres, psoas major, psoas minor, pyramidalis, quadratus femoris, quadratus lumborum, quadratus plantae, rectus abdominis, rectus capitus anterior, rectus capitus lateralis, rectus capitus posterior major, rectus capitus posterior minor, rectus femoris, rhomboid major, rhomboid minor, risorius, salpingopharyngeus, sartorius, scalenus anterior, scalenus medius, scalenus minimus, scalenus posterior, semimembranosus, semitendinosus, serratus anterior, serratus posterior inferior, serratus posterior superior, soleus, sphincter ani, sphincter urethrae, splenius capitis, splenius cervicis, stapedius, sternocleidomastoideohyoid, sternothyroid, styloglossus, stylohyoid, stylohyoid (anterior view), stylopharyngeus, subclavius, subcostalis, subscapularis, superficial transverse perinei, superior oblique, superior rectus, supinator, supraspinatus, temporalis, temporoparietalis, tensor fasciae lata, tensor tympani, tensor veli palatini, teres major, teres minor, thyro-arytenoid & vocalis, thyro-epiglotticus, thyrohyoid, tibialis anterior, tibialis posterior, transverse arytenoid, transversospinalis-multifidus, transversospinalis-rotatores, transversospinalis-semispinalis, transversus abdominis, transversus thoracis, trapezius, triceps, vastus intermedius, vastus lateralis, vastus medialis, zygomaticus major, or zygomaticus minor. In some embodiments, the cell is a myocyte. In some embodiments, the cell is a muscle cell. In some embodiments, the muscle cell is a skeletal muscle cell. In some embodiments, the skeletal muscle cell is a red (slow) skeletal muscle cell, a white (fast) skeletal muscle cell or an intermediate skeletal muscle cell.

Methods of editing described herein may comprise contacting cells with compositions or systems described herein. In some embodiments, the contacting comprises

Methods of editing described herein may be performed in a subject. In some embodiments, the methods comprise administering compositions described herein to the subject. In some embodiments, the subject is a human. In some embodiments, the subject is a mammal (e.g., rat, mouse, cow, dog, pig, sheep, horse). In some embodiments, the subject is a vertebrate or an invertebrate. In some embodiments, the subject is a laboratory animal. In some embodiments, the subject is a patient. In some embodiments, the subject is at risk of developing, suffering from, or displaying symptoms of a disease. In some embodiments, the subject may have a mutation associated with a gene described herein. In some embodiments, the subject may display symptoms associated with a mutation of a gene described herein.

In some aspects, disclosed herein are modified cells or populations of modified cells, wherein the modified cell comprises an effector protein described herein, a nucleic acid encoding an effector protein described herein, or a combination thereof. In some embodiments, the modified cell comprises a fusion effector protein described herein, a nucleic acid encoding an effector protein described herein, or a combination thereof. In some embodiments, the modified cell is a modified prokaryotic cell. In some embodiments, the modified cell is a modified eukaryotic cell. A modified cell may be a modified fungal cell. In some embodiments, the modified cell is a modified vertebrate cell. In some embodiments, the modified cell is a modified invertebrate cell. In some embodiments, the modified cell is a modified mammalian cell. In some embodiments, the modified cell is a modified human cell. In some embodiments, the modified cell is in a subject. A modified cell may be in vitro. A modified cell may be in vivo. A modified cell may be ex vivo. A modified cell may be a cell in a cell culture. A modified cell may be a cell obtained from a biological fluid, organ or tissue of a subject and modified with a composition and/or method described herein. Non-limiting examples of biological fluids are blood, plasma, serum, and cerebrospinal fluid. Non-limiting examples of tissues and organs are bone marrow, adipose tissue, skeletal muscle, smooth muscle, spleen, thymus, brain, lymph node, adrenal gland, prostate gland, intestine, colon, liver, kidney, pancreas, heart, lung, bladder, ovary, uterus, breast, and testes. Non-limiting examples of cells that may be obtained from a subject are hepatocytes, epithelial cells, endothelial cells, neurons, cardiomyocytes, muscle cells and adipocytes. Non-limiting examples of cells that may be modified with compositions and methods described herein include immune cells, such as CAR T-cells, T-cells, B-cells, NK cells, granulocytes, basophils, eosinophils, neutrophils, mast cells, monocytes, macrophages, dendritic cells, microglia, Kuppfer cells, antigen-presenting cells (APC), or adaptive cells.

Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells. A cell may be a pluripotent cell.

Non-limiting examples of cells that may be engineered or modified with compositions and methods described herein include include plant cells, such as parenchyma, sclerenchyma, collenchyma, xylem, phloem, germline (e.g., pollen). Cells from lycophytes, ferns, gymnosperms, angiosperms, bryophytes, charophytes, chlorophytes, rhodophytes, or glaucophytes.

### XIII. Methods of Detecting a Target Nucleic Acid

Provided herein are methods of detecting target nucleic acids. In some embodiments, the methods comprise detecting a target nucleic acid with compositions or systems described herein. In some embodiments, the methods of detecting a target nucleic acid comprising: a) contacting the target nucleic acid with a composition comprising an effector protein as described herein, a guide nucleic acid as described herein, and a reporter nucleic acid that is cleaved in the presence of the effector protein, the guide nucleic acid, and the target nucleic acid; and b) detecting a signal produced by cleavage of the reporter nucleic acid, thereby detecting the target nucleic acid in the sample. In some embodiments, the methods result in cis cleavage of the reporter nucleic acid. In some embodiments, the reporter nucleic acid is a single stranded nucleic acid. In some embodiments, the reporter comprises a detection moiety. In some embodiments, the reporter nucleic acid is capable of being cleaved by the effector protein. In some embodiments, a cleaved reporter nucleic acid generates a first detectable signal. In some embodiments, the first detectable signal is a change in color. In some embodiments, the change is color is measured indicating presence of the target nucleic acid. In some embodiments, the first detectable signal is measured on a support medium.

In some embodiments, methods of detecting comprise contacting a target nucleic acid, a cell comprising the target nucleic acid, or a sample comprising a target nucleic acid with an effector protein that comprises an amino acid sequence that is at least is at least 70%, at least 75%, at least 80%, at least 85%, at least 90%, at least 92%, at least 95%, at least 97%, at least 98%, at least 99%, or 100% identical to any one of SEQ ID NOS: 1-10,484 or 15,022-24, 165. In some embodiments, the amino acid sequence of the effector protein is at least 70%, at least 75%, at least 80%, at least 85%, at least 90%, at least 92%, at least 95%, at least 97%, at least 98%, at least 99%, or 100% identical to any one of SEQ ID NOS: 1-10,484 or 15,022-24,165.

In some embodiments, the methods comprise contacting the sample to a composition as described herein; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample.

In some embodiments, methods comprise contacting the sample comprising the target nucleic acid with a guide nucleic acid targeting a target nucleic acid segment, an effector protein capable of being activated when complexed with the guide nucleic acid and the target nucleic acid segment, a single stranded nucleic acid of a reporter comprising a detection moiety, wherein the nucleic acid of a reporter is capable of being cleaved by the activated effector protein, thereby generating a first detectable signal, cleaving the single stranded nucleic acid of a reporter using the effector protein that cleaves as measured by a change in color, and measuring the first detectable signal on the support medium.

Methods may comprise contacting a sample or a cell with a composition described herein at a temperature of at least about 25° C., at least about 30° C., at least about 35° C., at least about 40° C., at least about 50° C., or at least about 65° C. In some embodiments, the temperature is not greater than 80° C. In some embodiments, the temperature is about 25° C., about 30° C., about 35° C., about 40° C., about 45° C., about 50° C., about 55° C., about 60° C., about 65° C., or about 70° C. In some embodiments, the temperature is about 25° C. to about 45° C., about 35° C. to about 55° C., or about 55° C. to about 65° C.

In some embodiments, methods of detecting a target nucleic acid are by a cleavage assay. In some embodiments, the target nucleic acid is a single-stranded target nucleic acid. In some embodiments, the cleavage assay comprises: a) contacting the target nucleic acid with a composition comprising an effector protein as described; and b) cleaving the target nucleic acid. In some embodiments, the cleavage assay comprises an assay designed to visualize, quantitate or identify cleavage of a nucleic acid. In some embodiments, the method is an in vitro trans-cleavage assay. In some embodiments, a cleavage activity is a trans-cleavage activity. In some embodiments, the method is an in vitro cis-cleavage assay. In some embodiments, a cleavage activity is a cis-cleavage activity. In some embodiments, the cleavage assay follows a procedure comprising: (i) providing a composition comprising an equimolar amounts of an effector protein as described herein, and a guide nucleic acid described herein, under conditions to form an RNP complex; (ii) adding a plasmid comprising a target nucleic acid, wherein the target nucleic acid is a linear dsDNA, wherein the target nucleic acid comprises a target sequence and a PAM (iii) incubating the mixture under conditions to enable cleavage of the plasmid; (iv) quenching the reaction with EDTA and a protease; and (v) analyzing the reaction products (e.g., viewing the cleaved and uncleaved linear dsDNA with gel electrophoresis).

In some embodiments, methods are not capable of detecting target nucleic acids that are present in a sample or solution at a concentration less than or equal to 10 nM. The term “threshold of detection” is used herein to describe the minimal amount of target nucleic acid that must be present in the sample in order for detection to occur. For example, in some embodiments, when a threshold of detection is 10 nM, then a signal can be detected when a target nucleic acid is present in the sample at a concentration of 10 nM or more. In such embodiments, the methods are not capable of detecting target nucleic acids that are present in a sample at a concentration less than 10 nM. In some embodiments, the threshold is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some embodiments, the threshold is in a range of from 1 aM to 1 nM, 1 aM to 500 pM, 1 aM to 200 pM, 1 aM to 100 pM, 1 aM to 10 PM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 pM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, 1 pM to 200 pM, 1 pM to 100 pM, or 1 pM to 10 pM. In some embodiments, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some embodiments, the threshold is in a range of from 2 aM to 100 pM, from 20 aM to 50 pM, from 50 aM to 20 pM, from 200 aM to 5 PM, or from 500 aM to 2 pM.

In some embodiments, a minimum concentration at which the methods detect a target nucleic acid a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some embodiments, a minimum concentration at which the methods detect in a sample is in a range of from 2 aM to 100 pM, from 20 aM to 50 pM, from 50 aM to 20 pM, from 200 aM to 5 PM, or from 500 aM to 2 pM. In some embodiments, a minimum concentration at which the methods detect a single stranded target nucleic acid in a sample is in a range of from 1 aM to 100 pM. In some embodiments, a minimum concentration at which the methods detect a target nucleic acid in a sample is in a range of from 1 fM to 100 pM. In some embodiments, a minimum concentration at which the methods detect a single stranded target nucleic acid in a sample is in a range of from 10 fM to 100 pM.

In some embodiments, a minimum concentration at which the methods detect a single stranded target nucleic acid in a sample is in a range of from 800 fM to 100 pM. In some embodiments, a minimum concentration at which the methods detect a single stranded target nucleic acid in a sample is in a range of from 1 pM to 10 pM. In some embodiments, the devices, systems, fluidic devices, kits, and methods described herein detect a single stranded target nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some embodiments, a minimum concentration at which the methods detect a target nucleic acid at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, a minimum concentration at which the methods detect a target nucleic acid at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 pM, from 1 μM to 10 μM, from 10 μM to 100 pM, from 10 nM to 100 nM, from 10 nM to 1 μM, from 10 nM to 10 UM, from 10 nM to 100 μM, from 100 nM to 1 μM, from 100 nM to 10 pM, from 100 nM to 100 μM, or from 1 μM to 100 μM. In some embodiments, a minimum concentration at which the methods detect a target nucleic acid at a concentration of from 20 nM to 5 μM, from 50 nM to 20 μM, or from 200 nM to 5 μM.

In some embodiments, methods detect a target nucleic acid in less than 60 minutes. In some embodiments, methods detect a target nucleic acid in less than about 120 minutes, less than about 110 minutes, less than about 100 minutes, less than about 90 minutes, less than about 80 minutes, less than about 70 minutes, less than about 60 minutes, less than about 55 minutes, less than about 50 minutes, less than about 45 minutes, less than about 40 minutes, less than about 35 minutes, less than about 30 minutes, less than about 25 minutes, less than about 20 minutes, less than about 15 minutes, less than about 10 minutes, less than about 5 minutes, less than about 4 minutes, less than about 3 minutes, less than about 2 minutes, or less than about 1 minute.

In some embodiments, methods require at least about 120 minutes, at least about 110 minutes, at least about 100 minutes, at least about 90 minutes, at least about 80 minutes, at least about 70 minutes, at least about 60 minutes, at least about 55 minutes, at least about 50 minutes, at least about 45 minutes, at least about 40 minutes, at least about 35 minutes, at least about 30 minutes, at least about 25 minutes, at least about 20 minutes, at least about 15 minutes, at least about 10 minutes, or at least about 5 minutes to detect a target nucleic acid. In some embodiments, the sample is contacted with the reagents for from 5 minutes to 120 minutes, from 5 minutes to 100 minutes, from 10 minutes to 90 minutes, from 15 minutes to 45 minutes, or from 20 minutes to 35 minutes.

In some embodiments, methods of detecting are performed in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some embodiments, methods of detecting are performed in about 5 minutes to about 10 hours, about 10 minutes to about 8 hours, about 15 minutes to about 6 hours, about 20 minutes to about 5 hours, about 30 minutes to about 2 hours, or about 45 minutes to about 1 hour.

In some embodiments, methods comprise detection of a detectable signal. In some embodiments, the detection occurs within 5 minutes of contacting a sample and/or a target nucleic acid with a composition described herein. In some embodiments, the detection occurs within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the target nucleic acid. In some embodiments, the detection occurs within 1 to 120, 5 to 100, 10 to 90, 15 to 80, 20 to 60, or 30 to 45 minutes of contacting the target nucleic acid.

### Amplification

In some embodiments, methods of detecting comprise amplifying a target nucleic acid for detection using any of the compositions or systems described herein. Amplifying may comprise changing the temperature of the amplification reaction, also known as thermal amplification (e.g., PCR). Amplifying may be performed at essentially one temperature, also known as isothermal amplification. Amplifying may improve at least one of sensitivity, specificity, or accuracy of the detection of the target nucleic acid.

In some embodiments, amplifying comprises subjecting a target nucleic acid to an amplification reaction selected from transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

In some embodiments, amplification of the target nucleic acid comprises modifying the sequence of the target nucleic acid. For example, in some embodiments, the methods are used for inserting a PAM sequence into a target nucleic acid that lacks a PAM sequence. In some embodiments, the methods are used for increasing the homogeneity of a target nucleic acid in a sample. For example, in some embodiments, the methods are used for removing a nucleic acid variation that is not of interest in the target nucleic acid.

In some embodiments, methods of amplifying a nucleic acid takes 1, 2, 3, 4, 5, 6, 7, 8, 9, 10, 11, 12, 13, 14, 15, 16, 17, 18, 19, 20, 25, 30, 40, 50, or 60 minutes. In some embodiments, the methods performed at a temperature of around 20-45° C. In some embodiments, the methods are performed at a temperature of less than about 20° C., less than about 25° C., less than about 30° C., less than about 35° C., less than about 37° C., less than about 40° C., or less than about 45° C. In some embodiments, the methods are performed at a temperature of at least about 20° C., at least about 25° C., at least about 30° C., at least about 35° C., at least about 37° C., at least about 40° C., or at least about 45° C.

### XIV. Methods of Treating a Disorder

Described herein are methods for treating a disease in a subject by editing a target nucleic acid associated with a gene or expression of a gene related to the disease. In some embodiments, the methods comprise methods of editing nucleic acid described herein.

In some embodiments, methods for treating a disease in a subject comprises administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, editing, modifying, replacing, transposing, or affecting the regulation of a genomic sequence of a patient in need thereof. In some embodiments, the methods of treating, preventing, or inhibiting a disease or disorder may involve modulating gene expression.

Described herein are compositions and methods for treating a disease in a subject by editing a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions and methods described herein may be used to treat, prevent, or inhibit a disease or syndrome in a subject. In some embodiments, the disease is a genetic disease. The term “genetic disease” refers to a disease, disorder, condition, or syndrome associated with or caused by one or more mutations in the DNA of an organism having genetic disease. In some embodiments, the disease is a liver disease, a lung disease, an eye disease, or a muscle disease. Exemplary diseases and syndromes include but are not limited to the diseases and syndromes listed in TABLE 4.

TABLE 4
EXEMPLARY DISEASES
Exemplary Diseases and Syndromes

11-hydroxylase deficiency; 17,20-desmolase deficiency; 17-hydroxylase deficiency; 3-
hydroxyisobutyrate aciduria; 3-hydroxysteroid dehydrogenase deficiency; 46,XY gonadal dysgenesis;
AAA syndrome; ABCA3 deficiency; ABCC8-associated hyperinsulinism; aceruloplasminemia;
acromegaly; achondrogenesis type 2; acral peeling skin syndrome; acrodermatitis enteropathica;
adrenocortical micronodular hyperplasia; adrenoleukodystrophies; adrenomyeloneuropathies; Aicardi-
Goutieres syndrome; Alagille disease (also called Alagille Syndrome); Alexander Disease; Alpers
syndrome; alpha-1 antitrypsin deficiency (AATD); alpha-mannosidosis; Alstrom syndrome;
Alzheimer's disease; amebic dysentery; amelogenesis imperfecta; amish type microcephaly;
amyotrophic lateral sclerosis (ALS); anaplastic large cell lymphoma; anauxetic dysplasia; androgen
insensitivity syndrome; angiopathic thrombosis; antiphospholipid syndrome; Antley-Bixler syndrome;
APECED; Apert syndrome; aplasia of lacrimal and salivary glands; arginase-1 deficiency;
argininosuccinic aciduria; argininemia; arrhythmogenic right ventricular dysplasia; Arts syndrome;
ARVD2; arylsulfatase deficiency type metachromatic leukodystrophy; ataxia telangiectasia;
atherosclerotic cardiovascular disease; autoimmune lymphoproliferative syndrome; autoimmune
polyglandular syndrome type 1; autosomal dominant anhidrotic ectodermal dysplasia; autosomal
dominant deafness; autosomal dominant polycystic kidney disease; autosomal recessive microtia;
autosomal recessive renal glucosuria; autosomal visceral heterotaxy; babesiosis; balantidial dysentery;
Bardet-Biedl syndrome; Bartter syndrome; basal cell nevus syndrome; Batten disease; benign recurrent
intrahepatic cholestasis; beta-mannosidosis; β-thalassemia; Bethlem myopathy; Blackfan-Diamond
anemia; bleeding disorder (coagulation); blepharophimosis; Byler disease; C syndrome; CADASIL;
calcific aortic stenosis; calcification of joints and arteries; carbamoyl phosphate synthetase I deficiency;
cardiofaciocutaneous syndrome; Carney triad; carnitine palmitoyltransferase deficiencies; cartilage-hair
hypoplasia; cblC type of combined methylmalonic aciduria; CD18 deficiency; CD3Z-associated
primary T-cell immunodeficiency; CD40L deficiency; CDAGS syndrome; CDG1A; CDG1B;
CDG1M; CDG2C; CEDNIK syndrome; central core disease; centronuclear myopathy; cerebral
capillary malformation; cerebrooculofacioskeletal syndrome type 4; cerebrooculogacioskeletal
syndrome; cerebrotendinous xanthomatosis; Chaga's Disease; Charcot Marie Tooth Disesase;
cherubism; CHILD syndrome; chronic granulomatous disease; chronic recurrent multifocal
osteomyelitis; cirrhosis; citrin deficiency; citrullinemia type I; citrullinemia type II; classic
hemochromatosis; CNPPB syndrome; cobalamin C disease; Cockayne syndrome; coenzyme Q10
deficiency; Coffin-Lowry syndrome; Cohen syndrome; combined deficiency of coagulation factors V;
common variable immune deficiency 3; complement hyperactivation; complete androgen insentivity;
cone rod dystrophies; conformational diseases; congenital adrenal hyperplasia; congenital bile adid
synthesis defect type 1; congenital bile adid synthesis defect type 2; congenital defect in bile acid
synthesis type; congenital erythropoietic porphyria; congenital generalized osteosclerosis; Congenital
muscular dystrophy; Cornelia de Lange syndrome; coronary heart disease; Cousin syndrome; Cowden
disease; COX deficiency; Cri du chat syndrome; Crigler-Najjar disease; Crigler-Najjar syndrome type
1; Crisponi syndrome; Crouzon syndrome; Currarino syndrome; Curth-Macklin type ichthyosis hystrix;
cutis laxa; cystic fibrosis; cystinosis; d-2-hydroxyglutaric aciduria; DDP syndrome; Dejerine-Sottas
disease; Denys-Drash syndrome; Dercum disease; desmin cardiomyopathy; desmin myopathy;
DGUOK-associated mitochondrial DNA depletion; diabetes Type I; diabetes Type II; disorders of
glutamate metabolism; distal spinal muscular atrophy type 5; DNA repair diseases; dominant optic
atrophy; Doyne honeycomb retinal dystrophy; Dravet Syndrome; Duchenne muscular dystrophy;
dyskeratosis congenita; Ehlers-Danlos syndrome type 4; Ehlers-Danlos syndromes; Elejalde disease;
Ellis-van Creveld disease; Emery-Dreifuss muscular dystrophies; encephalomyopathic mtDNA
depletion syndrome; encephalitis; enzymatic diseases; EPCAM-associated congenital tufting
enteropathy; epidermolysis bullosa with pyloric atresia; epilepsy; fabry disease; facioscapulohumeral
muscular dystrophy; Factor V Leiden thrombophilia; Faisalabad histiocytosis; familial atypical
mycobacteriosis; familial capillary malformation-arteriovenous; Familial Creutzfeld-Jakob disease;
familial esophageal achalasia; familial glomuvenous malformation; familial hemophagocytic
lymphohistiocytosis; familial mediterranean fever; familial megacalyces; familial schwannomatosis;
familial spina bifida; familial splenic asplenia/hypoplasia; familial thrombotic thrombocytopeni
purpura; Fanconi disease (Fanconi anemia); Feingold syndrome; FENIB; fibrodysplasia ossificans
progressiva; FKTN; Fragile X syndrome; Francois-Neetens fleck corneal dystrophy; Frasier syndrome;
Friedreich's ataxia; FTDP-17; Fuchs corneal dystrophy; fucosidosis; G6PD deficiency;
galactosialidosis; Galloway syndrome; Gardner syndrome; Gaucher disease; Gitelman syndrome;
GLUT1 deficiency; GM2-Gangliosidoses (e.g., Tay Sachs Disease, Sandhoff Disease) glycogen
storage disease type 1b; glycogen storage disease type 2; glycogen storage disease type 3; glycogen
storage disease type 4; glycogen storage disease type 9a; glycogen storage diseases; GM1-
gangliosidosis; Greenberg syndrome; Greig cephalopolysyndactyly syndrome; hair genetic diseases;
hairy cell leukemia; HANAC syndrome; harlequin type ichtyosis congenita; HDR syndrome; hearing
loss; hemochromatosis type 3; hemochromatosis type 4; hemolytic anemia; hemolytic uremic
syndrome; hemophilia A; hemophilia B; hereditary angioedema type 3; hereditary angioedemas;
hereditary hemorrhagic telangiectasia; hereditary hypofibrinogenemia; hereditary intraosseous vascular
malformation; hereditary leiomyomatosis and renal cell cancer; hereditary neuralgic amyotrophy;
hereditary sensory and autonomic neuropathy type; Hermansky-Pudlak disease; HHH syndrome;
HHT2; hidrotic ectodermal dysplasia type 1; hidrotic ectodermal dysplasias; histiocytic sarcoma;
HNF4A-associated hyperinsulinism; HNPCC; homozygous familial hypercholesterolemia; human
immunodeficiency with microcephaly; Human monkeypox (MPX); human papilloma virus (HPV)
infection; Huntington's disease; hyper-IgD syndrome; hyperinsulinism-hyperammonemia syndrome;
hypercholesterolemia; hypertrophy of the retinal pigment epithelium; hypochondrogenesis;
hypohidrotic ectodermal dysplasia; ICF syndrome; idiopathic congenital intestinal pseudo-obstruction;
immunodeficiency 13; immunodeficiency 17; immunodeficiency 25; immunodeficiency with hyper-
IgM type 1; immunodeficiency with hyper-IgM type 3; immunodeficiency with hyper-IgM type 4;
immunodeficiency with hyper-IgM type 5; immunoglobulin alpha deficiency; inborn errors of thyroid
metabolism; infantile myofibromatosis; infantile visceral myopathy; infantile X-linked spinal muscular
atrophy; intrahepatic cholestasis of pregnancy; IPEX syndrome; IRAK4 deficiency; isolated congenital
asplenia; Jeune syndrome; Johanson-Blizzard syndrome; Joubert syndrome; JP-HHT syndrome;
juvenile hemochromatosis; juvenile hyalin fibromatosis; juvenile nephronophthisis; Kabuki mask
syndrome; Kallmann syndromes; Kartagener syndrome; KCNJ11-associated hyperinsulinism; Kearns-
Sayre syndrome; Kostmann disease; Kozlowski type of spondylometaphyseal dysplasia; Krabbe
disease; LADD syndrome; late infantile-onset neuronal ceroid lipofuscinosis; LCK deficiency; LDHCP
syndrome; Leber Congenital Amaurosis Teyp 10; Legius syndrome; Leigh syndrome; lethal congenital
contracture syndrome 2; lethal congenital contracture syndromes; lethal contractural syndrome type 3;
lethal neonatal CPT deficiency type 2; lethal osteosclerotic bone dysplasia; leukocyte adhesion
deficiency; Li Fraumeni syndrome; LIG4 syndrome; Limb Girdle Muscular Dystrophies;
lipodystrophy; lissencephaly type 1; lissencephaly type 3; Loeys-Dietz syndrome; low phospholipid-
associated cholelithiasis; Lynch Syndrome; lysinuric protein intolerance; a lysosomal storage disease
(e.g., Hunter syndrome, Hurler syndrome); macular dystrophy; Maffucci syndrome; Majeed syndrome;
mannose-binding protein deficiency; mantle cell lymphoma; Marfan disease; Marshall syndrome;
MASA syndrome; mastocytosis; MCAD deficiency; McCune-Albright syndrome; MCKD2; Meckel
syndrome; MECP2 Duplication Syndrome; Meesmann corneal dystrophy; megacystis-microcolon-
intestinal hypoperistalsis; megaloblastic anemia type 1; MEHMO; MELAS; Melnick-Needles
syndrome; MEN2s; meningitis; Menkes disease; metachromatic leukodystrophies; methymalonic
acidemia due to transcobalamin receptor defect; methylmalonic acidurias; methylvalonic aciduria;
microcoria-congenital nephrosis syndrome; microvillous atrophy; migraine; mitochondrial
neurogastrointestinal encephalomyopathy; monilethrix; monosomy X; mosaic trisomy 9 syndrome;
Mowat-Wilson syndrome; mucolipidosis type 2; mucolipidosis type Ma; mucolipidosis type IV;
mucopolysaccharidoses; mucopolysaccharidosis type 3A; mucopolysaccharidosis type 3C;
mucopolysaccharidosis type 4B; multiminicore disease; multiple acyl-CoA dehydrogenation
deficiency; multiple cutaneous and mucosal venous malformations; multiple endocrine neoplasia type
1; multiple sulfatase deficiency; mycosis fungoides; myotonic dystrophy; NAIC; nail-patella
syndrome; nemaline myopathies; neonatal diabetes mellitus; neonatal surfactant deficiency;
nephronophtisis; Netherton disease; neurofibromatoses; neurofibromatosis type 1; Niemann-Pick
disease type A; Niemann-Pick disease type B; Niemann-Pick disease type C; NKX2E; non-alcoholic
fatty liver disease (NAFLD); non-alcoholic steatohepatitis (NASH); Noonan syndrome; North
American Indian childhood cirrhosis; NROB1 duplication-associated DSD; ocular genetic diseases;
oculo-auricular syndrome; OLEDAID; oligomeganephronia; oligomeganephronic renal hypolasia;
Ollier disease; Opitz-Kaveggia syndrome; orofaciodigital syndrome type 1; orofaciodigital syndrome
type 2; osseous Paget disease; osteogenesis imperfecta; otopalatodigital syndrome type 2; OXPHOS
diseases; palmoplantar hyperkeratosis; panlobar nephroblastomatosis; Parkes-Weber syndrome;
Parkinson's disease; partial deletion of 21q22.2-q22.3; Pearson syndrome; Pelizaeus-Merzbacher
disease; Pendred syndrome; pentalogy of Cantrell; peroxisomal acyl-CoA-oxidase deficiency; Peutz-
Jeghers syndrome; Pfeiffer syndrome; Pierson syndrome; pigmented nodular adrenocortical disease;
pipecolic acidemia; Pitt-Hopkins syndrome; plasmalogens deficiency; platelet glycoprotein IV
deficiency; pleuropulmonary blastoma and cystic nephroma; polycystic kidney disease; polycystic
ovarian disease; polycystic lipomembranous osteodysplasia; Pompe disease; including infantile onset
Pompe disease (IOPD) and late onset Pompe disease (LOPD); porphyrias; PRKAG2 cardiac syndrome;
premature ovarian failure; primary erythermalgia; primary hemochromatoses; primary hyperoxaluria;
progressive familial intrahepatic cholestasis; propionic acidemia; protein-losing enteropathy; pyruvate
decarboxylase deficiency; RAPADILINO syndrome; renal cystinosis; retinitis pigmentosa; Rett
Syndrome; rhabdoid tumor predisposition syndrome; Rieger syndrome; ring chromosome 4; Roberts
syndrome; Robinow-Sorauf syndrome; Rothmund-Thomson syndrome; severe combined
immunodeficiency disorder (SCID); Saethre-Chotzen syndrome; Sandhoff disease; SC phocomelia
syndrome; SCAS; Schinzel phocomelia syndrome; severe hypertriglyceridemia; short rib-polydactyly
syndrome type 1; short rib-polydactyly syndrome type 4; short-rib polydactyly syndrome type 2; short-
rib polydactyly syndrome type 3; Shwachman disease; Shwachman-Diamond disease; sickle cell
anemia; Silver-Russell syndrome; Simpson-Golabi-Behmel syndrome; Smith-Lemli-Opitz syndrome;
SPG7-associated hereditary spastic paraplegia; spherocytosis; spinocerebellar ataxia; spinal muscular
atrophy; split-hand/foot malformation with long bone deficiencies; spondylocostal dysostosis;
sporadic visceral myopathy with inclusion bodies; storage diseases; Stargardt macular dystrophy;
STRA6-associated syndrome; stroke; Tay-Sachs disease; thanatophoric dysplasia; thrombophilia due
to antithrombin III deficiency; thyroid metabolism diseases; Tourette syndrome; transcarbamylase
deficiency; transthyretin-associated amyloidosis; trisomy 13; trisomy 22; trisomy 2p syndrome;
tuberous sclerosis; tufting enteropathy; Ullrich Congenital Muscular Dystrophy; urea cycle diseases;
Usher Syndrome; Van Den Ende-Gupta syndrome; Van der Woude syndrome; variegated mosaic
aneuploidy syndrome; VLCAD deficiency; von Hippel-Lindau disease; von Willebrand disease;
Waardenburg syndrome; WAGR syndrome; Walker-Warburg syndrome; Werner syndrome; Wilson
disease; Wiskott-Aldrich Syndrome; Wolcott-Rallison syndrome; Wolfram syndrome; X-linked
agammaglobulinemia; X-linked chronic idiopathic intestinal pseudo-obstruction; X-linked cleft palate
with ankyloglossia; X-linked dominant chondrodysplasia punctata; X-linked ectodermal dysplasia; X-
linked Emery-Dreifuss muscular dystrophy; X-linked lissencephaly; X-linked lymphoproliferative
disease; X-linked visceral heterotaxy; xanthinuria type 1; xanthinuria type 2; xeroderma pigmentosum;
XPV; and Zellweger disease.

In some embodiments, compositions and methods edit at least one gene associated with a disease described herein or the expression thereof. In some embodiments, the disease is Alzheimer's disease and the gene is selected from APP, BACE-1, PSD95, MAPT, PSEN1, PSEN2, and APOE&4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C9ORF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCHII, APOA1, APOL1, ARH, CDKN2B, CFB, CXCL12, FXI, FXII, GATA-4, MIA3, MKL2, MTHFD1L, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSD17B13, PSD3, GPAM, CIDEB, DGAT2 and PNPLA3. In some embodiments, the disease is NASH/cirrhosis and the gene is MARCI. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASS1. In some embodiments, the disease is citrullinemia type I and the gene is SLC25A13. In some embodiments, the disease is citrullinemia type I and the gene is ASS1. In some embodiments, the disease is arginase-1 deficiency and the gene is ARG1. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD. LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHD1. In some embodiments, the disease comprises Fanconi anemia and the gene is selected from FANCA, FANCB, FANCC, FANCD1, FANCD2, FANCE, FANCF. FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2. MLH1. MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E. ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNA1A, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USHIC. CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, compositions, systems or methods described herein edit at least one gene associated with a cancer or the expression thereof. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; anal cancer; appendix cancer; astrocytoma; atypical teratoid/rhabdoid tumor; basal-cell carcinoma; bile duct cancer; bladder cancer; bone osteosarcoma; brain cancer; brain tumor; brainstem glioma; breast cancer; bronchial adenoma, carcinoid, or tumor; Burkitt lymphoma; carcinomacervical cancer; chronic lymphocytic leukemia; chronic myelogenous leukemia; chronic myeloid leukemia; colon cancer; colorectal cancer; emphysema; endometrial cancer; esophageal cancer; Ewing sarcoma; gallbladder cancer; gastric (stomach) cancer; gastrointestinal tumor; gliomahairy cell leukemia; head and neck cancer; liver cancer; Hodgkin's lymphoma; hypopharyngeal cancer; Kaposi Sarcoma; kidney cancer lip and oral cavity cancer; liposarcoma; lung cancer, non-small cell lung cancer; Waldenström; melanoma; mesotheliomamyelogenous leukemia; myeloid leukemia; myeloma; nasopharyngeal carcinoma; neuroblastoma; non-Hodgkin's lymphoma; ovarian cancer; pancreatic cancer; pineal cancer; pituitary tumor; prostate cancer; rectal cancer; renal cell carcinomaretinoblastoma; spinal cord tumor; squamous cell carcinoma; squamous neck cancer; T-cell lymphoma, cutaneous (Mycosis Fungoides and Sézary syndrome); testicular cancer; throat cancer; thyroid cancer; urethral cancer; uterine cancervaginal cancer; and Wilms Tumor. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CML), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, compositions, systems or methods described herein edit at least one mutation in a target nucleic acid, wherein the at least one mutation is associated with cancer or causative of cancer. In some embodiments, the target nucleic acid comprises a gene associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, combinations thereof, or portions thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAKI, JUN, KIT, KS3, K-SAM, LBC, LCK, LMO1, LMO2, L-MYC, LYL-1, LYT-10, LYT-10/Cal, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1, Non-limiting examples of oncogenes are KRAS, NRAS, BRAF, MYC, CTNNB1, and EGFR, In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN.

### Infections

In some embodiments, compositions, systems or methods described herein treats an infection in a subject. In some embodiments, the infections are caused by a pathogen (e.g., bacteria, viruses, fungi, and parasites). In some embodiments, compositions, systems or methods described herein modifies a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions, systems or methods described herein treats an infection caused by one or more viral pathogens. Non-limiting examples of viral pathogens include adenovirus, blue tongue virus, chikungunya, coronavirus (e.g., SARS-COV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein treats an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### EXAMPLES

The following examples are included for illustrative purposes only and are not intended to limit the scope of the invention.

### Example 1. PAM Screening for Effector Proteins

Effector proteins and guide RNA combinations described herein are screened by an in vitro enrichment (IVE) assay to determine PAM recognition by each effector protein-guide RNA complex. Briefly, effector proteins are complexed with corresponding guide RNAs for 15 minutes at 37° C. The complexes are added to an IVE reaction mix. PAM screening reactions use 10 μl of RNP in 100 μl reactions with 1,000 ng of a 5′ PAM library in 1× Cutsmart buffer and are carried out for 15 minutes at 25° C., 45 minutes at 37° C., and 15 minutes at 45° C. Reactions are terminated with 1 μl of proteinase K and 5 μl of 500 mM EDTA for 30 minutes at 37° C. Next generation sequencing is performed on cut sequences to identify enriched PAMs.

### Example 2. Effector Proteins Edit Genomic DNA in Mammalian Cells

Effector proteins are tested for their ability to produce indels in a mammalian cell line (e.g., HEK293T cells). Briefly, a plasmid encoding the effector proteins and a guide RNA are delivered by lipofection to the mammalian cells. This is performed with a variety of guide RNAs targeting several loci adjacent to biochemically determined PAM sequences. Indels in the loci are detected by next generation sequencing of PCR amplicons at the targeted loci and indel percentage is calculated as the fraction of sequencing reads containing insertions or deletions relative to an unedited reference sequence. “No plasmid” and Cas9 are included as negative and positive controls, respectively.

### Example 3. Base Editing

A nucleic acid vector encoding a fusion protein is constructed for base editing. The fusion protein comprises a catalytically inactive variant of an effector protein fused to a deaminase. The fusion protein and at least one guide nucleic acid is tested for its ability to edit a target sequence in eukaryotic cells. Cells are transfected with the nucleic acid vector and guide nucleic acid. After sufficient incubation, DNA is extracted from the transfected cells. Target sequences are PCR amplified and sequenced by NGS and MiSeq. The presence of base modifications are analyzed from sequencing data. Results are recorded as a change in % base call relative to the negative control.

### Example 4. Activation of Gene Expression with Cas Effector Fusion Polypeptide

A single stranded reporter nucleic acid encoding a fluorescent protein (e.g., enhanced green fluorescent protein (EGFP)) and a eukaryotic promoter is generated with a target sequence that is known to be recognized by complexes of effector proteins disclosed herein and corresponding guide nucleic acids. A nucleic acid vector encoding the Cas effector fused to a transcriptional activator; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 5. Reduction of Gene Expression with with Cas Effector Fusion Polypeptide

A single stranded reporter nucleic acid encoding a fluorescent protein (e.g., enhanced green fluorescent protein (EGFP)) and a pSV40 promoter that drives constitutive expression of EGFP is generated with a target sequence that is known to be recognized by complexes of effector proteins disclosed herein and corresponding guide nucleic acids. A nucleic acid vector encoding the Cas effector fused to a transcriptional repressor; a guide nucleic acid; and the single stranded reporter nucleic acid encoding EGFP are introduced to eukaryotic cells via lipofection and EGFP expression is quantified by flow cytometry. Relative amounts of RNA, indicative of relative gene expression, are quantified with RT-qPCR.

### Example 6. Generating a Catalytically Inactive Variant of a CRISPR Cas Effector Protein

Extensive work has been done to evaluate the overall domain structure of the CRISPR Cas enzymes in the last decade. These data can be an effective reference when trying to identify a catalytic residue of a Cas nuclease. By selecting the residue of a Cas nuclease of interest that aligns at the same relative location as the catalytic residue of a known nuclease when the Cas nuclease and known nuclease are aligned for maximal sequence identity, one can identify the catalytic residue of the Cas nuclease.

Sequence or structural analogs of a Cas nuclease provide an additional or supplemental way to predict the catalytic residues of the novel Cas nuclease relative to the previous description in this Example. Catalytic residues are usually highly conserved and can be identified in this manner.

Alternatively, or additionally to the description already provided in this Example, computational software may be used to predict the structure of a Cas nuclease.

What is claimed is:

1. A composition comprising an effector protein and a guide nucleic acid, wherein the effector protein comprises an amino acid sequence that is at least 75%, at least 80%, at least 85%, at least 90%, at least 95%, at least 98%, at least 99% or 100% identical to any one of SEQ ID NOS: 1-10,484 or 15,022-24,165.

2. A composition comprising an effector protein and a guide nucleic acid, wherein the amino acid sequence of the effector protein is at least 75%, at least 80%, at least 85%, at least 90%, at least 95%, at least 98%, at least 99% or 100% identical to any one of SEQ ID NOS: 1-10,484 or 15,022-24,165.

3. A composition comprising an effector protein and a guide nucleic acid, wherein the effector protein comprises about 100, about 120, about 140, about 160, about 180, about 200, about 220, about 240, about 260, about 280, about 300, about 320, about 340, about 360, about 380, about 400, about 420, about 440, about 460, about 480, about 500, about 520, about 540, about 560, about 580, about 600, about 620, about 640, about 660, about 680, about 700, about 720, about 740, about 760, about 780, about 800, about 820, about 840, about 860, about 880, about 900, about 920, about 940, about 960, about 980, about 1000, about 1020, about 1040, about 1060, about 1080, about 1100, about 1120, about 1140, about 1160, about 1180, about 1200, about 1220, about 1240, about 1260, about 1280, about 1300, about 1320, about 1340, about 1360, about 1380, about 1400, about 1420, about 1440, about 1460, about 1480, about 1490, about 1500, about 1520, about 1540, about 1560, about 1580, about 1600, about 1620, about 1640, about 1660, about 1680, about 1700, about 1720, about 1740, about 1760, about 1780, about 1800, about 1820, about 1840, about 1860, about 1880, about 1900, or about 1920 contiguous amino acids of a sequence selected from SEQ ID NOS: 1-10,484 or 15,022-24,165.

4. A composition comprising an effector protein and a guide nucleic acid, wherein the effector protein comprises the amino acid sequence located at positions 1-100, 150-250, 101-200, 250-350, 201-300, 350-450, 301-400, 350-450, 401-500, 450-550, 501-600, 550-650, 601-700, 650-750, 701-800, 750-850, 801-900, 850-950, 901-1000, 950-1050, 1001-1100, 1050-1150, 1101-1200, 1150-1250, 1201-1300, 1250-1350, 1301-1400, 1350-1450, 1401-1500, 1450-1550, 1501-1600, 1550-1650, 1601-1700, 1650-1750, 1701-1800, 1850-1950, 1801-1900, or 1850-1950 of a sequence selected from SEQ ID NOS: 1-10,484 or 15,022-24,165.

5. A composition comprising an effector protein and a guide nucleic acid, wherein the effector protein comprises an amino acid sequence that is at least 90%, at least 95%, or 100% identical to a portion of a sequence selected from SEQ ID NOS: 1-10,484 or 15,022-24,165, and wherein the length of the portion is at least about 100, at least about 200, at least about 300, at least about 400, at least about 500, or at least about 600 linked amino acids in length.

6. The composition of claim 5, wherein the portion of the sequence is about 30%, about 40%, about 50%, about 60%, about 70%, about 80%, or about 90% of a sequence selected from SEQ ID NOS: 1-10,484 or 15,022-24,165.

7. A composition comprising an effector protein, and a guide nucleic acid, wherein
a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A1 of TABLE 1; and
b) at least a portion of the engineered guide nucleic acid comprises a nucleobase sequence that is:
i) at least 50%, at least 55%, at least 60%, at least 65%, at least 70%, at least 75%, at least 80%, at least 85%, at least 90%, at least 95%, or 100% identical to a sequence selected from Column B1 of TABLE 1, wherein the sequence from Column A1 and the sequence from Column B1 are in the same row of TABLE 1, or
ii) at least 50%, at least 55%, at least 60%, at least 65%, at least 70%, at least 75%, at least 80%, at least 85%, at least 90%, at least 95%, or 100% reverse complementary to a sequence selected from Column B1 of TABLE 1, wherein the sequence from Column A1 and the sequence from Column B1 are in the same row of TABLE 1.

8. A composition comprising an effector protein, and an engineered guide nucleic acid, wherein
a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A2 of TABLE 1; and
b) at least a portion of the engineered guide nucleic acid comprises a nucleobase sequence that is:
i) at least 50%, at least 55%, at least 60%, at least 65%, at least 70%, at least 75%, at least 80%, at least 85%, at least 90%, at least 95%, or 100% identical to a sequence selected from Column B2 of TABLE 1, wherein the sequence from Column A2 and the sequence from Column B2 are in the same row of TABLE 1, or
ii) at least 50%, at least 55%, at least 60%, at least 65%, at least 70%, at least 75%, at least 80%, at least 85%, at least 90%, at least 95%, or 100% reverse complementary to a sequence selected from Column B2 of TABLE 1, wherein the sequence from Column A2 and the sequence from Column B2 are in the same row of TABLE 1.

9. A composition comprising an effector protein, and an engineered guide nucleic acid, wherein
a) the amino acid sequence of the effector protein is at least 50%, at least 55%, at least 60%, at least 65%, at least 70%, at least 75%, at least 80%, at least 85%, at least 90%, at least 95%, at least 97%, at least 98%, at least 99% or 100% identical to a sequence selected from Column A3 of TABLE 1; and
b) at least a portion of the engineered guide nucleic acid comprises a nucleobase sequence that is:
i) at least 50%, at least 55%, at least 60%, at least 65%, at least 70%, at least 75%, at least 80%, at least 85%, at least 90%, at least 95%, or 100% identical to a sequence selected from Column B3 of TABLE 1, wherein the sequence from Column A3 and the sequence from Column B3 are in the same row of TABLE 1, or
ii) at least 50%, at least 55%, at least 60%, at least 65%, at least 70%, at least 75%, at least 80%, at least 85%, at least 90%, at least 95%, or 100% reverse complementary to a sequence selected from Column B3 of TABLE 1, wherein the sequence from Column A3 and the sequence from Column B3 are in the same row of TABLE 1.

10. A composition comprising an effector protein, and an engineered guide nucleic acid, wherein the amino acid sequence of the effector protein is at least 50% identical to a sequence selected from Column A1 of TABLE 1, and wherein at least a portion of the engineered guide nucleic acid comprises a nucleobase sequence that is at least 50% identical or at least 50% reverse complementary to a sequence selected from Column B1 of TABLE 1, wherein the sequence from Column A1 and the sequence from Column B1 are in the same row of TABLE 1.

11. A composition comprising an effector protein, and an engineered guide nucleic acid, wherein the amino acid sequence of the effector protein is at least 60% identical to a sequence selected from Column A1 of TABLE 1, and wherein at least a portion of the engineered guide nucleic acid comprises a nucleobase sequence that is at least 60% identical or at least 60% reverse complementary to a sequence selected from Column B1 of TABLE 1, wherein the sequence from Column A1 and the sequence from Column B1 are in the same row of TABLE 1.

12. A composition comprising an effector protein, and an engineered guide nucleic acid, wherein the amino acid sequence of the effector protein is at least 70% identical to a sequence selected from Column A1 of TABLE 1, and wherein at least a portion of the engineered guide nucleic acid comprises a nucleobase sequence that is at least 70% identical or at least 70% reverse complementary to a sequence selected from Column B1 of TABLE 1, wherein the sequence from Column A1 and the sequence from Column B1 are in the same row of TABLE 1.

13. A composition comprising an effector protein, and an engineered guide nucleic acid, wherein the amino acid sequence of the effector protein is at least 80% identical to a sequence selected from Column A1 of TABLE 1, and wherein at least a portion of the engineered guide nucleic acid comprises a nucleobase sequence that is at least 80% identical or at least 80% reverse complementary to a sequence selected from Column B1 of TABLE 1, wherein the sequence from Column A1 and the sequence from Column B1 are in the same row of TABLE 1.

14. A composition comprising an effector protein, and an engineered guide nucleic acid, wherein the amino acid sequence of the effector protein is at least 90% identical to a sequence selected from Column A1 of TABLE 1, and wherein at least a portion of the engineered guide nucleic acid comprises a nucleobase sequence that is at least 90% identical or at least 90% reverse complementary to a sequence selected from Column B1 of TABLE 1, wherein the sequence from Column A1 and the sequence from Column B1 are in the same row of TABLE 1.

15. A composition comprising an effector protein, and an engineered guide nucleic acid, wherein the amino acid sequence of the effector protein is at least 95% identical to a sequence selected from Column A1 of TABLE 1, and wherein at least a portion of the engineered guide nucleic acid comprises a nucleobase sequence that is at least 95% identical or at least 95% reverse complementary to a sequence selected from Column B1 of TABLE 1, wherein the sequence from Column A1 and the sequence from Column B1 are in the same row of TABLE 1.

16. A composition comprising an effector protein, and an engineered guide nucleic acid, wherein the amino acid sequence of the effector protein is 100% identical to a sequence selected from Column A1 of TABLE 1, and wherein at least a portion of the engineered guide nucleic acid comprises a nucleobase sequence that is 100% identical or 100% reverse complementary to a sequence selected from Column B1 of TABLE 1, wherein the sequence from Column A1 and the sequence from Column B1 are in the same row of TABLE 1.

17. A composition comprising an effector protein, and an engineered guide nucleic acid, wherein the amino acid sequence of the effector protein is at least 50% identical to a sequence selected from Column A2 of TABLE 1, and wherein at least a portion of the engineered guide nucleic acid comprises a nucleobase sequence that is at least 50% identical or at least 50% reverse complementary to a sequence selected from Column B2 of TABLE 1, wherein the sequence from Column A2 and the sequence from Column B2 are in the same row of TABLE 1.

18. A composition comprising an effector protein, and an engineered guide nucleic acid, wherein the amino acid sequence of the effector protein is at least 60% identical to a sequence selected from Column A2 of TABLE 1, and wherein at least a portion of the engineered guide nucleic acid comprises a nucleobase sequence that is at least 60% identical or at least 60% reverse complementary to a sequence selected from Column B2 of TABLE 1, wherein the sequence from Column A2 and the sequence from Column B2 are in the same row of TABLE 1.

19. A composition comprising an effector protein, and an engineered guide nucleic acid, wherein the amino acid sequence of the effector protein is at least 70% identical to a sequence selected from Column A2 of TABLE 1, and wherein at least a portion of the engineered guide nucleic acid comprises a nucleobase sequence that is at least 70% identical or at least 70% reverse complementary to a sequence selected from Column B2 of TABLE 1, wherein the sequence from Column A2 and the sequence from Column B2 are in the same row of TABLE 1.

20. A composition comprising an effector protein, and an engineered guide nucleic acid, wherein the amino acid sequence of the effector protein is at least 80% identical to a sequence selected from Column A2 of TABLE 1, and wherein at least a portion of the engineered guide nucleic acid comprises a nucleobase sequence that is at least 80% identical or at least 80% reverse complementary to a sequence selected from Column B2 of TABLE 1, wherein the sequence from Column A2 and the sequence from Column B2 are in the same row of TABLE 1.

21. A composition comprising an effector protein, and an engineered guide nucleic acid, wherein the amino acid sequence of the effector protein is at least 90% identical to a sequence selected from Column A2 of TABLE 1, and wherein at least a portion of the engineered guide nucleic acid comprises a nucleobase sequence that is at least 90% identical or at least 90% reverse complementary to a sequence selected from Column B2 of TABLE 1, wherein the sequence from Column A2 and the sequence from Column B2 are in the same row of TABLE 1.

22. A composition comprising an effector protein, and an engineered guide nucleic acid, wherein the amino acid sequence of the effector protein is at least 95% identical to a sequence selected from Column A2 of TABLE 1, and wherein at least a portion of the engineered guide nucleic acid comprises a nucleobase sequence that is at least 95% identical or at least 95% reverse complementary to a sequence selected from Column B2 of TABLE 1, wherein the sequence from Column A2 and the sequence from Column B2 are in the same row of TABLE 1.

23. A composition comprising an effector protein, and an engineered guide nucleic acid, wherein the amino acid sequence of the effector protein is 100% identical to a sequence selected from Column A2 of TABLE 1, and wherein at least a portion of the engineered guide nucleic acid comprises a nucleobase sequence that is 100% identical or 100% reverse complementary to a sequence selected from Column B2 of TABLE 1, wherein the sequence from Column A2 and the sequence from Column B2 are in the same row of TABLE 1.

24. A composition comprising an effector protein, and an engineered guide nucleic acid, wherein the amino acid sequence of the effector protein is at least 50% identical to a sequence selected from Column A3 of TABLE 1, and wherein at least a portion of the engineered guide nucleic acid comprises a nucleobase sequence that is at least 50% identical or at least 50% reverse complementary to a sequence selected from Column B3 of TABLE 1, wherein the sequence from Column A3 and the sequence from Column B3 are in the same row of TABLE 1.

25. A composition comprising an effector protein, and an engineered guide nucleic acid, wherein the amino acid sequence of the effector protein is at least 60% identical to a sequence selected from Column A3 of TABLE 1, and wherein at least a portion of the engineered guide nucleic acid comprises a nucleobase sequence that is at least 60% identical or at least 60% reverse complementary to a sequence selected from Column B3 of TABLE 1, wherein the sequence from Column A3 and the sequence from Column B3 are in the same row of TABLE 1.

26. A composition comprising an effector protein, and an engineered guide nucleic acid, wherein the amino acid sequence of the effector protein is at least 70% identical to a sequence selected from Column A3 of TABLE 1, and wherein at least a portion of the engineered guide nucleic acid comprises a nucleobase sequence that is at least 70% identical or at least 70% reverse complementary to a sequence selected from Column B3 of TABLE 1, wherein the sequence from Column A3 and the sequence from Column B3 are in the same row of TABLE 1.

27. A composition comprising an effector protein, and an engineered guide nucleic acid, wherein the amino acid sequence of the effector protein is at least 80% identical to a sequence selected from Column A3 of TABLE 1, and wherein at least a portion of the engineered guide nucleic acid comprises a nucleobase sequence that is at least 80% identical or at least 80% reverse complementary to a sequence selected from Column B3 of TABLE 1, wherein the sequence from Column A3 and the sequence from Column B3 are in the same row of TABLE 1.

28. A composition comprising an effector protein, and an engineered guide nucleic acid, wherein the amino acid sequence of the effector protein is at least 90% identical to a sequence selected from Column A3 of TABLE 1, and wherein at least a portion of the engineered guide nucleic acid comprises a nucleobase sequence that is at least 90% identical or at least 90% reverse complementary to a sequence selected from Column B3 of TABLE 1, wherein the sequence from Column A3 and the sequence from Column B3 are in the same row of TABLE 1.

29. A composition comprising an effector protein, and an engineered guide nucleic acid, wherein the amino acid sequence of the effector protein is at least 95% identical to a sequence selected from Column A3 of TABLE 1, and wherein at least a portion of the engineered guide nucleic acid comprises a nucleobase sequence that is at least 95% identical or at least 95% reverse complementary to a sequence selected from Column B3 of TABLE 1, wherein the sequence from Column A3 and the sequence from Column B3 are in the same row of TABLE 1.

30. A composition comprising an effector protein, and an engineered guide nucleic acid, wherein the amino acid sequence of the effector protein is 100% identical to a sequence selected from Column A3 of TABLE 1, and wherein at least a portion of the engineered guide nucleic acid comprises a nucleobase sequence that is 100% identical or 100% reverse complementary to a sequence selected from Column B3 of TABLE 1, wherein the sequence from Column A3 and the sequence from Column B3 are in the same row of TABLE 1.

31. A composition comprising an effector protein, and an engineered guide nucleic acid, wherein the amino acid sequence of the effector protein is at least 50% identical to a sequence selected from Column D1 of TABLE 2, and wherein at least a portion of the engineered guide nucleic acid comprises a nucleobase sequence that is at least 50% identical or at least 50% reverse complementary to a sequence selected from Column C1 of TABLE 2, wherein the sequence from Column D1 and the sequence from Column C1 are in the same row of TABLE 2.

32. A composition comprising an effector protein, and an engineered guide nucleic acid, wherein the amino acid sequence of the effector protein is at least 60% identical to a sequence selected from Column D1 of TABLE 2, and wherein at least a portion of the engineered guide nucleic acid comprises a nucleobase sequence that is at least 60% identical or at least 60% reverse complementary to a sequence selected from Column C1 of TABLE 2, wherein the sequence from Column D1 and the sequence from Column C1 are in the same row of TABLE 2.

33. A composition comprising an effector protein, and an engineered guide nucleic acid, wherein the amino acid sequence of the effector protein is at least 70% identical to a sequence selected from Column D1 of TABLE 2, and wherein at least a portion of the engineered guide nucleic acid comprises a nucleobase sequence that is at least 70% identical or at least 70% reverse complementary to a sequence selected from Column C1 of TABLE 2, wherein the sequence from Column D1 and the sequence from Column C1 are in the same row of TABLE 2.

34. A composition comprising an effector protein, and an engineered guide nucleic acid, wherein the amino acid sequence of the effector protein is at least 80% identical to a sequence selected from Column D1 of TABLE 2, and wherein at least a portion of the engineered guide nucleic acid comprises a nucleobase sequence that is at least 80% identical or at least 80% reverse complementary to a sequence selected from Column C1 of TABLE 2, wherein the sequence from Column D1 and the sequence from Column C1 are in the same row of TABLE 2.

35. A composition comprising an effector protein, and an engineered guide nucleic acid, wherein the amino acid sequence of the effector protein is at least 90% identical to a sequence selected from Column D1 of TABLE 2, and wherein at least a portion of the engineered guide nucleic acid comprises a nucleobase sequence that is at least 90% identical or at least 90% reverse complementary to a sequence selected from Column C1 of TABLE 2, wherein the sequence from Column D1 and the sequence from Column C1 are in the same row of TABLE 2.

36. A composition comprising an effector protein, and an engineered guide nucleic acid, wherein the amino acid sequence of the effector protein is at least 95% identical to a sequence selected from Column D1 of TABLE 2, and wherein at least a portion of the engineered guide nucleic acid comprises a nucleobase sequence that is at least 95% identical or at least 95% reverse complementary to a sequence selected from Column C1 of TABLE 2, wherein the sequence from Column D1 and the sequence from Column C1 are in the same row of TABLE 2.

37. A composition comprising an effector protein, and an engineered guide nucleic acid, wherein the amino acid sequence of the effector protein is 100% identical to a sequence selected from Column D1 of TABLE 2, and wherein at least a portion of the engineered guide nucleic acid comprises a nucleobase sequence that is 100% identical or 100% reverse complementary to a sequence selected from Column C1 of TABLE 2, wherein the sequence from Column D1 and the sequence from Column C1 are in the same row of TABLE 2.

38. A composition comprising an effector protein, and an engineered guide nucleic acid, wherein the amino acid sequence of the effector protein is at least 50% identical to a sequence selected from Column D2 of TABLE 2, and wherein at least a portion of the engineered guide nucleic acid comprises a nucleobase sequence that is at least 50% identical or at least 50% reverse complementary to a sequence selected from Column C2 of TABLE 2, wherein the sequence from Column D2 and the sequence from Column C2 are in the same row of TABLE 2.

39. A composition comprising an effector protein, and an engineered guide nucleic acid, wherein the amino acid sequence of the effector protein is at least 60% identical to a sequence selected from Column D2 of TABLE 2, and wherein at least a portion of the engineered guide nucleic acid comprises a nucleobase sequence that is at least 60% identical or at least 60% reverse complementary to a sequence selected from Column C2 of TABLE 2, wherein the sequence from Column D2 and the sequence from Column C2 are in the same row of TABLE 2.

40. A composition comprising an effector protein, and an engineered guide nucleic acid, wherein the amino acid sequence of the effector protein is at least 70% identical to a sequence selected from Column D2 of TABLE 2, and wherein at least a portion of the engineered guide nucleic acid comprises a nucleobase sequence that is at least 70% identical or at least 70% reverse complementary to a sequence selected from Column C2 of TABLE 2, wherein the sequence from Column D2 and the sequence from Column C2 are in the same row of TABLE 2.

41. A composition comprising an effector protein, and an engineered guide nucleic acid, wherein the amino acid sequence of the effector protein is at least 80% identical to a sequence selected from Column D2 of TABLE 2, and wherein at least a portion of the engineered guide nucleic acid comprises a nucleobase sequence that is at least 80% identical or at least 80% reverse complementary to a sequence selected from Column C2 of TABLE 2, wherein the sequence from Column D2 and the sequence from Column C2 are in the same row of TABLE 2.

42. A composition comprising an effector protein, and an engineered guide nucleic acid, wherein the amino acid sequence of the effector protein is at least 90% identical to a sequence selected from Column D2 of TABLE 2, and wherein at least a portion of the engineered guide nucleic acid comprises a nucleobase sequence that is at least 90% identical or at least 90% reverse complementary to a sequence selected from Column C2 of TABLE 2, wherein the sequence from Column D2 and the sequence from Column C2 are in the same row of TABLE 2.

43. A composition comprising an effector protein, and an engineered guide nucleic acid, wherein the amino acid sequence of the effector protein is at least 95% identical to a sequence selected from Column D2 of TABLE 2, and wherein at least a portion of the engineered guide nucleic acid comprises a nucleobase sequence that is at least 95% identical or at least 95% reverse complementary to a sequence selected from Column C2 of TABLE 2, wherein the sequence from Column D2 and the sequence from Column C2 are in the same row of TABLE 2.

44. A composition comprising an effector protein, and an engineered guide nucleic acid, wherein the amino acid sequence of the effector protein is 100% identical to a sequence selected from Column D2 of TABLE 2, and wherein at least a portion of the engineered guide nucleic acid comprises a nucleobase sequence that is 100% identical or 100% reverse complementary to a sequence selected from Column C2 of TABLE 2, wherein the sequence from Column D2 and the sequence from Column C2 are in the same row of TABLE 2.

45. A composition comprising an effector protein, and an engineered guide nucleic acid, wherein the amino acid sequence of the effector protein is at least 50% identical to a sequence selected from Column D3 of TABLE 2, and wherein at least a portion of the engineered guide nucleic acid comprises a nucleobase sequence that is at least 50% identical or at least 50% reverse complementary to a sequence selected from Column C3 of TABLE 2, wherein the sequence from Column D3 and the sequence from Column C3 are in the same row of TABLE 2.

46. A composition comprising an effector protein, and an engineered guide nucleic acid, wherein the amino acid sequence of the effector protein is at least 60% identical to a sequence selected from Column D3 of TABLE 2, and wherein at least a portion of the engineered guide nucleic acid comprises a nucleobase sequence that is at least 60% identical or at least 60% reverse complementary to a sequence selected from Column C3 of TABLE 2, wherein the sequence from Column D3 and the sequence from Column C3 are in the same row of TABLE 2.

47. A composition comprising an effector protein, and an engineered guide nucleic acid, wherein the amino acid sequence of the effector protein is at least 70% identical to a sequence selected from Column D3 of TABLE 2, and wherein at least a portion of the engineered guide nucleic acid comprises a nucleobase sequence that is at least 70% identical or at least 70% reverse complementary to a sequence selected from Column C3 of TABLE 2, wherein the sequence from Column D3 and the sequence from Column C3 are in the same row of TABLE 2.

48. A composition comprising an effector protein, and an engineered guide nucleic acid, wherein the amino acid sequence of the effector protein is at least 80% identical to a sequence selected from Column D3 of TABLE 2, and wherein at least a portion of the engineered guide nucleic acid comprises a nucleobase sequence that is at least 80% identical or at least 80% reverse complementary to a sequence selected from Column C3 of TABLE 2, wherein the sequence from Column D3 and the sequence from Column C3 are in the same row of TABLE 2.

49. A composition comprising an effector protein, and an engineered guide nucleic acid, wherein the amino acid sequence of the effector protein is at least 90% identical to a sequence selected from Column D3 of TABLE 2, and wherein at least a portion of the engineered guide nucleic acid comprises a nucleobase sequence that is at least 90% identical or at least 90% reverse complementary to a sequence selected from Column C3 of TABLE 2, wherein the sequence from Column D3 and the sequence from Column C3 are in the same row of TABLE 2.

50. A composition comprising an effector protein, and an engineered guide nucleic acid, wherein the amino acid sequence of the effector protein is at least 95% identical to a sequence selected from Column D3 of TABLE 2, and wherein at least a portion of the engineered guide nucleic acid comprises a nucleobase sequence that is at least 95% identical or at least 95% reverse complementary to a sequence selected from Column C3 of TABLE 2, wherein the sequence from Column D3 and the sequence from Column C3 are in the same row of TABLE 2.

51. A composition comprising an effector protein, and an engineered guide nucleic acid, wherein the amino acid sequence of the effector protein is 100% identical to a sequence selected from Column D3 of TABLE 2, and wherein at least a portion of the engineered guide nucleic acid comprises a nucleobase sequence that is 100% identical or 100% reverse complementary to a sequence selected from Column C3 of TABLE 2, wherein the sequence from Column D3 and the sequence from Column C3 are in the same row of TABLE 2.

52. A composition comprising an effector protein, and an engineered guide nucleic acid, wherein the amino acid sequence of the effector protein is at least 50% identical to a sequence selected from Column D4 of TABLE 2, and wherein at least a portion of the engineered guide nucleic acid comprises a nucleobase sequence that is at least 50% identical or at least 50% reverse complementary to a sequence selected from Column C4 of TABLE 2, wherein the sequence from Column D4 and the sequence from Column C4 are in the same row of TABLE 2.

53. A composition comprising an effector protein, and an engineered guide nucleic acid, wherein the amino acid sequence of the effector protein is at least 60% identical to a sequence selected from Column D4 of TABLE 2, and wherein at least a portion of the engineered guide nucleic acid comprises a nucleobase sequence that is at least 60% identical or at least 60% reverse complementary to a sequence selected from Column C4 of TABLE 2, wherein the sequence from Column D4 and the sequence from Column C4 are in the same row of TABLE 2.

54. A composition comprising an effector protein, and an engineered guide nucleic acid, wherein the amino acid sequence of the effector protein is at least 70% identical to a sequence selected from Column D4 of TABLE 2, and wherein at least a portion of the engineered guide nucleic acid comprises a nucleobase sequence that is at least 70% identical or at least 70% reverse complementary to a sequence selected from Column C4 of TABLE 2, wherein the sequence from Column D4 and the sequence from Column C4 are in the same row of TABLE 2.

55. A composition comprising an effector protein, and an engineered guide nucleic acid, wherein the amino acid sequence of the effector protein is at least 80% identical to a sequence selected from Column D4 of TABLE 2, and wherein at least a portion of the engineered guide nucleic acid comprises a nucleobase sequence that is at least 80% identical or at least 80% reverse complementary to a sequence selected from Column C4 of TABLE 2, wherein the sequence from Column D4 and the sequence from Column C4 are in the same row of TABLE 2.

56. A composition comprising an effector protein, and an engineered guide nucleic acid, wherein the amino acid sequence of the effector protein is at least 90% identical to a sequence selected from Column D4 of TABLE 2, and wherein at least a portion of the engineered guide nucleic acid comprises a nucleobase sequence that is at least 90% identical or at least 90% reverse complementary to a sequence selected from Column C4 of TABLE 2, wherein the sequence from Column D4 and the sequence from Column C4 are in the same row of TABLE 2.

57. A composition comprising an effector protein, and an engineered guide nucleic acid, wherein the amino acid sequence of the effector protein is at least 95% identical to a sequence selected from Column D4 of TABLE 2, and wherein at least a portion of the engineered guide nucleic acid comprises a nucleobase sequence that is at least 95% identical or at least 95% reverse complementary to a sequence selected from Column C4 of TABLE 2, wherein the sequence from Column D4 and the sequence from Column C4 are in the same row of TABLE 2.

58. A composition comprising an effector protein, and an engineered guide nucleic acid, wherein the amino acid sequence of the effector protein is 100% identical to a sequence selected from Column D4 of TABLE 2, and wherein at least a portion of the engineered guide nucleic acid comprises a nucleobase sequence that is 100% identical or 100% reverse complementary to a sequence selected from Column C4 of TABLE 2, wherein the sequence from Column D4 and the sequence from Column C4 are in the same row of TABLE 2.

59. The composition of any one of claims 1-58, wherein at least a portion of the guide nucleic acid binds the effector protein.

60. The composition of any one of claims 1-59, wherein the guide nucleic acid comprises a crRNA.

61. The composition of any one of claims 1-60, wherein the guide nucleic acid comprises a tracrRNA.

62. The composition of any one of claims 1-60, wherein the composition does not comprise a tracrRNA.

63. The composition of any one of claims 1-61, wherein the guide nucleic acid comprises a crRNA covalently linked to a tracrRNA.

64. The composition of any one of claims 1-63, wherein the guide nucleic acid comprises a first sequence and a second sequence, wherein the first sequence is heterologous with the second sequence.

65. The composition of claim 64, wherein the first sequence comprises at least five amino acids and the second sequence comprises at least five amino acids.

66. The composition of any one of claims 1-65, wherein at least one of the effector protein, the guide nucleic acid, and the combination thereof, are not naturally occurring.

67. The composition of any one of claims 1-66, wherein at least one of the effector protein and the guide nucleic acid is recombinant or engineered.

68. The composition of any one of claims 1-67, wherein the guide nucleic acid comprises a nucleotide sequence that is at least 80%, at least 85%, at least 90%, at least 95%, at least 98% or 100% identical to a nucleotide sequence selected from SEQ ID NOS: 10,485-15,015 or 24,166-31,319.

69. The composition of any one of claims 1-68, wherein the guide nucleic acid comprises at least 8, at least 9, at least 10, at least 11, at least 12, at least 13, at least 14, at least 15, at least 16, at least 17, at least 18, at least 19, or at least 20 contiguous nucleotides of a nucleotide sequence selected from SEQ ID NOS: 10,485-15,015 or 24,166-31,319.

70. The composition of any one of claims 1-69, wherein the guide nucleic acid comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, or at least 220 contiguous nucleotides of a nucleotide sequence selected from SEQ ID NOS: 10,485-15,015 or 24,166-33,309.

71. The composition of any one of claims 1-70, wherein the guide nucleic acid comprises a sequence that hybridizes to a target sequence of a target nucleic acid, and wherein the target nucleic acid comprises a protospacer adjacent motif (PAM).

72. The composition of claim 71, wherein the PAM is located within 1, 5, 10, 15, 20, 40, 60, 80 or 100 nucleotides of the 5′ end of the target sequence.

73. The composition of any one of claims 1-72, wherein the effector protein comprises a nuclear localization signal.

74. The composition of any one of claims 1-73, comprising a donor nucleic acid.

75. The composition of any one of claims 1-74, comprising a fusion partner protein linked to the effector protein.

76. The composition of claim 75, wherein the fusion partner protein is directly fused to the N terminus or C terminus of the effector protein via an amide bond.

77. The composition of claim 75, wherein the fusion partner protein is directly fused to the N terminus or C terminus of the effector protein via a peptide linker.

78. The composition of any one of claims 75-77, wherein the fusion partner protein comprises a polypeptide selected from a deaminase, a transcriptional activator, a transcriptional repressor, or a functional domain thereof.

79. The composition of any one of claims 1-78, wherein the effector protein comprises at least one mutation that reduces its nuclease activity relative to the effector protein without the mutation as measured in a cleavage assay, optionally wherein the effector protein is a catalytically inactive nuclease.

80. A composition comprising a nucleic acid expression vector, wherein the nucleic acid vector encodes at least one of the effector protein and the guide nucleic acid of the composition of any one of claims 1-79.

81. The composition of claim 80, comprising a donor nucleic acid, optionally wherein the donor nucleic acid is encoded by the nucleic acid expression vector or an additional nucleic acid expression vector.

82. The composition of claim 80 or 81, wherein the nucleic acid expression vector is a viral vector.

83. The composition of claim 82, wherein the viral vector is an adeno associated viral (AAV) vector.

84. A composition comprising a virus, wherein the virus comprises the composition of any one of claims 80-83.

85. A pharmaceutical composition, comprising the composition of any one of claims 1-84, and a pharmaceutically acceptable excipient.

86. A system comprising the composition of any one of claims 1-84, and at least one detection reagent for detecting a target nucleic acid.

87. The system of claim 86, wherein the at least one detection reagent is selected from a reporter nucleic acid, a detection moiety, an additional effector protein, or a combination thereof, optionally wherein the reporter nucleic acid comprises a fluorophore, a quencher, or a combination thereof.

88. The system of claim 86 or 87, comprising at least one amplification reagent for amplifying a target nucleic acid.

89. The system of claim 88, wherein the at least one amplification reagent is selected from the group consisting of a primer, a polymerase, a deoxynucleoside triphosphate (dNTP), a ribonucleoside triphosphate (rNTP), and combinations thereof.

90. The system of any one of claims 86-89, wherein the system comprises a device with a chamber or solid support for containing the composition, target nucleic acid, detection reagent or combination thereof.

91. A method of detecting a target nucleic acid in a sample, comprising the steps of:
(a) contacting the sample with:
(i) the composition of any one of claims 1-84 or the system of any one of claims 86-89; and
(ii) a reporter nucleic acid comprising a detectable moiety that produces a detectable signal in the presence of the target nucleic acid and the composition or system, and
(b) detecting the detectable signal.

92. The method of claim 91, wherein the reporter nucleic acid comprises a fluorophore, a quencher, or a combination thereof, and wherein the detecting comprises detecting a fluorescent signal.

93. The method of claim 91 or 92, comprising reverse transcribing the target nucleic acid, amplifying the target nucleic acid, in vitro transcribing the target nucleic acid, or any combination thereof.

94. The method of any one of claims 91-93, comprising reverse transcribing the target nucleic acid and/or amplifying the target nucleic acid before contacting the sample with the composition.

95. The method of any one of claims 91-93, comprising reverse transcribing the target nucleic acid and/or amplifying the target nucleic acid after contacting the sample with the composition.

96. The method of any one of claims 93-95, wherein amplifying comprises isothermal amplification.

97. The method of any one of claims 91-96, wherein the target nucleic acid is from a pathogen.

98. The method of claim 97, wherein the pathogen is a virus.

99. The method of any one of claims 91-98, wherein the target nucleic acid comprises RNA.

100. The method of any one of claims 91-99, wherein the target nucleic acid comprises DNA.

101. A method of modifying a target nucleic acid, the method comprising contacting the target nucleic acid with the composition of any one of claims 1-84, or the system of any one of claims 86-90, thereby modifying the target nucleic acid.

102. The method of claim 101, wherein modifying the target nucleic acid comprises cleaving the target nucleic acid, deleting a nucleotide of the target nucleic acid, inserting a nucleotide into the target nucleic acid, substituting a nucleotide of the target nucleic acid with an alternative nucleotide or an additional nucleotide, or any combination thereof.

103. The method of claim 101 or 102, comprising contacting the target nucleic acid with a donor nucleic acid.

104. The method of any one of claims 101-103, wherein the target nucleic acid comprises a mutation associated with a disease.

105. The method of claim 104, wherein the disease is selected from an autoimmune disease, a cancer, an inherited disorder, an ophthalmological disorder, a metabolic disorder, or a combination thereof.

106. The method of claim 104, wherein the disease is cystic fibrosis, thalassemia, Duchenne muscular dystrophy, myotonic dystrophy Type 1, or sickle cell anemia.

107. The method of any one of claims 101-106, wherein contacting the target nucleic acid comprises contacting a cell, wherein the target nucleic acid is located in the cell.

108. The method of claim 107, wherein the contacting occurs in vitro.

109. The method of claim 107, wherein the contacting occurs in vivo.

110. The method of claim 107, wherein the contacting occurs ex vivo.

111. A cell comprising the composition of any one of claims 1-84.

112. A cell modified by the composition of any one of claims 1-84.

113. A cell modified by the system of any one of claims 86-90.

114. A cell comprising a modified target nucleic acid, wherein the modified target nucleic acid is a target nucleic acid modified according to any one of the methods of claims 101-110.

115. The cell of any one of claims 111-114, wherein the cell is a eukaryotic cell.

116. The cell of any one of claims 111-114, wherein the cell is a mammalian cell.

117. The cell of any one of claims 111-114, wherein the cell is a prokaryotic cell.

118. The cell of any one of claims 111-114, wherein the cell is a plant cell.

119. The cell of any one of claims 111-114, wherein the cell is an animal cell.

120. The cell of claim 119, wherein the cell is a T cell, optionally wherein the T cell is a natural killer T cell (NKT).

121. The cell of claim 115, wherein the cell is a chimeric antigen receptor T cell (CAR T-cell).

122. The cell of claim 115, wherein the cell is an induced pluripotent stem cell (iPSC).

123. A population of cells according to any one of claims 111-122.

124. A method of producing a protein, the method comprising,
(i) contacting a cell comprising a target nucleic acid with the composition of any one of claims 1-84, thereby editing the target nucleic acid to produce a modified cell comprising a modified target nucleic acid; and
(ii) producing a protein from the cell that is encoded, transcriptionally affected, or translationally affected by the modified nucleic acid.

125. A method of treating a disease comprising administering to a subject in need thereof a composition according to any one of claims 1-84, or a cell according to any one of claims 111-122.
